(19) United States
(12) Patent Application Publication
(10) Pub. No.: US 2026/0013913 A1
(43) Pub. Date: Jan. 15, 2026

(54) FRACTURE PLATING SYSTEMS AND METHODS

(71) Applicant: Costa Surgical Inc., Chandler, AZ (US)

(72) Inventors: Jeffery D. ARNETT, Louisa, VA (US); Nicholas SLATER, Chandler, AZ (US); Lane M. JOHNSON, Gilbert, AZ (US); Joseph D. FORRESTER, Redwood City, CA (US); David J. WEATHERBY, Lake Elmo, MN (US)

(21) Appl. No.: 19/327,661

(22) Filed: Sep. 12, 2025

Related U.S. Application Data
(63) Continuation-in-part of application No. 19/067,944, filed on Mar. 2, 2025.
(60) Provisional application No. 63/560,219, filed on Mar. 1, 2024.
(60) Provisional application No. 63/572,491, filed on Apr. 1, 2024.
(60) Provisional application No. 63/693,769, filed on Sep. 12, 2024.
(60) Provisional application No. 63/748,317, filed on Jan. 22, 2025.
(60) Provisional application No. 63/828,556, filed on Jun. 23, 2025.

(51) Int. Cl. A61B 17/80; A61B 17/56; A61B 17/86; A61B 90/00

(52) U.S. Cl. CPC A61B 17/80; A61B 17/8605; A61B 17/863; A61B 90/03; A61B 2017/564; A61B 2090/035

(57) ABSTRACT

A fracture plating system may be configured to stabilize a fracture of a bone. The bone may include an interior surface facing toward an interior body cavity of the patient, and an exterior surface facing away from the interior body cavity. The fracture plating system may include a plate formed of a biocompatible polymer and configured to span the fracture. The plate may include an exterior plate surface configured to abut the interior surface, an interior plate surface opposite the exterior plate surface, and a slot passing between the exterior plate surface and the interior plate surface. The fracture plating system may further include a tether configured to apply a force to the plate to draw the exterior plate surface against the interior surface of the bone, and a bracing member securable to the plate to restrict flexure of the plate parallel to a width of the slot.

### CROSS-REFERENCE TO RELATED APPLICATIONS

The present application claims the benefit of U.S. Provisional Patent Application Ser. No. 63/693,769 filed on Sep. 12, 2024, entitled INTERNAL RIB PLATING SYSTEMS AND METHODS.

The present application also claims the benefit of U.S. Provisional Patent Application Ser. No. 63/748,317 filed on Jan. 22, 2025, entitled RIB FIXATION SYSTEMS AND METHODS.

The present application also claims the benefit of U.S. Provisional Patent Application Ser. No. 63/828,556 filed on Jun. 23, 2025, entitled FRACTURE PLATING SYSTEMS AND METHODS.

The present application is also a continuation-in-part of U.S. patent application Ser. No. 19/067,944, filed on Mar. 2, 2025 and entitled FRACTURE PLATING SYSTEMS AND METHODS, which claims the benefit of U.S. Provisional Application Ser. No. 63/560,219 filed on Mar. 1, 2024, entitled FRACTURE PLATING SYSTEMS AND METHODS, and U.S. Provisional Application Ser. No. 63/572,491 filed on Apr. 1, 2024, entitled FRACTURE PLATING SYSTEMS AND METHODS.

The foregoing are hereby incorporated by reference as though set forth herein in their entirety.

### TECHNICAL FIELD

The present disclosure relates generally to surgical systems and methods, and more particularly, to systems and methods for fracture plating and stabilization.

### BACKGROUND

Bone fractures, particularly in anatomical regions such as the ribs, can present significant challenges in treatment due to their anatomical complexity, constant motion during respiration, and the difficulty of achieving stable fixation. Rib fractures, flail chest, and other thoracic injuries often result from trauma and can lead to severe pain, respiratory complications, and impaired pulmonary function. Proper stabilization of fractured rib segments is crucial to facilitate healing, reduce pain, and restore respiratory mechanics.

Conventional methods of treating rib fractures often involve conservative management, which may include pain control and respiratory therapy. However, in cases of severe fractures, such as flail chest or displaced rib fractures, surgical intervention may be necessary. Traditional open surgical approaches for rib fixation typically require large incisions, displacement of muscle tissue, and exposure of the fracture site, which can result in increased morbidity, prolonged recovery times, and additional complications such as infection and tissue damage.

Percutaneous fixation techniques have emerged as a less invasive alternative, offering potential advantages such as reduced surgical trauma, shorter recovery times, and lower complication rates. However, existing percutaneous bone fixation systems are often limited in their ability to securely stabilize bone segments, particularly in regions like the ribs where anatomical curvature and constant mechanical forces present additional challenges. Many current devices and methods lack the necessary adaptability and stability to accommodate complex fracture patterns and ensure proper alignment and fixation of bone segments. There is a need for an improved bone repair system designed specifically for percutaneous fixation of bone segments, such as rib bones.

### SUMMARY

The various systems and methods of the present disclosure have been developed in response to the present state of the art, and in particular, in response to the problems and needs in the art that have not yet been fully solved by currently available fracture plating systems and methods.

In some embodiments, a fracture plating system may be configured to stabilize a first fracture of a bone of a patient. The bone may have an interior surface facing toward an interior body cavity of the patient, and an exterior surface facing away from the interior body cavity. The fracture plating system may include a plate formed of a biocompatible polymer and configured to span the first fracture. The plate may include an exterior plate surface configured to abut the interior surface, an interior plate surface opposite the exterior plate surface, and a slot passing between the exterior plate surface and the interior plate surface. The fracture plating system may further include a tether configured to apply a force to the plate to draw the exterior plate surface against the interior surface of the bone, and a bracing member securable to the plate to restrict flexure of the plate parallel to a width of the slot.

In the fracture plating system of any preceding paragraph, the bracing member may be incorporated into a first fastener including a proximal end with a head portion. The bracing member may include a first wing extending from the head portion. The first fastener may further include a distal end. The distal end may be insertable through the slot to secure the plate to the interior surface such that the first wing abuts the interior plate surface.

In the fracture plating system of any preceding paragraph, the bracing member may further include a second wing extending from the head portion. The first wing and the second wing may cooperate to extend along substantially an entire width of the of the interior plate surface, transverse to the slot.

In the fracture plating system of any preceding paragraph, each of the first wing and the second wing may include a tip that extends along a longitudinal axis of the first fastener such that, with the first wing and the second wing abutting the interior plate surface, the tips extend alongside the plate such that the plate is positioned between the tips to prevent flexure of the plate in a manner that would cause the slot to expand.

In some embodiments, a fracture plating system may be configured to stabilize a first fracture of a bone of a patient. The bone may have an interior surface facing toward an interior body cavity of the patient, and an exterior surface facing away from the interior body cavity. The fracture plating system may include a plate configured to span the first fracture. The plate may have an exterior plate surface configured to abut the interior surface, an interior plate surface opposite the exterior plate surface, and a slot passing between the exterior plate surface and the interior plate surface. The fracture plating system may further include a first fastener with a proximal end with a head portion, a distal end, and a shank that extends between the head portion and the distal end. The fracture plating system may further include a tether configured to apply a force to guide the first fastener and the plate through the interior body cavity to the interior surface of the bone. The slot may have an expanded configuration in which the distal end is insertable through the slot, and an unexpanded configuration in which the shank is accommodated within the slot but the distal end is not withdrawable through the slot.

In the fracture plating system of any preceding paragraph, the plate may be configured to flex such that the slot is deformable along a direction transverse to the slot, from the unexpanded configuration to the expanded configuration.

In the fracture plating system of any preceding paragraph, the fracture plating system may further include an expansion tool configured to engage the plate to deform the slot from the unexpanded configuration to the expanded configuration.

In the fracture plating system of any preceding paragraph, the expansion tool may include a cam surface with a cam width smaller than a slot width of the slot, and a cam length longer than the slot width such that rotation of the cam surface within the slot urges deformation of the slot from the unexpanded configuration to the expanded configuration.

In the fracture plating system of any preceding paragraph, the distal end may be threaded, and the shank may have a smallest shank width smaller than a distal end width of the distal end.

In the fracture plating system of any preceding paragraph, the shank may have threads, and unthreaded flats that interrupt the threads and define the smallest shank width such that, in the unexpanded configuration. The unthreaded flats may abut interior walls of the slot to prevent rotation of the shank within the slot.

According to some embodiments, a method may be used to stabilize a first fracture of a bone of a patient. The bone may have an interior surface facing toward an interior body cavity of the patient, and an exterior surface facing away from the interior body cavity. The method may include positioning a plate near the bone, wherein the plate is formed of a biocompatible polymer and is configured to span the first fracture. The plate may have an exterior plate surface, an interior plate surface opposite the exterior plate surface, and a slot passing between the exterior plate surface and the interior plate surface. The method may further include urging the exterior plate surface against the interior surface of the bone such that the plate flexes to cause the exterior plate surface to conform to the interior surface, and with at least a first fastener, securing the plate to the bone.

In the method of any preceding paragraph, the method may further include passing a first end of a tether through the bone. Urging the exterior plate surface against the interior surface of the bone may include using the tether to pull exterior plate surface against the interior surface of the bone.

In the method of any preceding paragraph, the first fastener may have a proximal end with a head portion, and wings extending from the head portion. The fastener may further have a distal end. Securing the plate to the bone may include inserting the distal end through the slot, and further advancing the first fastener toward the bone such that the wings abut the plate interior surface.

In the method of any preceding paragraph, each of the wings may include a tip that extends along a longitudinal axis of the first fastener such that, with the wings abutting the interior plate surface, the tips extend alongside the plate such that the plate is positioned between the tips to prevent flexure of the plate in a manner that would cause the slot to expand.

In the method of any preceding paragraph, the interior surface of the bone may include a bone curvature, and the exterior plate surface may have one of a planar shape, and a plate curvature with a larger radius than the bone curvature.

In the method of any preceding paragraph, the plate may further have a first end, a second end opposite the first end, and a central portion that connects the first end and the second end. Urging the exterior plate surface against the interior surface of the bone may include contacting the interior surface of the bone with the first end and the second end, and bending the plate to cause the central portion to come into contact with the interior surface.

According to some embodiments, a method may be used to assemble a system for stabilizing a first fracture of a bone of a patient. The bone may have an interior surface facing toward an interior body cavity of the patient, and an exterior surface facing away from the interior body cavity. The method may include positioning a first fastener proximate a plate, wherein the plate is configured to span the first fracture. The plate may have an exterior plate surface configured to abut the interior surface of the bone, an interior plate surface opposite the exterior plate surface, and a slot passing between the exterior plate surface and the interior plate surface. The first fastener may have a proximal end including a head portion, a distal end, and a shank that extends between the head portion and the distal end. The method may further include moving the slot from an unexpanded configuration to an expanded configuration, with the slot in the expanded configuration, inserting the distal end through the slot, and allowing the slot to return to the unexpanded configuration such that the shank is accommodated within the slot but the distal end is not withdrawable through the slot.

In the method of any preceding paragraph, moving the slot from the unexpanded configuration to the expanded configuration may include inserting a cam surface of a tool into the slot. The cam surface may have a cam width smaller than a slot width of the slot, and a cam length longer than the slot width. The method may further include rotating the cam surface within the slot to urge deformation of the slot from the unexpanded configuration to the expanded configuration.

In the method of any preceding paragraph, the distal end may be threaded, and the shank may have a smallest shank width smaller than a distal end width of the distal end.

In the method of any preceding paragraph, the shank may have threads, and unthreaded flats that interrupt the threads and define the smallest shank width. Allowing the slot to return to the unexpanded configuration may include positioning the unthreaded flats to abut interior walls of the slot to prevent rotation of the shank within the slot.

According to some embodiments, a fracture plating system may be configured to stabilize a first fracture of a bone of a patient. The bone may have an interior surface facing toward an interior body cavity of the patient, and an exterior surface facing away from the interior body cavity. The fracture plating system may include a plate configured to span the first fracture. The plate may have an exterior plate surface configured to abut the interior surface, an interior plate surface opposite the exterior plate surface, and a slot passing between the exterior plate surface and the interior plate surface. The fracture plating system may further include a first fastener with a proximal end including a head portion, and a distal end. The fracture plating system may further include a tether configured to apply a force to the first fastener to guide the first fastener such that the distal end enters the slot, and draw the first fastener such that the distal end passes through the slot and the head portion engages the interior plate surface.

In the fracture plating system of any preceding paragraph, the tether may be configured to enable a knot to be tied in the tether such that the knot persists until untied. The tether may be configured to apply the force to the first fastener by abutting the proximal end of the first fastener with the knot.

In the fracture plating system of any preceding paragraph, the fracture plating system may further include a bead configured to be permanently secured to the tether. The tether may be configured to apply the force to the first fastener by abutting the proximal end of the first fastener with the bead.

In the fracture plating system of any preceding paragraph, the first fastener may have a longitudinal axis, and the first fastener may be configured to engage the plate such that the first fastener is retained at a fixed orientation about the longitudinal axis, relative to the plate.

In the fracture plating system of any preceding paragraph, the first fastener may have a threaded portion extending along the longitudinal axis. The threaded portion may have a flat configured to engage the slot to retain the first fastener at the fixed orientation relative to the plate.

In the fracture plating system of any preceding paragraph, the proximal end may further have a wing feature extending from the head portion such that, with the head portion engaging the interior plate surface, the wing feature engages the interior plate surface to retain the first fastener at the fixed orientation relative to the plate.

In the fracture plating system of any preceding paragraph, the fracture plating system may further include a second fastener, and a third fastener. The second fastener and the third fastener may be configured to secure the plate to the bone prior to engagement of the head portion of the first fastener with the interior plate surface.

According to some embodiments, a method may be used to stabilize a first fracture of a bone of a patient. The bone may have an interior surface facing toward an interior body cavity of the patient, and an exterior surface facing away from the interior body cavity. The method may include securing a plate to the interior surface such that the plate spans the first fracture. The plate may have an interior plate surface, an exterior plate surface, and a slot. The method may further include, with the plate secured to the interior surface such that the exterior plate surface abuts the interior surface, using a tether to apply a force to a first fastener to draw the first fastener such that a distal end of the first fastener enters the slot. The method may further include, with the plate secured to the interior surface, using the tether to draw the first fastener such that the distal end passes through the slot and a head portion of a proximal end of the first fastener engages the interior plate surface.

In the method of any preceding paragraph, the method may further include, prior to using the tether to apply the force to the first fastener, tying a knot in the tether. Using the tether to apply the force to the first fastener may include engaging the first fastener with the knot.

In the method of any preceding paragraph, the method may further include, prior to using the tether to apply the force to the first fastener, permanently securing a bead to the tether. Using the tether to apply the force to the first fastener may include engaging the first fastener with the bead.

In the method of any preceding paragraph, the first fastener may have a longitudinal axis, and the method further may include engaging the plate with the first fastener such that the first fastener is retained at a fixed orientation about the longitudinal axis, relative to the plate.

In the method of any preceding paragraph, the first fastener may have a threaded portion extending along the longitudinal axis. The threaded portion may include a flat. Engaging the plate with the first fastener may include engaging the slot to retain the first fastener at the fixed orientation relative to the plate.

In the method of any preceding paragraph, the proximal end may further have a wing feature extending from the head portion. Engaging the plate with the first fastener may include engaging the interior plate surface with the wing feature to retain the first fastener at the fixed orientation relative to the plate.

In the method of any preceding paragraph, the method may further include, prior to engaging the interior plate surface with the head portion, securing the plate to the bone with a second fastener and a third fastener.

In the method of any preceding paragraph, the bone may further have a second fracture defining a flail segment between the first fracture and the second fracture. Securing the plate to the bone with the second fastener and the third fastener may include securing the second fastener and the third fastener outside the flail segment and on opposite sides of the flail segment. The method may further include securing the first fastener to the flail segment.

According to some embodiments, a method may be used to stabilize a first fracture of a bone of a patient. The bone may have an interior surface facing toward an interior body cavity of the patient, and an exterior surface facing away from the interior body cavity. The method may include passing a first end of a tether through the bone, passing the first end through a slot of a plate, the plate including an interior plate surface and an exterior plate surface, passing the first end through a first fastener, tying a knot in the tether, pulling the tether such that the knot engages the first fastener to draw the first fastener toward the bone, and, with the plate on the bone such that the exterior plate surface abuts the interior surface, securing the first fastener to the plate and/or the bone.

In the method of any preceding paragraph, the method may further include, after passing the first end of the tether through the bone and prior to passing the first end of the tether through the slot and through the first fastener, passing the first end of the tether out of the interior body cavity.

In the method of any preceding paragraph, tying the knot in the tether may include tying an overhand knot.

In the method of any preceding paragraph, the method may further include securing the plate to the bone with a second fastener after drawing the first fastener to the bone. Drawing the first fastener to the bone may include drawing the plate to the bone with the first fastener.

In the method of any preceding paragraph, the method may further include securing the plate to the bone with a second fastener prior to drawing the first fastener to the bone. Drawing the first fastener to the bone may include causing a distal end of the first fastener to enter the slot. The method may further include, with the plate secured to the interior surface, using the tether to draw the first fastener such that the distal end passes through the slot and a head portion of the first fastener engages the interior plate surface.

According to some embodiments, a fracture plating system may be configured to stabilize a first fracture of a bone of a patient. The bone may have an interior surface facing toward an interior body cavity of the patient, and an exterior surface facing away from the interior body cavity. The fracture plating system may include a plate configured to span the first fracture. The plate may have an exterior plate surface configured to abut the interior surface, an interior plate surface opposite the exterior plate surface, and a slot passing between the exterior plate surface and the interior plate surface. The fracture plating system may further include a first fastener with a proximal end with a head portion, and a distal end. The fracture plating system may further include a first tether, a first drill bit, and a first low-profile driver configured to rotate the first drill bit to form a first hole in the bone such that the first tether is insertable through the first hole. The bone may include a rib. The first low-profile driver may have a height along an axis of rotation of the first drill bit. The height may be limited such that the first low-profile driver is operable within a space between the rib and an adjacent scapula without requiring sufficient distraction of the space to damage tissues within the space.

In the fracture plating system of any preceding paragraph, the first low-profile driver may include a first angled driver including a shaft oriented transversely to the axis of rotation.

In the fracture plating system of any preceding paragraph, the first drill bit may be cannulated, the shaft may be cannulated such that the shaft and the first drill bit cooperate to define a channel extending continuously through the shaft and the first drill bit, and the first tether may be configured to pass through the channel and through the first hole.

In the fracture plating system of any preceding paragraph, the fracture plating system may further include a first cannulated cap configured to receive the first tether and to be coupled to the first fastener to secure the first fastener and the plate to the bone.

In the fracture plating system of any preceding paragraph, the fracture plating system may further include a first cannulated driver bit configured to be coupled to the first cannulated cap. The first low-profile driver may be configured to rotate the first cannulated driver bit into engagement with the first fastener. The first low-profile driver may cooperate with the first cannulated driver bit to define a channel extending continuously through the first low-profile driver and the first cannulated driver bit. The first tether may be configured to pass through the channel and through the first hole.

In the fracture plating system of any preceding paragraph, the fracture plating system may further include a second fastener, a second tether, a second drill bit, and a second low-profile driver configured to rotate the second drill bit to form a second hole in the bone such that the second tether is insertable through the second hole.

In the fracture plating system of any preceding paragraph, with the first tether inserted through the first hole and the second tether inserted through the second hole, the first tether and the second tether may be configured to cooperate to draw the first fastener, the second fastener, and the plate toward the interior surface of the bone.

In the fracture plating system of any preceding paragraph, the fracture plating system may further include a first flexible tube configured to receive the first tether and to be guided through the first hole by the first tether, a second flexible tube configured to receive the second tether and to be guided through the second hole by the second tether, and a third tether. The third tether may be configured to be received in the first flexible tube to be guided through the first hole by the first flexible tube, be received in the second flexible tube to be guided through the second hole by the second flexible tube, and draw the first fastener, the second fastener, and the plate toward the interior surface of the bone.

According to some embodiments, a method may be used to stabilize a first fracture of a bone of a patient. The bone may have an interior surface facing toward an interior body cavity of the patient, and an exterior surface facing away from the interior body cavity. The method may include using a first low-profile driver to form a first hole in the bone with a first drill bit, passing a first tether through the first hole, and guiding a first fastener and a plate toward the interior surface of the bone. The bone may be a rib. The first low-profile driver may have a first height along an axis of rotation of the first drill bit. The first height may be limited such that the first low-profile driver is operable within a space between the rib and an adjacent scapula without requiring sufficient distraction of the space to damage tissues within the space.

In the method of any preceding paragraph, the first low-profile driver may include a first angled driver including a shaft, and forming the first hole in the bone may include, during rotation of the first drill bit, holding the shaft with the shaft oriented transversely to the axis of rotation.

In the method of any preceding paragraph, the first drill bit may be cannulated, the shaft may be cannulated such that the shaft and the first drill bit cooperate to define a channel extending continuously through the shaft and the first drill bit, and passing the first tether through the first hole may include passing the first tether through the first low-profile driver via the channel such that the first tether passes through the first hole.

In the method of any preceding paragraph, the method may further include receiving the first tether in a first cannulated cap to guide the first cannulated cap to the first hole, and coupling the first cannulated cap to the first fastener to secure the first fastener and the plate to the bone.

In the method of any preceding paragraph, the method may further include coupling a first cannulated driver bit to the first cannulated cap. Coupling the first cannulated cap to the first fastener to secure the first fastener and the plate to the bone may include, with the first low-profile driver, rotating the first cannulated driver bit into engagement with the first fastener. The first low-profile driver may cooperate with the first cannulated driver bit to define a channel extending continuously through the first low-profile driver and the first cannulated driver bit. Receiving the first tether in the first cannulated cap may include passing the first tether through the channel.

In the method of any preceding paragraph, the method may further include using a second low-profile driver to form a second hole in the bone with a second drill bit, passing a second tether through the second hole, and guiding a second fastener toward the interior surface of the bone. The bone may be a rib. The second low-profile driver may have a second height along an axis of rotation of the second drill bit. The second height may be limited such that the second low-profile driver is operable within the space without requiring sufficient distraction of the space to damage tissues within the space.

In the method of any preceding paragraph, the method may further include, with the first tether inserted through the first hole and the second tether inserted through the second hole, using the first tether and the second tether to draw the first fastener, the second fastener, and the plate toward the interior surface of the bone.

In the method of any preceding paragraph, the method may further include receiving the first tether in a first flexible tube, guiding the first flexible tube through the first hole with the first tether, receiving the second tether in a second flexible tube, guiding the second flexible tube through the second hole with the second tether, receiving a third tether in the first flexible tube, guiding the third tether through the first hole with the first flexible tube, receiving the third tether in the second flexible tube, guiding the third tether through the second hole with the second flexible tube, and drawing the first fastener, the second fastener, and the plate toward the interior surface of the bone with the third tether.

According to some embodiments, a method may be used to stabilize a first fracture of a bone of a patient. The bone may have an interior surface facing toward an interior body cavity of the patient, and an exterior surface facing away from the interior body cavity. The method may include passing a first tether through a first hole in the bone, inserting the first tether into a first flexible tube, guiding the first flexible tube through the first hole with the first tether, receiving a second tether in the first flexible tube, guiding the second tether through the first hole with the first flexible tube, and drawing a first fastener and a plate toward the interior surface of the bone with the second tether.

In the method of any preceding paragraph, the method may further include using a first low-profile driver to form the first hole in the bone with a first drill bit. The bone may be a rib. The first low-profile driver may have a first height along an axis of rotation of the first drill bit. The first height may be limited such that the first low-profile driver is operable within a space between the rib and an adjacent scapula without requiring sufficient distraction of the space to damage tissues within the space.

In the method of any preceding paragraph, the method may further include receiving the first tether in a first cannulated cap to guide the first cannulated cap to the first hole, and coupling the first cannulated cap to the first fastener to secure the first fastener and the plate to the bone.

In the method of any preceding paragraph, the method may further include passing a third tether through a second hole in the bone, inserting the third tether into a second flexible tube, guiding the second flexible tube through the second hole with the third tether, receiving the second tether in the second flexible tube, and guiding the second tether through the second hole with the second flexible tube. Drawing the first fastener and the plate toward the interior surface of the bone with the second tether may further include, with the second tether, drawing the first fastener toward the first hole, and drawing a second fastener toward the second hole.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1 is a perspective view of a fracture plating system according to an embodiment of the present disclosure.

FIG. 2A is a front view of a plate of the fracture plating system of FIG. 1 according to an embodiment of the present disclosure.

FIG. 2B is a bottom view of the plate of FIG. 2A.

FIG. 3A is a front view of an anti-rotation fastener of the fracture plating system of FIG. 1 according to an embodiment of the present disclosure.

FIG. 3B is a side view of the anti-rotation fastener of FIG. 3A.

FIG. 4A is a front view of a circular head fastener of the fracture plating system of FIG. 1 according to an embodiment of the present disclosure.

FIG. 4B is a side view of the circular head fastener of FIG. 4A.

FIG. 5A is a perspective view of a locking cap of the fracture plating system of FIG. 1 according to an embodiment of the present disclosure.

FIG. 5B is a top view of the locking cap of FIG. 5A.

FIG. 5C is a side view of the locking cap of FIG. 5A.

FIG. 6A is a perspective view of a washer of the fracture plating system of FIG. 1 according to an embodiment of the present disclosure.

FIG. 6B is a top view of the washer of FIG. 6A.

FIG. 6C is a side view of the washer of FIG. 6A.

FIG. 7 is a perspective view of the fracture plating system of FIG. 1 in a lower profile configuration.

FIG. 8 is a perspective view of the fracture plating system of FIG. 1 spanning an exemplary bone fracture.

FIG. 9 is a perspective view of the fracture plating system of FIG. 1 in a lower profile configuration.

FIG. 10A is a perspective view of the plate of the fracture plating system of FIG. 1 according to an embodiment of the present disclosure.

FIG. 10B is a top perspective partial view of the plate of FIG. 10A.

FIG. 11 is a front view of the fracture plating system of FIG. 1 in a first pre-assembled configuration.

FIG. 12 is a front view of the fracture plating system of FIG. 1 in a second pre-assembled configuration.

FIG. 13 is a front partial section view of the fracture plating system of FIG. 12.

FIG. 14 is a front section view of the fracture plating system of FIG. 12 in a third pre-assembled configuration.

FIG. 15 is a front view of the fracture plating system of FIG. 1 in an assembled configuration.

FIG. 16 is a front section view of the fracture plating system of FIG. 15.

FIG. 17 is a front section view of the fracture plating system of FIG. 1 in a lower profile configuration.

FIG. 18 is a perspective view of the fracture plating system of FIG. 1 in a partially deployed configuration spanning an exemplary bone fracture.

FIG. 19 is a front section view of the fracture plating system of FIG. 18 in a partially deployed configuration spanning an exemplary bone fracture.

FIG. 20 is a perspective view of the fracture plating system of FIG. 18 in a partially deployed configuration spanning an exemplary bone fracture.

FIG. 21 is a front section view of the fracture plating system of FIG. 20 in a partially deployed configuration spanning an exemplary bone fracture.

FIG. 22 is a front section view of the fracture plating system of FIG. 20 in a partially deployed configuration spanning an exemplary bone fracture.

FIG. 23 is a perspective view of the fracture plating system of FIG. 20 in a partially deployed configuration spanning an exemplary bone fracture.

FIG. 24 is a perspective view of the fracture plating system of FIG. 1 in a partially deployed configuration spanning an exemplary bone fracture.

FIG. 25 is a front view of the fracture plating system of FIG. 24 in a partially deployed configuration spanning an exemplary bone fracture.

FIG. 26 is a perspective view of the fracture plating system of FIG. 1 in a partially deployed configuration spanning an exemplary bone fracture.

FIG. 27 is a perspective view of the fracture plating system of FIG. 26 in a partially deployed configuration spanning an exemplary bone fracture.

FIG. 28 is a perspective view of the fracture plating system of FIG. 26 in a deployed configuration spanning an exemplary bone fracture.

FIG. 29 is a perspective view of a fracture plating system according to an embodiment of the present disclosure.

FIG. 30 is a perspective section view of the fracture plating system of FIG. 29.

FIG. 31 is a partial bottom perspective view of the fracture plating system of FIG. 29.

FIG. 32A is a top view of a circular head fixed hinge fastener of the fracture plating system of FIG. 29 according to an embodiment of the present disclosure.

FIG. 32B is a front view of the circular head fixed hinge fastener of FIG. 32A.

FIG. 32C is a side view of the circular head fixed hinge fastener of FIG. 32A.

FIG. 33A is a top view of a fixed hinge fastener of the fracture plating system of FIG. 29 according to an embodiment of the present disclosure.

FIG. 33B is a front view of the fixed hinge fastener of FIG. 33A.

FIG. 33C is a side view of the fixed hinge fastener of FIG. 33A.

FIG. 34 is a perspective view of a fracture plating system according to an embodiment of the present disclosure.

FIG. 35 is a bottom perspective view of the fracture plating system of FIG. 34 in a pre-assembled configuration.

FIG. 36A is a front view of a plate of the fracture plating system of FIG. 34 according to an embodiment of the present disclosure.

FIG. 36B is a bottom view of the plate of FIG. 36A.

FIG. 37A is a front view of a fixed hinge fastener of the fracture plating system of FIG. 34 according to an embodiment of the present disclosure.

FIG. 37B is a side view of the fixed hinge fastener of FIG. 37A.

FIG. 38A is a front view of the anti-rotation fastener of FIG. 3A.

FIG. 38B is a side view of the anti-rotation fastener of FIG. 38A.

FIG. 39 is a perspective view of a fracture plating system according to an embodiment of the present disclosure.

FIG. 40 is a bottom perspective view of the fracture plating system of FIG. 39.

FIG. 41A is a front view of a plate of the fracture plating system of FIG. 39 according to an embodiment of the present disclosure.

FIG. 41B is a bottom view of the plate of FIG. 41A.

FIG. 42A is a front view of the circular head fastener of FIG. 4A.

FIG. 42B is a side view of the circular head fastener of FIG. 42A

FIG. 43A is a front view of a ball-headed fastener of the fracture plating system of FIG. 39 according to an embodiment of the present disclosure.

FIG. 43B is a side view of the ball-headed fastener of FIG. 43A.

FIG. 44A is a perspective view of a fracture plating system according to an embodiment of the present disclosure.

FIG. 44B is a partial perspective view of the fracture plating system of FIG. 44A.

FIG. 45 is a bottom perspective view of the fracture plating system of FIG. 44A.

FIG. 46A is a top view of a plate of the fracture plating system of FIG. 44A according to an embodiment of the present disclosure.

FIG. 46B is a front view of the plate of FIG. 46A.

FIG. 47A is a top view of a pin fastener of the fracture plating system of FIG. 44A according to an embodiment of the present disclosure.

FIG. 47B is a front view of the pin fastener of FIG. 47A.

FIG. 47C is a side view of the pin fastener of FIG. 47A.

FIG. 48A is a front view of a fracture plating system in a partially deployed configuration according to an embodiment of the present disclosure spanning a plurality of exemplary bone fractures.

FIG. 48B is a perspective view of a fracture plating system secured to a bone with multiple fractures according to an embodiment of the present disclosure.

FIG. 48C is a perspective view of the fracture plating system of FIG. 48B.

FIG. 48D is a perspective view of a fracture plating system secured to a bone with multiple fractures according to an embodiment of the present disclosure.

FIG. 48E is a perspective view of the fracture plating system of FIG. 48D.

FIG. 49 is a front view of a fracture plating system in a partially deployed configuration according to an embodiment of the present disclosure spanning a plurality of exemplary bone fractures.

FIG. 50 is a bottom perspective view of the fracture plating system of FIG. 49 in a partially deployed configuration spanning a plurality of exemplary bone fractures.

FIG. 51 is a bottom perspective view of a fracture plating system in a partially deployed configuration according to an embodiment of the present disclosure spanning an exemplary bone fracture.

FIG. 52 is a partial bottom perspective view of the fracture plating system of FIG. 51 spanning an exemplary bone fracture.

FIG. 53 is a front view of a fracture plating system in a partially deployed configuration according to an embodiment of the present disclosure spanning an exemplary bone fracture.

FIG. 54 is a partial front view of the fracture plating system of FIG. 53 spanning an exemplary bone fracture.

FIG. 55 is a partial bottom perspective view of the fracture plating system of FIG. 53 spanning an exemplary bone fracture.

FIG. 56 is a front view of a fracture plating system according to an embodiment of the present disclosure spanning a plurality of exemplary bone fractures.

FIG. 57 is a bottom perspective view of the fracture plating system of FIG. 56 spanning a plurality of exemplary bone fractures.

FIG. 58A is a front view of a plate of the fracture plating system of FIG. 56 according to an embodiment of the present disclosure.

FIG. 58B is a bottom view of the plate of FIG. 58A.

FIG. 59 is a perspective view of a fracture plating system according to an embodiment of the present disclosure spanning a plurality of exemplary bone fractures.

FIG. 60 is a perspective view of the fracture plating system of FIG. 59 spanning a plurality of exemplary bone fractures.

FIG. 61 is a bottom perspective view of the fracture plating system of FIG. 59 spanning a plurality of exemplary bone fractures.

FIG. 62A is a front view of a plate of the fracture plating system of FIG. 59 according to an embodiment of the present disclosure.

FIG. 62B is bottom view of the plate of FIG. 62A.

FIG. 63A is a front view of a threaded fastener of the fracture plating system of FIG. 59 according to an embodiment of the present disclosure.

FIG. 63B is a side view of the threaded fastener of FIG. 63A.

FIG. 64A front view of a toggle fastener in an insertion configuration of a fracture plating system according to an embodiment of the present disclosure.

FIG. 64B is a front view of the toggle fastener of FIG. 64A in a deployed configuration.

FIG. 65 is a perspective view of a pair of toggle fasteners of FIG. 64A in a partially deployed configuration spanning an exemplary bone fracture.

FIG. 66 is a perspective view of a pair of toggle fasteners of FIG. 64A in a partially deployed configuration spanning an exemplary bone fracture.

FIG. 67 is a perspective view of a pair of toggle fasteners of FIG. 64A in a partially deployed configuration spanning an exemplary bone fracture.

FIG. 68 is a perspective view of a pair of toggle fasteners of FIG. 64A in a partially deployed configuration spanning an exemplary bone fracture.

FIG. 69 is a perspective view of a fracture plating system which may include the toggle fastener of FIG. 64A according to an embodiment of the present disclosure spanning an exemplary bone fracture.

FIG. 70 is a bottom perspective view of the fracture plating system of FIG. 69 spanning an exemplary bone fracture.

FIG. 71 is a bottom view of the plate of FIG. 2A.

FIG. 72 is a perspective view of a fracture plating system according to an embodiment of the present disclosure in a partially assembled configuration.

FIG. 73 is a perspective view of the fracture plating system of FIG. 72 in a partially assembled configuration.

FIG. 74A is a front view of a post fastener of the fracture plating system of FIG. 72 according to an embodiment of the present disclosure.

FIG. 74B is a side view of the post fastener of FIG. 74A.

FIG. 75A is a front view of a cap of the fracture plating system of FIG. 72 according to an embodiment of the present disclosure.

FIG. 75B is a side view of the cap of FIG. 75A.

FIG. 75C is a perspective view of the cap of FIG. 75A.

FIG. 76A is top view of a pin fastener of a fracture plating system of FIG. 81 according to an embodiment of the present disclosure.

FIG. 76B is a perspective view of the pin fastener of FIG. 76A.

FIG. 76C is a front view of the pin fastener of FIG. 76A.

FIG. 76D is a side view of the pin fastener of FIG. 76A.

FIG. 77A is a top view of a plate of the fracture plating system of FIG. 81 according to an embodiment of the present disclosure.

FIG. 77B is a front view of the plate of FIG. 77A.

FIG. 78 is a partial top view of the fracture plating system of FIG. 81 in a partially assembled configuration.

FIG. 79 is a partial perspective view of the fracture plating system of FIG. 81 in a partially assembled configuration.

FIG. 80 is a perspective section view of the fracture plating system of FIG. 81.

FIG. 81 is a perspective view of a fracture plating system according to an embodiment of the present disclosure.

FIG. 82 is a partial perspective view of the fracture plating system of FIG. 81.

FIG. 83 is a partial perspective view of the fracture plating system of FIG. 81.

FIG. 84 is a perspective view of an exemplary rig cage with an exemplary bone fracture.

FIG. 85 is a partial perspective view of the exemplary rib cage of FIG. 84.

FIG. 86 is a partial perspective view of the exemplary rib cage of FIG. 84 illustrating a step in a method of deploying a fracture plating system according to an embodiment of the present disclosure.

FIG. 87 is a partial perspective view of the exemplary rib cage of FIG. 84 illustrating a step in a method of deploying a fracture plating system according to an embodiment of the present disclosure.

FIG. 88 is a partial perspective view of the exemplary rib cage of FIG. 84 illustrating a step in a method of deploying a fracture plating system according to an embodiment of the present disclosure.

FIG. 89A is a front view of a set of differently sized plates according to an embodiment of the present disclosure.

FIG. 89B is a front view of a set of differently sized fasteners according to an embodiment of the present disclosure.

FIG. 89C is a front view of a set of differently sized locking caps according to an embodiment of the present disclosure.

FIG. 89D is a partial perspective view of an exemplary rib cage showing an exemplary fracture.

FIG. 90 is a front view of a fracture plating system illustrating a step in a method of deploying the fracture plating system according to an embodiment of the present disclosure.

FIG. 91 is a front view of the fracture plating system of FIG. 90 illustrating a step in a method of deploying the fracture plating system according to an embodiment of the present disclosure.

FIG. 92 is a front view of the fracture plating system of FIG. 90 illustrating a step in a method of deploying the fracture plating system according to an embodiment of the present disclosure.

FIG. 93 is a partial perspective view of the exemplary rib cage of FIG. 84 illustrating a step in a method of deploying a fracture plating system according to an embodiment of the present disclosure.

FIG. 94 is a partial perspective view of the exemplary rib cage of FIG. 84 illustrating a step in a method of deploying a fracture plating system according to an embodiment of the present disclosure.

FIG. 95 is a partial perspective view of the exemplary rib cage of FIG. 84 illustrating a step in a method of deploying a fracture plating system according to an embodiment of the present disclosure.

FIG. 96 is a partial perspective view of the exemplary rib cage of FIG. 84 illustrating a step in a method of deploying a fracture plating system according to an embodiment of the present disclosure.

FIG. 97 is a partial perspective view of the exemplary rib cage of FIG. 84 illustrating a step in a method of deploying a fracture plating system according to an embodiment of the present disclosure.

FIG. 98 is a partial perspective view of the exemplary rib cage of FIG. 84 illustrating a step in a method of deploying a fracture plating system according to an embodiment of the present disclosure.

FIG. 99 is a partial perspective view of the exemplary rib cage of FIG. 84 illustrating a step in a method of deploying a fracture plating system according to an embodiment of the present disclosure.

FIG. 100 is a partial perspective view of the exemplary rib cage of FIG. 84 illustrating a step in a method of deploying a fracture plating system according to an embodiment of the present disclosure.

FIG. 101 is a partial perspective view of the exemplary rib cage of FIG. 84 illustrating a step in a method of deploying a fracture plating system according to an embodiment of the present disclosure.

FIG. 102 is a perspective view of a fracture plating system according to an embodiment of the present disclosure spanning an exemplary bone fracture.

FIG. 103 is a perspective view of a fracture plating system of FIG. 102.

FIG. 104A is a front view of an anti-rotation fastener of the fracture plating system of FIG. 102 according to an embodiment of the present disclosure.

FIG. 104B is a side view of the anti-rotation fastener of FIG. 104A.

FIG. 105A is a front view of a locking cap of the fracture plating system of FIG. 102 according to an embodiment of the present disclosure.

FIG. 105B is a side view of the locking cap of FIG. 105A.

FIG. 106A is a perspective view of a driver of the fracture plating system of FIG. 102 according to an embodiment of the present disclosure.

FIG. 106B is a side view of the driver of FIG. 106A.

FIG. 106C is a front view of the driver of FIG. 106A.

FIG. 107 is a front perspective view of a spinal fixation plating system, secured to an exemplary portion of a spine, according to an embodiment of the present disclosure.

FIG. 108 is a rear perspective view of the spinal fixation plating system of FIG. 107 secured to an exemplary portion of a spine.

FIG. 109 is a front perspective view of the spinal fixation plating system of FIG. 107 secured to an exemplary portion of a spine.

FIG. 110 is a rear perspective view of the spinal fixation plating system of FIG. 107 secured to an exemplary portion of a spine.

FIG. 111A is a front view of a plate of the spinal fixation plating system of FIG. 107 according to an embodiment of the present disclosure.

FIG. 111B is a bottom view of the plate of FIG. 111A.

FIG. 112A is a perspective view of a locking cap of the spinal fixation plating system of FIG. 107 according to an embodiment of the present disclosure.

FIG. 112B is a front view of the locking cap of FIG. 112A.

FIG. 112C is a side view of the locking cap of FIG. 112A.

FIG. 113A is a perspective view of an anti-rotation fastener of the spinal fixation plating system of FIG. 107 according to an embodiment of the present disclosure.

FIG. 113B is a front view of the anti-rotation fastener of FIG. 113A.

FIG. 113C is a side view of the anti-rotation fastener of FIG. 113A.

FIG. 114 is a front view of a fracture plating system according to an embodiment of the present disclosure spanning an exemplary bone fracture.

FIG. 115 is a partial perspective view of the fracture plating system of FIG. 114.

FIG. 116 is a partial section view of the fracture plating system of FIG. 114.

FIG. 117 is a front section view of the fracture plating system of FIG. 114.

FIG. 118A is a bottom perspective view of a ratchet cap of the fracture plating system of FIG. 114 according to an embodiment of the present disclosure.

FIG. 118B is front perspective view of the ratchet cap of FIG. 118A.

FIG. 118C is a front view of the ratchet cap of FIG. 118A.

FIG. 118D is a side view of the ratchet cap of FIG. 118A.

FIG. 119A is a perspective view of a ratchet fastener of the fracture plating system of FIG. 114 according to an embodiment of the present disclosure.

FIG. 119B is a front view of the ratchet fastener of FIG. 119A.

FIG. 119C is a side view of the ratchet fastener of FIG. 119A.

FIG. 120A is a perspective view of a fracture repair system in an undeformed configuration according to an embodiment of the present disclosure.

FIG. 120B is a front view of the fracture repair system of FIG. 120A in an undeformed configuration.

FIG. 120C is a side view of the fracture repair system of FIG. 120A in an undeformed configuration.

FIG. 121 is a front view of the fracture repair system of FIG. 120A in an undeformed configuration.

FIG. 122 is a front view of the fracture repair system of FIG. 120A in an expanded configuration according to an embodiment of the present disclosure.

FIG. 123 is a front section view of an exemplary rib cage with the fracture repair system of FIG. 122 spanning exemplary fractures.

FIG. 124 is a front section view of an exemplary rib cage with the fracture repair system of FIG. 120A compressing exemplary fractures.

FIG. 125 is a front section view of an exemplary rib cage with the fracture repair system of FIG. 120A compressing exemplary fractures.

FIG. 126A is a perspective view of an application instrument of a fracture repair system according to an embodiment of the present disclosure.

FIG. 126B is a side view of the application instrument of FIG. 126A.

FIG. 126C is a front view of the application instrument of FIG. 126A.

FIG. 127 is a balloon of a fracture repair system according to an embodiment of the present disclosure.

FIG. 128 is a partial perspective view of an exemplary rib cage with an exemplary bone fracture.

FIG. 129 is a partial perspective view of the exemplary rib cage of FIG. 128 illustrating a step in a method of deploying a fracture repair system according to an embodiment of the present disclosure.

FIG. 130 is a partial perspective view of the exemplary rib cage of FIG. 128 illustrating a step in a method of deploying a fracture repair system according to an embodiment of the present disclosure.

FIG. 131 is a partial perspective view of the exemplary rib cage of FIG. 128 illustrating a step in a method of deploying a fracture repair system according to an embodiment of the present disclosure.

FIG. 132 is a partial perspective view of the exemplary rib cage of FIG. 128 illustrating a step in a method of deploying a fracture repair system according to an embodiment of the present disclosure.

FIG. 133 is a partial perspective view of the exemplary rib cage of FIG. 128 illustrating a step in a method of deploying a fracture repair system according to an embodiment of the present disclosure.

FIG. 134 is a partial perspective view of the exemplary rib cage of FIG. 128 illustrating a step in a method of deploying a fracture repair system according to an embodiment of the present disclosure.

FIG. 135 is a partial perspective section view of the exemplary rib cage of FIG. 128 illustrating a step in a method of deploying a fracture repair system according to an embodiment of the present disclosure.

FIG. 136 is a partial perspective view of the exemplary rib cage of FIG. 128 illustrating a step in a method of deploying a fracture repair system according to an embodiment of the present disclosure.

FIG. 137 is a perspective view of a fracture plating system in a partially deployed configuration spanning exemplary bone fractures according to an embodiment of the present disclosure.

FIG. 138 is a perspective view of an exemplary axial cross-section view of a spine and ribs.

FIG. 139 is a perspective view of a fracture plating system in a partially deployed configuration spanning exemplary bone fractures according to an embodiment of the present disclosure.

FIG. 140 is a perspective view of the fracture plating system of FIG. 139 in a partially deployed configuration spanning exemplary bone fractures according to an embodiment of the present disclosure.

FIG. 141 is a perspective view of the fracture plating system of FIG. 139 in a deployed configuration spanning exemplary bone fractures according to an embodiment of the present disclosure.

FIG. 142 is a perspective view of a plate of the fracture plating system of FIG. 139 according to an embodiment of the present disclosure.

FIG. 143A is a perspective view of the fracture plating system of FIG. 139 in a deployed configuration spanning exemplary bone fractures according to an embodiment of the present disclosure.

FIG. 143B is a perspective view of the fracture plating system of FIG. 143A.

FIG. 144A is a perspective view of the fracture plating system of FIG. 137 in a deployed configuration spanning exemplary bone fractures according to an embodiment of the present disclosure.

FIG. 144B is a perspective view of the fracture plating system of FIG. 144A.

FIG. 144C is a perspective view of a plate of the fracture plating system of FIG. 137 according to an embodiment of the present disclosure.

FIG. 145A is a perspective view of an exemplary axial cross-section view of a spine and a rib with a fracture.

FIG. 145B is a perspective view of an exemplary axial cross-section view of a spine and a rib with multiple fractures.

FIG. 145C is a perspective view of an exemplary axial cross-section view of a spine and a rib with multiple fractures.

FIG. 145D is a perspective view of an exemplary axial cross-section view of a spine and a rib with multiple fractures.

FIG. 146A is a perspective view of a fracture plating system in a deployed configuration spanning exemplary bone fractures according to an embodiment of the present disclosure.

FIG. 146B is a perspective view of the fracture plating system of FIG. 146A in a deployed configuration spanning exemplary bone fractures.

FIG. 147A is a perspective view of a fracture plating system of FIG. 146A in a deployed configuration spanning exemplary bone fractures.

FIG. 147B is a perspective view of a fracture plating system in a deployed configuration spanning exemplary bone fractures according to an embodiment of the present disclosure.

FIG. 147C is a perspective view of a fracture plating system of FIG. 146B in a deployed configuration spanning exemplary bone fractures.

FIG. 148A is a perspective view of a fracture plating system in a partially deployed configuration spanning an exemplary bone fracture according to an embodiment of the present disclosure.

FIG. 148B is a perspective view of a barrel nut, a low-profile fastener, and a tether of the fracture plating system of FIG. 148A in a partially deployed configuration according to an embodiment of the present disclosure.

FIG. 148C is a perspective view of the fracture plating system of FIG. 148A in a deployed configuration spanning an exemplary bone fracture according to an embodiment of the present disclosure.

FIG. 148D is a perspective view of the fracture plating system of FIG. 148B in a deployed configuration spanning an exemplary bone fracture.

FIG. 148E is a perspective view of the fracture plating system of FIG. 148B in a deployed configuration spanning an exemplary bone fracture.

FIG. 149A is a perspective view of a fracture plating system in a partially deployed configuration spanning an exemplary bone fracture according to an embodiment of the present disclosure.

FIG. 149B is a perspective view of the fracture plating system of FIG. 149A in a partially deployed configuration spanning an exemplary bone fracture.

FIG. 149C is a perspective view of the fracture plating system of FIG. 149B in a partially deployed configuration spanning an exemplary bone fracture.

FIG. 150 is a perspective view of a barrel cap of the fracture plating system of FIG. 149A according to an embodiment of the present disclosure.

FIG. 151A is a perspective view of the fracture plating system of FIG. 149A in a partially deployed configuration according to an embodiment of the present disclosure.

FIG. 151B is a perspective view of the fracture plating system of FIG. 151A in a partially deployed configuration according to an embodiment of the present disclosure.

FIG. 152A is a perspective view of a fracture plating system in a partially deployed configuration spanning an exemplary bone fracture according to an embodiment of the present disclosure.

FIG. 152B is a perspective view of the fracture plating system of FIG. 152A in a partially deployed configuration according to an embodiment of the present disclosure.

FIG. 153A is a perspective view of a fracture plating system in a partially deployed configuration spanning an exemplary bone fracture according to an embodiment of the present disclosure.

FIG. 153B is a perspective view of the fracture plating system of FIG. 153A in a deployed configuration according to an embodiment of the present disclosure.

FIG. 154A is a perspective view of a fastener of the fracture plating system of FIG. 153A according to an embodiment of the present disclosure.

FIG. 154B is a perspective view of a fastener of the fracture plating system of FIG. 153A according to an embodiment of the present disclosure.

FIG. 155A is a perspective view of the fracture plating system of FIG. 152A in a partially deployed configuration spanning an exemplary fracture according to an embodiment of the present disclosure.

FIG. 155B is a perspective view of the fracture plating system of FIG. 155A in a deployed configuration spanning an exemplary fracture according to an embodiment of the present disclosure.

FIG. 156 is a perspective view of a step in a method of deploying a fracture plating system which may include drilling a hole in a bone according to an embodiment of the present disclosure.

FIG. 157 is a perspective view of a step in a method of deploying the fracture plating system of FIG. 156 which may include passing a tether of the fracture plating system of FIG. 156 through the hole in the bone according to an embodiment of the present disclosure.

FIG. 158 is a perspective view of a step in a method of deploying the fracture plating system of FIG. 156 which may include passing a fastener of the fracture plating system of FIG. 156 through the hole in the bone according to an embodiment of the present disclosure.

FIG. 159 is a perspective view of a step in a method of deploying the fracture plating system of FIG. 156 which may include passing a plate of the fracture plating system of FIG. 156 over the tether of FIG. 157 according to an embodiment of the present disclosure.

FIG. 160 is a perspective view of a step in a method of deploying the fracture plating system of FIG. 156 which may include positioning the plate of FIG. 159 on the bone according to an embodiment of the present disclosure.

FIG. 161 is a perspective view of a step in a method of deploying the fracture plating system of FIG. 156 which may include passing a cap of the fracture plating system of FIG. 156 over the tether of FIG. 157 according to an embodiment of the present disclosure.

FIG. 162 is a perspective view of a step in a method of deploying the fracture plating system of FIG. 156 which may include securing the cap of FIG. 161 onto the fastener of FIG. 157 with a driver according to an embodiment of the present disclosure.

FIG. 163 is a perspective view of a step in a method of deploying the fracture plating system of FIG. 156 which may include withdrawing the driver of FIG. 162 according to an embodiment of the present disclosure.

FIG. 164 is a perspective view of a step in a method of deploying the fracture plating system of FIG. 156 which may include withdrawing the tether of FIG. 157 from the fastener of FIG. 157 according to an embodiment of the present disclosure.

FIG. 165 is a perspective view of a step in a method of deploying a fracture plating system using a minimally invasive technique, which may include drilling a hole in a bone according to an embodiment of the present disclosure.

FIG. 166 is a perspective view of a step in a method of deploying the fracture plating system of FIG. 165 which may include passing a tether of the fracture plating system of FIG. 165 through the hole in the bone according to an embodiment of the present disclosure.

FIG. 167 is a perspective view of a fastener of the fracture plating system of FIG. 165 according to an embodiment of the present disclosure.

FIG. 168A is a perspective view of a step in a method of deploying the fracture plating system of FIG. 165 which may include passing a fastener of the fracture plating system of FIG. 165 through the hole in the bone according to an embodiment of the present disclosure.

FIG. 168B is a perspective view of a step in a method of deploying the fracture plating system of FIG. 165 which may include passing the fastener of FIG. 168A through the hole in the bone.

FIG. 169 is a perspective view of a step in a method of deploying the fracture plating system of FIG. 165 which may include passing the tether of FIG. 166 through a transverse skin tunnel and through a first soft tissue tunnel according to an embodiment of the present disclosure.

FIG. 170 is a perspective view of a step in a method of deploying the fracture plating system of FIG. 165 which may include passing a plate of the fracture plating system of FIG. 165 over the tether of FIG. 166 according to an embodiment of the present disclosure.

FIG. 171 is a perspective view of a step in a method of deploying the fracture plating system of FIG. 165 which may include passing the tether of FIG. 166 back through the first soft tissue tunnel of FIG. 169 according to an embodiment of the present disclosure.

FIG. 172 is a perspective view of a step in a method of deploying the fracture plating system of FIG. 165 which may include passing the tether of FIG. 166 back through the transverse skin tunnel of FIG. 169 according to an embodiment of the present disclosure.

FIG. 173 is a perspective view of a step in a method of deploying the fracture plating system of FIG. 165 which may include passing the tether of FIG. 166 through a second soft tissue tunnel according to an embodiment of the present disclosure.

FIG. 174 is a perspective view of a step in a method of FIG. 173 of deploying the fracture plating system of FIG. 165.

FIG. 175 is a perspective view of a step in a method of FIG. 173 of deploying the fracture plating system of FIG. 165.

FIG. 176 is a perspective view of a step in a method of deploying the fracture plating system of FIG. 165 which may include passing the plate of FIG. 166 through the transverse skin tunnel according to an embodiment of the present disclosure.

FIG. 177 is a perspective view of a step in a method of FIG. 176 of deploying the fracture plating system of FIG. 165.

FIG. 178 is a perspective view of a step in a method of FIG. 176 of deploying the fracture plating system of FIG. 165.

FIG. 179 is a perspective view of a step in a method of deploying the fracture plating system of FIG. 165 which may include positioning the plate of FIG. 170 on the bone according to an embodiment of the present disclosure.

FIG. 180 is a perspective view of a step in a method of deploying the fracture plating system of FIG. 165 which may include passing a cap of the fracture plating system of FIG. 165 over the tether of FIG. 166 according to an embodiment of the present disclosure.

FIG. 181 is a perspective view of a step in a method of deploying the fracture plating system of FIG. 165 which may include securing the cap of FIG. 180 onto the fastener of FIG. 166 with a driver according to an embodiment of the present disclosure.

FIG. 182 is a perspective view of a step in a method of deploying the fracture plating system of FIG. 165 which may include withdrawing the driver of FIG. 181 according to an embodiment of the present disclosure.

FIG. 183 is a perspective view of a step in a method of deploying the fracture plating system of FIG. 165 which may include withdrawing the tether of FIG. 166 from the fastener of FIG. 166 according to an embodiment of the present disclosure.

FIG. 184 is a perspective view of a fracture plating system in a partially deployed configuration spanning an exemplary bone fracture according to an embodiment of the present disclosure.

FIG. 185 is a perspective view of the fracture plating system of FIG. 184 in a deployed configuration spanning an exemplary bone fracture according to an embodiment of the present disclosure.

FIG. 186 is a perspective view of a step in a method of deploying the fracture plating system of FIG. 185 which may include passing a tether of the fracture plating system of FIG. 185 through a hole in a bone according to an embodiment of the present disclosure.

FIG. 187 is a perspective view of a step in a method of deploying the fracture plating system of FIG. 185 which may include passing a second plate of the fracture plating system of FIG. 185 over the tether of FIG. 186 to an exterior portion of the bone according to an embodiment of the present disclosure.

FIG. 188 is a perspective view of a step in a method of deploying the fracture plating system of FIG. 185 which may include positioning the second plate of FIG. 187 on the exterior portion of the bone according to an embodiment of the present disclosure.

FIG. 189 is a perspective view of a step in a method of deploying the fracture plating system of FIG. 185 which may include passing a cap of the fracture plating system of FIG. 185 over the tether of FIG. 186 according to an embodiment of the present disclosure.

FIG. 190 is a perspective view of a step in a method of deploying the fracture plating system of FIG. 185 which may include securing a cap of the fracture plating system FIG. 185 onto a fastener of the fracture plating system of FIG. 185 with a driver according to an embodiment of the present disclosure.

FIG. 191 is a perspective view of a step in a method of deploying the fracture plating system of FIG. 185 which may include withdrawing the driver of FIG. 190 according to an embodiment of the present disclosure.

FIG. 192 is a perspective view of a step in a method of deploying the fracture plating system of FIG. 185 which may include withdrawing the tether of FIG. 186 from the fastener of FIG. 190 according to an embodiment of the present disclosure.

FIG. 193 is a perspective view of a fracture plating system in a deployed configuration spanning exemplary fractures according to an embodiment of the present disclosure.

FIG. 194 is a perspective view of a fracture plating system in a deployed configuration spanning an exemplary fracture according to an embodiment of the present disclosure.

FIG. 195 is a perspective view of the fracture plating system of FIG. 194 in a deployed configuration.

FIG. 196 is a perspective view of a fracture plating system according to an embodiment of the present disclosure.

FIG. 197A is a perspective view of a fastener of the fracture plating system of FIG. 196 according to an embodiment of the present disclosure.

FIG. 197B is a perspective view of a fracture plating system in a deployed configuration on an exemplary bone according to an embodiment of the present disclosure.

FIG. 197C is a perspective view of a fastener of the fracture plating system of FIG. 208 in a neutral configuration and a deployed configuration according to an embodiment of the present disclosure.

FIG. 197D is a perspective view of the fracture plating system of FIG. 208 in a partially deployed configuration according to an embodiment of the present disclosure.

FIG. 197E is a perspective view of the fracture plating system of FIG. 208 in a deployed configuration on an exemplary bone according to an embodiment of the present disclosure.

FIG. 198 is a perspective view of a fracture plating system according to an embodiment of the present disclosure.

FIG. 199 is a perspective view of a fracture plating system according to an embodiment of the present disclosure.

FIG. 200A is a perspective view of a fracture plating system according to an embodiment of the present disclosure.

FIG. 200B is a perspective view of the fracture plating system of FIG. 200A.

FIG. 201 is a perspective view of the fracture plating system of FIG. 200A in a deployed configuration spanning exemplary fractures according to an embodiment of the present disclosure.

FIG. 202A is a perspective view of a fracture plating system according to an embodiment of the present disclosure.

FIG. 202B is a partial perspective view of the fracture plating system of FIG. 202A.

FIG. 203 is a front view of a fracture plating system in a deployed configuration spanning an exemplary fracture according to an embodiment of the present disclosure.

FIG. 204 is a back view of the fracture plating system of FIG. 203.

FIG. 205 is a front view of a fracture plating system in a deployed configuration spanning an exemplary fracture according to an embodiment of the present disclosure.

FIG. 206 is a side view of a fasten of the fracture plating system of FIG. 205 deployed in a hole in an exemplary bone according to an embodiment of the present disclosure.

FIG. 207 is a perspective view of the fracture plating system of FIG. 205.

FIG. 208 is a perspective view of a plate of a fracture plating system according to an embodiment of the present disclosure.

FIG. 209 is a top view of a plate of a fracture plating system according to an embodiment of the present disclosure.

FIG. 210 is a partial front view of the plate of FIG. 213.

FIG. 211A is a top view of a plate of a fracture plating system according to an embodiment of the present disclosure.

FIG. 211B is a front view of the plate of FIG. 211A.

FIG. 212 is a top view of a plate of a fracture plating system according to an embodiment of the present disclosure.

FIG. 213 is a top view of a plate of a fracture plating system according to an embodiment of the present disclosure.

FIG. 214 is a side view of the plate of FIG. 213.

FIG. 215 is a transparent top view of the plate of FIG. 213.

FIG. 216 is a transparent front view of the plate of FIG. 215.

FIG. 217 is a front view of the plate of FIG. 213 in a pre-bent configuration according to an embodiment of the present disclosure.

FIG. 218 is a transparent front view of the plate of FIG. 217 in a bent configuration according to an embodiment of the present disclosure.

FIG. 219 is a front view of the plate of FIG. 218 in a bent configuration.

FIG. 220 is a side view of a fracture plating system in a partially deployed configuration and a deployed configuration according to an embodiment of the present disclosure.

FIG. 221 is a front view of a fracture plating system according to an embodiment of the present disclosure.

FIG. 222 is front views of a fracture plating system in a partially deployed configuration according to an embodiment of the present disclosure.

FIG. 223 is perspective views of the fracture plating system of FIG. 222 according to an embodiment of the present disclosure.

FIG. 224 is a sequence of perspective views of a fracture plating system and method for deploying the same according to an embodiment of the present disclosure.

FIG. 225 is a perspective view of a fracture plating system in a partially deployed configuration on an exemplary bone according to an embodiment of the present disclosure.

FIG. 226 is a perspective view of a fastener of the fracture plating system of FIG. 225 according to an embodiment of the present disclosure.

FIG. 227 is a perspective view of a step in a method of deploying the fracture plating system of FIG. 225 which may include selecting the fasteners of FIG. 222 and a plate according to an embodiment of the present disclosure.

FIG. 228 is a perspective view of a step in a method of deploying the fracture plating system of FIG. 225 which may include positioning the fasteners proximate the plate of FIG. 227 according to an embodiment of the present disclosure.

FIG. 229 is a perspective view of a step in a method of deploying the fracture plating system of FIG. 225 which may include passing the fasteners through the plate according to an embodiment of the present disclosure.

FIG. 230A is a perspective view of a step in a method of deploying the fracture plating system of FIG. 229 which may include positioning the fasteners in the plate according to an embodiment of the present disclosure.

FIG. 230B is a perspective view of a step in a method of deploying the fracture plating system of FIG. 230A which may include positioning the fasteners in the plate according to an embodiment of the present disclosure.

FIG. 231 is a partial perspective view of the fracture plating system of FIG. 230B.

FIG. 232 is a perspective view of the fastener of FIG. 226.

FIG. 233 is a section view of the fracture plating system of FIG. 230B.

FIG. 234 is a perspective view of a step in a method of deploying the fracture plating system of FIG. 230A according to an embodiment of the present disclosure.

FIG. 235 is a perspective view of a step in a method of deploying the fracture plating system of FIG. 230A according to an embodiment of the present disclosure.

FIG. 236 a perspective view of a step in a method of deploying the fracture plating system of FIG. 230A which may include using a driver to engage a cap according to an embodiment of the present disclosure.

FIG. 237 is a perspective view of a fracture plating system according to an embodiment of the present disclosure.

FIG. 238 is a partial perspective view of the fracture plating system of FIG. 237.

FIG. 239 is a section view of the fracture plating system of FIG. 238.

FIG. 240 is a section view of the fracture plating system of FIG. 238.

FIG. 241 is a partial bottom view of the fracture plating system of FIG. 237.

FIG. 242 is a partial bottom view of the fracture plating system of FIG. 237.

FIG. 243 is a partial bottom view of the fracture plating system of FIG. 242.

FIG. 244 is a partial bottom view of the fracture plating system of FIG. 243.

FIG. 245 is a perspective view of a fastener of a fracture plating system according to an embodiment of the present disclosure.

FIG. 246 is a perspective view of the fracture plating system of FIG. 245 in a partially deployed configuration according to an embodiment of the present disclosure.

FIG. 247 is a perspective view of the fracture plating system of FIG. 245 in a deployed configuration according to an embodiment of the present disclosure.

FIG. 248 is a section view of the fracture plating system of FIG. 246.

FIG. 249 is a section view of the fracture plating system of FIG. 247.

FIG. 250 is a perspective view of the fracture plating system of FIG. 246 according to an embodiment of the present disclosure.

FIG. 251 is a section view of the fracture plating system of FIG. 250.

FIG. 252 is a perspective view of the fracture plating system of FIG. 246 in a deployed configuration according to an embodiment of the present disclosure.

FIG. 253 is a perspective view of the fracture plating system of FIG. 252.

FIG. 254 is a perspective view of a fastener of a fracture plating system according to an embodiment of the present disclosure.

FIG. 255 is a side view of the fastener of FIG. 254.

FIG. 256 is a perspective view of the fracture plating system of FIG. 254 in a partially deployed configuration according to an embodiment of the present disclosure.

FIG. 257 is a perspective view of the fracture plating system of FIG. 254 in a partially deployed configuration according to an embodiment of the present disclosure.

FIG. 258 is a perspective view of the fracture plating system of FIG. 254 in a partially deployed configuration according to an embodiment of the present disclosure.

FIG. 259 is a perspective view of the fracture plating system of FIG. 254 in a partially deployed configuration according to an embodiment of the present disclosure.

FIG. 260 is a perspective view of a fracture plating system according to an embodiment of the present disclosure.

FIG. 261 is a perspective view of a step in a method of deploying the fracture plating system of FIG. 260 which may include passing a tether through a plate and a bone according to an embodiment of the present disclosure.

FIG. 262 is a perspective view of a step in a method of deploying the fracture plating system of FIG. 261 which may include drawing the plate toward the bone according to an embodiment of the present disclosure.

FIG. 263 is a perspective view of a step in a method of deploying the fracture plating system of FIG. 262 which may include using the tether to bend the plate to engage the bone according to an embodiment of the present disclosure.

FIG. 264 is a perspective view of a step in a method of deploying the fracture plating system of FIG. 263 which may include passing a cap onto the tether according to an embodiment of the present disclosure.

FIG. 265 is a perspective view of a step in a method of deploying the fracture plating system of FIG. 264 which may include using a driver to engage the cap according to an embodiment of the present disclosure.

FIG. 266 is a perspective view of the fracture plating system of FIG. 260 in a deployed configuration according to an embodiment of the present disclosure.

FIG. 267 is a perspective view of a fracture plating system which may include a plate, a cap, a fastener, and a tether according to an embodiment of the present disclosure.

FIG. 268 is a perspective view of a step in a method of deploying the fracture plating system of FIG. 267 which may include engaging the cap with the fastener and drawing the plate toward a bone according to an embodiment of the present disclosure.

FIG. 269 is a perspective view of a step in a method of deploying the fracture plating system of FIG. 267 which may include engaging a driver with the cap according to an embodiment of the present disclosure.

FIG. 270 is a perspective view of a step in a method of deploying the fracture plating system of FIG. 269 which may include using the driver, the cap, and the fastener to bend the plate to engage the bone according to an embodiment of the present disclosure.

FIG. 271 is a perspective view of the fracture plating system of FIG. 267 in a deployed configuration according to an embodiment of the present disclosure.

FIG. 272 is a method of deploying a fracture plating system according to an embodiment of the present disclosure.

FIG. 273 is a perspective view of a threaded anchor of a fracture plating according to an embodiment of the present disclosure.

FIG. 274 is a top view of a cap of the of the fracture plating system of FIG. 273 according to an embodiment of the present disclosure.

FIG. 275 is a front view of a ratchet anchor of the of the fracture plating system of FIG. 273 according to an embodiment of the present disclosure.

FIG. 276A is a perspective view of an angled driver of a fracture plating system according to an embodiment of the present disclosure.

FIG. 276B is a perspective view of the angled driver of FIG. 276A.

FIG. 276C is a section view of a drill bit of the fracture plating system of FIG. 276A.

FIG. 276D is a perspective view of the angled driver of FIG. 276A.

FIG. 277 is a perspective view of a step in a method of deploying the fracture plating system of FIG. 276A which may include positioning the angled driver of FIG. 276A proximate a subscapular rib fracture according to an embodiment of the present disclosure.

FIG. 278 is a perspective view of a step in a method of deploying the fracture plating system of FIG. 276A which may include drilling a first hole in a rib proximate the subscapular rib fracture according to an embodiment of the present disclosure.

FIG. 279 is a perspective view of a step in a method of deploying the fracture plating system of FIG. 276A which may include passing a first tether through the first hole of FIG. 278 according to an embodiment of the present disclosure.

FIG. 280 is a perspective view of a step in a method of deploying the fracture plating system of FIG. 276A which may include passing the first tether through a chest cavity to an exterior portion of the patient according to an embodiment of the present disclosure.

FIG. 281 is a perspective view of a step in a method of deploying the fracture plating system of FIG. 276A which may include drilling a second hole in the rib and passing a second tether through the second hole according to an embodiment of the present disclosure.

FIG. 282 is a perspective view of a step in a method of deploying the fracture plating system of FIG. 276A which may include passing the second tether through the chest cavity to the exterior portion of the patient according to an embodiment of the present disclosure.

FIG. 283 is a perspective view of a step in a method of deploying the fracture plating system of FIG. 276A which may include positioning the first tether and second tether proximate a first fastener and a second fastener of the fracture plating system of FIG. 276A according to an embodiment of the present disclosure.

FIG. 284 is a perspective view the step of FIG. 283.

FIG. 285 is a perspective view of a step in a method of deploying the fracture plating system of FIG. 276A which may include passing the first tether and second tether through the first fastener and the second fastener of the fracture plating system of FIG. 276A according to an embodiment of the present disclosure.

FIG. 286 is a perspective view of a step in a method of deploying the fracture plating system of FIG. 276A which may include securing a first bead and a second bead on the first tether and the second tether according to an embodiment of the present disclosure.

FIG. 287 is a perspective view of a step in a method of deploying the fracture plating system of FIG. 276A which may include advancing the first tether and the second tether so that the first bead and the second bead contact the first fastener and the second fastener according to an embodiment of the present disclosure.

FIG. 288 is a perspective view of a step in a method of deploying the fracture plating system of FIG. 276A which may include withdrawing the first angled driver and the second angled driver from the first tether and the second tether according to an embodiment of the present disclosure.

FIG. 289 is a perspective view of a step in a method of deploying the fracture plating system of FIG. 276A which may include the first tether being received in the first hole and the second tether being received in the second hole according to an embodiment of the present disclosure.

FIG. 290A is a perspective view of a step in a method of deploying the fracture plating system of FIG. 276A which may include coupling a first hex driver bit to the first angled driver according to an embodiment of the present disclosure.

FIG. 290B is a perspective view of a step in a method of deploying the fracture plating system of FIG. 276A which may include coupling a first cap with the first hex driver bit according to an embodiment of the present disclosure.

FIG. 290C is a perspective view of the first cap coupled with the first hex driver bit according to an embodiment of the present disclosure.

FIG. 291 is a cross section view of the first cap coupled with the first hex driver bit according to an embodiment of the present disclosure.

FIG. 292 is a perspective view of a step in a method of deploying the fracture plating system of FIG. 276A which may include passing the first tether into the first cap and first hex driver bit according to an embodiment of the present disclosure.

FIG. 293 is a perspective view of a step in a method of deploying the fracture plating system of FIG. 276A which may include passing the first tether end through the second angled driver according to an embodiment of the present disclosure.

FIG. 294 is a perspective view of a step in a method of deploying the fracture plating system of FIG. 276A which may include using the first tether to guide the first angled driver and the first cap to the first hole according to an embodiment of the present disclosure.

FIG. 295 is a perspective view of a step in a method of deploying the fracture plating system of FIG. 276A which may include using the first tether and second tether to draw the plate to an interior cavity according to an embodiment of the present disclosure.

FIG. 296 is a perspective view of a step in a method of deploying the fracture plating system of FIG. 276A which may include using the first tether and second tether to draw the plate to the subscapular rib fracture according to an embodiment of the present disclosure.

FIG. 297 is a perspective view of a step in a method of deploying the fracture plating system of FIG. 276A which may include using the first angled driver and the second angled driver to threadably engage the first cap and the second cap with the first fastener and the second fastener according to an embodiment of the present disclosure.

FIG. 298 is a perspective view of a step in a method of deploying the fracture plating system of FIG. 276A which may include using the first angled driver and the second angled driver to secure the first cap and the second cap to the first fastener and the second fastener according to an embodiment of the present disclosure.

FIG. 299 is a perspective view of a step in a method of deploying the fracture plating system of FIG. 276A which may include withdrawing the first tether, the second tether, the first angled driver, and the second angled driver according to an embodiment of the present disclosure.

FIG. 300 is a perspective view of a step in a method of deploying the fracture plating system of FIG. 276A which may include drilling a first hole and a second hole in the rib and passing a first tether and a second tether through the first hole and the second hole according to an embodiment of the present disclosure.

FIG. 301 is a perspective view of a step in a method of deploying the fracture plating system of FIG. 276A which may include passing the first tether and the second tether through a chest cavity to an exterior portion of the patient according to an embodiment of the present disclosure.

FIG. 302 is a perspective view of a step in a method of deploying the fracture plating system of FIG. 276A which may include passing a first tube over the first tether and a second tube over the second tether according to an embodiment of the present disclosure.

FIG. 303 is a perspective view of a step in a method of deploying the fracture plating system of FIG. 276A which may include advancing the first tube and the second tube to the first hole and the second hole according to an embodiment of the present disclosure.

FIG. 304 is a perspective view of a step in a method of deploying the fracture plating system of FIG. 276A which may include passing the first tube and the second tube through the first hole and the second hole according to an embodiment of the present disclosure.

FIG. 305 is a perspective view of a step in a method of deploying the fracture plating system of FIG. 276A which may include passing the first tube and the second tube to an exterior portion of the patient and withdrawing the first tether and the second tether according to an embodiment of the present disclosure.

FIG. 306 is a perspective view of a step in a method of deploying the fracture plating system of FIG. 276A which may include passing a third tether through a first fastener and a second fastener of the fracture plating system of FIG. 276A according to an embodiment of the present disclosure.

FIG. 307 is a perspective view of a step in a method of deploying the fracture plating system of FIG. 276A which may include positioning the third tether proximate the first tube and the second tube according to an embodiment of the present disclosure.

FIG. 308 is a perspective view of a step in a method of deploying the fracture plating system of FIG. 276A which may include passing the third tether through the first tube and the second tube according to an embodiment of the present disclosure.

FIG. 309A is a perspective view of a step in a method of deploying the fracture plating system of FIG. 276A which may include coupling a first hex driver bit to the first angled driver according to an embodiment of the present disclosure.

FIG. 309B is a perspective view of a step in a method of deploying the fracture plating system of FIG. 276A which may include coupling a first cap with the first hex driver bit according to an embodiment of the present disclosure.

FIG. 309C is a perspective view of the first cap coupled with the first hex driver bit according to an embodiment of the present disclosure.

FIG. 310 is a perspective view of a step in a method of deploying the fracture plating system of FIG. 276A which may include passing the third tether into a first cap and first hex driver bit according to an embodiment of the present disclosure.

FIG. 311 is a perspective view of a step in a method of deploying the fracture plating system of FIG. 276A which may include passing the third tether through the first angled driver according to an embodiment of the present disclosure.

FIG. 312 is a perspective view of a step in a method of deploying the fracture plating system of FIG. 276A which may include using the third tether to guide the first angled driver and the first cap to the first hole according to an embodiment of the present disclosure.

FIG. 313 is a perspective view of a step in a method of deploying the fracture plating system of FIG. 276A which may include using the third tether to draw the plate to an interior cavity according to an embodiment of the present disclosure.

FIG. 314 is a perspective view of a step in a method of deploying the fracture plating system of FIG. 276A which may include using the third tether to draw the plate to the subscapular rib fracture according to an embodiment of the present disclosure.

FIG. 315 is a perspective view of a step in a method of deploying the fracture plating system of FIG. 276A which may include using the first angled driver and the second angled driver to threadably engage and secure the first cap and the second cap with the first fastener and the second fastener according to an embodiment of the present disclosure.

FIG. 316 is a perspective view of a step in a method of deploying the fracture plating system of FIG. 276A which may include withdrawing the third tether, the first angled driver, and the second angled driver according to an embodiment of the present disclosure.

FIG. 317 is a perspective view of a fracture plating system deployed on an exemplary fracture of a rib according to an embodiment of the present disclosure.

FIG. 318 is a perspective view of the fracture plating system of FIG. 317.

FIG. 319 is a perspective view of a step in a method of deploying the fracture plating system of FIG. 317 which may include inserting a guide wire into the rib according to an embodiment of the present disclosure.

FIG. 320 is a perspective view of a step in a method of deploying the fracture plating system of FIG. 317 which may include advancing the guide wire through the rib according to an embodiment of the present disclosure.

FIG. 321 is a perspective view of the method step of FIG. 320.

FIG. 322 is a perspective view of a step in a method of deploying the fracture plating system of FIG. 317 which may include advancing a cannulated drill bit along the guide wire according to an embodiment of the present disclosure.

FIG. 323 is a perspective view of a step in a method of deploying the fracture plating system of FIG. 317 which may include using the cannulated drill bit to drill a hole through the rib according to an embodiment of the present disclosure.

FIG. 324 is a cross section view of the method step of FIG. 323.

FIG. 325 is a perspective view of a step in a method of deploying the fracture plating system of FIG. 317 which may include withdrawing the guide wire from the cannulated drill bit according to an embodiment of the present disclosure.

FIG. 326 is a perspective view of a step in a method of deploying the fracture plating system of FIG. 317 which may include positioning a tether proximate the cannulated drill bit according to an embodiment of the present disclosure.

FIG. 327A is a perspective view of a step in a method of deploying the fracture plating system of FIG. 317 which may include passing the tether through the cannulated drill bit and through the rib according to an embodiment of the present disclosure.

FIG. 327B is a cross section view of the method step of FIG. 327A.

FIG. 328 is a perspective view of a step in a method of deploying the fracture plating system of FIG. 317 which may include withdrawing the cannulated drill bit according to an embodiment of the present disclosure.

FIG. 329 is a perspective view of a step in a method of deploying the fracture plating system of FIG. 317 which may include passing the tether through a chest cavity to an exterior portion of the patient according to an embodiment of the present disclosure.

FIG. 330 is a perspective view of a step in a method of deploying the fracture plating system of FIG. 317 which may include positioning a rescue fastener proximate the tether according to an embodiment of the present disclosure.

FIG. 331 is a perspective view of a step in a method of deploying the fracture plating system of FIG. 317 which may include passing the tether through the rescue fastener according to an embodiment of the present disclosure.

FIG. 332 is a perspective view of a step in a method of deploying the fracture plating system of FIG. 317 which may include using the tether to guide the rescue fastener to the rib according to an embodiment of the present disclosure.

FIG. 333 is a perspective view of a step in a method of deploying the fracture plating system of FIG. 317 which may include using the tether to guide the rescue fastener through the hole according to an embodiment of the present disclosure.

FIG. 334 is a perspective view of a step in a method of deploying the fracture plating system of FIG. 317 which may include using the tether to guide the rescue fastener to engage the plate according to an embodiment of the present disclosure.

FIG. 335 is a perspective view of a step in a method of deploying the fracture plating system of FIG. 317 which may include using the tether to guide a cap to the rescue fastener according to an embodiment of the present disclosure.

FIG. 336 is a perspective view of a step in a method of deploying the fracture plating system of FIG. 317 which may include engaging the cap with the rescue fastener according to an embodiment of the present disclosure.

FIG. 337 is a perspective view of a step in a method of deploying the fracture plating system of FIG. 317 which may include withdrawing the tether according to an embodiment of the present disclosure.

FIG. 338 is a perspective view of the fracture plating system of FIG. 317 according to an embodiment of the present disclosure.

FIG. 339 is a perspective view of the fracture plating system of FIG. 338.

FIG. 340 is a perspective view of the fracture plating system of FIG. 338.

FIG. 341A is a perspective view of an expansion tool according to an embodiment of the present disclosure.

FIG. 341B is a top view of a plate in an unexpanded configuration according to an embodiment of the present disclosure.

FIG. 341C is a top view of the plate of FIG. 341B in the unexpanded configuration and the expansion tool of FIG. 341A.

FIG. 341D is a top view of the plate of FIG. 341B in an expanded configuration and the expansion tool of FIG. 341A according to an embodiment of the present disclosure.

FIG. 341E is a top view of the plate of FIG. 341B in the unexpanded configuration, the expansion tool of FIG. 341A, and a fastener according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis ofa body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to fracture plating devices, systems, and methods. Those skilled in the art will recognize that the following description is merely illustrative of the principles of the technology, which may be applied in various ways to provide many alternative embodiments. The present disclosure illustrates devices for plating systems for one or more fractures of a rib for the purposes of illustrating the concepts of the present design. However, it will be understood that other variations and uses are contemplated which may include, but not limited to, fractures of metatarsals, fractures of phalanges, metacarpals, and carpals, fractures of a fibula, fractures of an ulna, and other bone fractures.

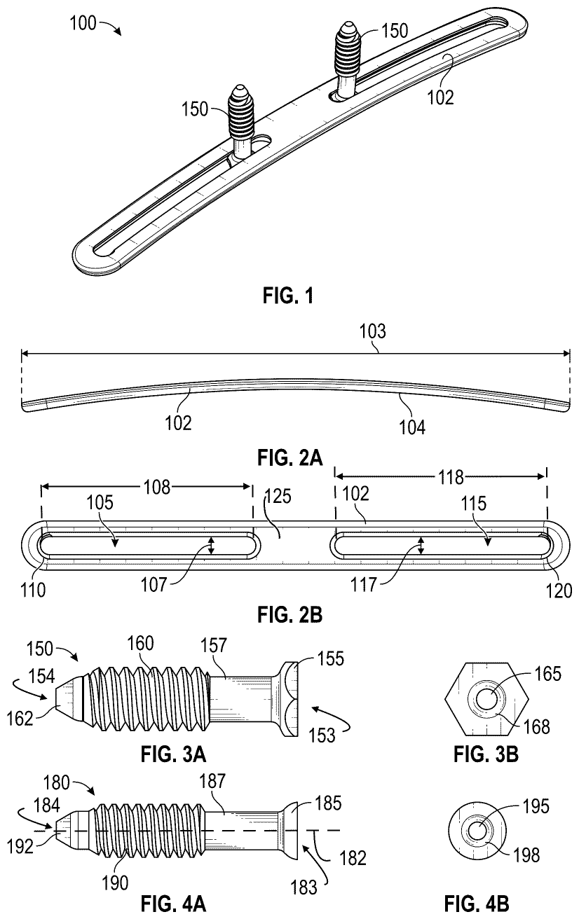

FIG. 1 is a perspective view of a fracture plating system 100 according to an embodiment of the present disclosure. The fracture plating system 100 may be configured to stabilize bone fractures through intra-thoracic plating. The fracture plating system 100 may be configured to be secured to an interior surface of a bone, an exterior surface of a bone, or both an interior surface of a bone and an exterior surface of a bone. The fracture plating system 100 may beneficially require a smaller incision in the chest of a patient as compared to a plating system that is only configured to be secured to an exterior surface of a bone. The fracture plating system 100 may be configured to be introduced into an intra-thoracic cavity, or interior cavity, through one or more portals, commonly used for Video-assisted Thoracic Surgery (VATS) and/or a thoracoscopic procedure. Additionally, or alternatively, the fracture plating system 100 may be configured to be introduced into an intra-thoracic cavity, or interior body cavity, using a trans-intercostal approach. The trans-intercostal approach may include a surgical technique that involves making incisions between two adjacent ribs to access the thoracic cavity, or interior cavity.

The fracture plating system 100 may include a plate and a plurality of fasteners. The plates may be one of a set of differently sized implants. The fasteners may be of a set of differently sized implants. The fracture plating system 100 may be modular in that a specific sized plate may be chosen by a user based on patient anatomy and quantity and location of fractures. Additionally, a plurality of fasteners may be selected by a user based on patient anatomy and quantity and location of fractures. The selection of fasteners may include fasteners of the same or different lengths and/or diameters. After a selection is made, the plate and plurality of fasteners may be assembled by a user, for example, during a surgical procedure, to best address a specific patient's indications. The fracture plating system 100 may be configured to achieve the fixation of a fracture plate to a portion of bone using a single tether to pull the plate against the bone.

The fracture plating system 100 may include a plate 102, an anti-rotation fastener 150 and/or a circular head fastener 180, a locking cap 30, and a washer 50. FIG. 2A is a front view of a plate 102 of the fracture plating system 100 according to an embodiment of the present disclosure. FIG. 2B is a bottom view of the plate 102. The plate 102 may be configured to stabilize and/or facilitate reduction of a fracture as a component of the fracture plating system 100. The plate 102 may include a plate length 103 and a plate radius 104. The plate length 103 may be configured to span one or more fractures.

The plate 102 may be configured such that the plate length 103 is within a range of lengths from 30 mm to 300 mm. The plate 102 may be one of a set of differently-sized implants, each having a different plate length 103. The plate radius 104 may generally match a contour of an interior surface of a rib. The plate 102 may further be one of a set of differently-sized implants, each having a different plate radius 104.

The plate 102 may further include a central portion 125 and a first slot 105 extending along a longitudinal axis of the plate 102 and having a first slot width 107, a first slot length 108, and a first threaded feature 110. The plate 102 may also include a second slot 115 extending along a longitudinal axis of the plate 102 and having a second slot width 117, a second slot length 118, and a second threaded feature 120. The central portion 125 may be generally in the center of the plate 102 and may separate the first slot 105 and the second slot 115.

The plate 102 may be fabricated from titanium alloy, titanium, stainless steel, cobalt-chrome, PEEK, PEAK, UHMWPE, a resorbable polymer, or any other biocompatible material with sufficient tensile strength.

The first slot width 107 and the second slot width 117 may each be configured to receive an anti-rotation fastener 150 and/or a circular head fastener 180. The first slot width 107 and the second slot width 117 may further be configured to allow translation of the anti-rotation fastener 150 and/or circular head fastener 180 along the length of the first slot and the second slot respectively.

FIG. 3A is a front view of an anti-rotation fastener 150 of the fracture plating system 100 according to an embodiment of the present disclosure. FIG. 3B is a side view of the anti-rotation fastener 150. The anti-rotation fastener 150 may be configured to secure the plate 102 to a bone portion. The anti-rotation fastener 150 may also be configured to be captively received within the plate 102 while still being allow to translate along the length of the first slot 105 and/or the second slot 115. The anti-rotation fastener 150 may be configured so that, when a head portion 155 engages the first slot 105 and/or the second slot 115, the anti-rotation fastener 150 is prevented from rotating in relation to the plate 102.

The anti-rotation fastener 150 may include a shank portion 157, a threaded portion 160, a tip portion 162, a cannulation 165, and a rounded edge 168. The anti-rotation fastener 150 may further include a proximal end 153 which may include a head portion 155, and a distal end 154 opposite the proximal end 153. The head portion 155 may be configured to be received within the first slot 105 and/or the second slot 115. The head portion 155 may lack protruding features that are configured to engage with the plate 102. The threaded portion 160 may be larger than a width of the first slot 105 and/or the second slot 115 to prevent the anti-rotation fastener 150 from disengaging from the plate 102. The first threaded feature 110 and the second threaded feature 120 may each be configured to threadably engage the threaded portion 160 to allow the threaded portion 160 to pass through the first threaded feature 110 and the second threaded feature 120 respectively.

The shank portion 157 may be smaller than a width of the first slot 105 and/or the second slot 115 to allow the anti-rotation fastener 150 to translate along the length of the first slot 105 and/or the second slot 115 respectively. The tip portion 162 may be configured to ease insertion of the anti-rotation fastener 150 into a hole in a bone portion. The cannulation 165 may be configured to slidably receive a tether 60. The rounded edge 168 may reduce stress on the tether 60 when the tether 60 exerts a force on the anti-rotation fastener 150.

FIG. 4A is a front view of a circular head fastener 180 of the fracture plating system 100 according to an embodiment of the present disclosure. FIG. 4B is a side view of the circular head fastener 180. The circular head fastener 180 may be configured to secure the plate 102 to a bone portion. The circular head fastener 180 may also be configured to be captively received within the plate 102 while still being allow to translate along the length of the first slot 105 and/or the second slot 115. The circular head fastener 180 may be configured so that, when a head portion 185 engages the first slot 105 and/or the second slot 115, the circular head fastener 180 is not prevented from rotating in relation to the plate 102.

The circular head fastener 180 may include a shank portion 187, a threaded portion 190, a tip portion 192, a cannulation 195, and a rounded edge 198. The circular head fastener 180 may include a proximal end 183 which may include a head portion 185, and a distal end 184 opposite the proximal end 183. The head portion 185 may be configured to be received within the first slot 105 and/or the second slot 115. The threaded portion 190 may be larger than a width of the first slot 105 and/or the second slot 115 to prevent the circular head fastener 180 from disengaging from the plate 102.

The shank portion 187 may be smaller than a width of the first slot 105 and/or the second slot 115 to allow the circular head fastener 180 to translate along the length of the first slot 105 and/or the second slot 115 respectively. The tip portion 192 may be configured to ease insertion of the circular head fastener 180 into a hole in a bone portion. The cannulation 195 may be configured to slidably receive a tether 60. The rounded edge 198 may reduce stress on the tether 60 when the tether 60 exerts a force on the circular head fastener 180.

The anti-rotation fastener 150 and/or the circular head fastener 180 may be configured within a range of lengths from 5 mm to 30 mm and within a range of diameters from 3 mm to 8 mm. The anti-rotation fastener 150 and/or the circular head fastener 180 may be one of a set of differently-sized implants, each having a different length and/or diameter.

The fracture plating system 100 may further include two or more locking caps 30 and a two or more washers 50. The locking cap 30 may be configured to threadably engage the anti-rotation fastener 150 and/or the circular head fastener 180 to secure the plate 102 to a portion of a rib. The locking cap 30 may be configured to receive the anti-rotation fastener 150 and/or the circular head fastener 180 and to cooperate with the anti-rotation fastener 150 and/or the circular head fastener 180 to secure the plate 102 to the bone. The washer 50 may be configured to be place between the locking cap 30 and a surface of a portion of a rib. The washer 50 may reduce damage to the surface of the portion of the rib due to rotation of the locking cap 30. Additionally, or alternatively, the washer 50 may provide a greater contact area with the surface of the portion of the rib resulting in greater securing of the plate 102 with the portion of the rib.

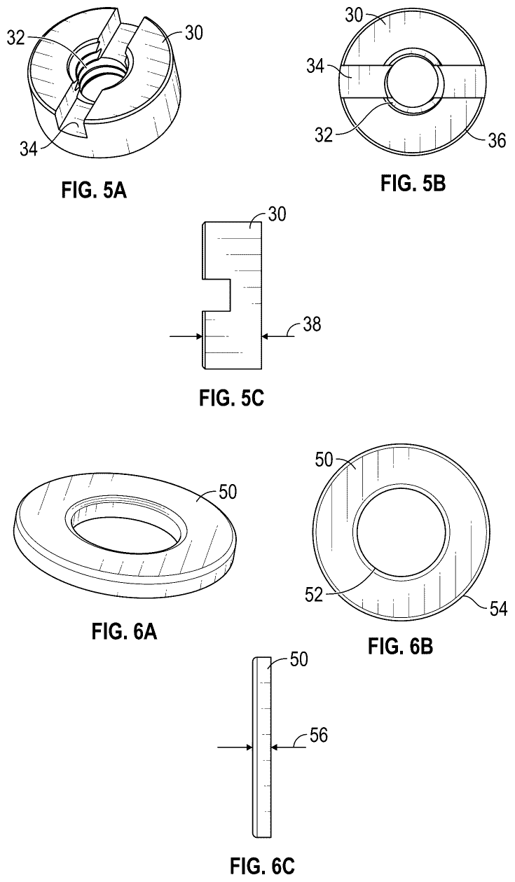

FIG. 5A is a perspective view of a locking cap 30 of the fracture plating system 100 according to an embodiment of the present disclosure. FIG. 5B is a top view of the locking cap 30 and FIG. 5C is a side view of the locking cap 30. The locking cap 30 may include a threaded portion 32, a slot 34, an outside diameter 36, and a thickness 38. The threaded portion 32 may be configured to threadably engage the threaded portion 160 of the anti-rotation fastener 150 and/or the threaded portion 190 of the circular head fastener 180. The slot 34 may allow engagement of a driver (not shown) to threadably engage the locking cap 30 with the anti-rotation fastener 150 and/or the circular head fastener 180.

The outside diameter 36 may be larger than a hole sized to receive the anti-rotation fastener 150 and/or the circular head fastener 180. The thickness 38 may be configured so that there is sufficient thread engagement to secure the plate 102 to the portion of the bone.

FIG. 6A is a perspective view of a washer 50 of the fracture plating system 100 according to an embodiment of the present disclosure. FIG. 6B is a top view of the washer 50 and FIG. 6C is a side view of the washer 50. The washer 50 may include an inside diameter 52, an outside diameter 54, and a thickness 56. The inside diameter 52 may be configured to slidably receive the threaded portion 160 of the anti-rotation fastener 150 and/or the threaded portion 190 of the circular head fastener 180. The outside diameter 54 may be configured to be greater than or equal to than the outside diameter 36 of the locking cap 30.

The thickness 56 may be configured so that when the washer 50 is placed between the surface of the portion of the rib and the locking cap 30, a sufficient length of the threaded portion 160 of the anti-rotation fastener 150 and/or the threaded portion 190 of the circular head fastener 180 is exposed to allow sufficient thread engagement between the locking cap 30 and the anti-rotation fastener 150 and/or the circular head fastener 180 to secure the plate 102 to the portion of the rib.

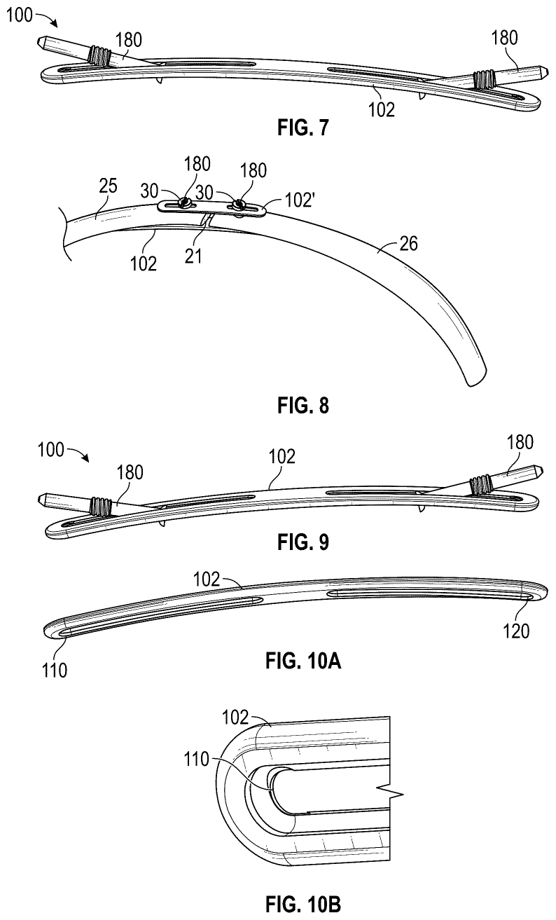

FIG. 7 is a perspective view of the fracture plating system 100 in a lower profile configuration. The fracture plating system 100 may advantageously be configured so that, when the anti-rotation fastener 150 and/or the circular head fastener 180 are captive received within the first slot 105 and/or the second slot 115, the anti-rotation fastener 150 and/or the circular head fastener 180 may be flattened against the plate 102 to make the construct lower profile and easier to insert into a patient.

FIG. 8 is a perspective view of the fracture plating system of 100 spanning an exemplary first fracture 21. The fracture plating system 100 may include a second plate 102′, securable to an exterior surface of a rib. The same fasteners may engage both a first plate 102 and a second plate 102′.

FIG. 9 is a perspective view of the fracture plating system 100 in a lower profile configuration. FIG. 10A is a perspective view of the plate 102 of the fracture plating system of 100 according to an embodiment of the present disclosure. FIG. 10B is a top perspective partial view of the plate 102. The first threaded feature 110 and the second threaded feature 120 may each be configured to threadably engage the threaded portion 190 to allow the threaded portion 190 to pass through the first threaded feature 110 and the second threaded feature 120 respectively. The circular head fastener 180 may be further configured so that, with the circular head fastener 180 captively received in the first slot 105 and/or the second slot 115, the circular head fastener 180 may be moveable relative to the plate 102 along a longitudinal axis 182 of the circular head fastener 180.

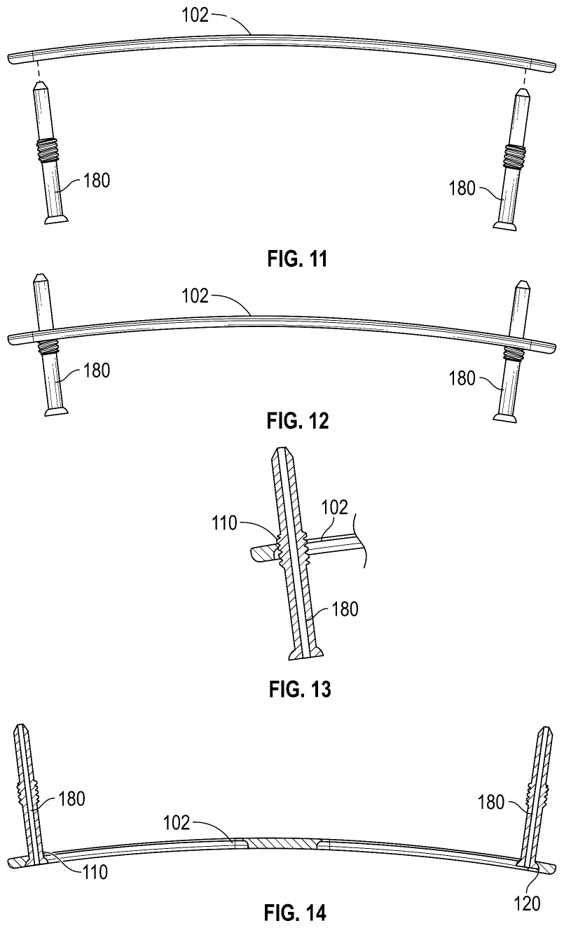

FIG. 11 is a front view of the fracture plating system 100 in a first pre-assembled configuration. The anti-rotation fastener 150 and/or the circular head fastener 180 may be aligned with the first threaded feature 110 and/or the second threaded feature 120 of the plate 102.

FIG. 12 is a front view of the fracture plating system of 100 in a second pre-assembled configuration. The tip portion 162 of the anti-rotation fastener 150 and/or the tip portion 192 of the circular head fastener 180 may be slidably received within the first threaded feature 110 and/or the second threaded feature 120 of the plate 102.

FIG. 13 is a front partial section view of the fracture plating system 100. The threaded portion 160 of the anti-rotation fastener 150 and/or the threaded portion 190 of the circular head fastener 180 may threadably engage the first threaded feature 110 and/or the second threaded feature 120 of the plate 102.

FIG. 14 is a front section view of the fracture plating system 100 in a third pre-assembled configuration. The head portion 155 of the anti-rotation fastener and/or the head portion 185 of the circular head fastener 180 may be configured to not pass through the first threaded feature 110 and/or the second threaded feature 120 of the plate 102 thereby capturing the anti-rotation fastener 150 and/or the circular head fastener 180 within the first slot 105 and/or the second slot 115 of the plate 102.

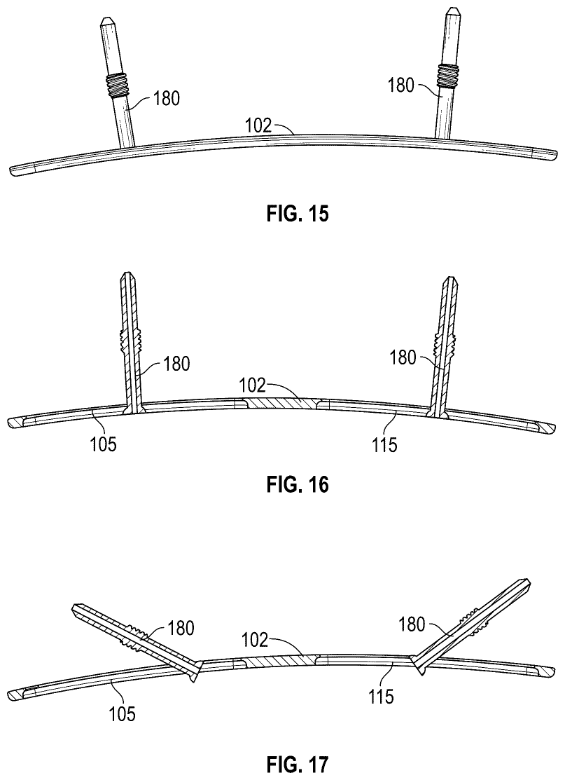

FIG. 15 is a front view of the fracture plating system of 100 in an assembled configuration. FIG. 16 is a front section view of the fracture plating system 100 in an assembled configuration. In the assembled configuration, the fracture plating system 100 may include a plate assembly which may include a plate 102, a first fastener, and a second fastener, wherein each of the first fastener and the second fastener may be chosen from the anti-rotation fastener and/or the head portion 185 of the circular head fastener 180. The shank portion 157 of the anti-rotation fastener 150 and/or the shank portion 187 of the circular head fastener 180 may be configured to allow the anti-rotation fastener 150 and/or the circular head fastener 180 to translate along the first slot 105 and/or the second slot 115 of the plate 102.

FIG. 17 is a front section view of the fracture plating system 100 in a lower profile configuration. The plate 102, and the anti-rotation fastener 150 and/or the circular head fastener 180 may be configured so that the anti-rotation fastener 150 and/or the circular head fastener 180 may be flattened against the plate 102 to make the construct lower profile and easier to insert into an intra-thoracic cavity of a patient. The anti-rotation fastener 150 and/or the circular head fastener 180 may be configured to reside captive within the first slot 105 and/or the second slot 115 independently of engagement of any protruding feature of the head portion 155 and/or the head portion 185 with the plate 102.

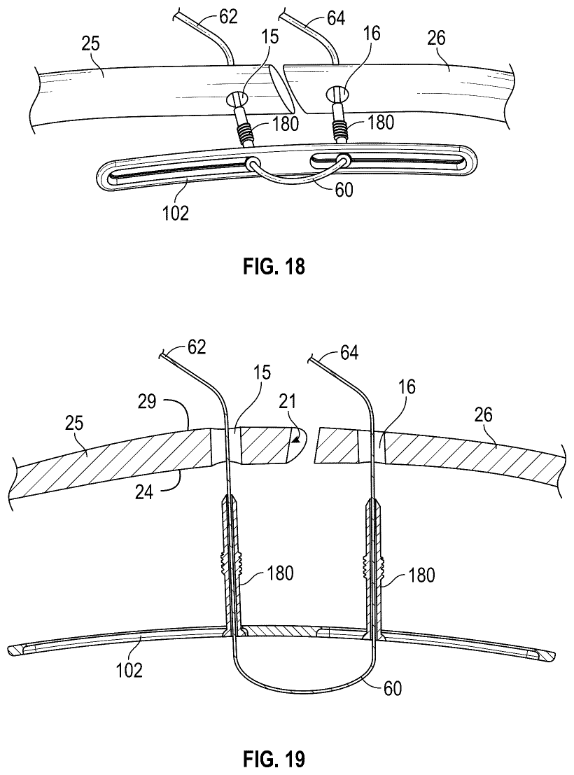

FIG. 18 is a perspective view of the fracture plating system 100 in a partially deployed configuration spanning an exemplary first fracture 21. FIG. 19 is a front section view of the fracture plating system 100 in a partially deployed configuration spanning an exemplary first fracture 21. The bone may include an interior surface 24 facing toward an interior body cavity of a patient, and an exterior surface 29 facing away from the interior body cavity of the patient.

The cannulation 165 of the anti-rotation fastener 150 and/or the cannulation 195 of the circular head fastener 180 may be configured to slidably receive a tether 60. A tether 60 may be configured to apply a force directly to one or more fasteners to guide the one or more fasteners to the interior surface of the bone and through a hole in the interior surface of the bone.

A circular head fastener 180 length may be chosen from a range of fastener lengths such that the threaded portion 190 exposed beyond the exterior surface of the portion of bone may be greater than or equal to the thickness 38 of the locking cap 30. Alternatively, a circular head fastener 180 length may be chosen from a range of fastener lengths such that the threaded portion 190 exposed beyond the exterior surface of the portion of bone may be greater than or equal to the thickness 38 of the locking cap 30 plus the thickness 56 of the washer 50.

The tether 60 may be configured to guide the anti-rotation fastener 150 and/or the circular head fastener 180 through a first hole 15 and a second hole 16 from an interior side of a portion of a bone to an exterior side of a portion of a bone. The first hole 15 and the second hole 16 may be located on opposite sides of a first fracture 21. The distance between the first hole 15 and the second hole 16 may be greater than the central portion 125 of the plate 102.

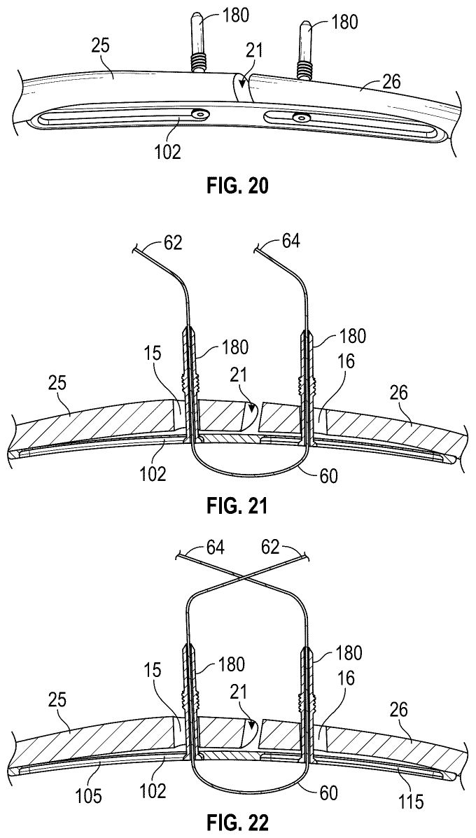

FIG. 20 is a perspective view of the fracture plating system 100 in a partially deployed configuration spanning an exemplary first fracture 21. FIG. 21 is a front section view of the fracture plating system 100 in a partially deployed configuration spanning an exemplary first fracture 21. The tether 60 may be configured to pull the plate 102 against the interior side of a portion of a bone. The tether 60 may further be utilized to hold the plate 102 and the anti-rotation fastener 150 and/or the circular head fastener 180 in position so that the washer 50 and/or the locking cap 30 may be secured to the anti-rotation fastener 150 and/or the circular head fastener 180 to secure the fracture plating system 100 in place.

The fracture plating system 100 and the tether 60 may be configured so that a single tether 60 may be used to guide two fasteners through two holes in a portion of bone. The fracture plating system 100 and the tether 60 may further be configured so that a single tether 60 may be used to pull the plate 102 against an interior surface of a fractured rib.

FIG. 22 is a front section view of the fracture plating system 100 in a partially deployed configuration spanning an exemplary first fracture 21. The tether 60 may include a first tether end 62 and a second tether end 64. The first tether end 62 may configured to be advanced through the cannulation 195 of a first circular head fastener 180 and the second tether end 64 may be configured to be advanced through the cannulation 195 of a second circular head fastener 180.

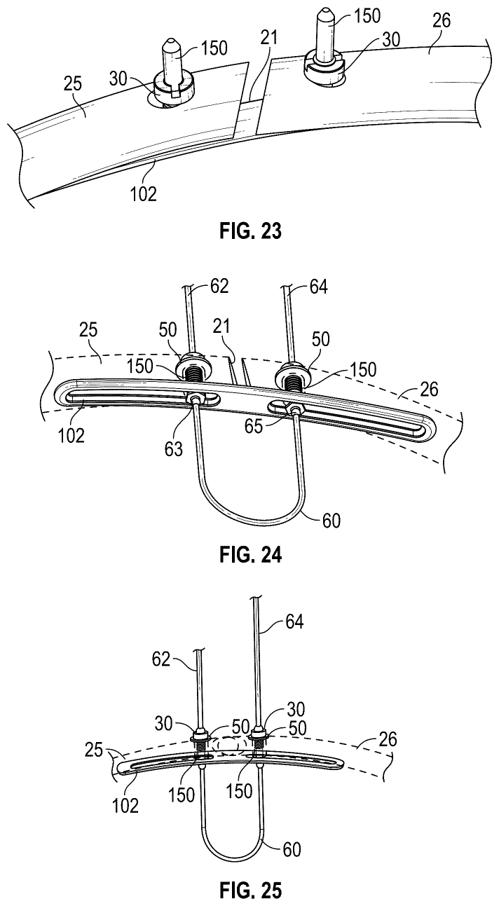

FIG. 23 is a perspective view of the fracture plating system 100 in a partially deployed configuration spanning an exemplary first fracture 21. The first tether end 62 may be pulled in the direction of the second circular head fastener 180 and the second tether end 64 may be pulled in the direction of the first circular head fastener, thereby applying compression to the first fracture 21 to facilitate reduction of the first fracture 21 by urging the first fastener and the second fastener towards each other. A locking cap 30 may be secured to each of the first and second circular head fastener 180 while compression is being applied to the fracture to secure the fracture plating system 100 in place.

FIG. 24 is a perspective view of the fracture plating system 100 in a partially deployed configuration spanning an exemplary first fracture 21. FIG. 25 is a front view of the fracture plating system 100 in a partially deployed configuration spanning an exemplary first fracture 21. The tether 60 may include a first bead 63 and a second bead 65. The first bead 63 and the second bead 65 may be configured to engage the head portion 155 of the anti-rotation fastener 150 and/or the head portion 185 of the circular head fastener 180. The first bead 63 and the second bead 65 may be configured as a solid spherical section of larger diameter.

Additionally, the first bead 63 and the second bead 65 may be larger than the cannulation 165 of the anti-rotation fastener 150 and/or the cannulation 195 of the circular head fastener 180. The first bead 63 and the second bead 65 may not be received within the cannulation 165 of the anti-rotation fastener 150 and/or the cannulation 195 of the circular head fastener 180.

The first bead 63, the second bead 65, and the anti-rotation fastener and/or the circular head fastener 180 may be configured so that, when the first bead 63 engages a first fastener, a tension force applied to the first tether end 62 is translated as a compression force applied to the first fastener. Similarly, when the second bead 65 engages a second fastener, a tension force applied to the second tether end 64 is translated as a compression force applied to the second fastener.

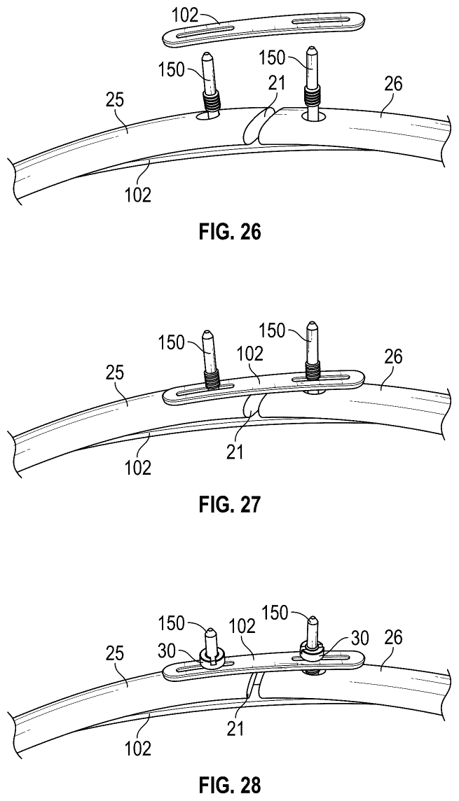

FIG. 26 is a perspective view of the fracture plating system 100 in a partially deployed configuration spanning an exemplary first fracture 21. In an embodiment, the fracture plating system 100 may include a second plate 102′. The second plate 102′ may be configured as an outer buttress plate. The second plate 102′ may be placed on an exterior surface of bone.

FIG. 27 is a perspective view of the fracture plating system 100 in a partially deployed configuration spanning an exemplary first fracture 21. The second plate 102′ may span a first fracture 21 and may be secured to a portion of bone using the same fasteners that are used to secure the plate 102 to the interior surface of a bone.

FIG. 28 is a perspective view of the fracture plating system 100 in a deployed configuration spanning an exemplary first fracture 21. The second plate 102′ may be secured to the exterior surface of bone with one or more locking caps 30. An anti-rotation fastener 150 length may be chosen from a range of fastener lengths such that, which the second plate 102′ proximate the exterior surface of the portion of bone, the threaded portion 160 exposed beyond the second plate 102′ may be greater than or equal to the thickness 38 of the locking cap 30. Alternatively, an anti-rotation fastener 150 length may be chosen from a range of fastener lengths such that, which the second plate 102′ proximate the exterior surface of the portion of bone, the threaded portion 160 exposed beyond the second plate 102′ may be greater than or equal to the thickness 38 of the locking cap 30 plus the thickness 56 of the washer 50.

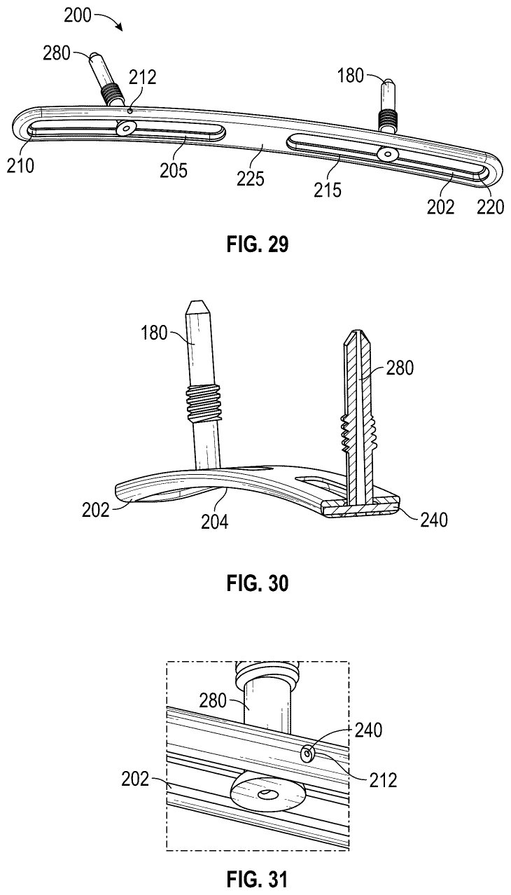

FIG. 29 is a perspective view of a fracture plating system 200 according to an embodiment of the present disclosure. The fracture plating system 200 may include similar features as the fracture plating system 100 previously described. The fracture plating system 200 may include a fixed hinge fastener. The fixed hinge fastener may be configured to provide a lagging effect to facilitate compression of a fracture. The fracture plating system 200 may include a first fastener in a fixed location within a first slot of a plate and a second fastener that may be translated within a second slot of a plate.

The fracture plating system 200 may include a first fastener configured as one of a fixed hinge fastener 250 and a circular head fixed hinge fastener 280. And a second fastener configured as one of an anti-rotation fastener 150 and a circular head fastener 180. The first fastener may be captively received within a first slot 205 of a plate 202 so that the first fastener may be coupled to the first slot. The second fastener may be captively received within a second slot 215 of the plate 202 so that the second fastener may be coupled to the second slot.

The fracture plating system 200 may include a plate 202. The plate 202 may include a plate radius 204, a first slot 205, a second slot 215, a pin aperture 212, and a central portion 225. The first slot 205 may include a first threaded feature 210 and the second slot 215 may include a second threaded feature 220. The central portion 225 may be generally in the center of the plate 202 and may separate the first slot 205 and the second slot 215.

The first threaded feature 210 may be configured to threadably engage a threaded portion 260 of the fixed hinge fastener 250 and/or a threaded portion 290 of the circular head fixed hinge fastener 280 to allow the threaded portion 260 and/or the threaded portion 290 to pass through the first slot 205. The second threaded feature 220 may be configured to threadably engage a threaded portion 160 of the anti-rotation fastener 150 and/or a threaded portion 190 of the circular head fastener 180 to allow the threaded portion 160 and/or the threaded portion 190 to pass through the second slot 215.

The plate 202 may be configured such that a plate length is within a range of lengths from 30 mm to 300 mm. The plate 202 may be one of a set of differently-sized implants, each having a different plate length. The plate radius 204 may generally match a contour of an interior surface of a rib. The plate 202 may further be one of a set of differently-sized implants, each having a different plate radius 204.

FIG. 30 is a perspective section view of the fracture plating system 200. FIG. 31 is a partial bottom perspective view of the fracture plating system 200. The pin aperture 212 may span the width of the plate 202 and may be located within the first slot 205. The pin aperture 212 may further be configured to receive at least one hinge pin 240. The pin aperture 212 may be configured to receive the at least one hinge pin 240 as a press fit so that the at least one hinge pin 240 may be held in place within the pin aperture 212. Additionally, or alternatively, the at least one hinge pin 240 may be secured to the plate 202 within the pin aperture 212 by welding, solder, adhesive, or other suitable means.

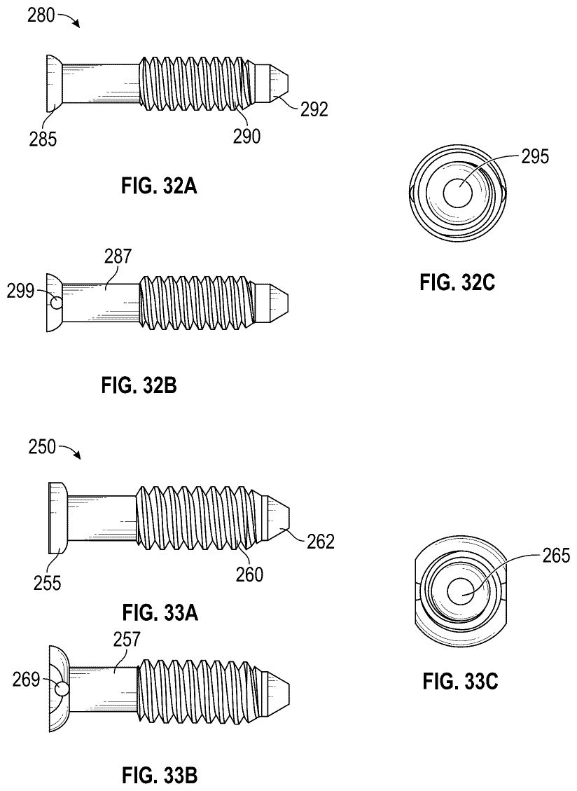

FIG. 32A is a top view of a circular head fixed hinge fastener 280 of the fracture plating system 200 according to an embodiment of the present disclosure. FIG. 32B is a front view of the circular head fixed hinge fastener 280. FIG. 32C is a side view of the circular head fixed hinge fastener 280. The circular head fixed hinge fastener 280 may include a head portion 285, a shank portion 287, a threaded portion 290, a tip portion 292, a cannulation 295, and a pin aperture 299.

The head portion 285 may be configured to be received within the first slot 205. The threaded portion 290 may be larger than a width of the first slot 205 to prevent the circular head fixed hinge fastener 280 from disengaging from the plate 202. The head portion 285 may include the pin aperture 299. The pin aperture 299 may be configured to slidably receive at least one hinge pin 240. The at least one hinge pin 240 may also be received within the pin aperture 212 and may prevent translation of the circular head fixed hinge fastener 280 within the first slot 205.

The shank portion 287 may be smaller than a width of the first slot 205 to allow the circular head fixed hinge fastener 280 to translate along the length of the first slot 205. The tip portion 292 may be configured to ease insertion of the circular head fixed hinge fastener 280 into a hole in a bone portion. The cannulation 295 may be configured to slidably receive a tether 60.

The at least one hinge pin 240 may be configured to engage the pin aperture 212 of the plate 202 and a pin aperture 299 of the circular head fixed hinge fastener 280 so that the at least one hinge pin 240 is not received within the cannulation 295 and does not impede the tether 60 from passing through the cannulation 295.

The circular head fixed hinge fastener 280 may be configured within a range of lengths from 5 mm to 30 mm and within a range of diameters from 3 mm to 8 mm. The circular head fixed hinge fastener 280 may be one of a set of differently-sized implants, each having a different length and/or diameter.

FIG. 33A is a top view of a fixed hinge fastener 250 of the fracture plating system 200 according to an embodiment of the present disclosure. FIG. 33B is a front view of the fixed hinge fastener 250. FIG. 33C is a side view of the fixed hinge fastener 250. The fixed hinge fastener 250 may include a head portion 255, a shank portion 257, a threaded portion 260, a tip portion 262, a cannulation 265, and a pin aperture 269.

The head portion 255 may be configured to be received within the first slot 205. The threaded portion 260 may be larger than a width of the first slot 205 to prevent the fixed hinge fastener 250 from disengaging from the plate 202. The head portion 255 may include the pin aperture 269. The pin aperture 269 may be configured to slidably receive at least one hinge pin 240. The at least one hinge pin 240 may also be received within the pin aperture 212 and may prevent translation of the fixed hinge fastener 250 within the first slot 205.

The shank portion 257 may be smaller than a width of the first slot 205 to allow the fixed hinge fastener 250 to translate along the length of the first slot 205. The tip portion 262 may be configured to ease insertion of the fixed hinge fastener 250 into a hole in a bone portion. The cannulation 265 may be configured to slidably receive a tether 60.

The at least one hinge pin 240 may be configured to engage the pin aperture 212 of the plate 202 and a pin aperture 269 of the fixed hinge fastener 250 so that the at least one hinge pin 240 is not received within the cannulation 265 and does not impede the tether 60 from passing through the cannulation 265.

The fixed hinge fastener 250 may be configured within a range of lengths from 5 mm to 30 mm and within a range of diameters from 3 mm to 8 mm. The fixed hinge fastener 250 may be one of a set of differently-sized implants, each having a different length and/or diameter.

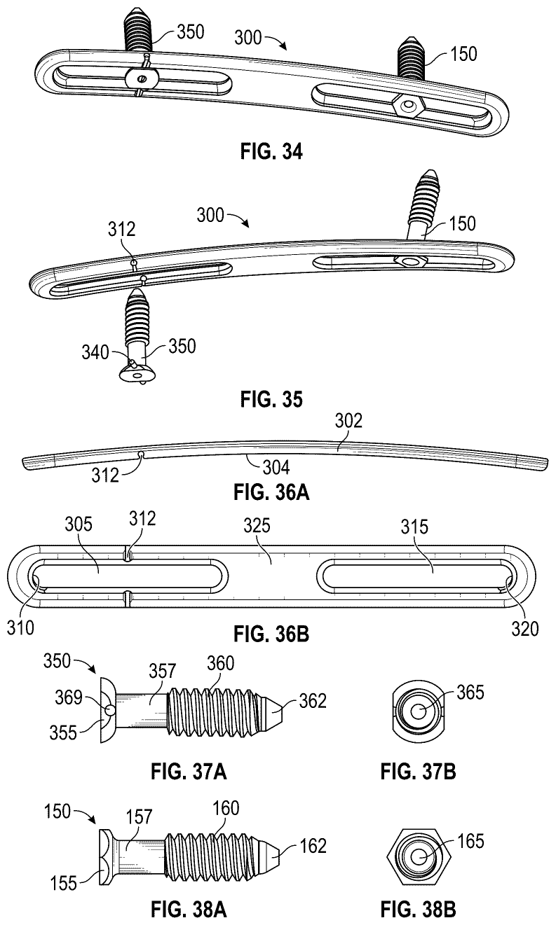

FIG. 34 is a perspective view of a fracture plating system 300 according to an embodiment of the present disclosure. The fracture plating system 300 may include similar features as the fracture plating system 100 and the fracture plating system 200 previously described. The fracture plating system 300 may include a fixed hinge fastener. The fixed hinge fastener may be configured to provide a lagging effect to facilitate compression of a fracture. The fracture plating system 300 may include a first fastener in a fixed location within a first slot of a plate and a second fastener that may be translated within a second slot of a plate.

The fracture plating system 300 may include a first fastener configured as a fixed hinge fastener 350. And a second fastener configured an anti-rotation fastener 150. The first fastener may be captively received within a first slot 305 of a plate 302. The second fastener may be captively received within a second slot 315 of the plate 302.

The fracture plating system 300 may include a plate 302. The plate 302 may include a plate radius 304, a first slot 305, a second slot 315, a pin channel 312, and a central portion 325. The first slot 305 may include a first threaded feature 310 and the second slot 315 may include a second threaded feature 320. The central portion 325 may be generally in the center of the plate 302 and may separate the first slot 305 and the second slot 315.

FIG. 35 is a bottom perspective view of the fracture plating system 300 in a pre-assembled configuration. The first threaded feature 310 may be configured to threadably engage a threaded portion 360 of the fixed hinge fastener 350 to allow the threaded portion 360 to pass through the first slot 305. The second threaded feature 320 may be configured to threadably engage a threaded portion 160 of the anti-rotation fastener 150 and/or a threaded portion 190 of the circular head fastener 180 to allow the threaded portion 160 and/or the threaded portion 190 to pass through the second slot 315.

The pin channel 312 may span the width of the plate 302 and may be located within the first slot 305. The pin channel 312 may further be configured to receive at least one hinge pin 340. The pin channel 312 may be configured to receive the at least one hinge pin 340 as a snap fit so that the at least one hinge pin 340 may be held in place within the pin channel 312. The pin channel 312 may be configured so that a fixed hinge fastener 350 may be snapped into the plate 302 during a surgical procedure, and may allow a user to tailor the length of the fixed hinge fastener 350 based on an anatomy of a patient while still having the benefits of a fixed fastener for applying compression to a fracture. The fixed hinge fastener 350, the plate 302, and the pin channel 312 may be configured so that the, when the hinge pin 340 is snapped into the pin channel 312, the fixed hinge fastener 350 may rotate about the hinge pin 340 but may not translate along the first slot 305.

FIG. 36A is a front view of a plate 302 of the fracture plating system 300 according to an embodiment of the present disclosure. FIG. 36B is a bottom view of the plate 302. The plate 302 may be configured such that a plate length is within a range of lengths from 30 mm to 300 mm. The plate 302 may be one of a set of differently-sized implants, each having a different plate length. A plate radius 304 may generally match a contour of an interior surface of a rib. The plate 302 may further be one of a set of differently-sized implants, each having a different plate radius 304.

FIG. 37A is a front view of a fixed hinge fastener 350 of the fracture plating system 300 according to an embodiment of the present disclosure. FIG. 37B is a side view of the fixed hinge fastener 350. FIG. 38A is a front view of the anti-rotation fastener 150 and FIG. 38B is a side view of the anti-rotation fastener 150. The fixed hinge fastener 350 may include a head portion 355, a shank portion 357, a threaded portion 360, a tip portion 362, a cannulation 365, and a pin aperture 369.

The head portion 355 may be configured to be received within the first slot 305. The threaded portion 360 may be larger than a width of the first slot 305 to prevent the fixed hinge fastener 350 from disengaging from the plate 302. The head portion 355 may include the pin aperture 369. The pin aperture 369 may be configured to slidably receive at least one hinge pin 340. The at least one hinge pin 340 may also be received within the pin channel 312 and may prevent translation of the fixed hinge fastener 350 within the first slot 305.

The shank portion 357 may be smaller than a width of the first slot 305 to allow the fixed hinge fastener 350 to translate along the length of the first slot 305. The tip portion 362 may be configured to ease insertion of the fixed hinge fastener 350 into a hole in a bone portion. The cannulation 365 may be configured to slidably receive a tether 60.

The at least one hinge pin 340 may be configured to engage the pin channel 312 of the plate 302 and a pin aperture 369 of the fixed hinge fastener 350 so that the at least one hinge pin 340 is not received within the cannulation 365 and does not impede the tether 60 from passing through the cannulation 365. The at least one hinge pin 340 may be fabricated as a separate component from the fixed hinge fastener 350. Alternatively, the at least one hinge pin 340 and the fixed hinge fastener 350 may be fabricated as a single integral component.

The fixed hinge fastener 350 may be configured within a range of lengths from 5 mm to 30 mm and within a range of diameters from 3 mm to 8 mm. The fixed hinge fastener 350 may be one of a set of differently-sized implants, each having a different length and/or diameter.

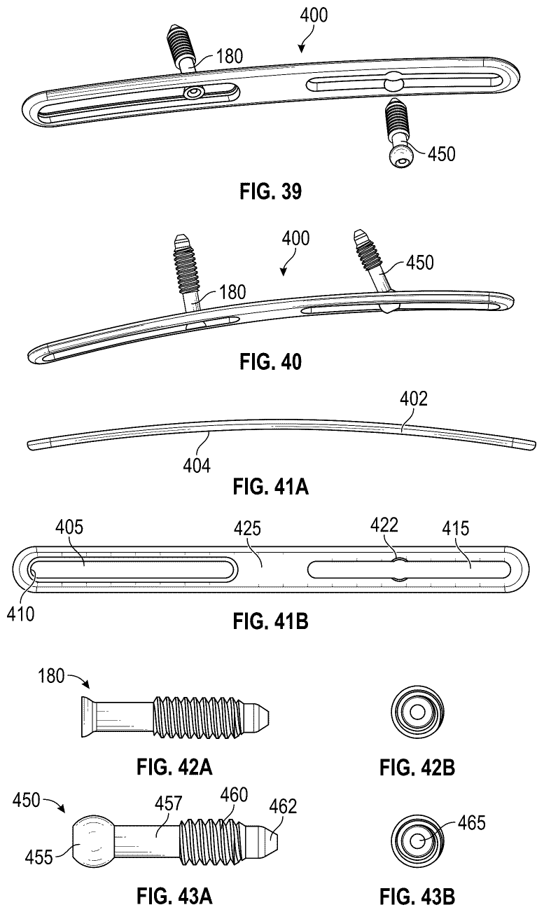

FIG. 39 is a perspective view of a fracture plating system 400 according to an embodiment of the present disclosure. The fracture plating system 400 may include similar features as the fracture plating system 100, the fracture plating system 200, and the fracture plating system 300 previously described. The fracture plating system 400 may include a ball-headed fastener 450. The ball-headed fastener 450 may be configured to provide a lagging effect and/or poly-axial rotation of the ball-headed fastener 450 to facilitate compression of a fracture. The fracture plating system 400 may include a first fastener that may be translated within a first slot of a plate and a second fastener in a fixed location within a first slot of a plate.

FIG. 40 is a bottom perspective view of the fracture plating system 400. The fracture plating system 400 may include a first fastener configured as a circular head fastener 180 and a second fastener configured as a ball-headed fastener 450. The first fastener may be captively received within a first slot 405 of a plate 402. The second fastener may be captively received within a second slot 415 of the plate 402.

FIG. 41A is a front view of a plate 402 of the fracture plating system 400 according to an embodiment of the present disclosure. FIG. 41B is a bottom view of the plate 402. The plate 402 may include a plate radius 404, a first slot 405, a second slot 415, a socket 422, and a central portion 425. The first slot 405 may include a first threaded feature 410 and the second slot 415 may include a socket 422. The central portion 425 may be generally in the center of the plate 402 and may separate the first slot 405 and the second slot 415.

The first threaded feature 410 may be configured to threadably engage a threaded portion 190 of the circular head fastener 180 to allow the threaded portion 190 to pass through the first slot 305.

The socket 422 may be located within the second slot 415. The socket 422 may further be configured to receive a head portion 455 of the ball-headed fastener 450. The socket 422 may be configured to receive the head portion 455 as a snap fit so that the head portion 455 may be held in place within the socket 422. The socket 422 may be configured so that a ball-headed fastener 450 may be snapped into the plate 402 during a surgical procedure, and may allow a user to tailor the length of the ball-headed fastener 450 based on an anatomy of a patient while still having the benefits of a fixed fastener for applying compression to a fracture. The ball-headed fastener 450, the plate 402, and the socket 422 may be configured so that the, when the head portion 455 is snapped into the socket 422, the ball-headed fastener 450 may rotate about the head portion 455 but may not translate along the second slot 415.

The plate 402 may be configured such that a plate length is within a range of lengths from 30 mm to 300 mm. The plate 402 may be one of a set of differently-sized implants, each having a different plate length. A plate radius 404 may generally match a contour of an interior surface of a rib. The plate 402 may further be one of a set of differently-sized implants, each having a different plate radius 404.

FIG. 42A is a front view of the circular head fastener 180 and FIG. 42B is a side view of the circular head fastener 180. FIG. 43A is a front view of a ball-headed fastener 450 of the fracture plating system 400 according to an embodiment of the present disclosure. FIG. 43B is a side view of the ball-headed fastener 450. The ball-headed fastener 450 may include a head portion 455, a shank portion 457, a threaded portion 460, a tip portion 462, and a cannulation 465.

The head portion 455 may be configured to be received within the socket 422 and may prevent translation of the ball-headed fastener 450 within the second slot 415. The shank portion 457 may be smaller than a width of the second slot 415 to allow the ball-headed fastener 450 to rotate about the head portion 455. The tip portion 462 may be configured to ease insertion of the ball-headed fastener 450 into a hole in a bone portion. The cannulation 465 may be configured to slidably receive a tether 60.

The ball-headed fastener 450 may be configured within a range of lengths from 5 mm to 30 mm and within a range of diameters from 3 mm to 8 mm. The ball-headed fastener 450 may be one of a set of differently-sized implants, each having a different length and/or diameter.

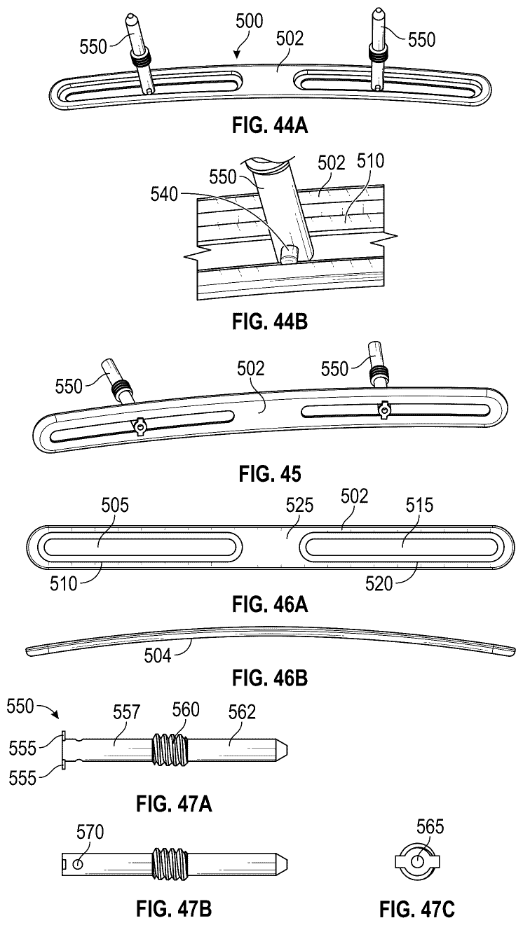

FIG. 44A is a perspective view of a fracture plating system 500 according to an embodiment of the present disclosure. FIG. 44B is a partial perspective view of the fracture plating system 500. The fracture plating system 500 may include similar features as other fracture plating systems previously described within the present disclosure. The fracture plating system 500 may include a pin fastener. The pin fastener may be configured to translate along a length of a slot in a plate to facilitate compression of a fracture. The fracture plating system 500 may include a first fastener and a second fastener that each may be translated within a slot of a plate.

FIG. 45 is a bottom perspective view of the fracture plating system 500. The fracture plating system 500 may include a first fastener configured as a pin fastener 550. And a second fastener configured as a pin fastener 550. The first fastener and the second fastener may be captively received within a first slot 505 and a second slot 515 of a plate 502, respectively.

FIG. 46A is a top view of a plate 502 of the fracture plating system 500 according to an embodiment of the present disclosure. FIG. 46B is a front view of the plate 502. The plate 502 may include a plate radius 504, a first slot 505, a second slot 515, and a central portion 525. The first slot 505 may include a first pocket 510 and the second slot 515 may include a second pocket 520. The central portion 225 may be generally in the center of the plate 202 and may separate the first slot 205 and the second slot 215.

The first pocket 510 and the second pocket 520 may be configured to receive a pin 540 received within a pin fastener 550. The plate 502 may be one of a set of differently-sized implants, each having a different plate length. A plate radius 504 may generally match a contour of an interior surface of a rib. The plate 502 may further be one of a set of differently-sized implants, each having a different plate radius 504.

FIG. 47A is a top view of a pin fastener 550 of the fracture plating system 500 according to an embodiment of the present disclosure. FIG. 47B is a front view of the pin fastener 550 and FIG. 47C is a side view of the pin fastener 550. The pin fastener may include a tab portion 555, a shank portion 557, a threaded portion 560, a tip portion 562, a cannulation 565, and a pin aperture 570.

The pin aperture 570 may be configured to securably receive at least one pin 540 such that, when the pin fastener 550 is assembled with the plate 502, the at least one pin 540 is received within one of the first pocket 510 and the second pocket 520. The tab portion 555 may be configured so that, when the at least one pin 540 is received with one of the first pocket 510 and the second pocket 520 and is aligned generally perpendicular to a long axis of the plate 502, the tab portion 555 engages a bottom surface of the plate 502 to hold the pin fastener 550 captively received within one of the first slot 505 and the second slot 515 while allowing the pin fastener 550 to translate along a length of one of the first slot 505 and the second slot 515.

The shank portion 557 may be smaller than a width of the first slot 505 and/or the second slot 515 to allow the pin fastener 550 to translate along the length of the first slot 505 and/or the second slot 515. The tip portion 562 may be configured to ease insertion of the pin fastener 550 into a hole in a bone portion. The cannulation 565 may be configured to slidably receive a tether 60. The threaded portion 560 may be configured to threadably receive a locking cap 30.

The at least one pin 540 may be configured to be received within the first pocket 510 and/or the second pocket 520 of the plate 502 and a pin aperture 570 of the pin fastener 550 so that the at least one pin 540 is not received within the cannulation 565 and does not impede the tether 60 from passing through the cannulation 565.

The pin fastener 550 may be configured within a range of lengths from 5 mm to 30 mm and within a range of diameters from 3 mm to 8 mm. The pin fastener 550 may be one of a set of differently-sized implants, each having a different length and/or diameter.

Each of the previously described fracture plating systems of the present disclosure may be utilized to treat multiple fractures of a single portion of bone. Each of the previously described fracture plating systems of the present disclosure may be assemblable in a modular fashion whereby a plate may be selectable from a range of differently sized plates, a plurality of fasteners may be selectable from a range of differently sized fasteners, and a locking cap may be selectable from a range of differently sized locking caps. The fracture plating system may be configured to be assembled prior to implantation within a patient. The fracture plating system may be configured to be assembled on a back table during a surgical procedure prior to implantation within a patient. The fracture plating system may be configured so that a plate, two or more fasteners, and two or more locking caps may be selected based on patient anatomy and/or the location and/or severity of one or more fractures. Additionally, or alternatively, the fracture plating system may be configured so that a plate, two or more fasteners, and two or more locking caps may be selected based surgical and/or radiographic assessment of the patient and/or the location and/or severity of the one or more fractures.

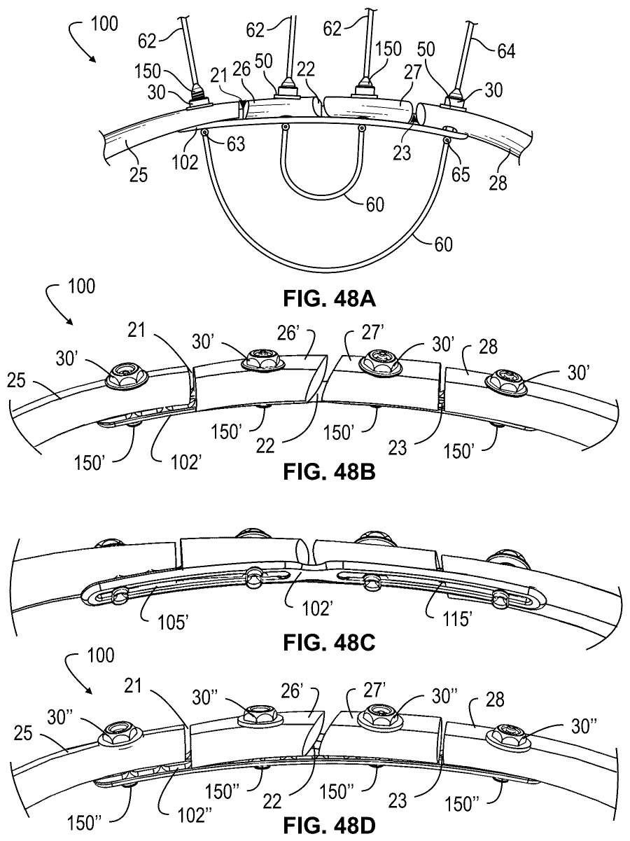

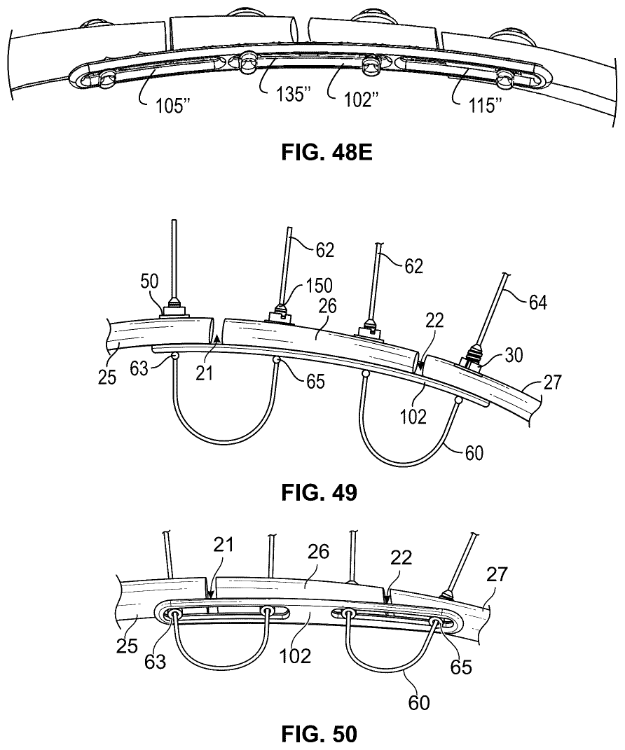

FIG. 48A is a front view of a fracture plating system 100 in a partially deployed configuration according to an embodiment of the present disclosure spanning a plurality of exemplary bone fractures. FIG. 48B is a perspective view of a fracture plating system 100 secured to a bone with multiple fractures according to an embodiment of the present disclosure. FIG. 48C is a perspective view of the fracture plating system 100 of FIG. 48B. The fracture plating system 100 may include a second plate 102′, fasteners 150′, and locking caps 30′.

The fracture plating system 100 may be configured to stabilize a multiple fractures of a bone of a patient, wherein the multiple fractures define one or more flail segments. For example, the fracture plating system 100 may be configured to stabilize a first fracture 21, a second fracture 22 and a third fracture 23 of a bone, wherein the first fracture 21 and the second fracture 22 define a first flail segment 26′ and the second fracture 22 and the third fracture 23 define a second flail segment 27′.

The fracture plating system may include a second plate 102′ having a first slot 105′ and a second slot 115′. The second plate 102′ may span the first fracture 21, the second fracture 22, and the third fracture 23 so that at least one fastener 150′ may be received in the first slot 105′ or the second slot 115′ and in the bone on both sides of each of the first fracture 21, the second fracture 22, and the third fracture 23. The first slot 105′ may span one or more of the first fracture 21, the second fracture 22, and the third fracture 23. Additionally, the second slot 115′ may also span one or more of the first fracture 21, the second fracture 22, and the third fracture 23.

FIG. 48D is a perspective view of a fracture plating system 100 secured to a bone with multiple fractures according to an embodiment of the present disclosure. FIG. 48E is a perspective view of the fracture plating system 100 of FIG. 48D. The fracture plating system 100 may include a plate 102″, fasteners 150″, and locking caps 30″.

The fracture plating system 100 may be configured to stabilize a multiple fractures of a bone of a patient, wherein the multiple fractures define one or more flail segments. For example, the fracture plating system 100 may be configured to stabilize a first fracture 21, a second fracture 22 and a third fracture 23 of a bone, wherein the first fracture 21 and the second fracture 22 define a first flail segment 26′ and the second fracture 22 and the third fracture 23 define a second flail segment 27′.

The fracture plating system may include a plate 102″ having a first slot 105″, a second slot 115″, and a third slot 135″. The second plate 102″ may span the first fracture 21, the second fracture 22, and the third fracture 23 so that at least one fastener 150″ may be received in the first slot 105″, the second slot 115′, or the third slot 135″ and in the bone on both sides of each of the first fracture 21, the second fracture 22, and the third fracture 23. The first slot 105″ may span one or more of the first fracture 21, the second fracture 22, and the third fracture 23. Additionally, the second slot 115″ may also span one or more of the first fracture 21, the second fracture 22, and the third fracture 23. Additionally, the third slot 135″ may also span one or more of the first fracture 21, the second fracture 22, and the third fracture 23.

FIG. 49 is a front view of a fracture plating system in a partially deployed configuration according to an embodiment of the present disclosure spanning a plurality of exemplary bone fractures. FIG. 50 is a bottom perspective view of the fracture plating system in a partially deployed configuration spanning a plurality of exemplary bone fractures.

The fracture plating system may be configured to stabilize a single fracture of a bone. Additionally, or alternatively, the fracture plating system may be configured to stabilize two fractures of a bone. Additionally, or alternatively, the fracture plating system may be configured to stabilize three or more fractures of a bone. Additionally, or alternatively, the fracture plating system may be configured to stabilize multiple fractures of a bone, wherein the multiple fractures define one or more flail segments. The fracture plating system may include multiple tethers, each configured to guide up to two fasteners to an interior surface of the bone and to one or more slots of the plate.

The fracture plating system previously described within the present disclosure may be used to stabilize multiple fractures of a bone. The multiple fractures may result in one or more flail segments and may result in flail chest. The fracture plating system previously described within the present disclosure may be used to stabilize one or more flail segments.

The fracture plating system previously described within the present disclosure may include a plate that may be configured to span the multiple fractures and four fasteners, each configured to be received in the bone and in one or more slots of the plate. The four fasteners may be configured to be received in the bone on opposite sides of each of the first fracture 21 and the second fracture 22. The fracture plating system may also include four locking caps configured to receive one of the four fasteners and cooperate with one of the four fasteners to secure the plate to the interior surface of the bone.

Additionally, or alternatively, the fracture plating system previously described within the present disclosure may include a plate that may be configured to span a first fracture 21 and a second fracture 22 and at least three fasteners, each configured to be received in the bone and in one or more slots of the plate. The at least three fasteners may be configured to be received in the bone on opposite sides of each of the first fracture 21 and the second fracture 22. The fracture plating system may also include at least three locking caps configured to receive one of the at least three fasteners and cooperate with one of the at least three fasteners to secure the plate to the interior surface of the bone.

The fracture plating system previously described within the present disclosure may be used to facilitate reduction of a first fracture 21, a second fracture 22, and a third fracture 23 whereby a first tether 60 may be received within a first fastener that is received within a first rib portion 25 and a fourth fastener that may be received within a fourth bone portion 28. A second tether 60 may be received within a second fastener that may be received within a second rib portion 26 and a third fastener that may be received within a third rib portion 27.

Applying a tension force to a first tether end 62 and a second tether end 64 of a first tether 60 and a first tether end 62 and a second tether end 64 of a second tether 60 may facilitate reduction of the first fracture 21, the second fracture 22, and the third fracture 23. The fracture plating system may then be secured by securing a locking cap to each of the fasteners.

The fracture plating system previously described within the present disclosure may be used to facilitate reduction of a first fracture 21 and a second fracture 22 whereby a first tether 60 is received within a first fastener that may be received within a first rib portion 25 and a second fastener that may be received within a second rib portion 26. A second tether 60 may be received within a third fastener that may be received within a third rib portion 27 and a fourth fastener that may be received within a fourth bone portion 28.

Applying a tension force to a first tether end 62 and a second tether end 64 of a first tether 60 and a first tether end 62 and a second tether end 64 of a second tether 60 may facilitate reduction of the first fracture 21 and the second fracture 22. The fracture plating system may then be secured by securing a locking cap to each of the fasteners.

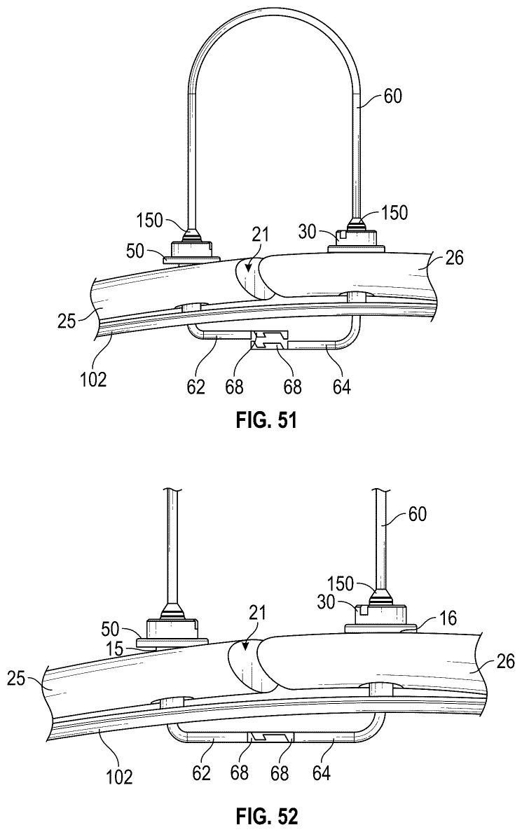

FIG. 51 is a bottom perspective view of a fracture plating system in a partially deployed configuration according to an embodiment of the present disclosure spanning an exemplary bone fracture. FIG. 52 is a partial bottom perspective view of the fracture plating system of FIG. 51 spanning an exemplary bone fracture. A tether 60 may be configured so that a first tether end 62 may releasably connect to a second tether end 64. The tether 60 may fed through two fasteners and a plate in the opposite direction, i.e.: the first tether end 62 passing through a tip portion of a first fastener, the second tether end 64 passing through a tip portion of a second fastener, and the first tether end 62 releasably connecting to the second tether end 64 on the bottom side of the plate. The first tether end 62 and the second tether end 64 tether ends may then be releasably connected together to create a single continuous loop of tether. The first tether end 62 and the second tether end 64 may each include a connection feature 68.

A first hole 15 may be drilled in a first rib portion 25 and a second hole 16 may be drilled in a second rib portion 26. The first tether end 62 may be passed through the first hole 15 and the second tether end 64 may be passed through the second hole 16. The first tether end 62 and the second tether end 64 may then be grabbed with an endoscopic grasper and pulled out thru a VATS port. The first tether end 62 may then be passed through the first fastener and a first slot of a plate and the second tether end 64 may then be passed through the second fastener and a second slot of the plate. The first tether end 62 may then be connected to the second tether end 64 using the connection feature 68. Once the connection is made, the tether 60 may then be pulled to place the plate again the interior surface of a portion of bone.

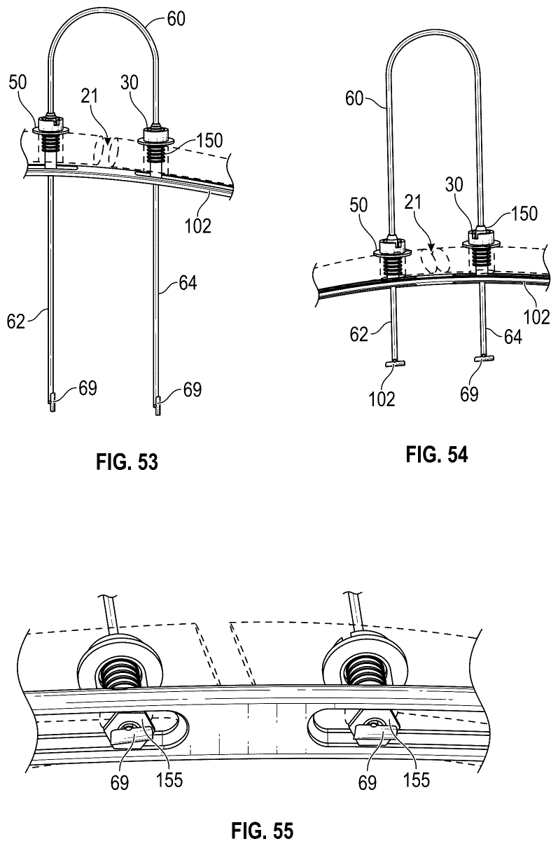

FIG. 53 is a front view of a fracture plating system in a partially deployed configuration according to an embodiment of the present disclosure spanning an exemplary bone fracture. FIG. 54 is a partial front view of the fracture plating system of FIG. 53 spanning an exemplary bone fracture. FIG. 55 is a partial bottom perspective view of the fracture plating system of FIG. 53 spanning an exemplary bone fracture.

A tether 60 may be configured so that a first tether end 62 and a second tether end 64 may each include a toggle feature 69. The tether 60 may fed through two fasteners and a plate in the opposite direction, i.e.: the first tether end 62 passing through a tip portion of a first fastener, the second tether end 64 passing through a tip portion of a second fastener, a first toggle feature 69 may be deployed so that the first tether end 62 may not return through the first fastener, and a second toggle feature may be deployed so that the second tether end 64 may not return through the second fastener.

A first hole 15 may be drilled in a first rib portion 25 and a second hole 16 may be drilled in a second rib portion 26. The first tether end 62 may be passed through the first hole 15 and the second tether end 64 may be passed through the second hole 16. The first tether end 62 and the second tether end 64 may then be grabbed with an endoscopic grasper and pulled out thru a VATS port. The first tether end 62 may then be passed through the first fastener and a first slot of a plate and the second tether end 64 may then be passed through the second fastener and a second slot of the plate. The toggle feature 69 of the first tether end 62 and the toggle feature 69 of the second tether end 64 may then be deployed. The tether 60 may then then be pulled to place the plate again the interior surface of a portion of bone.

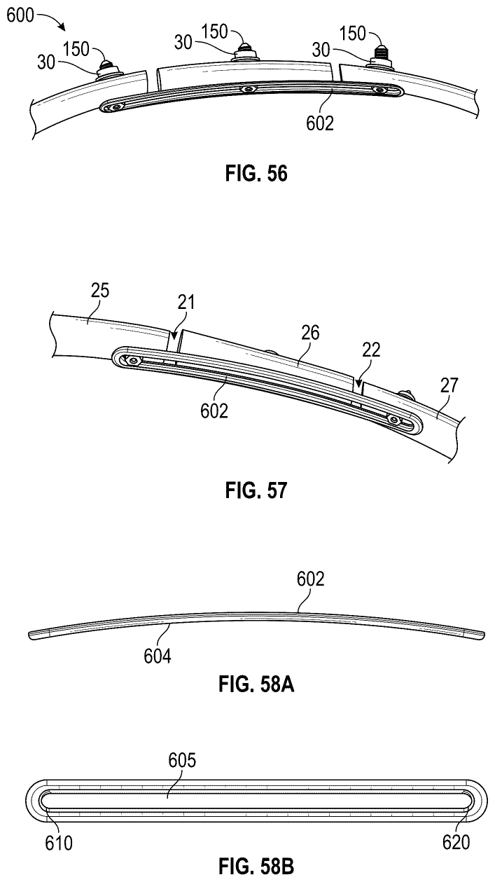

FIG. 56 is a front view of a fracture plating system 600 according to an embodiment of the present disclosure spanning a plurality of exemplary bone fractures. FIG. 57 is a bottom perspective view of the fracture plating system 600 spanning a plurality of exemplary bone fractures. The fracture plating system 600 may include similar features as other fracture plating systems previously described within the present disclosure. The fracture plating system 600 may include a plate 602 and may include two or more of an anti-rotation fastener 150 and/or a circular head fastener 180.

FIG. 58A is a front view of a plate 602 of the fracture plating system 600 according to an embodiment of the present disclosure. FIG. 58B is a bottom view of the plate 602. The plate 602 may include a plate radius 604, a slot 605, a first threaded feature 610, and a second threaded feature 620. Alternatively, the plate 602 may include a plate radius 604, a slot 605, and a first threaded feature 610 whereby a plurality of fasteners may threadably engage the first threaded feature 610 to allow the plurality of fasteners to be slidably received within the slot 605.

The plate 602 may have one continuous slot 605 that allows the user to choose the number of fasteners to be added to the fracture plating system 600 and may allow for more intraoperative flexibility of where a fastener may be located relative to the plate 602 and relative to a fracture. The number of fasteners may be customized to each patient to match their individual facture pattern. Similar to fracture plating system previously described in the present disclosure, the fasteners may be connected to the plate 602 during a surgical procedure by threading the two or more fasteners through the first threaded feature 610 and/or the second threaded feature 620 of the plate 602. Multiple lengths of fasteners may be selected and attached to the plate 602 to match the thickness of the rib in that region. Additional points of fixation (a 3 or more fasteners as shown) may accommodate for variations in curvature and/or thickness of a portion of bone.

The plate 602 may be one of a set of differently-sized implants, each having a different plate length. A plate radius 604 may generally match a contour of an interior surface of a rib. The plate 602 may further be one of a set of differently-sized implants, each having a different plate radius 604.

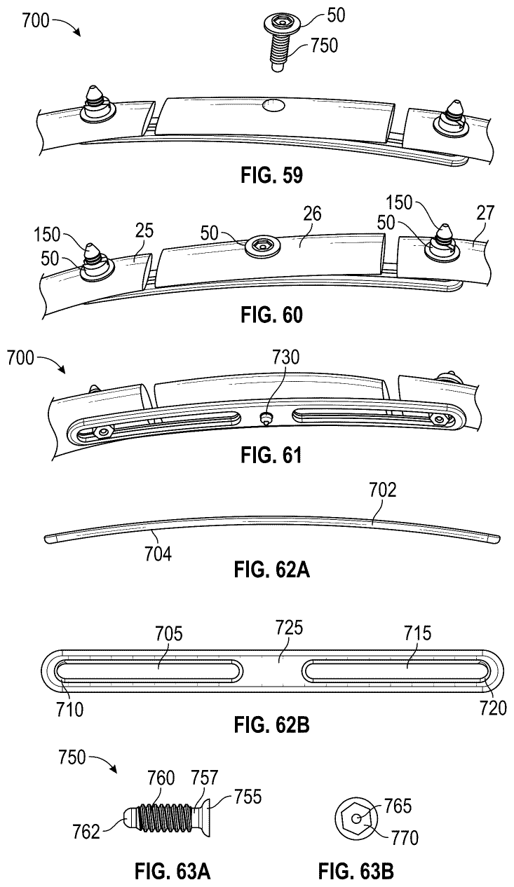

FIG. 59 is a perspective view of a fracture plating system 700 according to an embodiment of the present disclosure spanning a plurality of exemplary bone fractures. FIG. 60 is a perspective view of the fracture plating system 700 spanning a plurality of exemplary bone fractures. The fracture plating system 700 may include similar features as other fracture plating systems previously described within the present disclosure. The fracture plating system 700 may include a plate 702, a threaded fastener 750, and an anti-rotation fastener 150 and/or a circular head fastener 180. The fracture plating system 700 may allow a user to use a single plate 702 and a single tether 60 to stabilize two fractures in a single portion of bone.

FIG. 61 is a bottom perspective view of the fracture plating system 700 spanning a plurality of exemplary bone fractures. The plate 702 may be secured to a first rib portion 25 and a third rib portion 27 using components and methods previously described. The user may then drill a hole through the second rib portion 26 and through a central portion 725 of the plate 702 creating a central aperture 730 in the plate 702. A threaded fastener 750 may then be threadably inserted through the drilled hole and into central aperture 730 to secure the second rib portion to the plate 702.

FIG. 62A is a front view of a plate 702 of the fracture plating system 700 according to an embodiment of the present disclosure. FIG. 62B is bottom view of the plate 702. The plate 702 may include a plate radius 704, a first slot 705, a second slot 715, and a central portion 725. The first slot 705 may include a first threaded feature 710 and the second slot 715 may include a second threaded feature 720. The central portion 725 may be generally in the center of the plate 702 and may separate the first slot 705 and the second slot 715.

The first threaded feature 710 may be configured to threadably engage a threaded portion 190 of a circular head fastener 180 and/or the threaded portion 160 of an anti-rotation fastener 150 to allow the threaded portion 190 and/or the threaded portion 160 to pass through the first slot 705. The second threaded feature 720 may be configured to threadably engage a threaded portion 190 of a circular head fastener 180 and/or the threaded portion 160 of an anti-rotation fastener 150 to allow the threaded portion 190 and/or the threaded portion 160 to pass through the second slot 715.

The plate 702 may be configured such that a plate length is within a range of lengths from 30 mm to 300 mm. The plate 702 may be one of a set of differently-sized implants, each having a different plate length. A plate radius 704 may generally match a contour of an interior surface of a rib. The plate 702 may further be one of a set of differently-sized implants, each having a different plate radius 704.

FIG. 63A is a front view of a threaded fastener 750 of the fracture plating system 700 according to an embodiment of the present disclosure. FIG. 63B is a side view of the threaded fastener 750. The threaded fastener 750 may include a head portion 755, a shank portion 757, a threaded portion 760, a tip portion 762, a cannulation 765, and a drive portion 770.

The head portion 755 may include the drive portion 770. The drive portion may be configured to receive a driver instrument (not shown) configured to impart a torque to the threaded fastener 750. The drive portion 770 may be configured as a hex, a hexalobe, a square, a slot, or other drive geometry known in the art.

The cannulation 765 may be configured to receive a guide wire (not shown) to assist in placement location of the threaded fastener 750. The threaded portion 760 and the shank portion 757 may be configured to receive a washer 50. The tip portion 762 may be configured to guide the threaded fastener 750 into a hole drilled into a portion of bone. The threaded portion 760 may be configured to threadably engage the second rib portion 26 and the central aperture 730. Alternatively, a diameter of the hole drilled onto a portion bone may be larger than a thread diameter of the threaded fastener 750 and the threaded portion 760 may be configured to threadably engage the central aperture 730.

The threaded fastener 750 may be configured within a range of lengths from 5 mm to 20 mm and within a range of diameters from 3 mm to 8 mm. The threaded fastener 750 may be one of a set of differently-sized implants, each having a different length and/or diameter.

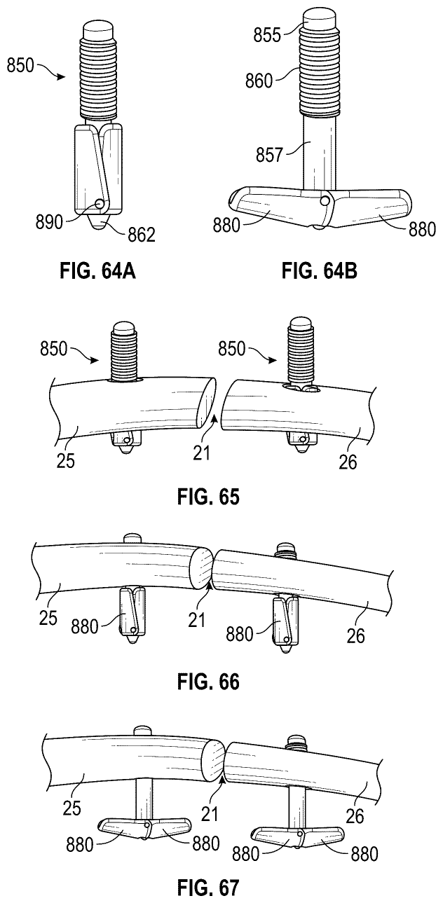

FIG. 64A front view of a toggle fastener 850 in an insertion configuration of a fracture plating system 800 according to an embodiment of the present disclosure. FIG. 64B is a front view of the toggle fastener 850 in a deployed configuration. The fracture plating system 800 may include similar features as other fracture plating systems previously described within the present disclosure. The fracture plating system 800 may include at least one toggle fastener 850, at least one locking cap 30 and a plate.

In an embodiment, at least one toggle fastener 850 may be inserted thru at least one hole in a bone. The at least one toggle fastener 850 may then be grabbed with an endoscopic grasper and pulled out a VATS port. The at least one toggle fastener 850 may then be inserted thru at least one slot in a properly configured plate and the at least one toggle fastener 850 may then be deployed to engage the plate. At least one tether 60 tethers may be pre-attached to a top of the toggle fastener. A user may pull the at least one tether 60 to tension the plate against the bone.

The toggle fastener 850 may include a head portion 855, a shank portion 857, a threaded portion 860, a tip portion 862, at least one toggle 880, and a pin 890. The head portion 855 may be configured to guide a washer 50 and/or a locking cap 30 onto the toggle fastener 850. The shank portion 857 may be configured to receive at least on toggle 880 when the at least one toggle is in an insertion configuration. The threaded portion 860 may be configured to threadably reactive a locking cap 30. The pin may be configured to rotatably secure at least one toggle 880 to the shank portion 857. The at least one toggle 880 may be configured so that, in a deployed configuration, the at least one toggle 880 extends beyond the threaded portion 860.

FIG. 65 is a perspective view of a pair of toggle fasteners 850 in a partially deployed configuration spanning an exemplary bone fracture. FIG. 66 is a perspective view of a pair of toggle fasteners 850 in a partially deployed configuration spanning an exemplary bone fracture. FIG. 67 is a perspective view of a pair of toggle fasteners 850 in a partially deployed configuration spanning an exemplary bone fracture. A first hole 15 may be drilled in a first rib portion 25 and a second hole 16 may be drilled in a second rib portion 26.

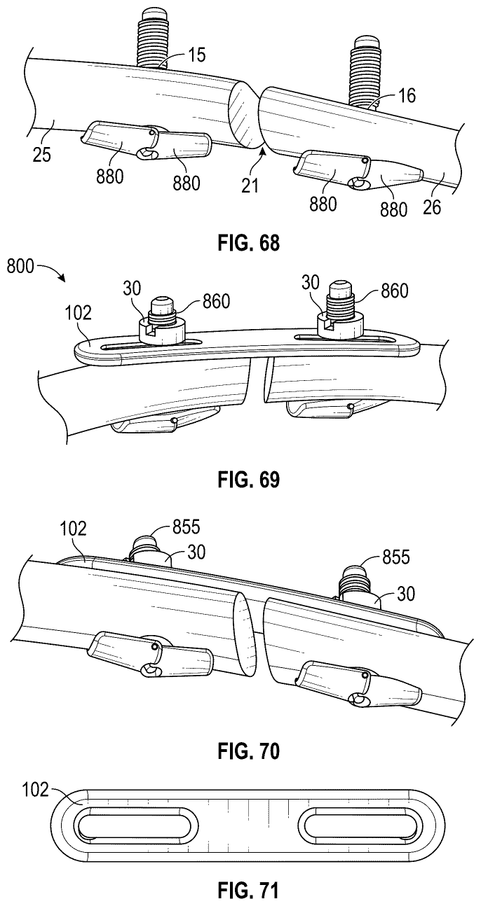

FIG. 68 is a perspective view of a pair of toggle fasteners 850 in a partially deployed configuration spanning an exemplary bone fracture. The toggle fastener 850 may include an insertion configuration in which a first toggle 880 and a second toggle 880 may be aligned generally parallel with a long axis of the toggle fastener 850. The toggle fastener 850 may include a deployed configuration in which a first toggle 880 and a second toggle 880 may be generally perpendicular to a long axis of the toggle fastener 850.

FIG. 69 is a perspective view of a fracture plating system 800 which may include the toggle fastener 850 according to an embodiment of the present disclosure spanning an exemplary bone fracture. A first toggle fastener 850, in an insertion configuration, may be inserted through the first hole, until the first toggle 880 and the second toggle 880 are past the inner surface of the bone. The first toggle fastener 850 may then translate from the insertion configuration to the deployed configuration and the toggle fastener 850 may be drawn upward and the toggle fastener 850 contacts the inside surface of the bone.

FIG. 70 is a bottom perspective view of the fracture plating system 800 spanning an exemplary bone fracture. The steps may be repeated for a second toggle fastener 850 being inserted into a second hole 16. The first toggle fastener 850 and the second toggle fastener 850 may be urged towards each other to reduce the facture.

FIG. 71 is a bottom view of the plate 102. A plate 102 may be applied to the tops of the first toggle fastener 850 and the second toggle fastener 850 fastener to provide rigid fixation that bridges the gap of the fracture. A washer 50 a locking cap 30 may be secured to each of the first toggle fastener 850 and the second toggle fastener 850 to tighten the construct in place.

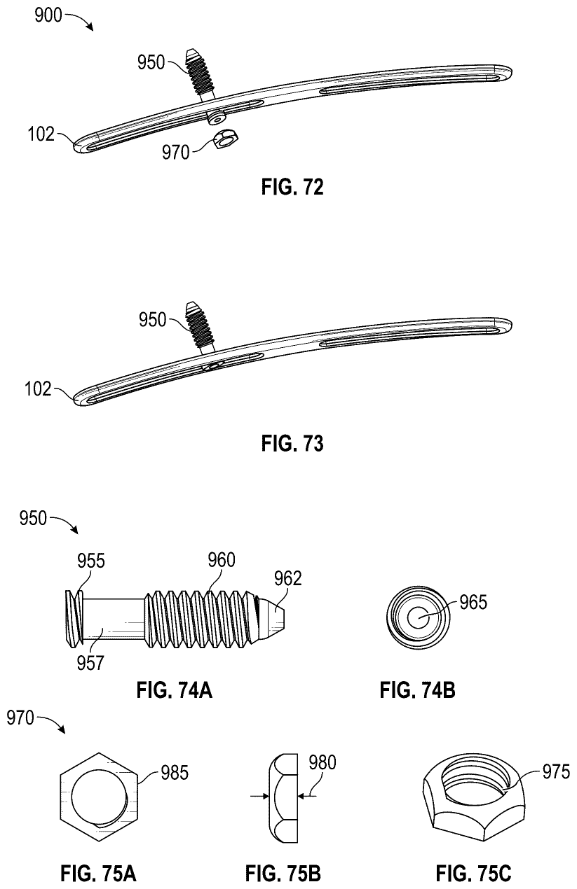

FIG. 72 is a perspective view of a fracture plating system 900 according to an embodiment of the present disclosure in a partially assembled configuration. FIG. 73 is a perspective view of the fracture plating system 900 in a partially assembled configuration. The fracture plating system 900 may include similar features as other fracture plating systems previously described within the present disclosure.

The fracture plating system 900 may include a post fastener 950, a locking cap 30, and one of any of the plates previously described, for example, plate 102. The post fastener 950 may be received in a first slot 105 and/or a second slot 115 of the plate 102. A cap 970 may then be threadably engaged with a second threaded portion 955 of the post fastener 950 to hold the post fastener 950 captively received within the plate 102 while still allowing translation of the post fastener 950 along a longitudinal axis of a first slot 105 and/or a second slot 115.

FIG. 74A is a front view of a post fastener 950 of the fracture plating system 900 according to an embodiment of the present disclosure. FIG. 74B is a side view of the post fastener 950. The post fastener 950 may include a first threaded portion 960, a second threaded portion 955, a shank portion 957, a tip portion 962, and a cannulation 965.

The shank portion 957 may be smaller than a width of the first slot 105 and/or the second slot 115 to allow the post fastener 950 to translate along the length of the first slot 105 and/or the second slot 115. The tip portion 962 may be configured to ease insertion of the post fastener 950 into a hole in a bone portion. The cannulation 965 may be configured to slidably receive a tether 60. The first threaded portion 960 may be configured to threadably receive a locking cap 30. The second threaded portion 955 may be configured to receive a cap 970.

FIG. 75A is a front view of a cap 970 of the fracture plating system 900 according to an embodiment of the present disclosure. FIG. 75B is a side view of the cap 970 and FIG. 75C is a perspective view of the cap 970. The cap 970 may include a third threaded portion 975, a height 980, and an outer portion 985. The third threaded portion 975 may be configured to threadably engage the second threaded portion 955 to capture the post fastener 950 within the first slot 105 and/orthe second slot 115 of the plate 102 while still allowing translation of the post fastener 950 along the first slot 105 and/or the second slot 115.

The height 980 may be configured to maximize threaded engagement between the second threaded portion 955 and the third threaded portion 975. The outer portion 985 may be configured as a not circular geometry to prevent rotation of the cap 970 when the cap 970 is received within the first slot 105 and/or the second slot 115.

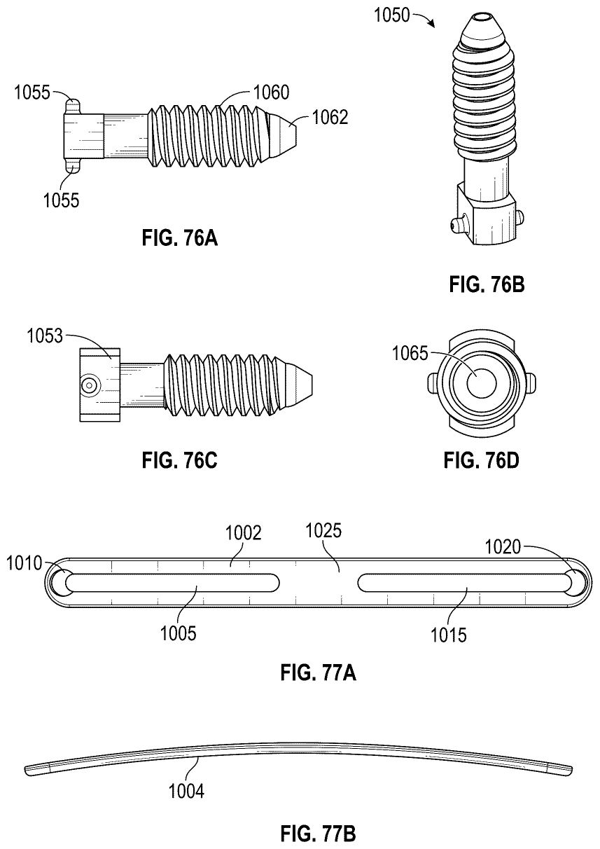

FIG. 76A is top view of a pin fastener 1050 of a fracture plating system 1000 according to an embodiment of the present disclosure. FIG. 76B is a perspective view of the pin fastener 1050, FIG. 76C is a front view of the pin fastener 1050, and FIG. 76D is a side view of the pin fastener 1050.

The fracture plating system 1000 may include similar features as other fracture plating systems previously described within the present disclosure. The fracture plating system 1000 may include a pin fastener 1050. The pin fastener 1050 may be configured to translate along a length of a first slot 1005 and/or a second slot 1015 in a plate 1002 to facilitate compression of a fracture. The fracture plating system 1000 may include a first pin fastener 1050 and a second pin fastener 1050 that each may translate within the first slot 1005 and/or the second slot 1015 of the plate 1002.

The pin fastener 1050 may include a head portion 1053, a threaded portion 1060, a tip portion 1062, and a cannulation 1065. The head portion 1053 may include at least one pin portion 1055. The head portion 1053 may be configured to be received within the first slot 1005 and/or the second slot 1015. The head portion 1053 may have a non-circular profile so that, when the head portion 1053 is received within the first slot 1005 and/or the second slot 1015, the pin fastener 1050 is prevented form rotating around the long axis of the pin fastener 1050, specifically when a locking cap 30 is threadably engaging the threaded portion 1060.

The pin portion 1055 may be configured to be received within a first groove 1012 and/or a second groove 1022 of the plate 1002. The pin portion 1055 may have a generally circular profile so that the pin fastener 1050 may translate along the length of the first slot 1005 and/or the second slot 1015 and the pin fastener 1050 may rotate about a long axis of the pin portion 1055.

The tip portion 1062 may be configured to ease insertion of the pin fastener 1050 into a hole in a bone portion. The cannulation 1065 may be configured to slidably receive a tether 60. The threaded portion 1060 may be configured to threadably receive a locking cap 30.

The pin fastener 1050 may be configured within a range of lengths from 5 mm to 30 mm and within a range of diameters from 3 mm to 8 mm. The pin fastener 1050 may be one of a set of differently-sized implants, each having a different length and/or diameter.

FIG. 77A is a top view of a plate 1002 of the fracture plating system 1000 according to an embodiment of the present disclosure. FIG. 77B is a front view of the plate 1002. The plate 1002 may include a plate radius 1004, a first slot 1005, a second slot 1015, and a central portion 1025. The first slot 1005 may include a first pocket 1010 and a first groove 1012. The second slot may include a second pocket 1020 and a second groove 1022.

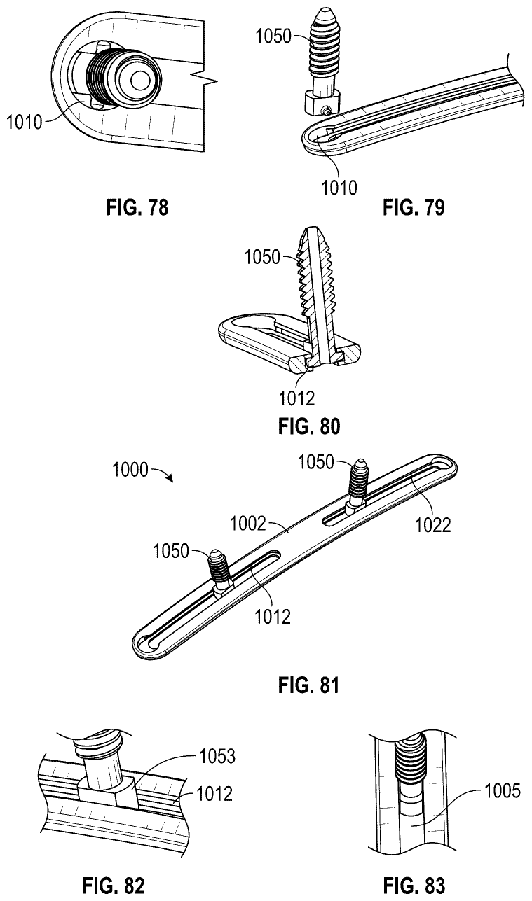

FIG. 78 is a partial top view of the fracture plating system 1000 in a partially assembled configuration. FIG. 79 is a partial perspective view of the fracture plating system 1000 in a partially assembled configuration. FIG. 80 is a perspective section view of the fracture plating system 1000. The central portion 1025 may be generally in the center of the plate 1002 and may separate the first slot 1005 and the second slot 1015.

FIG. 81 is a perspective view of a fracture plating system 1000 according to an embodiment of the present disclosure. FIG. 82 is a partial perspective view of the fracture plating system 1000. FIG. 83 is a partial perspective view of the fracture plating system 1000.

The first pocket 1010 may be configured to receive the head portion 1053 and the pin portion 1055 of the pin fastener 1050. After the pin portion 1055 is received within the first pocket 1010, the pin portion 1055 may be slidably received into the first groove 1012. The second pocket 1020 may be configured to receive the head portion 1053 and the pin portion 1055 of the pin fastener 1050. After the pin portion 1055 is received within the second pocket 1020, the pin portion 1055 may be slidably received into the second groove 1022.

The plate 1002 may be one of a set of differently-sized implants, each having a different plate length. A plate radius 1004 may generally match a contour of an interior surface of a rib. The plate 1002 may further be one of a set of differently-sized implants, each having a different plate radius 1004.

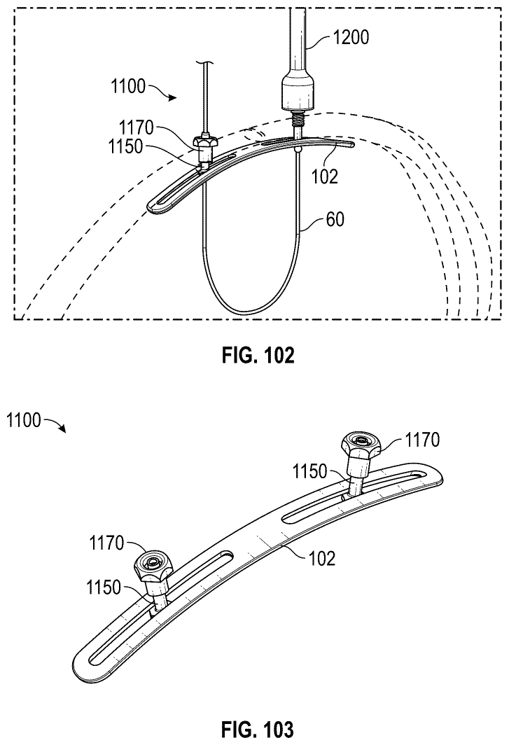

FIG. 102 is a perspective view of a fracture plating system 1100 according to an embodiment of the present disclosure spanning an exemplary bone fracture. FIG. 103 is a perspective view of a fracture plating system 1100. The fracture plating system 900 may include similar features as other fracture plating systems previously described within the present disclosure.

The fracture plating system 1100 may include an anti-rotation fastener 1150, a locking cap 1170, and one of any of the plates previously described, for example, plate 102. The anti-rotation fastener 1150 may be received in a first slot 105 and/or a second slot 115 of the plate 102. A locking cap 1170 may then be threadably engaged with a threaded portion 1160 of the anti-rotation fastener 1150 to hold the anti-rotation fastener 1150 captively received within the plate 102 while still allowing translation of the anti-rotation fastener 1150 along a longitudinal axis of the first slot 105 and/or a second slot 115. The anti-rotation fastener 1150 may include a head portion 1155, a shank portion 1157, a threaded portion 1160, a tip portion 1162, a cannulation 1165, and a rounded edge 1168.

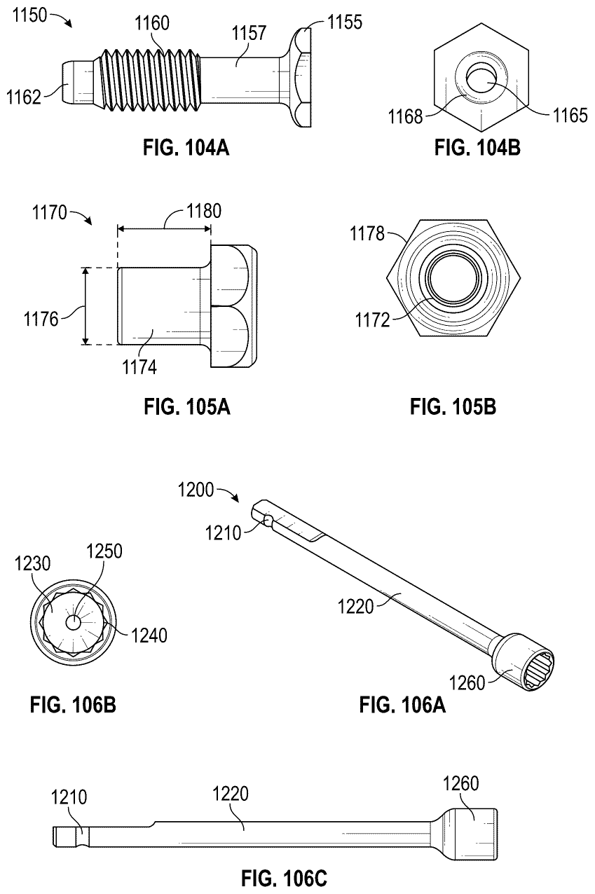

FIG. 104A is a front view of an anti-rotation fastener 1150 of the fracture plating system 1100 according to an embodiment of the present disclosure. FIG. 104B is a side view of the anti-rotation fastener 1150. The head portion 1155 may be configured to be received within the first slot 105 and/or the second slot 115. The threaded portion 1160 may be larger than a width of the first slot 105 and/or the second slot 115 to prevent the anti-rotation fastener 1150 from disengaging from the plate 102.

The shank portion 1157 may be smaller than a width of the first slot 105 and/or the second slot 115 to allow the anti-rotation fastener 1150 to translate along the length of the first slot 105 and/or the second slot 115. The tip portion 1162 may be configured to ease insertion of the anti-rotation fastener 1150 into a hole in a bone portion. The cannulation 1165 may be configured to slidably receive a tether 60. The threaded portion 1160 may be configured to threadably receive a locking cap 1170. The rounded edge 1168 may reduce stress on the tether 60 when the tether 60 exerts a force on the anti-rotation fastener 1150.

The anti-rotation fastener 1150 may be configured within a range of lengths from 5 mm to 30 mm and within a range of diameters from 2 mm to 8 mm. The anti-rotation fastener 1150 may be one of a set of differently-sized implants, each having a different length and/or diameter.

FIG. 105A is a front view of a locking cap 1170 of the fracture plating system 1100 according to an embodiment of the present disclosure. FIG. 105B is a side view of the locking cap 1170. The locking cap 1170 may be configured to threadably engage the anti-rotation fastener 1150 to secure the plate 102 to a portion of a rib.

The locking cap 1170 may include a threaded portion 1172, a body 1174, a body diameter 1176, a head profile 1178, and a body length 1180. The threaded portion 1172 may be configured to threadably engage the threaded portion 1160 of the anti-rotation fastener 1150. The head profile 1178 may allow engagement of a driver 1200 to threadably engage the locking cap 1170 with the anti-rotation fastener 1150. The head profile 1178 may be configured as a hex, a square, or other non-circular geometry.

The body diameter 1176 may be configured to be received in a hole drilled into a portion of bone. The body length 1180 may be configured to be less than a thickness of a portion of the bone.

FIG. 106A is a perspective view of a driver 1200 of the fracture plating system 1100 according to an embodiment of the present disclosure. FIG. 106B is a side view of the driver 1200 and FIG. 106C is a front view of the driver 1200. The driver 1200 may be configured to engage a locking cap 1170 and may transfer torque from a handle (not shown) to the locking cap 1170 thereby threadably securing the locking cap 1170 to an anti-rotation fastener 1150.

The driver 1200 may include a quick connect feature 1210, a head portion 1260, and a shaft configured to connect the quick connect feature 1210 and the head portion 1260. The shaft 1220 may include a cannulation 1250. The head portion 1260 may include a socket portion 1230 that may include a drive portion 1240. The cannulation 1250 may be configured to receive a guide wire (not shown) to assist in alignment of the driver 1200 with the anti-rotation fastener 1150.

The quick connect feature 1210 may be configured to be removably received by a handle (not shown) having a compatible quick connect feature. The quick connect feature 1210 may be configured as one of: AO connector, Hudson connector, trilobe connector, square connector, hex connector, Stryker connector, Stryker-Hall connector, or other quick connect mechanism known in the art.

The socket portion 1230 may be configured to receive the head profile 1178 of the locking cap 1170. The drive portion 1240 may be configured to engage the head profile 1178 and may be configured as a hex, a double hex, a square or other non-circular geometry that may engage the head profile 1178 of the locking cap 1170 in order to transfer torque from the driver 1200 to the locking cap 1170.

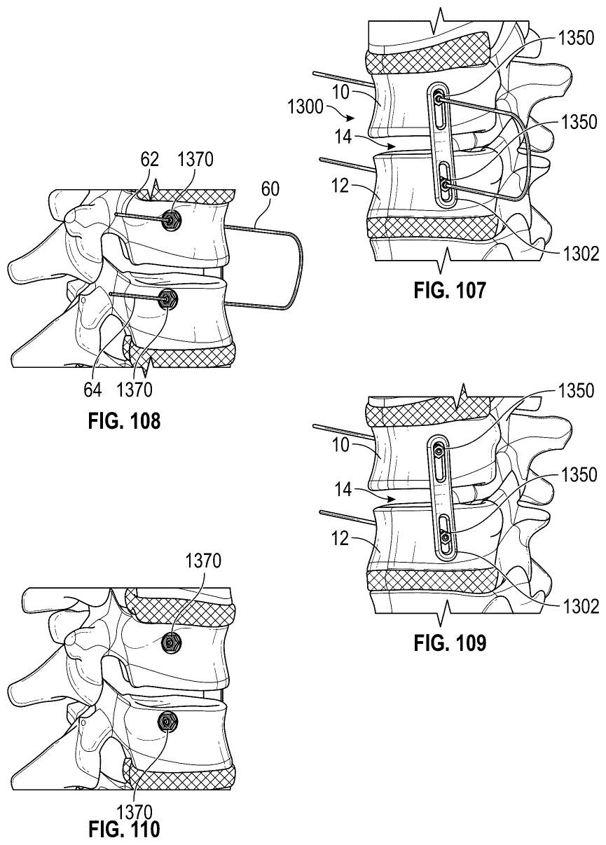

FIG. 107 is a front perspective view of a spinal fixation plating system 1300, secured to an exemplary portion of a spine, according to an embodiment of the present disclosure. The spinal fixation plating system 1300 may include a plate 1302, an anti-rotation fastener 1350, and a locking cap 1370. The plate 1302 may span an intervertebral space 14 and may be secured to a first vertebra 10 and a second vertebra 12.

The spinal fixation plating system 1300 may be configured to provide immobilization and stabilization of spinal segments. The spinal fixation plating system 1300 may be used as an adjunct to fusion in the treatment of the cervical, thoracic, lumbar, and/or sacral spine. Additionally, or alternatively, the spinal fixation plating system 1300 may be used in conjunction with other devices to support fusion of one or more spinal segments. The spinal fixation plating system 1300 may be implanted through an interior approach (ALIF), a posterior transforaminal approach (TLIF), a lateral approach (DLIF/XLIF), and/or an oblique approach (OLIF).

One or more spinal fixation plating system 1300 constructs may be used on a single spinal segment. The one or more spinal fixation plating system 1300 constructs may be implanted on opposite side of the spinal column. Additionally, or alternatively, a single spinal fixation plating system 1300 construct may span one or more spinal segments. Additionally, or alternatively, one or more spinal fixation plating system 1300 constructs may be configured to prevent expulsion and/or subsidence of one or more intervertebral implants. Additionally, or alternatively, the spinal fixation plating system 1300 may be configured to be implanted as an adjunct to a posterior rod/pedicle screw construct.

FIG. 108 is a rear perspective view of the spinal fixation plating system 1300 secured to an exemplary portion of a spine. Similar to the fracture plating systems previously described, the spinal fixation system may be configured to be deployed using a single tether 60 to pull the plate against the first vertebra 10 and the second vertebra 12. The tether 60 may be used to guide a first anti-rotation fastener 1350 through a hole in the first vertebra 10 and a second anti-rotation fastener 1350 through a hole in the second vertebra 12. A first locking cap 1370 may be guided along a first tether end 62 to threadably engage the first anti-rotation fastener 1350. A second locking cap 1370 may be guided along a second tether end 64 to threadably engage the second anti-rotation fastener 1350.

FIG. 109 is a front perspective view of the spinal fixation plating system 1300 secured to an exemplary portion of a spine. FIG. 110 is a rear perspective view of the spinal fixation plating system 1300 secured to an exemplary portion of a spine. After the first locking cap 1370 threadably engages the first anti-rotation fastener 1350 and the second locking cap 1370 threadably engages the second anti-rotation fastener, the tether 60 may be removed.

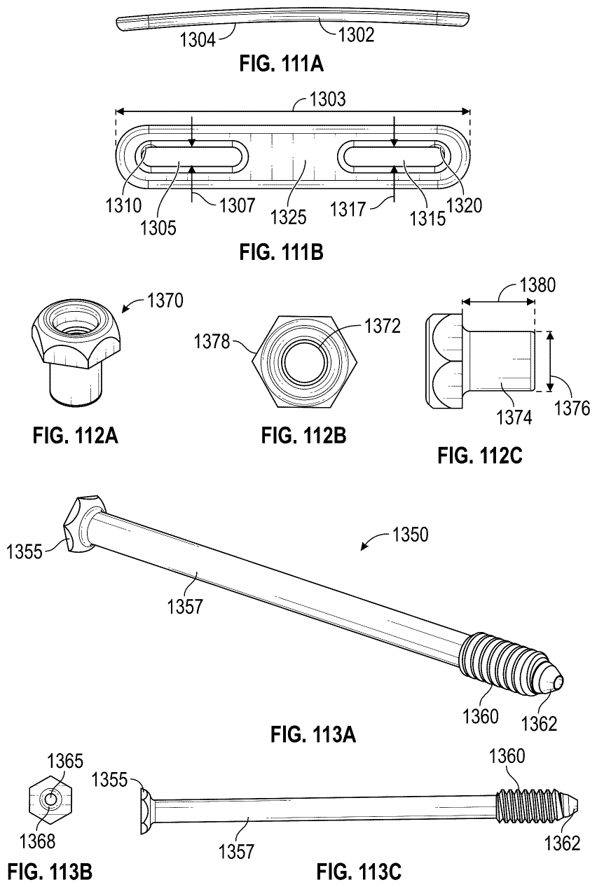

FIG. 111A is a front view of a plate 1302 of the spinal fixation plating system 1300 according to an embodiment of the present disclosure. FIG. 111B is a bottom view of the plate 1302. The plate 1302 may include a plate length 1303 and a plate radius 1304. The plate length 1303 may be configured to span one or more spinal segments.

The plate 1302 may be configured such that the plate length 1303 is within a range of lengths from 30 mm to 300 mm. The plate 1302 may be one of a set of differently-sized implants, each having a different plate length 1303. The plate radius 1304 may generally match a contour of a lateral and/or anterior portion of a vertebra. The plate 1302 may further be one of a set of differently-sized implants, each having a different plate radius 1304.

The plate 1302 may further include a central portion 1325 and a first slot 1305 having a first slot width 1307 and a first threaded feature 1310. The plate 1302 may also include a second slot 1315 having a second slot width 1317 and a second threaded feature 1320. The central portion 1325 may be generally in the center of the plate 1302 and may separate the first slot 1305 and the second slot 1315.

The first slot width 1307 and the second slot width 1317 may each be configured to receive an anti-rotation fastener 1350. The first slot width 1307 and the second slot width 1317 may further be configured to allow translation of the anti-rotation fastener 1350 along the length of the first slot 1305 and the second slot 1315 respectively.

FIG. 112A is a perspective view of a locking cap 1370 of the spinal fixation plating system 1300 according to an embodiment of the present disclosure. FIG. 112B is a front view of the locking cap 1370 and FIG. 112C is a side view of the locking cap 1370. The locking cap 1370 may be configured to threadably engage the anti-rotation fastener 1350 to secure the plate 1302 to a vertebra.

The locking cap 1370 may include a threaded portion 1372, a body 1374, a body diameter 1376, a head profile 1378, and a body length 1380. The threaded portion 1372 may be configured to threadably engage the threaded portion 1360 of the anti-rotation fastener 1350. The head profile 1378 may allow engagement of a driver 1200 to threadably engage the locking cap 1370 with the anti-rotation fastener 1350. The head profile 1378 may be configured as a hex, a square, or other non-circular geometry.

The body diameter 1376 may be configured to be received in a hole drilled into a vertebra. The body length 1380 may be configured to be less than a thickness of a vertebra.

FIG. 113A is a perspective view of an anti-rotation fastener 1350 of the spinal fixation plating system 1300 according to an embodiment of the present disclosure. FIG. 113B is a front view of the anti-rotation fastener 1350 and FIG. 113C is a side view of the anti-rotation fastener 1350. The anti-rotation fastener 1350 may include a head portion 1355, a shank portion 1357, a threaded portion 1360, a tip portion 1362, a cannulation 1365, and a rounded edge 1368.

The anti-rotation fastener 1350 may be configured to secure the plate 1302 to a vertebra. The anti-rotation fastener 1350 may also be configured to be captively received within the plate 1302 while still being allow to translate along a longitudinal axis of the first slot 1305 and/or the second slot 1315. The anti-rotation fastener 1350 may be configured so that, when a head portion 1355 engages the first slot 1305 and/or the second slot 1315, the anti-rotation fastener 1350 is prevented from rotating in relation to the plate 1302.

The head portion 1355 may be configured to be received within the first slot 1305 and/or the second slot 1315. The threaded portion 1360 may be larger than a width of the first slot 1305 and/or the second slot 1315 to prevent the anti-rotation fastener 1350 from disengaging from the plate 1302. The first threaded feature 1310 and the second threaded feature 1320 may each be configured to threadably engage the threaded portion 1360 to allow the threaded portion 1360 to pass through the first slot 1305 and the second slot 1315 respectively.

The shank portion 1357 may be smaller than a width of the first slot 1305 and/or the second slot 1315 to allow the anti-rotation fastener 1350 to translate along the length of the first slot 1305 and/or the second slot 1315 respectively. The tip portion 1362 may be configured to ease insertion of the anti-rotation fastener 1350 into a hole in a vertebra. The cannulation 1365 may be configured to slidably receive a tether 60. The rounded edge 1368 may reduce stress on the tether 60 when the tether 60 exerts a force on the anti-rotation fastener 1350.

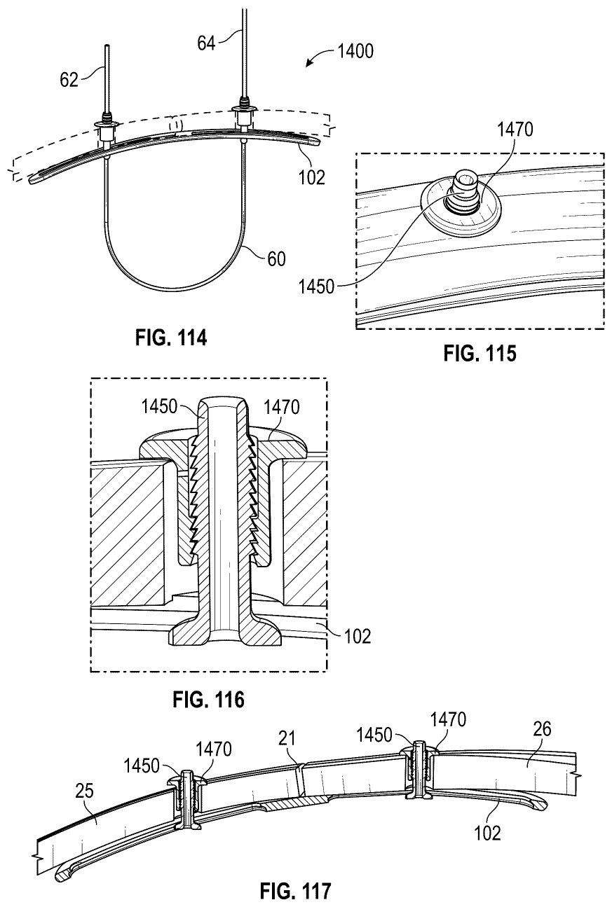

FIG. 114 is a front view of a fracture plating system 1400 according to an embodiment of the present disclosure spanning an exemplary bone fracture. FIG. 115 is a partial perspective view of the fracture plating system 1400. FIG. 116 is a partial section view of the fracture plating system 1400. FIG. 117 is a front section view of the fracture plating system 1400.

The fracture plating system 1400 may include similar features as other fracture plating systems previously described within the present disclosure. The fracture plating system 1400 may include a ratchet fastener 1450, a ratchet cap 1470, and one of any of the plates previously described, for example, plate 102.

The ratchet fastener 1450 may be received in a first slot 105 and/or a second slot 115 of the plate 102. A ratchet cap 1470 may then be engaged with a ratchet portion 1460 of the ratchet fastener 1450 to hold the ratchet fastener 1450 captively received within the plate 102 while still allowing translation of the ratchet fastener 1450 along a longitudinal axis of a first slot 105 and/or a second slot 115.

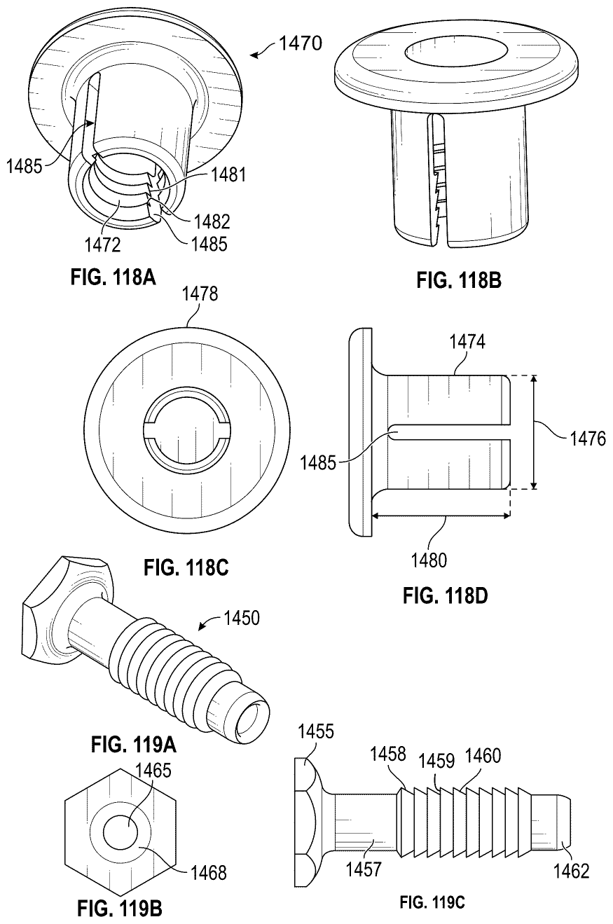

FIG. 118A is a bottom perspective view of a ratchet cap 1470 of the fracture plating system 1400 according to an embodiment of the present disclosure. FIG. 118B is front perspective view of the ratchet cap 1470, FIG. 118C is a front view of the ratchet cap 1470, and FIG. 118D is a side view of the ratchet cap 1470. The ratchet portion 1472 may be configured to engage the ratchet portion 1460 of the ratchet fastener 1450 to secure the plate 102 to a portion of a rib.

The ratchet cap 1470 may include a ratchet portion 1472, a body 1474, a body diameter 1476, a head diameter 1478, a body length 1480, and one or more slots 1485. The ratchet portion 1472 may be configured to engage the ratchet portion 1460 of the ratchet fastener 1450. The body diameter 1476 may be configured to be received in a hole drilled into a portion of bone. The body length 1480 may be configured to be less than a thickness of a portion of the bone.

The head diameter 1478 may be configured to be larger than a hole drilled into a portion of bone. The ratchet portion 1472 may include a plurality of grooves. Each of the plurality of grooves may have a leading angle 1481 and a trailing angle 1482. The leading angle 1481 may be within a range of 15° to 45°. More specifically, the leading angle 1481 may be within a range of 20° to 40°. More specifically, the leading angle 1481 may be 30°. The trailing angle 1482 may be generally perpendicular to a long axis of the body 1474. The one or more slots 1485 may be configured to allow the body diameter 1476 to temporarily increase as the ratchet portion 1472 of the ratchet cap 1470 engages and the ratchet portion 1460 of the ratchet fastener 1450.

FIG. 119A is a perspective view of a ratchet fastener 1450 of the fracture plating system 1400 according to an embodiment of the present disclosure. FIG. 119B is a front view of the ratchet fastener 1450 and FIG. 119C is a side view of the ratchet fastener 1450. The ratchet fastener 1450 may include a head portion 1455, a shank portion 1457, a ratchet portion 1460, a tip portion 1462, a cannulation 1465, and a rounded edge 1468. The head portion 1455 may be configured to be received within the first slot 105 and/or the second slot 115. The ratchet portion 1460 may be larger than a width of the first slot 105 and/or the second slot 115 to prevent the ratchet fastener 1450 from disengaging from the plate 102.

The shank portion 1457 may be smaller than a width of the first slot 105 and/or the second slot 115 to allow the ratchet fastener 1450 to translate along the length of the first slot 105 and/or the second slot 115. The tip portion 1462 may be configured to ease insertion of the ratchet fastener 1450 into a hole in a bone portion. The cannulation 1465 may be configured to slidably receive a tether 60. The ratchet portion 1460 may be configured to receive a ratchet cap 1470. The rounded edge 1468 may reduce stress on the tether 60 when the tether 60 exerts a force on the ratchet fastener 1450.

The ratchet portion 1460 may include a plurality of grooves. Each of the plurality of grooves may have a leading angle 1458 and a trailing angle 1459. The leading angle 1458 may be within a range of 15° to 45°. More specifically, the leading angle 1458 may be within a range of 20° to 40°. More specifically, the leading angle 1458 may be 30°. The trailing angle 1459 may be generally perpendicular to a long axis of the shank portion 1457.

The ratchet fastener 1450 may be configured within a range of lengths from 5 mm to 30 mm and within a range of diameters from 3 mm to 8 mm. The ratchet fastener 1450 may be one of a set of differently-sized implants, each having a different length and/or diameter. The ratchet cap 1470 may be one of a set of differently-sized implants, each having a different diameter configured to engage each of the differently sized ratchet fasteners 1450.

The ratchet fastener 1450 may be configured to receive the ratchet cap 1470 so that the ratchet portion 1460 of the ratchet fastener 1450 engages the ratchet portion 1472 of the ratchet cap 1470. More specifically, the leading angle 1481 of the ratchet cap 1470 and the leading angle 1458 of the ratchet fastener 1450 may be configured to allow the ratchet cap 1470 to advance along a length of the ratchet fastener 1450 from the tip portion 1462 towards the head portion 1455.

Moreover, the trailing angle 1482 of the ratchet cap 1470 and the trailing angle of the ratchet fastener 1450 may be configured to prevent translation of the ratchet cap 1470 along a length of the ratchet fastener 1450 from the head portion 1455 towards the tip portion 1462.

The ratchet cap 1470 may create compression at the interface similar to the locking cap 1370, but the advantage may be that it the ratchet cap 1470 may not require a drive feature to install the ratchet cap 1470 and secure the construct. The drive feature on the previous concepts may require the cap to have some thickness to engage with. The ratchet cap 1470 may not require a non-circular drive feature to apply torque. Thus, the ratchet cap 1470 may be much thinner than the locking cap 1370. A thinner cap may result in a lower profile construct on the bone and thus may reduce irritation of the surrounding soft tissues.

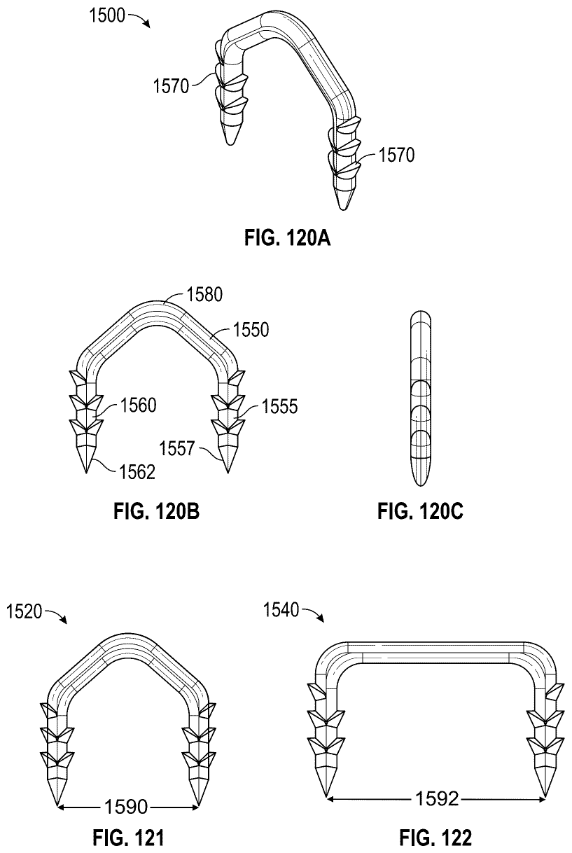

FIG. 120A is a perspective view of a fracture repair system 1500 in an undeformed configuration 1520 according to an embodiment of the present disclosure. FIG. 120B is a front view of the fracture repair system 1500 in an undeformed configuration 1520. FIG. 120C is a side view of the fracture repair system 1500 in an undeformed configuration 1520. The fracture repair system 1500 may be configured as a staple.

The fracture repair system 1500 may be configured to span a fracture in a portion of a bone. The fracture repair system 1500 may further be configured to provide compression of the fracture after implantation.

The fracture repair system 1500 may include a bridge portion 1550, a first leg 1555 having a first tip 1557, a second leg 1560 having a second tip 1562, a plurality of retaining features 1570, and a bridge apex 1580. The bridge portion 1550 may connect the first leg 1555 and the second leg 1560. Each of the first leg 1555 and the second leg 1560 may include a plurality of retaining features 1570. The plurality of retaining features 1570 may be configured to inhibit subsidence or back-out of the fracture repair system 1500 from a bone portion after implantation.

The first tip 1557 and the second tip 1562 may each be configured with a sharp point configured to penetrate a portion of bone. The fracture repair system 1500 may be configured to be embedded directly into a portion of bone. Additionally, or alternatively, the fracture repair system 1500 may be configured to be embedded into one or more apertures in a portion of bone and/or a portion of cartilage.

The fracture repair system 1500 may be fabricated from NITINOL, titanium, titanium alloy, stainless steel, cobalt-chrome, PEEK, PEAK, UHMWPE, a resorbable polymer, or any other biocompatible material with sufficient tensile strength and shape memory properties.

The fracture repair system 1500 may include an undeformed configuration 1520 and an expanded configuration 1540. FIG. 121 is a front view of the fracture repair system 1500 in an undeformed configuration 1520. The FIG. 122 is a front view of the fracture repair system 1500 in an expanded configuration 1540 according to an embodiment of the present disclosure.

In the undeformed configuration 1520 the bridge portion 1550 may include a bridge apex 1580. The bridge apex 1580 may be centrally located along the bridge portion 1550. In the undeformed configuration 1520 the fracture repair system 1500 may include a first leg spacing 1590. In the expanded configuration 1540 the fracture repair system 1500 may include a second leg spacing 1592. The first leg spacing 1590 may be less than the second leg spacing 1592.

The fracture repair system 1500 may be configured so that an external force is required to transition from the undeformed configuration 1520 to the expanded configuration 1540. Additionally, when the external force is removed, the fracture repair system 1500 will return from the expanded configuration 1540 to the undeformed configuration 1520 with no other externally applied forces.

An instrument/tool (not shown) may be used to elastically deform the fracture repair system 1500 from the undeformed configuration 1520 to the expanded configuration 1540. Once inserted into the tissue (bone and/or cartilage) the force of the instrument may be removed and the fracture repair system 1500 may return to the undeformed configuration 1520. The fracture repair system 1500 may be inserted to bridge a rib fracture in the expanded configuration 1540, and then may be used to compress the fractured bones back together as it moves back towards the undeformed configuration 1520.

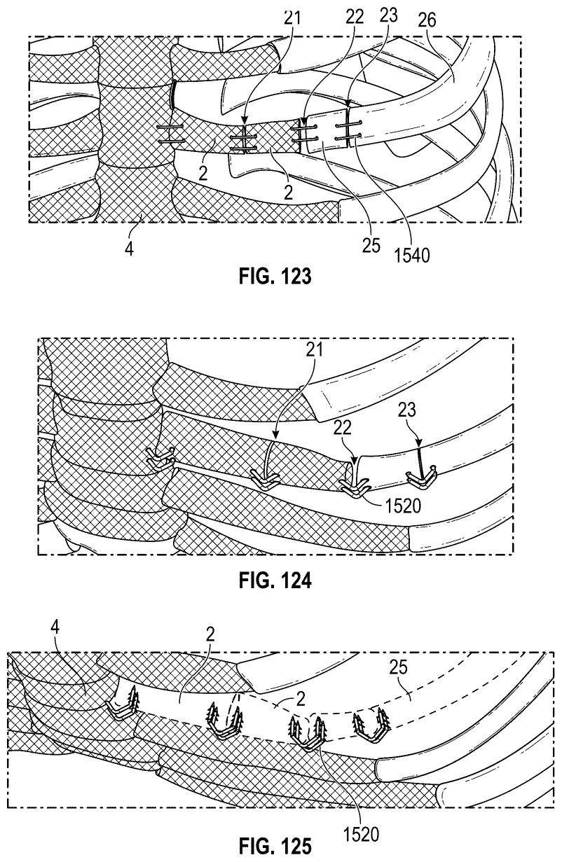

FIG. 123 is a front section view of an exemplary rib cage with the fracture repair system 1500 spanning exemplary fractures. FIG. 124 is a front section view of an exemplary rib cage with the fracture repair system 1500 compressing exemplary fractures. FIG. 125 is a front section view of an exemplary rib cage with the fracture repair system 1500 compressing exemplary fractures. The fracture repair system 1500 may be utilized to bridge fractures in ribs, in the costal cartilage, between sternum-to-costal cartilage, rib-to-costal cartilage, bone-to-cartilage, bone-to-bone, and/or cartilage-to-cartilage. One or more of the fracture repair systems 1500 may be applied to a single fracture and may provide additional stabilization and/or additional compression.

A fracture repair system 1600 may include a balloon 1610, cement, and an application instrument 1620. Human rib bones are generally hollow, similar to long bones, and may have an intramedullary canal. The balloon 1610 may be configured for repairing a fracture of any bone having an intramedullary canal. The balloon 1610 may be configured to be inserted into an intramedullary canal and span a fracture from an inside of a portion of bone. The balloon 1610 may then be filled with a bone cement 1650 know in the orthopedic arts. After a period of time and/or application of a hardening agent the bone cement 1650 may harden. The hardened bone cement 1650 and balloon may stabilize the fracture.

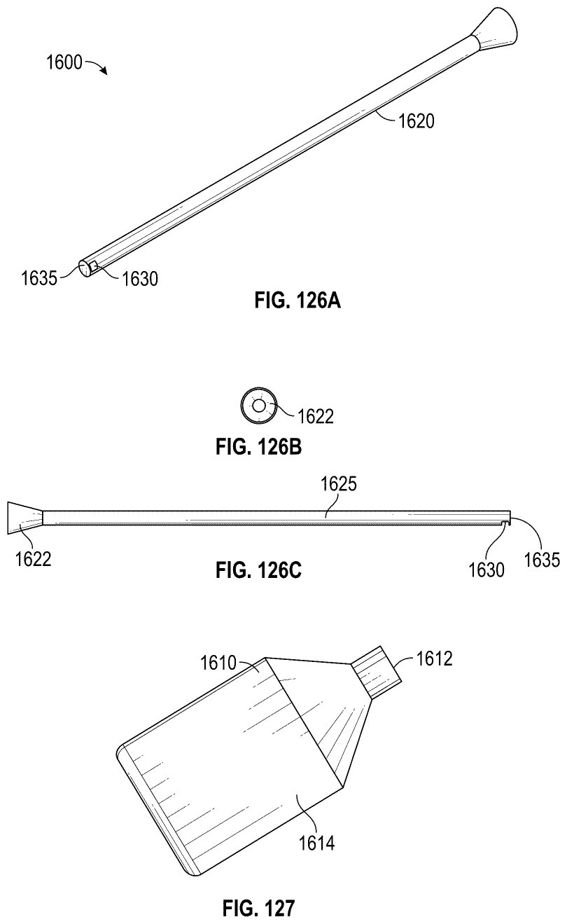

FIG. 126A is a perspective view of an application instrument 1620 of a fracture repair system 1600 according to an embodiment of the present disclosure. FIG. 126B is a side view of the application instrument 1620 and FIG. 126C is a front view of the application instrument 1620. The application instrument may include a funnel 1622, a shank 1625, an aperture 1630, and an end portion 1635.

The shank 1625 may be hollow and may connect the funnel 1622 with the aperture 1630. The funnel 1622 may be configured to ease insertion of the balloon 1610 and/or bone cement 1650. The end portion 1635 may be configured to prevent discharge from the shank 1625 through the end portion 1635. The application instrument 1620 may be configured so that the balloon 1610 and/or the bone cement 1650 may be introduced into the funnel, travel through the interior of the shank 1625 and exit through the aperture 1630. The aperture 1630 may be configured so that the balloon 1610 and/or the bone cement 1650 exit at a generally right angle with respect to an axis of the shank 1625.

FIG. 127 is a balloon 1610 of the fracture repair system 1600 according to an embodiment of the present disclosure. The balloon 1610 may be configured to be deployed through the application instrument 1620 into an intramedullary canal of a bone portion. The balloon 1610 may include an opening 1612 and a body 1614. The opening 1612 may be sized generally the same as, or slightly larger than, the aperture 1630 to facilitate introduction of bone cement 1650 through the aperture into the interior of the balloon 1610. The circumference of the balloon 1610 may be generally equal to or larger than the circumference of the intramedullary canal of the fractured bone portion.

The body 1614 may be configured to receive bone cement 1650 through the opening 1612 and expand upon introduction of the bone cement 1650. The balloon 1610 may be configured within a range of outer profiles from 8 mm to 25 mm and within a range of lengths from 5 mm to 500 mm. The balloon 1610 may be one of a set of differently-sized implants, each having a different outer profile and/or length. The balloon 1610 may be impregnated with barium sulfate or similar contrast agent. The outer profile may be generally equal to an outside perimeter of the balloon 1610. In one embodiment, the balloon 1610 may be circular in cross-section and the outer profile may equal the circumference of the cross-section.

A method for stabilizing one or more fractures of a portion of bone may include using a balloon 1610 and bone cement 1650. A method for stabilizing one or more fractures of a portion of bone may include the following steps:

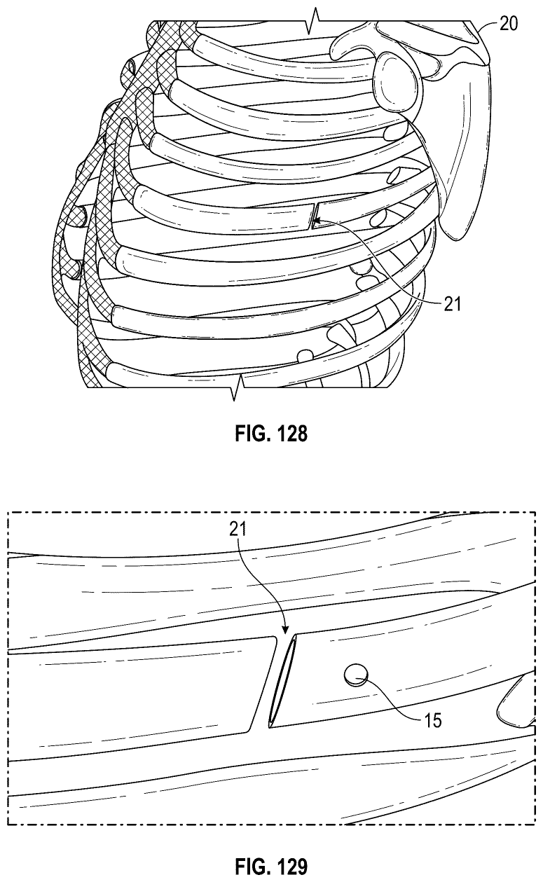

FIG. 128 is a partial perspective view of an exemplary rib cage 20 with an exemplary first fracture 21.

Step 1 may include finding a fracture 21 using ultrasound or other radiographic means.

FIG. 129 is a partial perspective view of the exemplary rib cage 20 illustrating a step in a method of deploying a fracture repair system 1600 according to an embodiment of the present disclosure.

Step 2 may include drilling a first hole 15 through the proximal cortex of the bone portion adjacent to the first fracture 21.

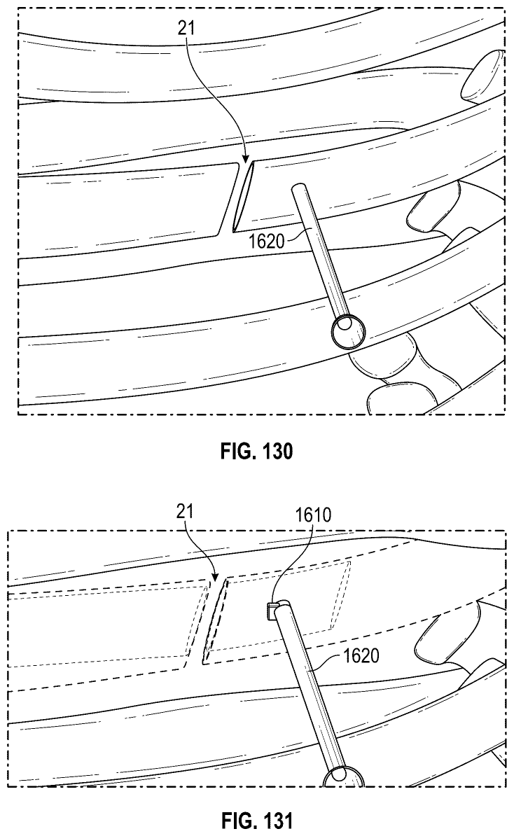

FIG. 130 is a partial perspective view of the exemplary rib cage 20 illustrating a step in a method of deploying a fracture repair system 1600 according to an embodiment of the present disclosure.

Step 3 may include inserting the application instrument 1620 into the hole 15 and orienting the aperture 1630 towards the first fracture 21.

FIG. 131 is a partial perspective view of the exemplary rib cage 20 illustrating a step in a method of deploying a fracture repair system 1600 according to an embodiment of the present disclosure.

Step 4 may include pressurizing the balloon 1610 with air, other gas, and/or saline through the application instrument 1620 into the intramedullary canal towards the first fracture 21. Verifying that the balloon 1610 has bridged the fracture and entered the opposite portion of the bone using fluoroscopy.

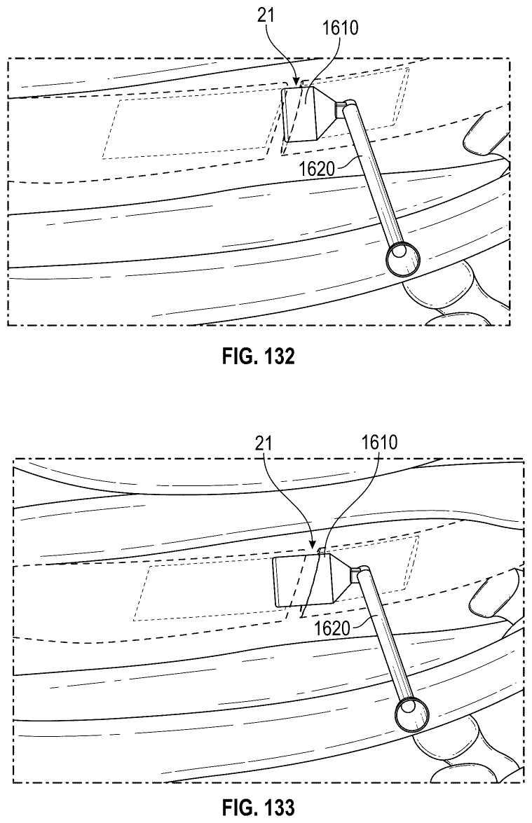

FIG. 132 is a partial perspective view of the exemplary rib cage 20 illustrating a step in a method of deploying a fracture repair system 1600 according to an embodiment of the present disclosure.

Step 5 may include pressurizing the balloon 1610 with bone cement 1650 introduced through the application instrument 1620.

FIG. 133 is a partial perspective view of the exemplary rib cage 20 illustrating a step in a method of deploying a fracture repair system 1600 according to an embodiment of the present disclosure.

Step 6 may include continuing to expand the balloon 1610 until the balloon 1610 if fully expanded and spans the first fracture 21, and using fluoroscopy to verify the position of the balloon 1610 in relation to the first fracture 21.

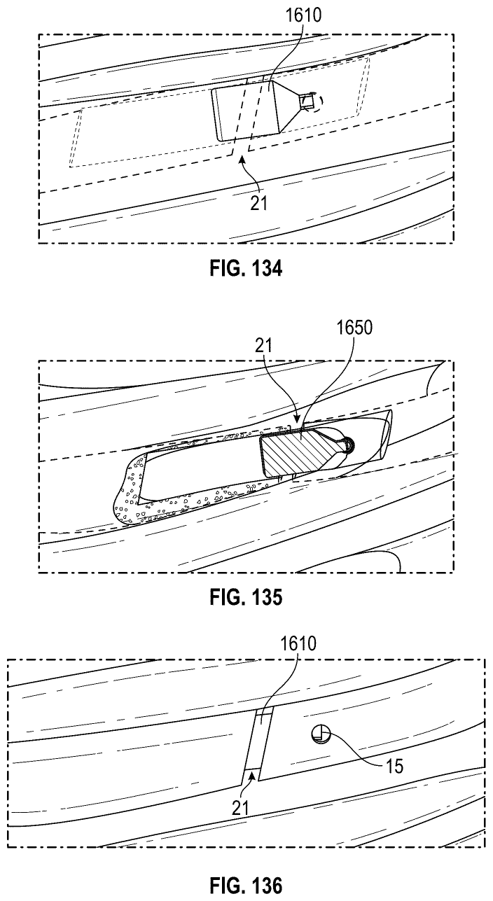

FIG. 134 is a partial perspective view of the exemplary rib cage 20 illustrating a step in a method of deploying a fracture repair system 1600 according to an embodiment of the present disclosure. The balloon 1610 is shown fully expanded within the intramedullary canal and spanning the fracture.

Step 7 may include removing the application instrument 1620 and allowing the bone cement 1650 to harden.

FIG. 135 is a partial perspective section view of the exemplary rib cage 20 illustrating a step in a method of deploying a fracture repair system 1600 according to an embodiment of the present disclosure. The section view of the balloon 1610 shows the bone cement 1650 spanning the first fracture 21.

FIG. 136 is a partial perspective view of the exemplary rib cage 20 illustrating a step in a method of deploying a fracture repair system 1600 according to an embodiment of the present disclosure. The final construct is shown with the balloon 1610 within the intramedullary canal of a portion of bone and spanning the first fracture 21.

A method for stabilizing one or more fractures of a bone may include using a single tether to advance a plate to an interior surface of a bone, using a single tether to advance a first fastener and/or a second fastener to the interior surface of the bone, and/or using a single tether to compress or reduce one or more fractures of a bone. Additionally, or alternatively, the method may include using a second tether to advance a third fastener and/or a fourth fastener to the interior surface of the bone.

A method for stabilizing one or more fractures of a bone may also include introducing a fracture plating system through a VATS portal. A method for stabilizing one or more fractures of a bone may also include introducing a fracture plating system, with a first fastener captive in a first slot of the plate and/or a second fastener captive in a second slot of the plate, through a VATS portal.

A method for stabilizing one or more fractures of a bone may also include receiving fasteners through the bone on opposite sides of each of the one or more fractures. Additionally, or alternatively, the method may include receiving two or more fasteners in a slot of the plate.

A method for stabilizing one or more fractures of a portion of bone may include the following steps:

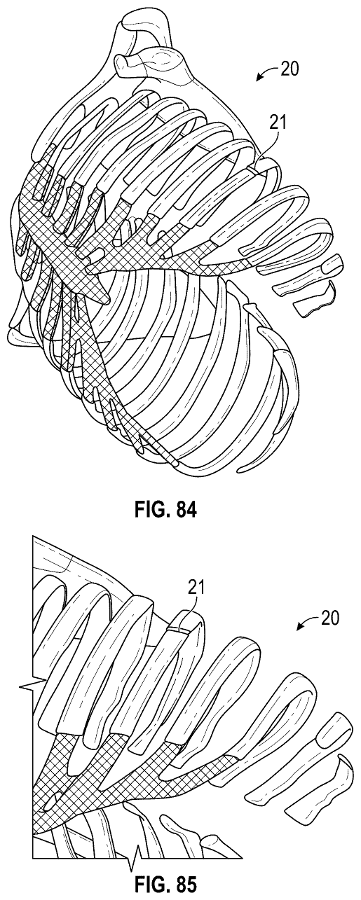

FIG. 84 is a perspective view of an exemplary rib cage 20 with an exemplary first fracture 21. FIG. 85 is a partial perspective view of the exemplary rib cage 20.

Step 1 may include identifying one or more fractures of a bone.

FIG. 86 is a partial perspective view of the exemplary rib cage 20 illustrating a step in a method of deploying a fracture plating system according to an embodiment of the present disclosure.

Step 2 may include drilling a first hole 15 and a second hole 16 wherein the first hole 15 and the second hole 16 are separated by a first fracture 21.

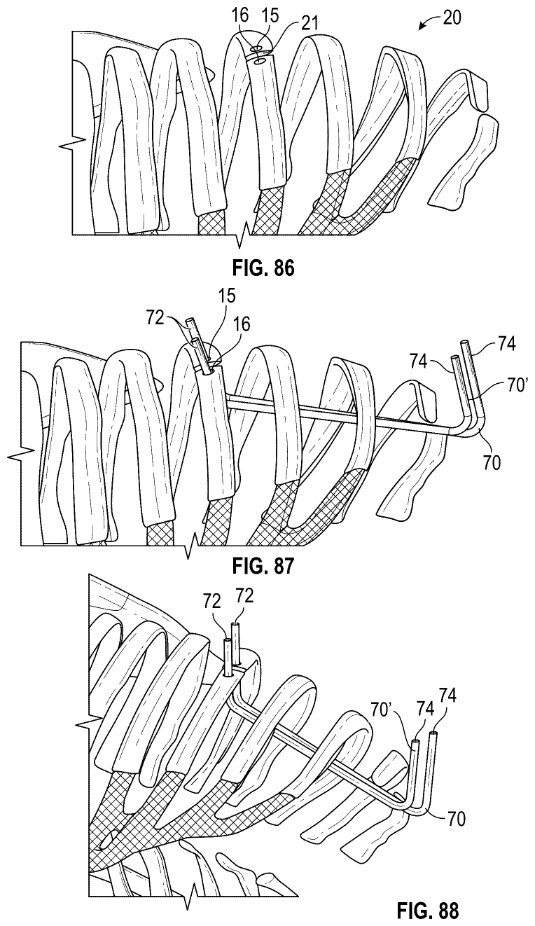

FIG. 87 is a partial perspective view of the exemplary rib cage 20 illustrating a step in a method of deploying a fracture plating system according to an embodiment of the present disclosure. FIG. 88 is a partial perspective view of the exemplary rib cage 20 illustrating a step in a method of deploying a fracture plating system according to an embodiment of the present disclosure.

Step 3 may include inserting a first end 72 of a first flexible guide tube 70 through the first hole 15. Inserting a first end 72 of a second flexible guide tube 70′ through the second hole 16. Pulling the first end 72 of the first flexible guide tube 70 and the first end of the second flexible guide tube out 70 through a VATS portal using an endoscopic grasper or similar instrument. Ensuring that a second end 74 of the first flexible guide tube 70 does not pass through the first hole 15 and ensuring that a second end 74 of the second flexible guide tube 70′ does not pass through the second hole 16. The flexible guide tube 70 may be fabricated of silicone rubber or other biocompatible elastomeric material.

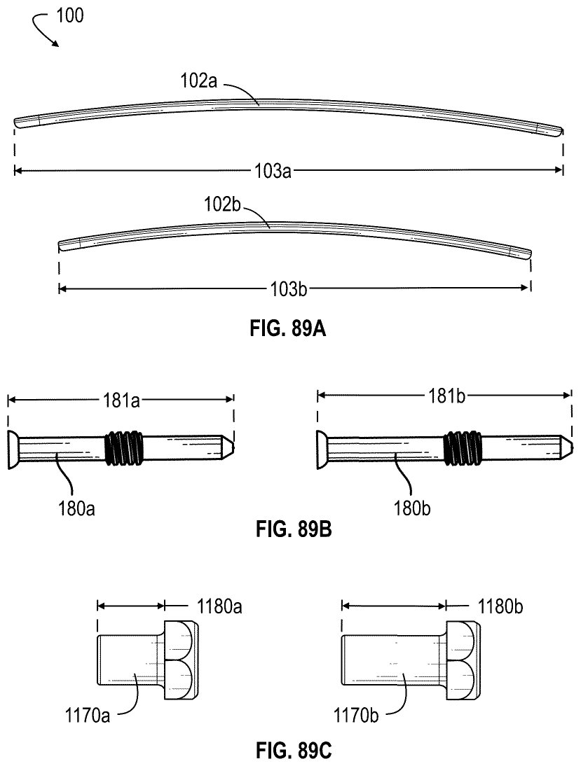

FIG. 89A is a front view of a set of differently sized plates 102 according to an embodiment of the present disclosure. The fracture plating system 100 may configured as a kit which may include a plurality of plates each having a different size and/or configuration, for example different plate length, different plate radius, different plate width, different number of slots, and/or different configuration of slots. The fracture plating system 100 may include a first plate 102a having a first plate length 103a, and a second plate 102b having a second plate length 103b, different from the first plate length 103a.

FIG. 89B is a front view of a set of differently sized circular head fasteners 180 according to an embodiment of the present disclosure. The fracture plating system kit described above may further include a plurality of circular head fasteners 180 each having a different size and/or configuration, for example, different length, different diameter, different thread configuration, and/or different head configuration. The fracture plating system 100 may include a first circular head fastener 180a having a first fastener length 181a, and a second circular head fastener 180b having a second fastener length 181b, different than the first fastener length 181a.

FIG. 89C is a front view of a set of differently sized locking caps 1170 according to an embodiment of the present disclosure. The fracture plating system kit described above may further include a plurality of locking caps 1170 each having a different size and/or configuration, for example, different length, different diameter, different thread configuration, and/or different head configuration. The fracture plating system 100 may include a first locking cap 1170a having a first body length 1180a, and a second locking cap 1170b having a second body length 1180b, different than the first body length 1180a. The first locking cap 1170a and the second locking cap 1170b are interchangeably securable to the first fastener 180a and the second fastener 180b.

FIG. 89D. Is a partial perspective view of an exemplary rib cage 20 showing an exemplary first fracture 21.

Step 4 may include measuring the rib thickness and other features of the portion of rib near the first fracture 21. Selecting a plate from a range of available sizes based on patient anatomy and a location of the fracture. Selecting two or more fasteners from a range of available sizes based on patient anatomy and a location of the fracture.

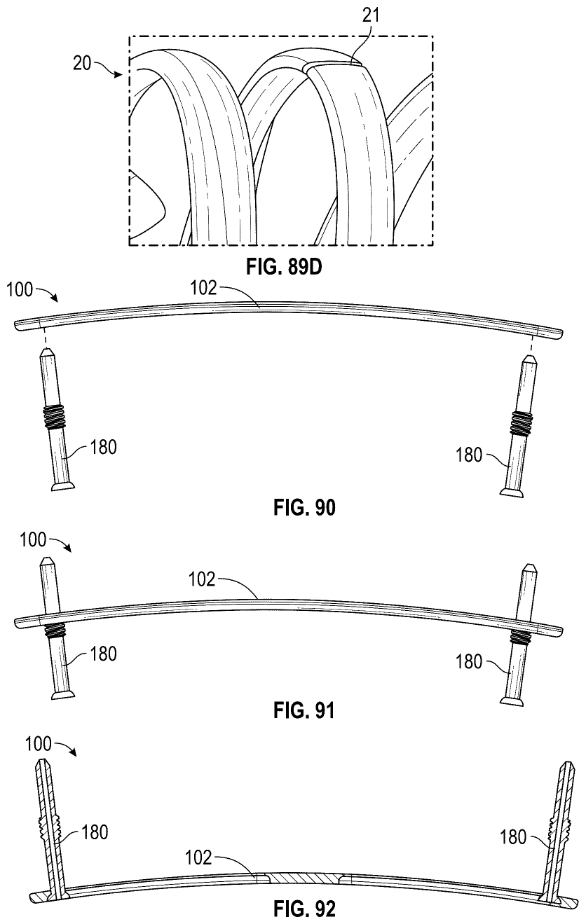

FIG. 90 is a front view of a fracture plating system illustrating a step in a method of deploying the fracture plating system according to an embodiment of the present disclosure. FIG. 91 is a front view of the fracture plating system of FIG. 90 illustrating a step in a method of deploying the fracture plating system according to an embodiment of the present disclosure. FIG. 92 is a front view of the fracture plating system of FIG. 90 illustrating a step in a method of deploying the fracture plating system according to an embodiment of the present disclosure.

Step 5 may include assembling the selected fasteners to the selected plate.

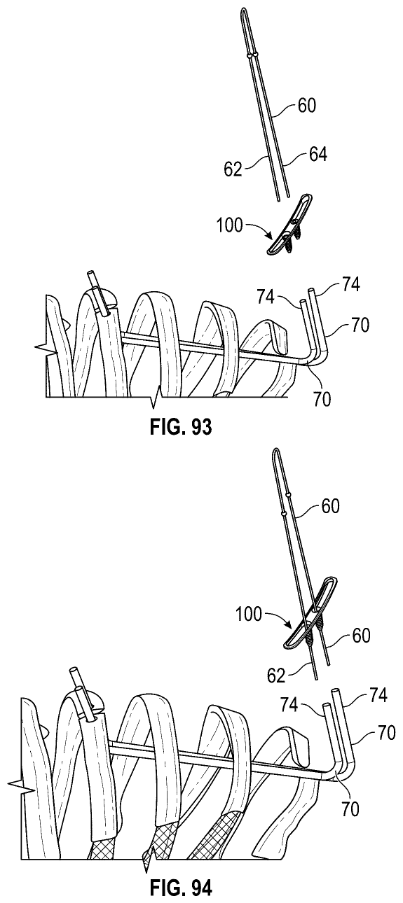

FIG. 93 is a partial perspective view of the exemplary rib cage 20 illustrating a step in a method of deploying a fracture plating system according to an embodiment of the present disclosure.

Step 6 may include inserting a first tether end 62 through a first fastener and a second tether end 64 through a second fastener.

FIG. 94 is a partial perspective view of the exemplary rib cage 20 illustrating a step in a method of deploying a fracture plating system according to an embodiment of the present disclosure.

Step 7 may include inserting a first tether end 62 into a second end 74 of the first flexible guide tube 70 and inserting the second tether end 64 into a second end 74 of the second flexible guide tube 70′.

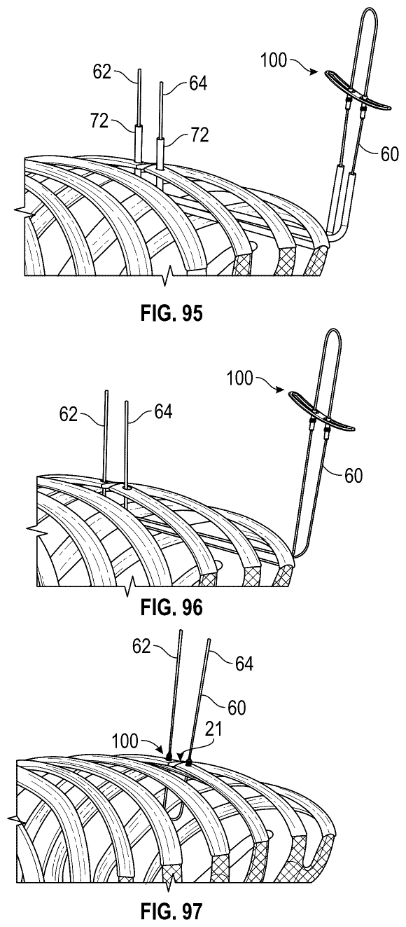

FIG. 95 is a partial perspective view of the exemplary rib cage 20 illustrating a step in a method of deploying a fracture plating system according to an embodiment of the present disclosure.

Step 8 may include feeding the first tether end 62 and the second tether end 64 through the first flexible guide tube 70 and the second flexible guide tube 70′, respectively until the first tether end 62 and the second tether end 64 protrude through the first end 72 of the first flexible guide tube 70 and the first end 72 of the second flexible guide tube 70′.

FIG. 96 is a partial perspective view of the exemplary rib cage 20 illustrating a step in a method of deploying a fracture plating system according to an embodiment of the present disclosure.

Step 9 may include removing the first flexible guide tube 70 and the second flexible guide tube 70′ from the surgical site by pulling the first end 72 of the first flexible guide tube 70 and the first end 72 of the second flexible guide tube 70′.

FIG. 97 is a partial perspective view of the exemplary rib cage 20 illustrating a step in a method of deploying a fracture plating system according to an embodiment of the present disclosure.

Step 10 may include pull the first tether end 62 and the second tether end 64 until the first fastener protrudes through the first hole 15 and the second fastener protrudes through the second hole 16. Optionally, continuing to pull until the fracture is reduced.

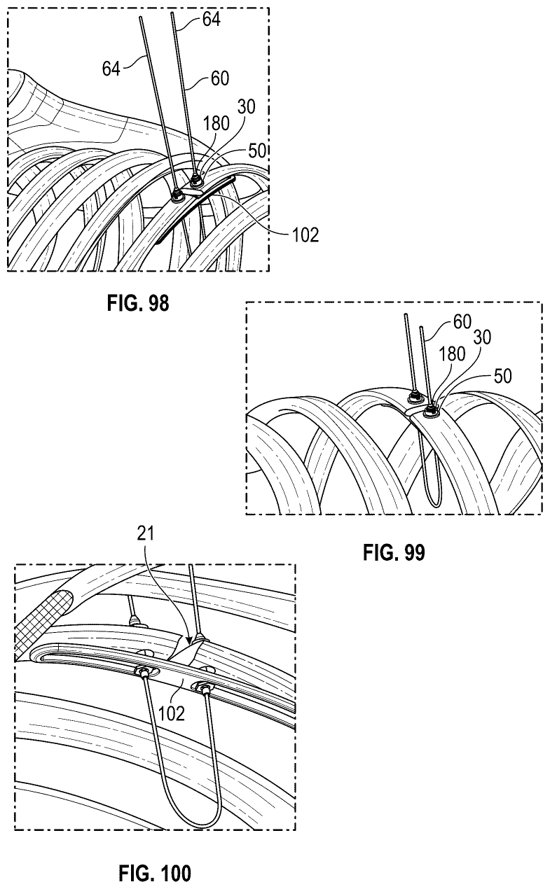

FIG. 98 is a partial perspective view of the exemplary rib cage 20 illustrating a step in a method of deploying a fracture plating system according to an embodiment of the present disclosure.

Step 11 may include placing a first washer over the first tether end 62 and then over the tip portion of the first fastener and placing a second washer over the second tether end 64 and then over the tip portion of the second fastener.

FIG. 99 is a partial perspective view of the exemplary rib cage 20 illustrating a step in a method of deploying a fracture plating system according to an embodiment of the present disclosure. FIG. 100 is a partial perspective view of the exemplary rib cage 20 illustrating a step in a method of deploying a fracture plating system according to an embodiment of the present disclosure.

Step 12 may include placing a first locking cap 30 over the first tether end 62 and threading and tightening the first locking cap onto the threaded portion of the first fastener. Placing a second locking cap 30 over the second tether end 64 and threading and tightening the second locking cap onto the threaded portion of the second fastener.

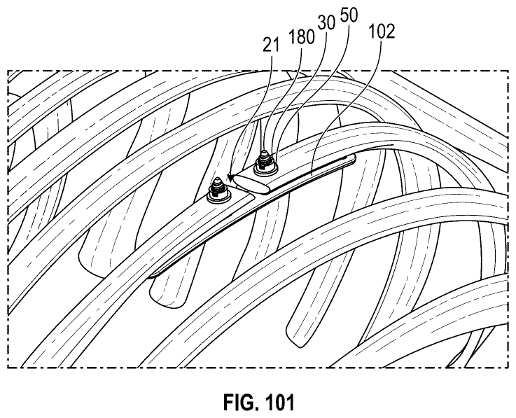

FIG. 101 is a partial perspective view of the exemplary rib cage 20 illustrating a step in a method of deploying a fracture plating system according to an embodiment of the present disclosure.

Step 13 may include removing the single tether 60 by pulling it with an endoscopic grasper, or similar instrument, through the VATS portal.

A method for stabilizing one or more fractures of a bone may include using a tether to advance a first fastener and/or a second fastener to the interior surface of the bone. The method may further include introducing the first fastener and/or the second fastener through a VATS portal. The method may also include introducing the plate to an exterior surface of the bone. The method may include advancing the fasteners through a hole in the bone so that a head portion contacts the interior surface and a distal end extends from the exterior surface of the bone. The method may further include receiving the distal end within a slot of the plate, and, using the fastener, securing the plate to the exterior surface of the bone.

A method for stabilizing one or more fractures of a portion of bone may include the following steps:

Step 1 may include identifying one or more fractures of a bone.

Step 2 may include drilling a first hole 15 and a second hole 16 wherein the first hole 15 and the second hole 16 are separated by a first fracture 21. The step may further include drilling additional holes through the bone on opposite sides of additional fractures, so that each fracture is between two holes in the bone.

Step 3 may include inserting a first end 72 of a first flexible guide tube 70 through the first hole 15. Inserting a first end 72 of a second flexible guide tube 70′ through the second hole 16. Pulling the first end 72 of the first flexible guide tube 70 and the first end of the second flexible guide tube out 70 through a VATS portal using an endoscopic grasper or similar instrument. Ensuring that a second end 74 of the first flexible guide tube 70 does not pass through the first hole 15 and ensuring that a second end 74 of the second flexible guide tube 70′ does not pass through the second hole 16. The flexible guide tube 70 may be fabricated of silicone rubber or other biocompatible elastomeric material.

Step 4 may include measuring the rib thickness and other features of patient anatomy and a location of the fracture. Selecting two or more fasteners from a range of available sizes based on patient anatomy and a location of the fracture. Selecting a plate from a range of available sizes based on patient anatomy and location/quantity of one or more fractures.

Step 5 may include inserting a first tether end 62 through a first fastener and a second tether end 64 through a second fastener.

Step 6 may include inserting a first tether end 62 into a second end 74 of the first flexible guide tube 70 and inserting the second tether end 64 into a second end 74 of the second flexible guide tube 70′.

Step 7 may include feeding the first tether end 62 and the second tether end 64 through the first flexible guide tube 70 and the second flexible guide tube 70′, respectively until the first tether end 62 and the second tether end 64 protrude through the first end 72 of the first flexible guide tube 70 and the first end 72 of the second flexible guide tube 70′.

Step 8 may include removing the first flexible guide tube 70 and the second flexible guide tube 70′ from the surgical site by pulling the first end 72 of the first flexible guide tube 70 and the first end 72 of the second flexible guide tube 70′.

Step 9 may include pulling the first tether end 62 and the second tether end 64 until the first fastener is received in the first hole 15 and the second fastener is received in the second hole 16.

Step 10 may optionally include repeating Step 3 through Step 9 to introduce additional fasteners through additional holes in the bone.

Step 11 may include placing the plate on an exterior surface of the bone so that the plate spans the one or more fractures and the fasteners are received within one or more slots of the plate. The plate may be guided to the exterior surface by receiving the first tether end 62 and the second tether end 64 through one or more slots of the plate. The plate may be placed on the exterior surface of the bone through an open incision and/or through a transverse skin tunnel connecting two vertical soft tissue tunnels.

Step 12 may include securing the plate to the exterior surface of the bone by placing a first locking cap 30 over the first tether end 62 and threading and tightening the first locking cap onto the threaded portion of the first fastener and placing a second locking cap 30 over the second tether end 64 and threading and tightening the second locking cap onto the threaded portion of the second fastener. Optionally, additional locking caps 30 may be used to engage additional fasteners.

Step 13 may include removing the single tether 60 by pulling it with an endoscopic grasper, or similar instrument, through the VATS portal and/or a trans-intercostal incision.

Those of skill in the art will recognize that this is only one of many potential methods that may be used to stabilize one or more fractures of a bone. In alternative embodiments, different methods may be used to implant the fracture plating system 100 or other fracture plating systems described above. Further, the method set forth in FIG. 84 though FIG. 101 may be used to implant other fracture plating systems besides those specifically disclosed herein.

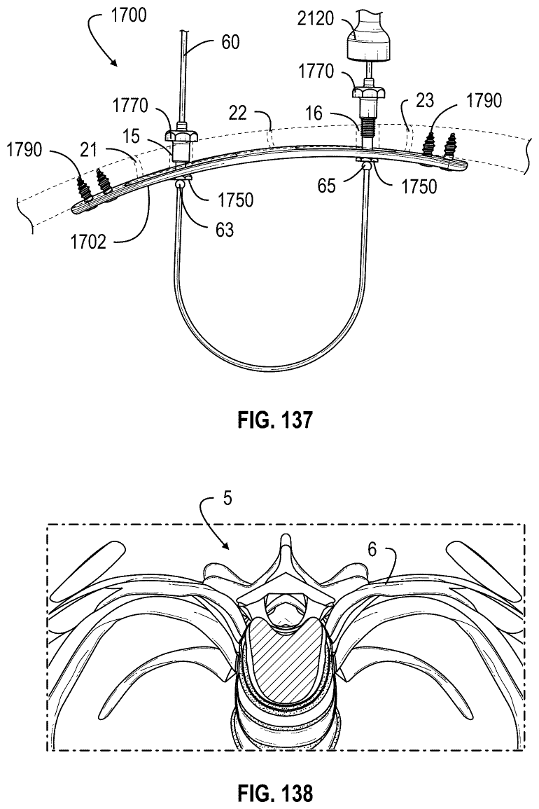

FIG. 137 is a perspective view of a fracture plating system 1700 in a partially deployed configuration spanning exemplary bone fractures according to an embodiment of the present disclosure. The fracture plating system 1700 may include similar features as other fracture plating systems previously described within the present disclosure. The fracture plating system 1700 may include a fastener 1750, a cap 1770, and a plate 1702.

The fracture plating system 1700 may be configured to secure a rib fracture that may be positioned near a spine 5. Additionally, or alternatively, the fracture plating system 1700 may be configured to receive a locking screw 1790 configured to secure the plate 1702 to the bone in addition to one or more fasteners 1750 and one or more caps 1770. The locking screw 1790 may allow a surgeon to fixate an additional rib fracture without having to drill a hole in the rib from the exterior side of the rib. The fracture plating system 1700 may include one or more locking screws 1790 configured to be received at one or more ends of the plate 1702.

The locking screw 1790 may be configured to threadably engage the bone without passing through to an opposite side of the bone. Additionally, the locking screw 1790 may be configured to be secured directly to the bone without the need for a nut and/or a cap.

FIG. 138 is a perspective view of an exemplary axial cross-section view of a spine 5 and ribs. A first fracture 21 may be present on a rib proximate the spine 5 and may be very difficult, if not impossible, to plate with previously known methods because the first fracture 21 may be hidden behind a transverse process 11 of the spine 5. Thus, a surgeon may be unable to access the first fracture 21 with instruments from an exterior approach.

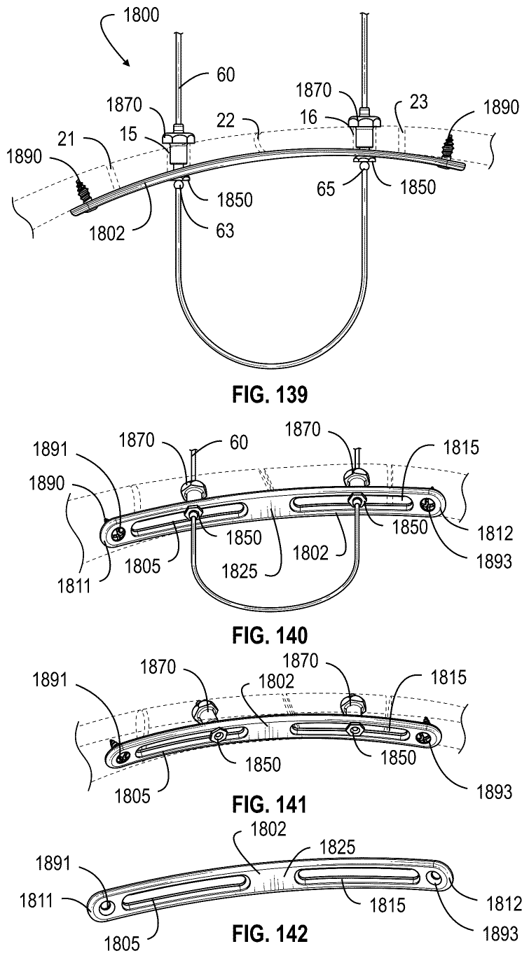

FIG. 139 is a perspective view of a fracture plating system 1800 in a partially deployed configuration spanning an exemplary first fracture 21, an exemplary second fracture 22, and an exemplary third fracture 23 according to an embodiment of the present disclosure. The fracture plating system 1800 may include similar features as other fracture plating systems previously described within the present disclosure. The fracture plating system 1800 may include a fastener 1850, a cap 1870, a plate 1802, and a locking screw 1890.

FIG. 140 is a perspective view of the fracture plating system 1800 in a partially deployed configuration spanning the exemplary first fracture 21, the exemplary second fracture 22, and the exemplary third fracture 23 according to an embodiment of the present disclosure. FIG. 141 is a perspective view of the fracture plating system of FIG. 139 in a deployed configuration spanning the exemplary first fracture 21, the exemplary second fracture 22, and the exemplary third fracture 23 according to an embodiment of the present disclosure. FIG. 142 is a perspective view of a plate of the fracture plating system of FIG. 139 according to an embodiment of the present disclosure.

The plate 1802 may include a first end 1811, a second end 1812 opposite the first end 1811, and a central portion 1825. The plate 1802 may further include a first slot 1805 positioned between the first end 1811 and the central portion 1825. The plate 1802 may further include a second slot 1815 positioned between the second end 1812 and the central portion 1825. The first slot 1805 and the second slot 1815 may be configured to receive a fastener 1850. Additionally, or alternatively, the first slot 1805 and the second slot 1815 may include similar features as other slots previously described within the present disclosure. Additionally, or alternatively, the first slot 1805 and the second slot 1815 may be configured to receive one or more of the fasteners previously described within the present disclosure.

The plate 1802 may further include a first screw aperture 1891 positioned between the first end 1811 and the first slot 1805. The plate 1802 may further include a second screw aperture 1893 positioned between the second end 1812 and the second slot 1815. The first screw aperture 1891 and the second screw aperture 1893 may be configured to receive a locking screw 1890. Additionally, the first screw aperture 1891, the second screw aperture 1893, and the locking screw 1890 may be configured to secure the plate 1802 to a bone. More specifically, the first screw aperture 1891, the second screw aperture 1893, and the locking screw 1890 may be configured to secure the plate 1802 to an internal surface of a rib.

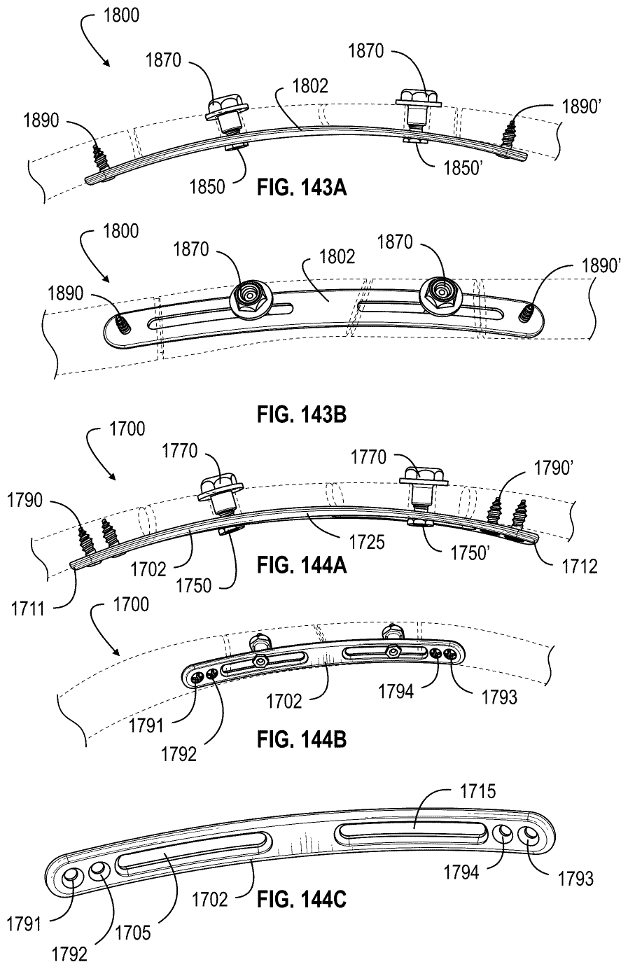

FIG. 143A is a perspective view of the fracture plating system 1800 in a deployed configuration spanning the exemplary first fracture 21, the exemplary second fracture 22, and the exemplary third fracture 23 according to an embodiment of the present disclosure. FIG. 143B is a perspective view of the fracture plating system 1800. The fracture plating system 1800 may be configured so that the first fracture 21 may be positioned between a first fastener 1850 and a second fastener 1850′, the second fracture 22 may be positioned between the first fastener 1850 and a first locking screw 1890, and the third fracture 23 may be positioned between the second fastener 1850′ and a second locking screw 1890′.

FIG. 144A is a perspective view of the fracture plating system of 1700 in a deployed configuration spanning exemplary bone fractures according to an embodiment of the present disclosure. FIG. 144B is a perspective view of the fracture plating system 1700. FIG. 144C is a perspective view of a plate 1702 of the fracture plating system 1700 according to an embodiment of the present disclosure. The fracture plating system 1700 may include similar features as the fracture plating system 1800. The fracture plating system 1700 may include a fastener 1750, a cap 1770, a plate 1702, and a locking screw 1790.

The plate 1702 may include a first end 1711, a second end 1712 opposite the first end 1711, and a central portion 1725. The plate 1702 may further include a first slot 1705 positioned between the first end 1711 and the central portion 1725. The plate 1702 may further include a second slot 1715 positioned between the second end 1712 and the central portion 1725

The plate 1702 may further include a first screw aperture 1791 and a third screw aperture 1792 positioned between the first end 1711 and the first slot 1705. The plate 1702 may further include a second screw aperture 1793 and a fourth screw aperture 1794 positioned between the second end 1712 and the second slot 1715. The first screw aperture 1791, the third screw aperture 1792, the second screw aperture 1793, and the fourth screw aperture 1794 may be configured to receive a locking screw 1790.

The fracture plating system 1700 may be configured so that the first fracture 21 may be positioned between a first fastener 1750 and a second fastener 1750′, the second fracture 22 may be positioned between the first fastener 1750 and first and third locking screws 1790, and the third fracture 23 may be positioned between the second fastener 1750′ and second and fourth locking screws 1790′. The fracture plating system 1700, more specifically the two locking screws 1790 positioned at an end of the plate 1702, may be configured to inhibit rotation of the rib about an axis of the fracture and/or about an axis of a locking screw.

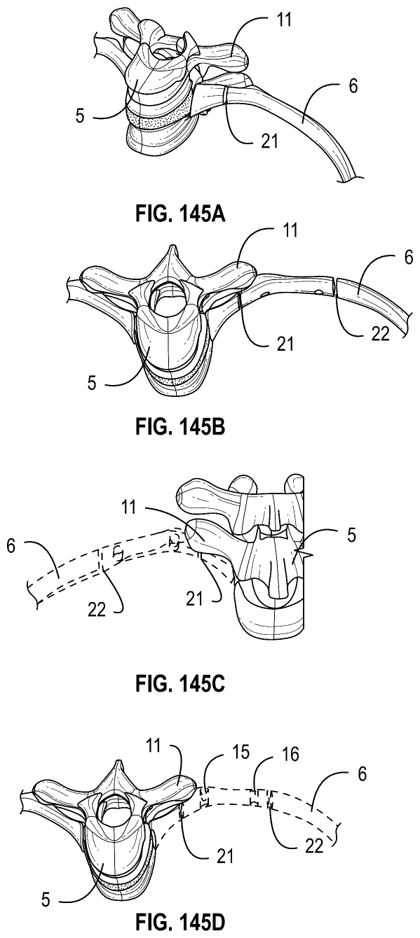

FIG. 145A is a perspective view of an exemplary axial cross-section view of a spine 5 and a rib 6 with a first fracture 21. FIG. 145B is a perspective view of an exemplary axial cross-section view of a spine 5 and a rib 6 with a first fracture 21 and a second fracture 22. FIG. 145C is a perspective view of an exemplary axial cross-section view of a spine 5 and a rib 6 with a first fracture 21 and a second fracture 22. FIG. 145D is a perspective view of an exemplary axial cross-section view of a spine 5 and a rib 6 with a first fracture 21 and a second fracture 22.

A rib fracture near the spine 5 may be difficult to fix because the transverse process 11 may not allow plates or screws to be installed from an exterior approach. The fracture plating system 1700 and/or the fracture plating system 1800 may be configured to secure a fracture located near the spine 5.

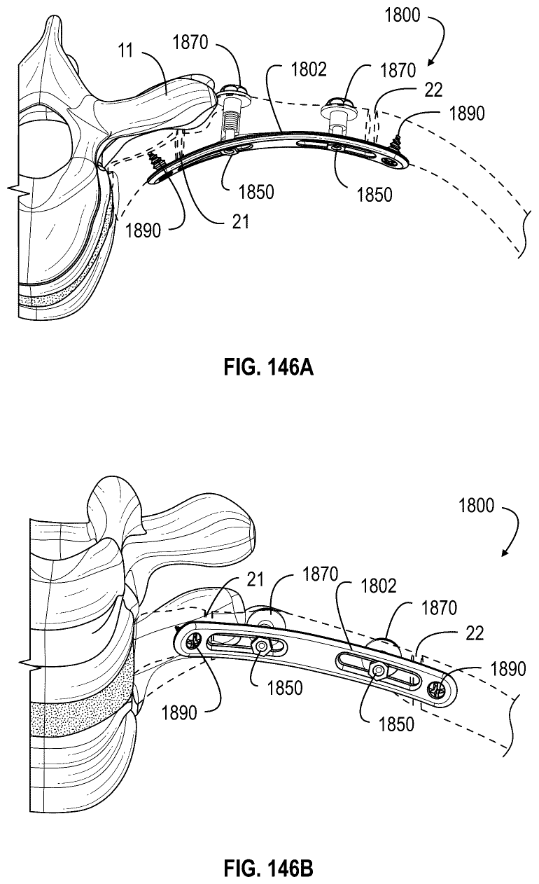

FIG. 146A is a perspective view of a fracture plating system 1800 in a deployed configuration spanning an exemplary first fracture 21 and an exemplary second fracture 22 according to an embodiment of the present disclosure. FIG. 146B is a perspective view of the fracture plating system 1800 in a deployed configuration spanning the exemplary first fracture 21 and the exemplary second fracture 22.

The fracture plating system 1800 may be configured so that the first fracture 21 may be positioned between a first fastener 1850 and the first screw aperture 1891 and the second fracture 22 may be positioned between a second fastener 1850′ and the second screw aperture 1893.

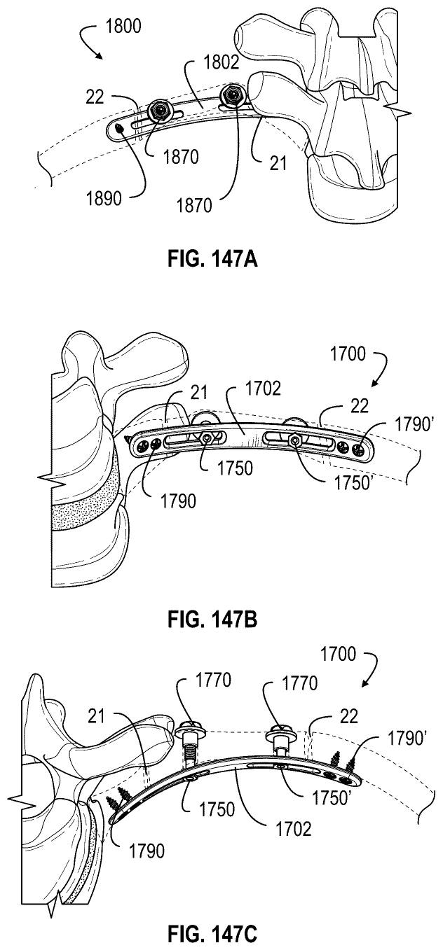

FIG. 147A is a perspective view of a fracture plating system 1800 in a deployed configuration spanning the exemplary first fracture 21 and the exemplary second fracture 22. The fracture plating system 1800 may be configured so that the first screw aperture 1891 is positioned proximate the spine 5. More specifically, the first screw aperture 1891 may be positioned between the first fracture 21 and the spine 5. Additionally, or alternatively, the first screw aperture 1891 may be positioned along a portion of the rib anterior to the transverse process 11.

FIG. 147B is a perspective view of a fracture plating system 1700 in a deployed configuration spanning an exemplary first fracture 21 and an exemplary second fracture 22 according to an embodiment of the present disclosure. FIG. 147C is a perspective view of a fracture plating system 1700 in a deployed configuration spanning the exemplary first fracture 21 and the exemplary second fracture 22. The fracture plating system 1700 may be positioned relative to the first fracture 21 and the spine 5 as previously described for the fracture plating system 1800. The fracture plating system 1700 may include additional screw apertures and additional locking screws as relative to the fracture plating system 1800. A first locking screw 1790 and a second locking screw 1790′ may be configured to inhibit rotation of a portion of the rib about an axis of the first locking screw 1790 and/or the second locking screw 1790.

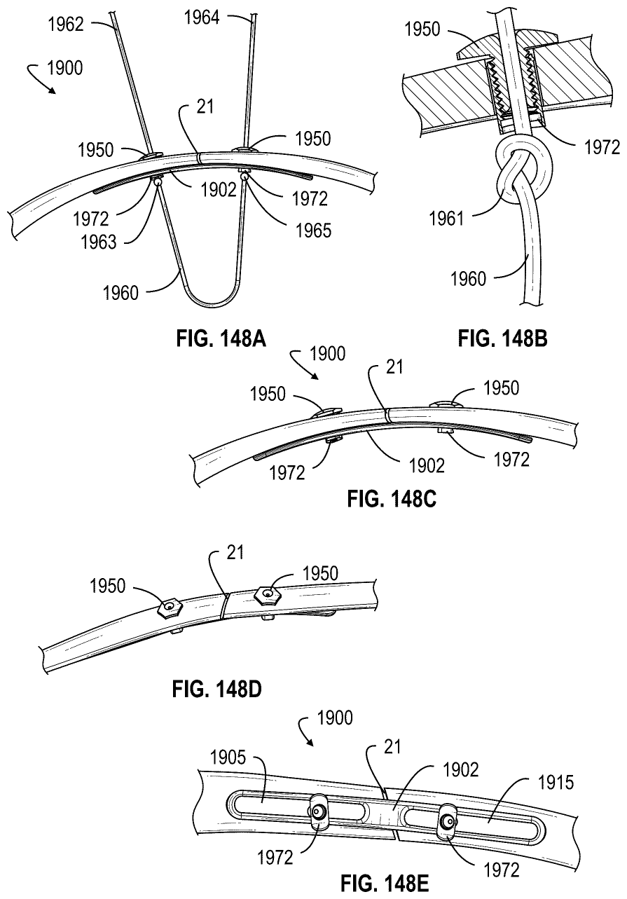

FIG. 148A is a perspective view of a fracture plating system 1900 in a partially deployed configuration spanning an exemplary first fracture 21 according to an embodiment of the present disclosure. The fracture plating system 1900 may include a barrel nut 1972 and a low-profile fastener 1950 that may combine a fastener, a nut and/or a washer previously described within the present disclosure into a lower profile single component. The fracture plating system 1900 may further include a plate 1902 and a tether 1960.

The tether 1960 may include a first tether end 1962, a first bead 1963, a second tether end 1964, and a second bead 1965. The tether 1960 may include similar features and may be configured for similar functions as the tether 60 previously described within the present disclosure. The first bead 1963 and the second bead 1965 may be configured to engage the low-profile fastener 1950 to guide the low-profile fastener 1950 and/or the plate 1902 toward the first fracture 21 so that the plate 1902 may span the first fracture 21.

The first bead 1963 and the second bead 1965 may be configured as a solid spherical section of larger diameter than the tether 1960. The first bead 1963 and/or the second bead 1965 may include a round, square, and/or irregular shape. Additionally, or alternatively, the first bead 1963 and the second bead 1965 may be configured to be secured to the tether 1960. The first bead 1963 and the second bead 1965 may be secured to the tether 1960 by crimping, welding, brazing, adhesive, and/or another method known in the art. Additionally, or alternatively, with the first bead 1963 and the second bead 1965 secured to the tether 1960, the first bead 1963 and the second bead 1965 may be configured to be immoveable relative to the tether 1960.

Additionally, or alternatively, the first bead 1963 and/or the second bead 1965 may be configured as a pin configured to pass through, and be secured to, the tether 1960. Additionally, or alternatively, the first bead 1963 and/or the second bead 1965 may be configured as a build-up of material on the tether 1960. Additionally, or alternatively, the first bead 1963 and/or the second bead 1965 may be configured as a localized increase in a cross-section of the tether 1960 created through an additive manufacturing process.

FIG. 148B is a perspective view of a barrel nut 1972, a low-profile fastener 1950, and a tether 1960 of the fracture plating system 1900 in a partially deployed configuration according to an embodiment of the present disclosure. The first bead 1963 and/or the second bead 1965 may be configured as a knot 1961 tied in the tether 1960. The knot 1961 may be configured to function in a manner similar to the first bead 1963 and/or the second bead 1965 as previously described. The fracture plating system 1900 may be configured so that the knot 1961 may be tied in the tether 1960 after the first tether end 1962 and/or the second tether end 1964 is passed through the barrel nut 1972. The knot 1961 may be configured to engage the barrel nut 1972 to guide the barrel nut 1972 and/or the plate 1902 toward the first fracture 21 so that the plate 1902 may span the first fracture 21. The knot 1961 may be further configured to inhibit the barrel nut 1972 from disengaging from the tether 1960 when the knot 1961 is present. Additionally, the knot 1961 may be configured so that it may be untied, thereby allowing the first tether end 1962 and/or the second tether end 1964 to be removed from the barrel nut 1972. The tether 1960, which may include the knot 1961, may be compatible with any fracture plating system previously described within the present disclosure.

FIG. 148C is a perspective view of the fracture plating system 1900 in a deployed configuration spanning the exemplary first fracture 21 according to an embodiment of the present disclosure. FIG. 148D is a perspective view of the fracture plating system 1900 in a deployed configuration spanning the exemplary first fracture 21. The low-profile fastener 1950 may be configured so that less material may extend proud on an outside of the bone and/or rib.

FIG. 148E is a perspective view of the fracture plating system 1900 in a deployed configuration spanning the exemplary first fracture 21. The low-profile fastener 1950 may be configured as a low-profile cap that may engage the barrel nut 1972 to secure a plate 1902 to a bone to secure a first fracture 21. The barrel nut 1972 and the low-profile fastener 1950 may be configured to be compatible with other plates previously described within the present disclosure.

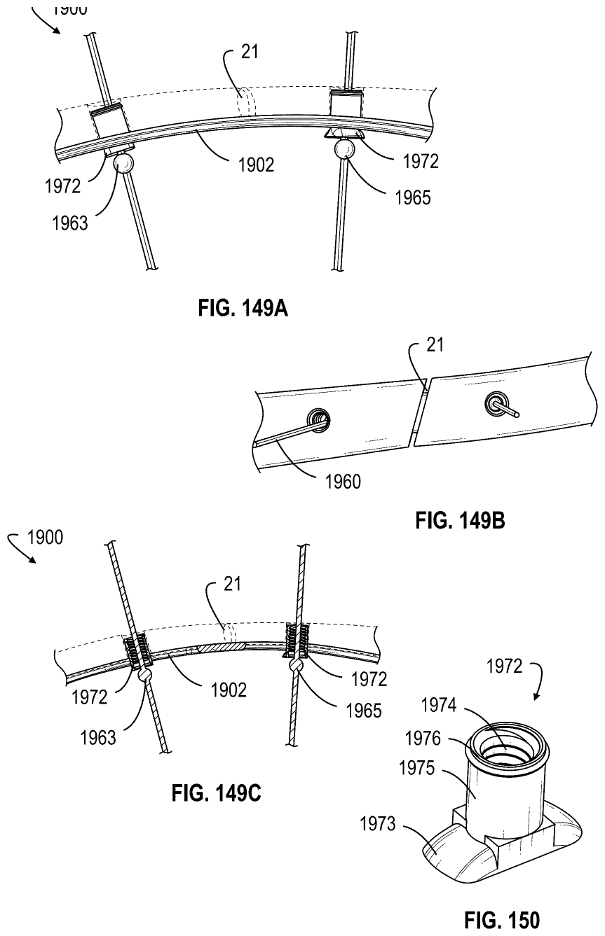

FIG. 149A is a perspective view of a fracture plating system 1900 in a partially deployed configuration spanning an exemplary first fracture 21 of a bone according to an embodiment of the present disclosure. FIG. 149B is a perspective view of the fracture plating system 1900 in a partially deployed configuration spanning an exemplary first fracture 21. FIG. 149C is a perspective view of the fracture plating system 1900 in a partially deployed configuration spanning an exemplary first fracture 21. FIG. 150 is a perspective view of a barrel cap of the fracture plating system 1900 according to an embodiment of the present disclosure.

The barrel nut 1972 may include a wing portion 1973, a threaded portion 1974, a body portion 1975, and a proximal end 1976 opposite the wing portion 1973. The body portion 1975 may be configured to be received in and pass through a first slot 1905 and/or a second slot 1915 of the plate 1902. Additionally, the body portion 1975 may be configured to be received in a hole 15 and/or a hole 16 proximate the first fracture 21. The body portion 1975 may be further configured so that, with the barrel nut 1972 received in the hole 15 or the hole 16, the proximal end 1976 may not protrude out of the hole 15 or the hole 16.

The threaded portion 1974 may be configured to receive the low-profile fastener 1950 to secure the plate 1902 to the bone. The wing portion 1973 may extend from the body portion 1975 to inhibit the barrel nut 1972 from passing all the way through the first slot 1905 and/or the second slot 1915. Additionally, or alternatively, the wing portion 1973 may be configured so that, with the barrel nut 1972 in a first orientation, the wing portion 1973 may inhibit the barrel nut 1972 from passing all the way through the first slot 1905 and/or the second slot 1915, and, with the barrel nut 1972 in a second orientation, 90° opposed to the first orientation, the wing portion 1973 may allow the barrel nut 1972 to pass all the way through the first slot 1905 and/or the second slot 1915.

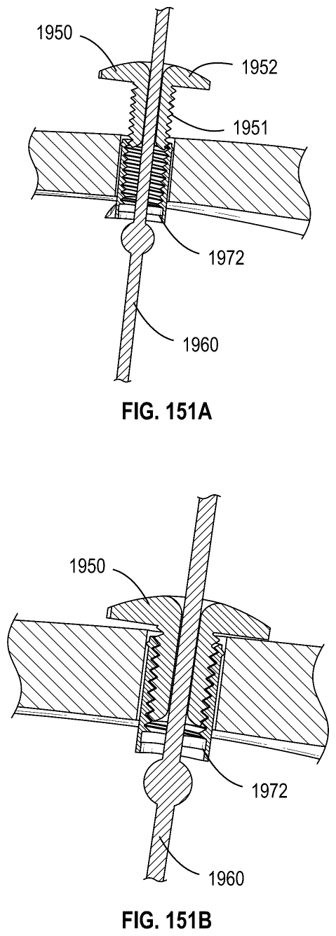

FIG. 151A is a perspective view of the fracture plating system 1900 in a partially deployed configuration according to an embodiment of the present disclosure. FIG. 151B is a perspective view of the fracture plating system 1900 in a partially deployed configuration according to an embodiment of the present disclosure. The low-profile fastener 1950 may include a threaded portion 1951 and a head portion 1952.

The threaded portion 1951 may be configured to be threadably received in the threaded portion 1974 to secure the plate 1902 to the bone. The head portion 1952 may be configured to abut an external surface of the bone. The head portion 1952 may include a non-circular profile to accommodate a driver that may be used to threadably engage the low-profile fastener 1950 with the barrel nut 1972.

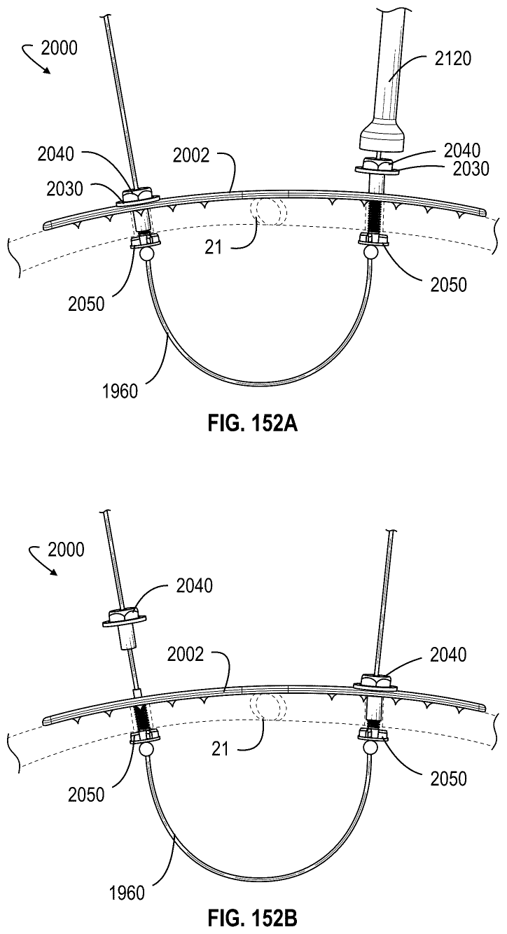

FIG. 152A is a perspective view of a fracture plating system 2000 in a partially deployed configuration spanning an exemplary first fracture 21 according to an embodiment of the present disclosure. The fracture plating system 2000 may include similar features as other fracture plating systems previously described within the present disclosure. The fracture plating system 2000 may include a plate 2002, a fastener 2050, a washer 2030, a cap 2040, and a tether 1960. The fracture plating system 2000 may be compatible with other fasteners, caps, nuts, washers, and/or tethers previous described within the present disclosure.

FIG. 152B is a perspective view of the fracture plating system 2000 in a partially deployed configuration according to an embodiment of the present disclosure. The fracture plating system 2000 may be configured the one or more fasteners 2050 may abut an interior portion of the bone and one or more washers 2030 and/or caps 2040 may abut the plate 2002. The one or more fasteners 2050 may be configured to threadably engage the one or more caps 2040 to secure the plate 2002 to the bone to secure the first fracture 21.

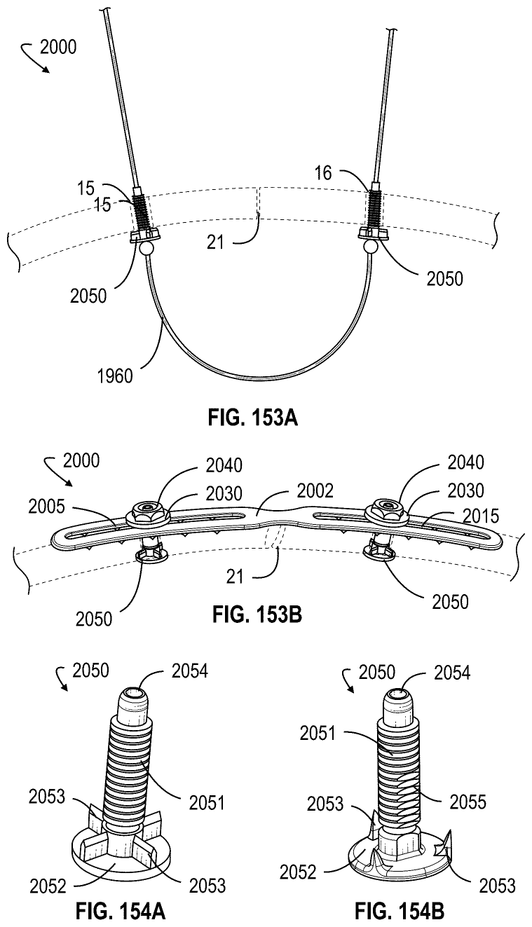

FIG. 153A is a perspective view of a fracture plating system 2000 in a partially deployed configuration spanning an exemplary first fracture 21 according to an embodiment of the present disclosure. FIG. 153B is a perspective view of the fracture plating system 2000 in a deployed configuration according to an embodiment of the present disclosure. The fracture plating system 2000 may be configured for external plate fixation of a fracture in a bone. The plate 2002 may be configured to secure the fracture by being secured to an external surface of the bone. The fracture plating system 2000 may further be configured so that one or more fasteners 2050 may be received on the tether 1960, and guided to the first fracture 21 through an interior portion of the patient. Additionally, the one or more fasteners 2050 may be guided to a one or more holes proximate the first fracture 21 through a VATS portal and/or an intercostal approach.

FIG. 154A is a perspective view of a fastener 2050 of the fracture plating system 2000 according to an embodiment of the present disclosure. The fastener 2050 may be configured to be received in a bone through a hole 15 proximate a first fracture 21. The fastener 2050 may be further configured to be guided to the hole 15 by the tether 1960. The tether 1960 may be configured to guide the fastener 2050 through the hole 15 in an interior to exterior direction. The tether 1960 may be further configured to guide the fastener 2050 through an internal portion of a patient and to the hole 15.

The fastener 2050 may include a threaded portion 2051, a head portion 2052, and a cannulation 2054 extending through the fastener 2050 along a longitudinal axis of the fastener 2050. The threaded portion 2051 may be configured to threadably engage the cap 2040 to secure the plate 2002 to the bone. The threaded portion 2051 may include one or more flats 2055 that may reduce a cross-sectional area of the threaded portion 2051. The one or more flats may be configured to cooperate with a first slot 2005 and/or a second slot 2015 of the plate 2002 to inhibit rotation of the fastener 2050 relative to the plate 2002.

The head portion 2052 may include one or more engagement features 2053. The one or more engagement features 2053 may be configured as one or more blades that may embed into an interior surface of the bone as the cap 2040 is tightened on the fastener 2050 to draw the head portion 2052 toward the interior surface of the bone.

FIG. 154B is a perspective view a fastener 2050 of the fracture plating system 2000 according to an embodiment of the present disclosure. In an embodiment, the one or more engagement features 2053 may be configured as one or more spikes that may embed into the interior surface of the bone. With the one or more engagement features embedded within the interior surface of the bone, the fastener 2050 may be inhibited from rotating relative to the bone.

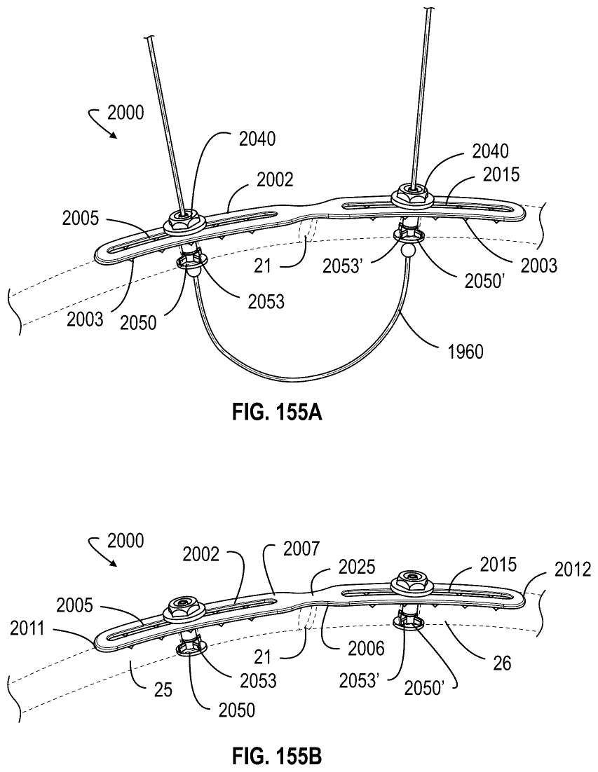

FIG. 155A is a perspective view of the fracture plating system 2000 in a partially deployed configuration spanning an exemplary first fracture 21 according to an embodiment of the present disclosure. FIG. 155B is a perspective view of the fracture plating system 2000 in a deployed configuration spanning an exemplary first fracture 21 according to an embodiment of the present disclosure. The plate 2002 may include a plurality of spikes 2003 extending from a bone facing side 2006 of the plate 2002. The plurality of spikes 2003 may be configured to engage with and/or embed in an exterior surface of the bone. More specifically, the plurality of spikes 2003 may be configured to engage a first rib portion 25 on a first side of the first fracture 21 and a second rib portion 26 on a second side of the first fracture 21.

With the plurality of spikes 2003 embedded in the first rib portion 25 and the second rib portion 26, the first rib portion 25 and/or the second rib portion 26 may be inhibited from moving relative to the plate 2002. Additionally, or alternatively, with a first engagement feature 2053 of a first fastener 2050 embedded in the first rib portion 25 and a second engagement feature 2053′ of a second fastener 2050′ embedded in the second rib portion 26, the first rib portion 25 and/or the second rib portion 26 may be inhibited from moving relative to the first fastener 2050 and/or the second fastener 2050′.

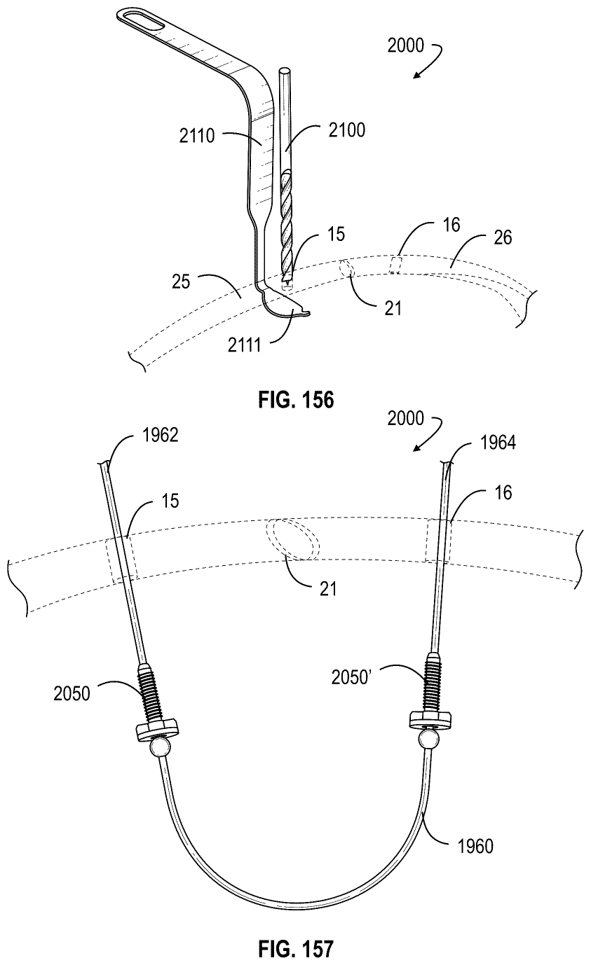

FIG. 156 is a perspective view of a step in a method of deploying a fracture plating system 2000 which may include drilling a hole in a bone according to an embodiment of the present disclosure. The method may include using a drill 2100 to drill a hole 15 in a first rib portion 25 on a first side of a first fracture 21. The method may further include using a soft tissue retractor 2110 to retract soft tissue to exposed the first rib portion 25. The method may further include using a drill stop 2111 of the soft tissue retractor 2110 to prevent over drilling with the drill 2100.

The method may further include using the drill 2100 to drill a hole 16 in a second rib portion 26 on a second side of the first fracture 21. The method may further include using the soft tissue retractor 2110 to retract soft tissue to exposed the second rib portion 26. The method may further include using the drill stop 2111 of the soft tissue retractor 2110 to prevent over drilling with the drill 2100.

FIG. 157 is a perspective view of a step in a method of deploying the fracture plating system 2000 which may include passing a tether 1960 of the fracture plating system 2000 through the hole 15 and the hole 16 according to an embodiment of the present disclosure. A first tether end 1962 of the tether 1960 may be passed through the hole 15 and a second tether end 1964 of the tether 1960 may be passed through the hole 16. The first tether end 1962 and the second tether end 1964 may be passed from an interior portion of the patient to an exterior of the patient. A first fastener 2050 may be received on the first tether end 1962 and a second fastener 2050′ may be received on the second tether end 1964 prior to the first tether end 1962 and the second tether end 1964 being passed through the hole 15 and the hole 16 respectively.

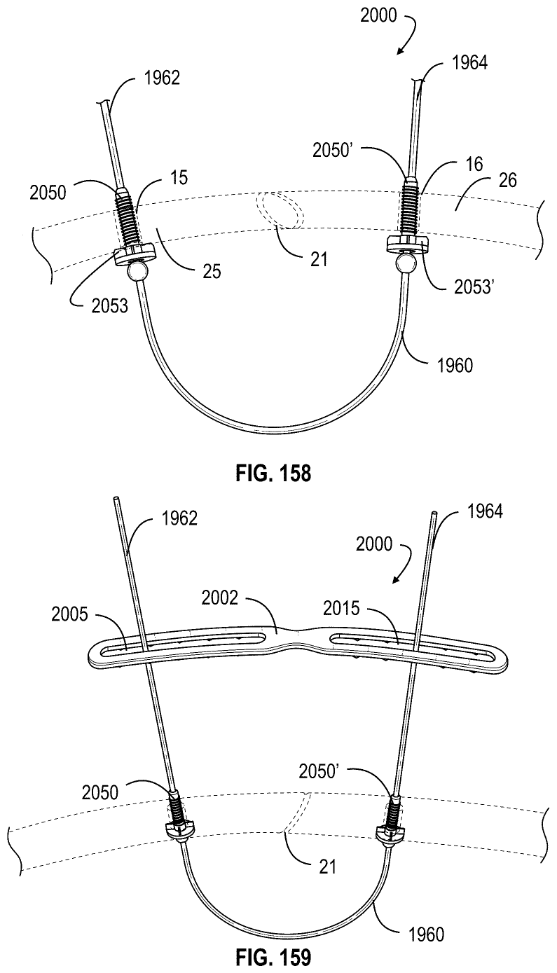

FIG. 158 is a perspective view of a step in a method of deploying the fracture plating system 2000 which may include passing the first fastener 2050 and the second fastener 2050′ through the hole 15 and the hole 16 respectively, according to an embodiment of the present disclosure. The method may further include using the tether 1960 to guide the first fastener 2050 and the second fastener 2050′ into the hole 15 and the hole 16 respectively so that a first engagement feature 2053 of the first fastener 2050 and a second engagement feature 2053′ of the second fastener 2050′ engage an interior surface of the first rib portion 25 and the second rib portion 26 respectively.

FIG. 159 is a perspective view of a step in a method of deploying the fracture plating system 2000 which may include passing a plate 2002 of the fracture plating system 2000 over the tether 1960 according to an embodiment of the present disclosure. The method may further include passing the first tether end 1962 through a first slot 2005 of the plate 2002 and the second tether end 1964 through a second slot 2015 of the plate 2002.

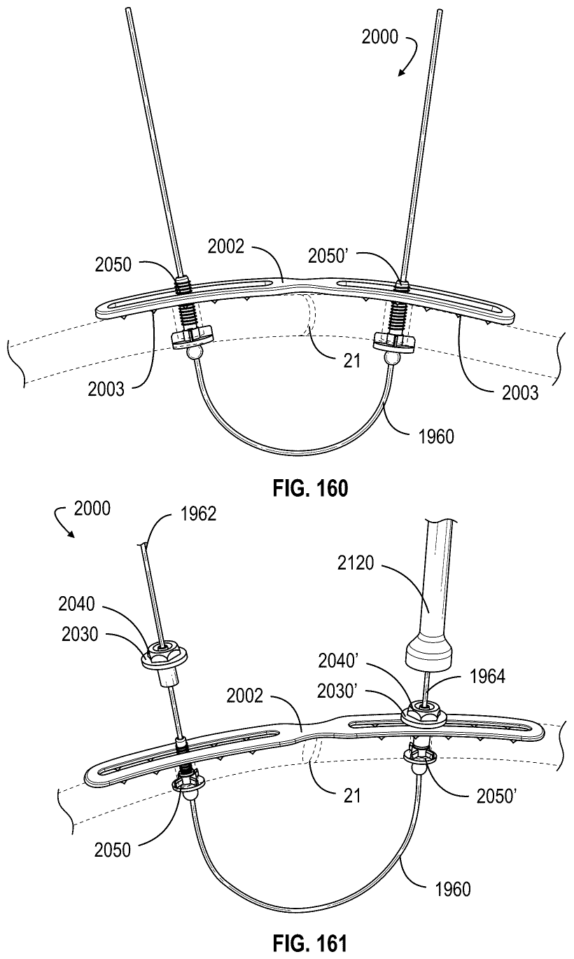

FIG. 160 is a perspective view of a step in a method of deploying the fracture plating system 2000 which may include positioning the plate 2002 on the bone according to an embodiment of the present disclosure. The method may further include using the tether 1960 to guide the plate 2002 to an exterior portion of the bone such that the plate 2002 spans the first fracture 21. The plurality of spikes 2003 may then engage the exterior portion of the bone.

FIG. 161 is a perspective view of a step in a method of deploying the fracture plating system 2000 which may include passing a first cap 2040 and a second cap 2040′ over the tether 1960 according to an embodiment of the present disclosure. A first cap 2040 and a first washer 2030 may be received on the first tether end 1962 and guided to the first pin fastener 1050. A second cap 2040′ and a second washer 2030′ may be received on the second tether end 1964 and guided to the second fastener 2050′.

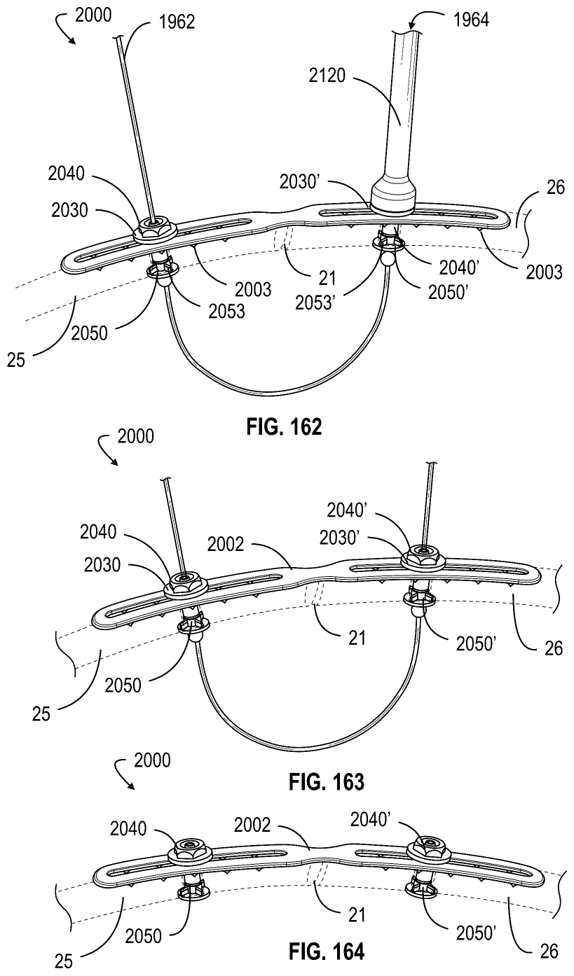

FIG. 162 is a perspective view of a step in a method of deploying the fracture plating system 2000 which may include securing the first cap 2040 and the second cap 2040′ onto the first fastener 2050 and the second fastener 2050′ respectively with a driver 2120 according to an embodiment of the present disclosure. The method may further include advancing the driver 2120 along the first tether end 1962 to engage the first cap 2040 and tightening the first cap 2040 to draw the first engagement feature 2053 into the interior side of the first rib portion 25 and at least some of the plurality of spikes 2003 into the exterior side of the first rib portion 25, thereby securing the plate 2002 to the first rib portion 25. Advancing the driver 2120 along the second tether end 1964 to engage the second cap 2040′ and tightening the second cap 2040′ to draw the second engagement feature 2053′ into the interior side of the second rib portion 26 and at least some of the plurality of spikes 2003 into the exterior side of the second rib portion 26, thereby securing the plate 2002 to the second rib portion 26.

FIG. 163 is a perspective view of a step in a method of deploying the fracture plating system 2000 which may include withdrawing the driver 2120 according to an embodiment of the present disclosure. The method may further include withdrawing the driver 2120 after the first cap 2040 is secured to the first fastener 2050 and the second cap 2040′ is secured to the second fastener 2050′ and the plate 2002 is secured to the first rib portion 25 and the second rib portion 26.

FIG. 164 is a perspective view of a step in a method of deploying the fracture plating system 2000 which may include withdrawing the tether 1960 from the first fastener 2050 and the second fastener 2050′ according to an embodiment of the present disclosure. The method may further include withdrawing the first tether end 1962 through the first fastener 2050 and the second tether end 1964 through the second fastener 2050′ respectively.

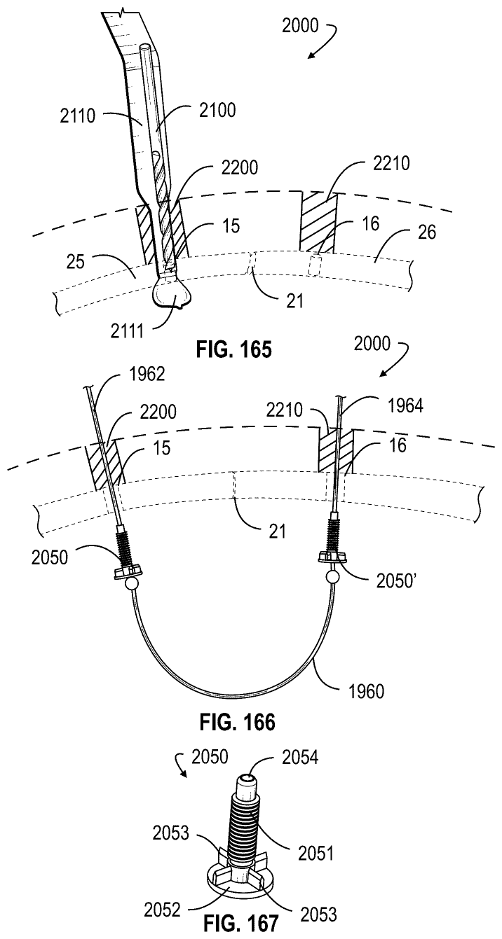

FIG. 165 is a perspective view of a step in a method of deploying a fracture plating system 2000 using a minimally invasive technique, which may include drilling a hole in a bone according to an embodiment of the present disclosure. The method may include creating a first soft tissue tunnel 2200 proximate the first rib portion 25 and using a soft tissue retractor 2110 to retract soft tissue to exposed the first rib portion 25. The method may include using a drill 2100 to drill a hole 15 in the first rib portion 25 on a first side of a first fracture 21. The method may further include using a drill stop 2111 of the soft tissue retractor 2110 to prevent over drilling with the drill 2100.

The method may further include using the drill 2100 to drill a hole 16 in a second rib portion 26 on a second side of the first fracture 21. The method may include creating a second soft tissue tunnel 2210 proximate the second rib portion 26 and using a soft tissue retractor 2110 to retract soft tissue to exposed the second rib portion 26. The method may include using the drill 2100 to drill a hole 16 in the second rib portion 26 on a second side of the first fracture 21. The method may further include using the drill stop 2111 of the soft tissue retractor 2110 to prevent over drilling with the drill 2100.

FIG. 166 is a perspective view of a step in a method of deploying the fracture plating system 2000 which may include passing a tether 1960 of the fracture plating system 2000 through the hole 15 and the hole 16 according to an embodiment of the present disclosure. The method may further include passing a first tether end 1962 of the tether 1960 through the hole 15 and a first soft tissue tunnel 2200 to an exterior portion of a patient. Passing a second tether end 1964 of the tether 1960 through the hole 16 and a second soft tissue tunnel 2210 to the exterior portion of the patient.

FIG. 167 is a perspective view of the fastener 2050 of the fracture plating system 2000 according to an embodiment of the present disclosure. The method may further include receiving a first fastener 2050 on the first tether end 1962 and a second fastener 2050′ on the second tether end 1964 prior to the first tether end 1962 and the second tether end 1964 being passed through the hole 15 and the hole 16 respectively.

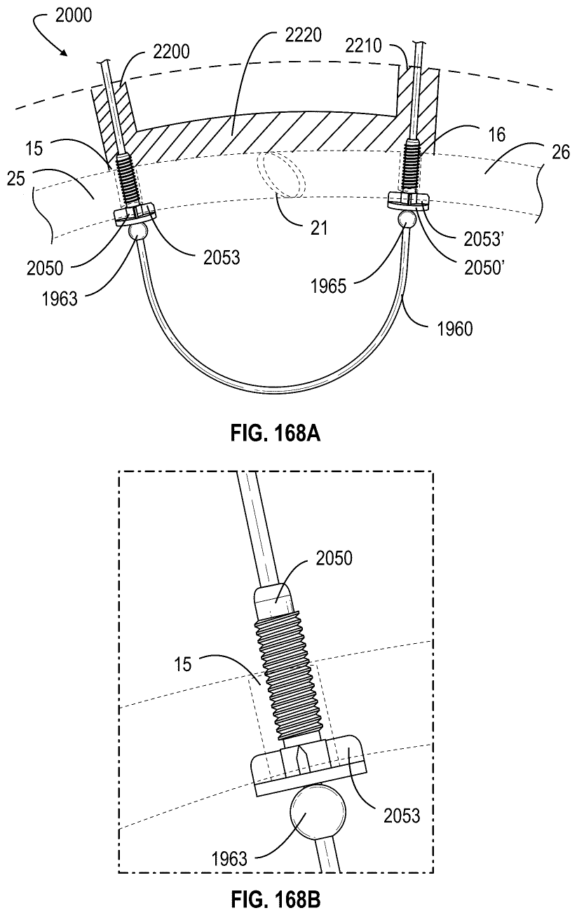

FIG. 168A is a perspective view of a step in a method of deploying the fracture plating system 2000 which may include passing the first fastener 2050 and the second fastener 2050′ through the hole 15 and the hole 16 respectively according to an embodiment of the present disclosure. FIG. 168B is a perspective view of a step in a method of deploying the fracture plating system 2000 which may include passing the first fastener 2050 and the second fastener 2050′ through the hole 15 and the hole 16 respectively. The method may further include using the tether 1960 to draw the first fastener 2050 into the hole 15 and the second fastener 2050′ into the hole 16. The method may further include creating a transverse skin tunnel 2220 just above the bone and below the skin using a blunt rigid or semi-rigid instrument. The transverse skin tunnel 2220 may extend generally between the first soft tissue tunnel 2200 and the second soft tissue tunnel 2210.

A first bead 1963 may engage the first fastener 2050 to convey a force from the tether 1960 to the first fastener 2050. Additionally, a second bead 1965 may engage the second fastener 2050′ to convey a force from the tether 1960 to the second fastener 2050. The method may further include applying sufficient force to the first fastener 2050 and the second fastener 2050′ via the tether 1960 to engage a first engagement feature 2053 of the first fastener 2050 with the first rib portion 25 and a second engagement feature 2053′ of the second fastener 2050′ with the second rib portion 26.

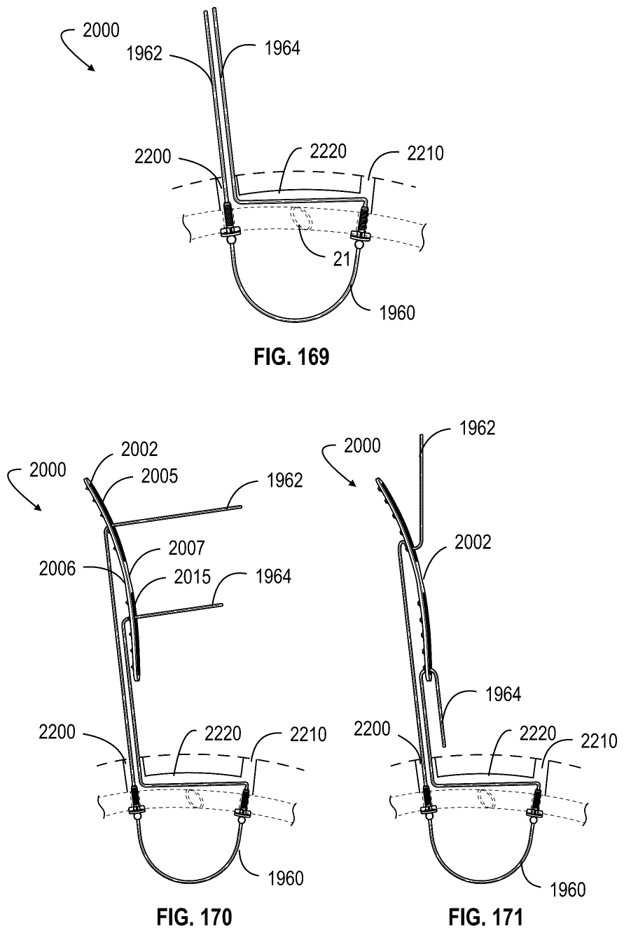

FIG. 169 is a perspective view of a step in a method of deploying the fracture plating system 2000 which may include passing the tether 1960 through a transverse skin tunnel 2220 and through the first soft tissue tunnel 2200 according to an embodiment of the present disclosure. The method may further include passing the second tether end 1964 through the second soft tissue tunnel 2210 toward the interior portion of the body, through the transverse skin tunnel 2220 toward the first soft tissue tunnel 2200 and through the first soft tissue tunnel 2200 toward an exterior portion of the body so that the first tether end 1962 and the second tether end 1964 exit the body through the first soft tissue tunnel 2200.

Passing the tether 1960 through the transverse skin tunnel 2220 may be done with a manual surgical instrument like an endoscopic grasper or any other general surgical instrument with a low profile. Passing the second tether end 1964 may occur by placing a manual instrument in the first soft tissue tunnel 2200, coming thru the transverse skin tunnel 2220 and then grasping the second tether end 1964 with the instrument and pulling it thru the transverse skin tunnel 2220.

FIG. 170 is a perspective view of a step in a method of deploying the fracture plating system 2000 which may include passing the plate 2002 of the fracture plating system 2000 over the tether 1960 according to an embodiment of the present disclosure. The method may further include passing the first tether end 1962 through a first slot 2005 of the plate 2002 and the second tether end 1964 through a second slot 2015 of the plate 2002. The first tether end 1962 and the second tether end 1964 may be passed through the first slot 2005 and the second slot 2015 respectively from a bone facing side 2006 of the plate 2002 toward an exterior facing side 2007, opposite the bone facing side 2006, of the plate 2002.

FIG. 171 is a perspective view of a step in a method of deploying the fracture plating system 2000 which may include passing the tether 1960 back through the first soft tissue tunnel 2200 according to an embodiment of the present disclosure. The method may further include passing the second tether end 1964 back into the first soft tissue tunnel 2200 toward the interior portion of the body.

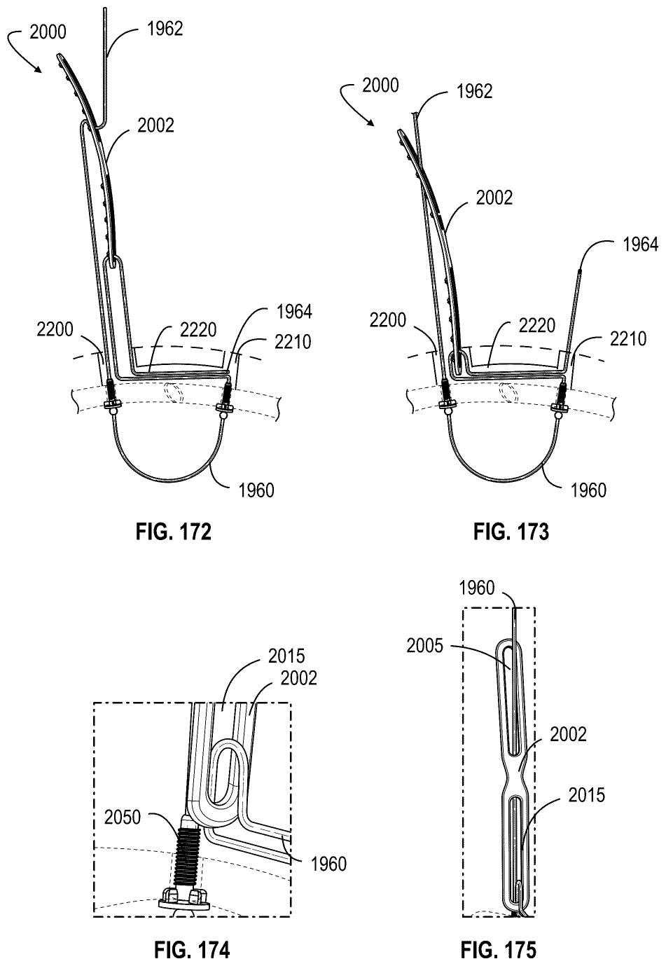

FIG. 172 is a perspective view of a step in a method of deploying the fracture plating system 2000 which may include passing the tether 1960 back through the transverse skin tunnel 2220 according to an embodiment of the present disclosure. The method may further include passing the second tether end 1964 back through the transverse skin tunnel 2220 toward the second soft tissue tunnel 2210.

FIG. 173 is a perspective view of a step in a method of deploying the fracture plating system 2000 which may include passing the tether 1960 through the second soft tissue tunnel 2210 according to an embodiment of the present disclosure. The method may further include passing the second tether end 1964 through the second soft tissue tunnel 2210 toward the exterior portion of the body.

FIG. 174 is a perspective view of a step in a method of deploying the fracture plating system 2000. FIG. 175 is a perspective view of a step in a method of deploying the fracture plating system 2000. As the tether 1960 is passed back through the transverse skin tunnel 2220 and through the second soft tissue tunnel 2210, tether 1960 may remain interwoven with the plate 2002 so that the tether 1960 may guide the plate 2002 into the transverse skin tunnel 2220.

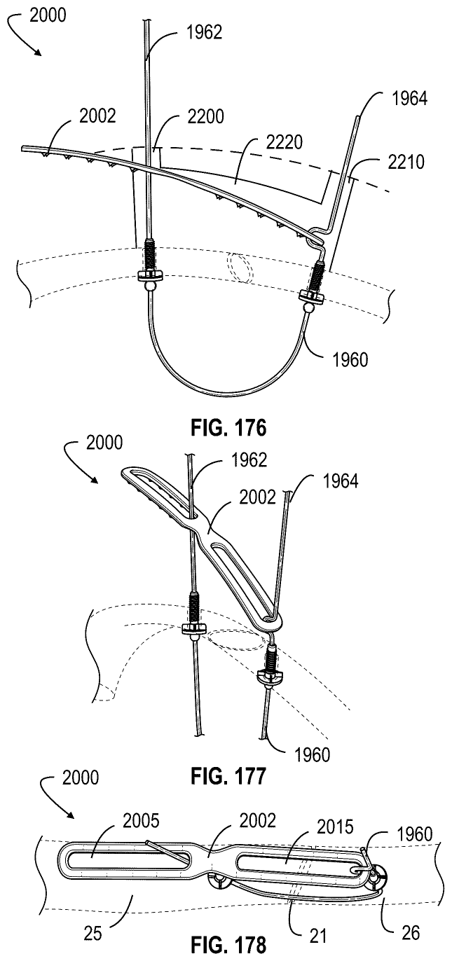

FIG. 176 is a perspective view of a step in a method of deploying the fracture plating system 2000 which may include passing the plate 2002 through the transverse skin tunnel 2220 according to an embodiment of the present disclosure. The method may further include using the tether 1960 to draw the plate 2002 into the transverse skin tunnel 2220 by holding the first tether end 1962 taut and pulling the second tether end 1964 to draw the plate 2002 into the transverse skin tunnel 2220.

The first soft tissue tunnel 2200, the second soft tissue tunnel, and/or the transverse skin tunnel 2220 may be able to expand as necessary to allow for passage of the plate 2002. The first soft tissue tunnel 2200, the second soft tissue tunnel, and/or the transverse skin tunnel 2220 are in soft tissue, so they may be able to stretch as necessary to allow the geometry of the plate 2002 to pass thru.

FIG. 177 is a perspective view of a step in the method of deploying the fracture plating system 2000. FIG. 178 is a perspective view of a step in the method of deploying the fracture plating system 2000. With the plate 2002 received in the transverse skin tunnel 2220, the method may further include positioning the plate 2002 on the first rib portion 25 and the second rib portion 26 so that the plate 2002 may span the first fracture 21.

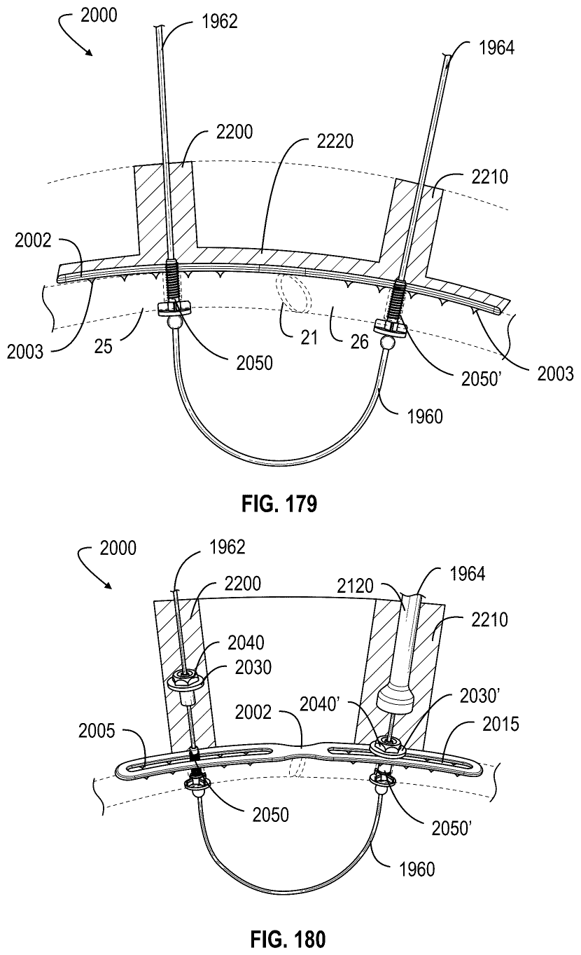

FIG. 179 is a perspective view of a step in a method of deploying the fracture plating system 2000 which may include positioning the plate 2002 on the bone according to an embodiment of the present disclosure. The method may further include positioning the plate 2002 so that the first fastener 2050 may be received in the first slot 2005 and the second fastener 2050′ may be received in the second slot 2015. The method may further include positioning the plate 2002 on an exterior portion of the bone so that a plurality of spikes 2003 of the plate 2002 engage the exterior portion of the bone.

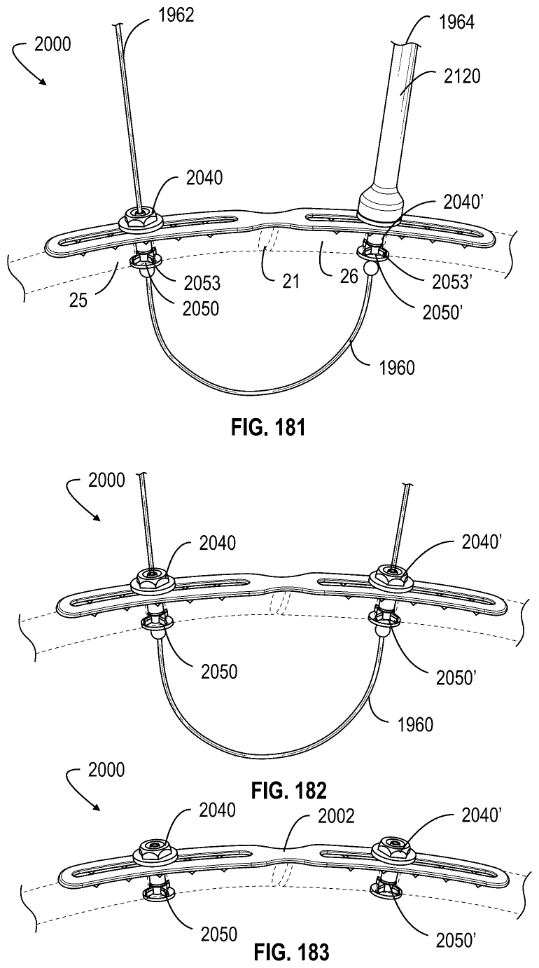

FIG. 180 is a perspective view of a step in a method of deploying the fracture plating system 2000 which may include passing a first cap 2040 and a second cap 2040′ of the fracture plating system 2000 over the first tether end 1962 and the second tether end 1964 respectively according to an embodiment of the present disclosure. The method may further include passing a first cap 2040, and optionally a first washer 2030, over the first tether end 1962 and advancing the first cap 2040 to engage the first fastener 2050. The method may further include passing a second cap 2040′, and optionally a second washer 2030′, over the second tether end 1964 and advancing the second cap 2040′ to engage the second fastener 2050′.

FIG. 181 is a perspective view of a step in a method of deploying the fracture plating system 2000 which may include securing the first cap 2040 onto the first fastener 2050 and the second cap 2040′ onto the second fastener 2050′ with a driver 2120 according to an embodiment of the present disclosure. The method may further include receiving the first cap 2040 in the driver 2120 and using the driver 2120 to secure the first cap 2040 to the first fastener 2050 thereby securing the plate 2002 to the first rib portion 25. The method may further include receiving the second cap 2040′ in the driver 2120 and using the driver 2120 to secure the second cap 2040′ to the second fastener 2050′ thereby securing the plate 2002 to the second rib portion 26. As the first cap 2040 is secured to the first fastener 2050, the first engagement feature 2053 may be further drawn into the first rib portion 25 to inhibit rotation of the first fastener 2050. As the second cap 2040′ is secured to the second fastener 2050′, the second engagement feature 2053′ may be further drawn into the second rib portion 26 to inhibit rotation of the second fastener 2050′.

FIG. 182 is a perspective view of a step in a method of deploying the fracture plating system 2000 which may include withdrawing the driver 2120 according to an embodiment of the present disclosure. The method may further include withdrawing the driver 2120 from the tether 1960.

FIG. 183 is a perspective view of a step in a method of deploying the fracture plating system 2000 which may include withdrawing the tether 1960 from the first fastener 2050 and the second fastener 2050′ according to an embodiment of the present disclosure. The method may further include withdrawing the tether 1960 from the first fastener 2050 and the second fastener 2050′. The tether 1960 may be withdrawn through the interior portion of the body. A benefit of the present method may be that it may be muscle sparing and may not require a large open incision as is customary with traditional plate fixation methods.

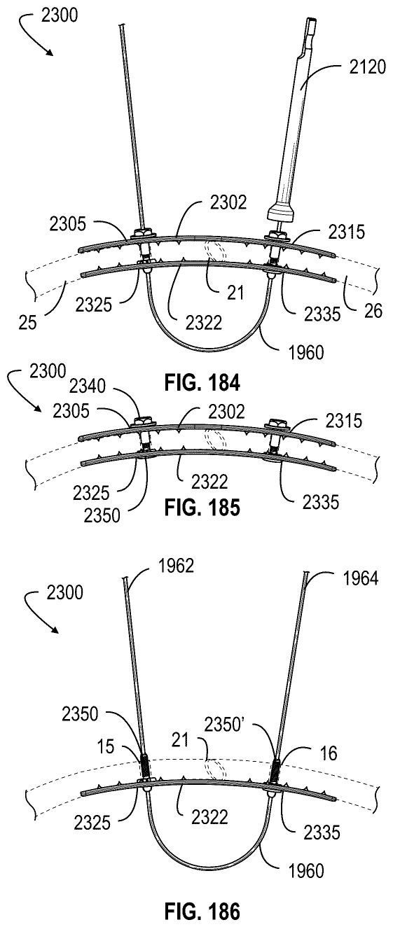

FIG. 184 is a perspective view of a fracture plating system 2300 in a partially deployed configuration spanning an exemplary first fracture according to an embodiment of the present disclosure. FIG. 185 is a perspective view of the fracture plating system of FIG. 184 in a deployed configuration spanning an exemplary bone fracture according to an embodiment of the present disclosure.

The fracture plating system 2300 may include similar features as other fracture plating systems previously described within the present disclosure. The fracture plating system 2300 may include a first plate 2302 configured to be received on an exterior portion of a bone and a second plate 2322 configured to be received on an interior portion of the bone. The first plate 2302 and the second plate 2322 may be configured to span the first fracture 21. The first plate 2302 may include a first slot 2305 and a second slot 2315. In some embodiments, the first slot 2305 and the second slot 2315 may be connected to form a single slot in the first plate 2302. The second plate 2322 may include a first slot 2325 and a second slot 2335. In some embodiments, the first slot 2325 and the second slot 2335 may be connected to form a single slot in the second plate 2322.

The fracture plating system 2300 may be configured for highly unstable fractures where additional rigidity and strength may be needed. The first plate 2302 and the second plate 2322 may sandwich the bone on the interior of the bone and the exterior of the bone and may be connected via the fasteners and caps. Having plates on interior portion and exterior portion of the bone may allow for the forces to be spread over a much larger area.

FIG. 186 is a perspective view of a step in a method of deploying the fracture plating system 2300 which may include passing a first tether end 1962 through a hole 15 and a second tether end 1964 through a hole 16 in the bone according to an embodiment of the present disclosure. The method may include passing a first tether end 1962 through a first fastener 2350, and a first slot 2325 of the second plate 2322. The method may further include passing a second tether end 1964 through a second fastener 2350′, and a second slot 2335 of the second plate 2322. The first tether end 1962 and the second tether end 1964 may then be passed through an interior portion of a body to the bone. The first tether end 1962 may be passed through the hole 15 and the second tether end 1964 may be passed through the hole 16. The tether 1960 may then guide the first fastener 2350 and the second fastener 2350′ to the hole 15 and the hole 16 respectively. The tether 1960 may also guide the second plate 2322 to the interior portion of the bone so that the second plate 2322 spans the first fracture 21.

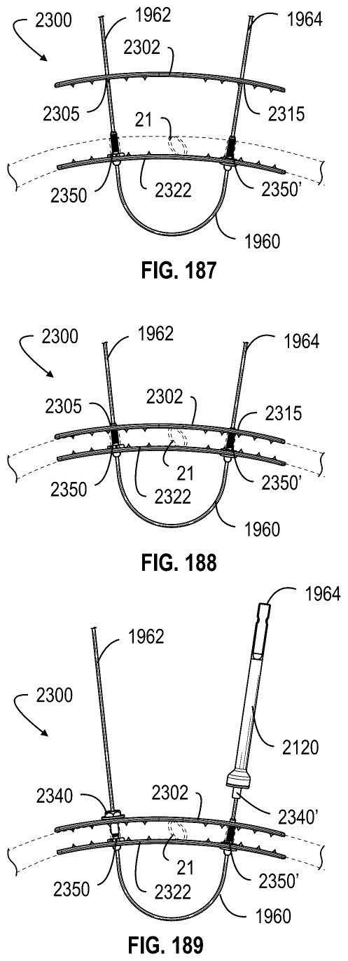

FIG. 187 is a perspective view of a step in a method of deploying the fracture plating system 2300 which may include passing the first plate 2302 of the fracture plating system 2300 over the tether 1960 to an exterior portion of the bone according to an embodiment of the present disclosure. The method may further include passing the first tether end 1962 through a first slot 2305 in the first plate 2302 and the second tether end 1964 through a second slot 2315 of the first plate 2302.

FIG. 188 is a perspective view of a step in a method of deploying the fracture plating system 2300 which may include positioning the first plate 2302 on the exterior portion of the bone according to an embodiment of the present disclosure. The method may further include using the tether 1960 to guide the first plate 2302 to the exterior portion of the bone so that the first plate 2302 spans the first fracture 21. The first fastener 2350 may be received in the first slot 2305 and the second fastener 2350′ may be received in the second slot 2315.

FIG. 189 is a perspective view of a step in a method of deploying the fracture plating system 2300 which may include passing a cap 2340 of the fracture plating system 2300 over the tether 1960 according to an embodiment of the present disclosure. The method may further include passing a first cap 2340 over the first tether end 1962 and advancing the first cap 2340 to engage the first fastener 2350. The method may further include passing a second cap 2340′ over the second tether end 1964 and advancing the second cap 2340′ to engage the second fastener 2350′.

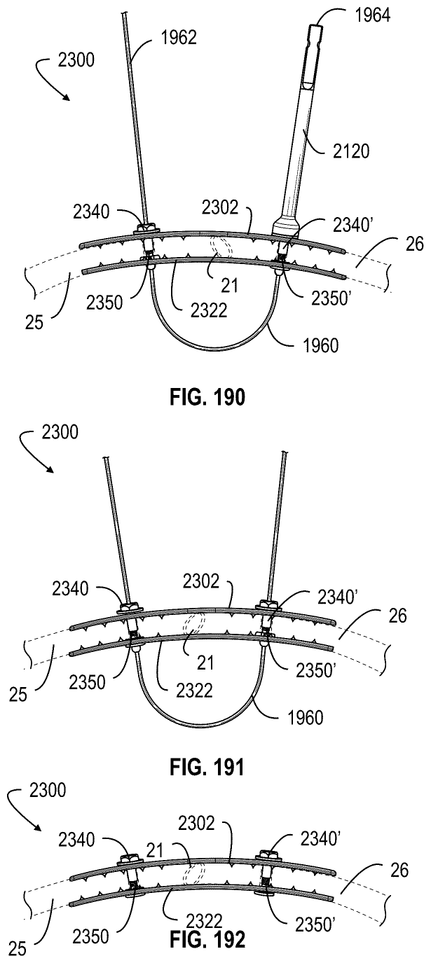

FIG. 190 is a perspective view of a step in a method of deploying the fracture plating system 2300 which may include securing the first cap 2340 onto the first fastener 2350 and the second cap 2340′ onto the second fastener 2350′ with a driver 2120 according to an embodiment of the present disclosure. The method may further include receiving the first cap 2340 in the driver 2120 and using the driver 2120 to secure the first cap 2340 to the first fastener 2350 thereby securing the first plate 2302 and the second plate 2322 to the first rib portion 25. The method may further include receiving the second cap 2340′ in the driver 2120 and using the driver 2120 to secure the second cap 2340′ to the second fastener 2350′ thereby securing the first plate 2302 and the second plate 2322 to the second rib portion 26.

FIG. 191 is a perspective view of a step in a method of deploying the fracture plating system 2300 which may include withdrawing the driver 2120 according to an embodiment of the present disclosure. The method may further include withdrawing the driver 2120 from the tether 1960.

FIG. 192 is a perspective view of a step in a method of deploying the fracture plating system 2300 which may include withdrawing the tether 1960 from the first fastener 2350 and the second fastener 2350′ according to an embodiment of the present disclosure. The method may further include withdrawing the tether 1960 from the first fastener 2350 and the second fastener 2350′. The tether 1960 may be withdrawn through the interior portion of the body.

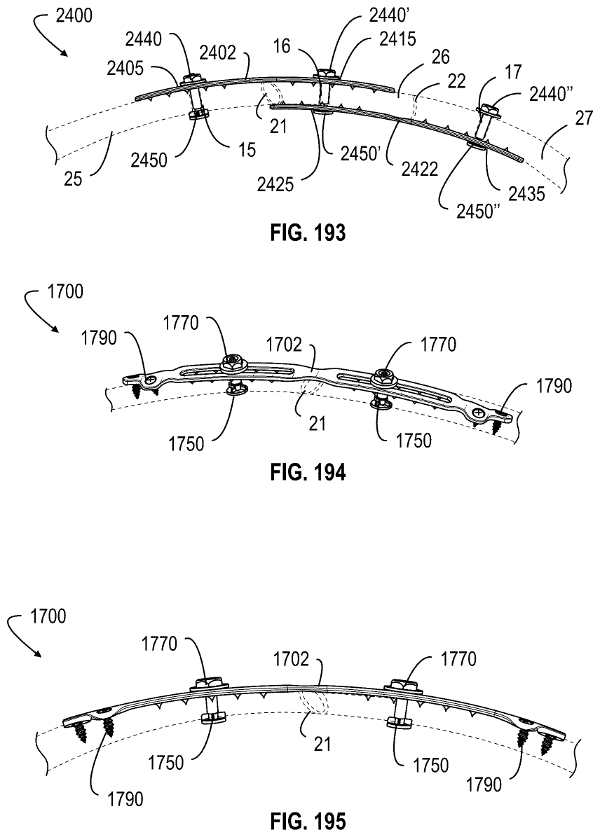

FIG. 193 is a perspective view of a fracture plating system 2400 in a deployed configuration spanning exemplary fractures according to an embodiment of the present disclosure. The fracture plating system 2400 may include similar features as other fracture plating systems previously described within the present disclosure. A method of deploying the fracture plating system 2400 may include similar steps as the method of deploying the fracture plating system 2300. The fracture plating system 2400 may include a first plate 2402 having a first slot 2405 and a second slot 2415. In some embodiments, the first slot 2405 and the second slot 2415 may be connected to form a single slot in the first plate 2402.

The fracture plating system 2400 may further include a second plate 2422 having a first slot 2425 and a second slot 2435. In some embodiments, the first slot 2425 and the second slot 2435 may be connected to form a single slot in the second plate 2422. The fracture plating system 2400 may also include a fastener 2450 and a cap 2440 configured to threadably engage the fastener 2450 to secure at least one of the first plate 2402 and the second plate 2422 to a bone.

The fracture plating system 2400 may be configured to span and secure a first fracture 21 and a second fracture 22 of the bone. The first plate 2402 may be configured to span the first fracture 21 and the second plate 2422 may be configured to span the second fracture 22.

The method of deploying the fracture plating system 2400 may include passing a first fastener 2450 through a hole 15 and a first slot 2405 of the first plate 2402 to secure the first plate 2402 to a first rib portion 25. The method may further include passing a second fastener 2450′ through a hole 16 a second slot 2415 of the first plate 2402, and a first slot 2425 of the second plate 2422 to secure the first plate 2402 and the second plate 2422 to a second rib portion 26. The method may further include passing a third fastener 2450″ through a third pilot hole 17 and a second slot 2435 of the second plate 2422. The method may further include securing a second cap 2440′ to the second fastener 2450′ and a third cap 2440″ to the third fastener 2450″ to secure the second plate 2422 to the second rib portion 26 and a third rib portion 27. A flail segment, such as second rib portion 26, may be fixated using the described method. A plate, a fastener, and a cap previously described in the present disclosure may be used for the method.

FIG. 194 is a perspective view of a fracture plating system 1700 in a deployed configuration spanning an exemplary first fracture 21 according to an embodiment of the present disclosure. FIG. 195 is a perspective view of the fracture plating system 1700 in a deployed configuration. The fracture plating system 1700 previously described in the present disclosure may be further configured to be secured to an exterior portion of a bone to secure a first fracture 21.

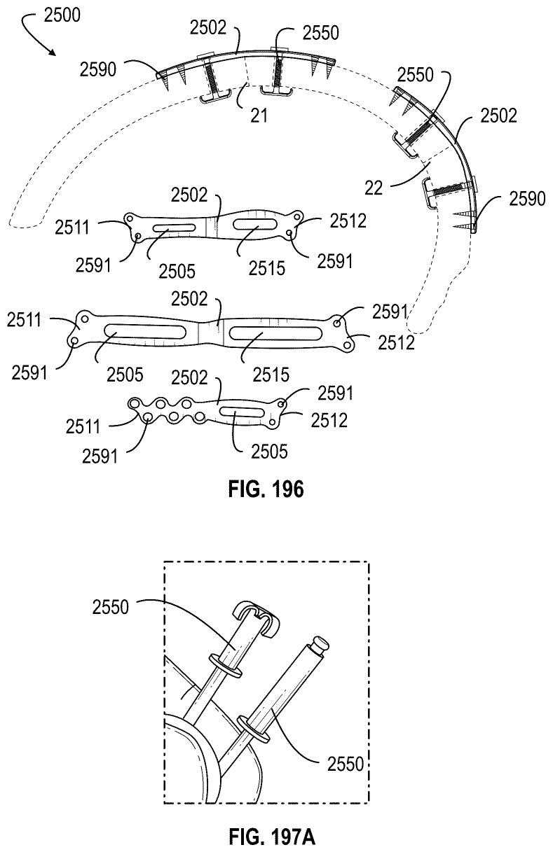

FIG. 196 is a perspective view of a fracture plating system 2500 according to an embodiment of the present disclosure. The fracture plating system 2500 may include a plate 2502 having a first slot 2505, a first end 2511, a one or more screw apertures 2591 positioned between the first slot 2505 and the first end 2511. The plate 2502 may further include a second slot 2515, a second end 2512, and one or more screw apertures 2591 positioned between the second slot 2515 and the second end 2512. The fracture plating system 2500 may include similar features as other fracture plating system previously described within the present disclosure.

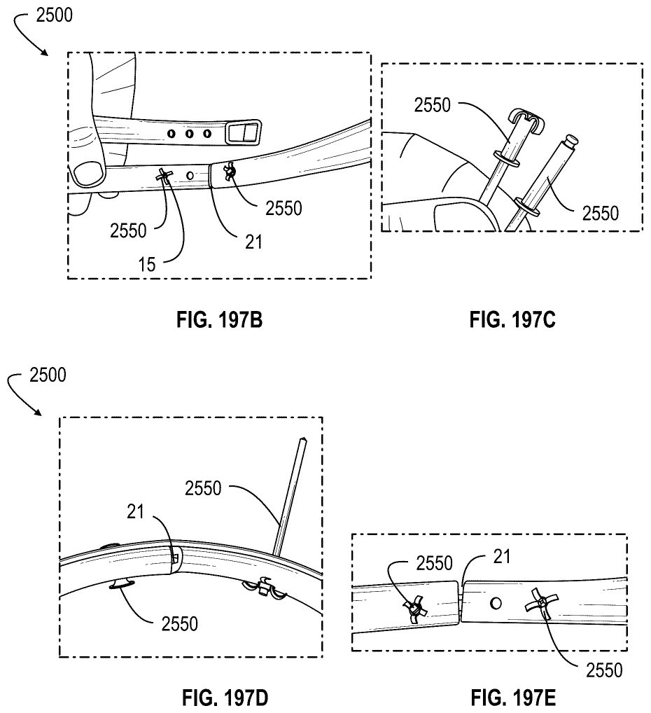

The fracture plating system 2500 may further include a fastener 2550 and a locking screw 2590. The locking screw 2590 may be configured to be received in the screw aperture 2591. The locking screw 2590 may include similar features and functions as the locking screw 1790 previously described. FIG. 197A is a perspective view of a fastener 2550 of the fracture plating system 2500 according to an embodiment of the present disclosure.

FIG. 197B is a perspective view of a fracture plating system 2500 in a deployed configuration on an exemplary bone according to an embodiment of the present disclosure. FIG. 197C is a perspective view of a fastener 2550 of the fracture plating system 2500 in a neutral configuration and a deployed configuration according to an embodiment of the present disclosure. FIG. 197D is a perspective view of the fracture plating system 2500 in a partially deployed configuration according to an embodiment of the present disclosure. FIG. 197E is a perspective view of the fracture plating system 2500 in a deployed configuration on an exemplary bone according to an embodiment of the present disclosure.

The fastener 2550 may be configured as a peel rivet. A peel type rivet may be a type of blind rivet designed for improved support in brittle, soft, or ductile materials. A mandrel of the peel rivet may be configured to split an end of a rivet body into multiple separate legs to create a large bearing surface that may abut an interior surface of the bone. With the plate 2502 positioned on an exterior surface of a bone and spanning one or more fractures, the fastener 2550 may be configured to be received in a slot of the plate 2502 and into a hole 15 in the bone. The fastener 2550 may be actuated from the exterior portion of the body to secure the plate 2502 to the bone.

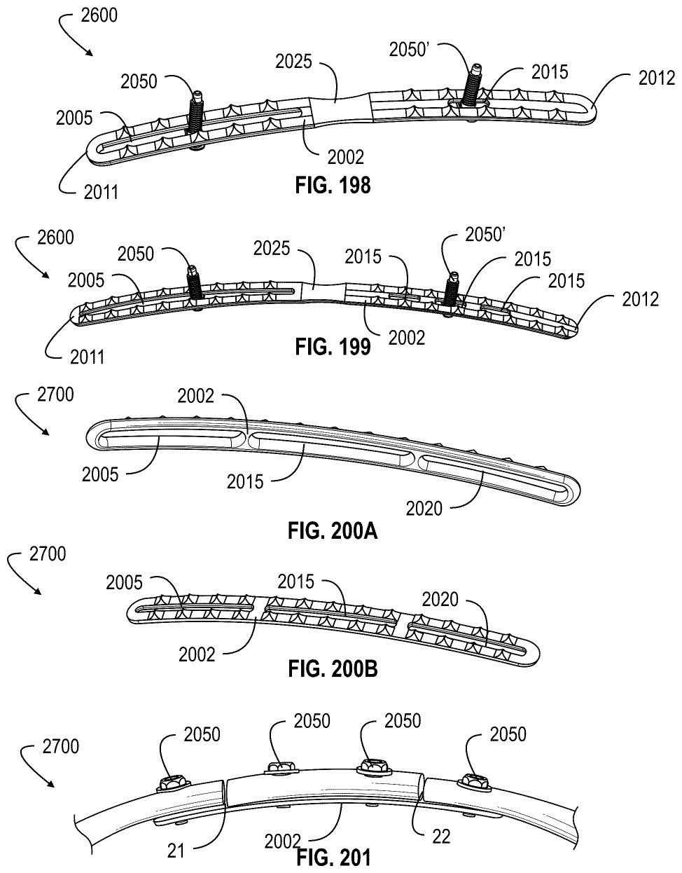

FIG. 198 is a perspective view of a fracture plating system 2600 according to an embodiment of the present disclosure. The fracture plating system 2600 may include similar features and functions as other fracture plating system previously described within the present disclosure. The fracture plating system 2600 may include the plate 2002 and one or more fasteners and/or one or more caps previously described with in the present disclosure. The plate 2002 may be configured as a self-centering plate.

In an embodiment, the plate 2002 may include a first slot 2005, a second slot 2015, a first end 2011, a second end 2012, and a central portion 2025. The first slot 2005 may be configured to receive a first fastener 2050 and the second slot 2015 may be configured to receive a second fastener 2050′. The first slot may extend a majority of a distance between the first end 2011 and the central portion 2025. The second slot 2015 may have a distance that is less than the first slot 2005. The second slot 2015 may be positioned so that, with the fastener 2050 received in the second slot 2015 and a hole 16 proximate a fracture, the central portion 2025 may be proximate the fracture thereby centering the plate 2002 relative to the fracture.

FIG. 199 is a perspective view of a fracture plating system 2600 according to an embodiment of the present disclosure. In an embodiment the plate 2002 may have a plurality of second slots 2015, each having generally the same length. The plurality of second slots 2015 may be spaced apart from each other and may be positioned between the second end 2012 and the central portion 2025. One of the plurality of second slots 2015 may be positioned so that, with the fastener 2050 received in the one of the plurality of second slots 2015 and a hole 16 proximate a fracture, the central portion 2025 may be proximate the fracture thereby centering the plate 2002 relative to the fracture.

FIG. 200A is a perspective view of a fracture plating system 2700 according to an embodiment of the present disclosure. FIG. 200B is a perspective view of the fracture plating system 2700. FIG. 201 is a perspective view of the fracture plating system 2700 in a deployed configuration spanning exemplary fractures according to an embodiment of the present disclosure. The fracture plating system 2700 may include similar features and functions as other fracture plating system previously described within the present disclosure. The fracture plating system 2700 may include the plate 2002 and one or more fasteners and/or one or more caps previously described with in the present disclosure. The fracture plating system 2700 may be configured to secure two or more fractures in a bone.

The plate may be configured to span two or more fractures. The plate 2002 may include a first slot 2005, a second slot 2015, and a third slot 2020. The first slot 2005, the second slot 2015 and the third slot 2020 may be configured to receive one or more fasteners 2050 to secure the plate 2002 to the bone.

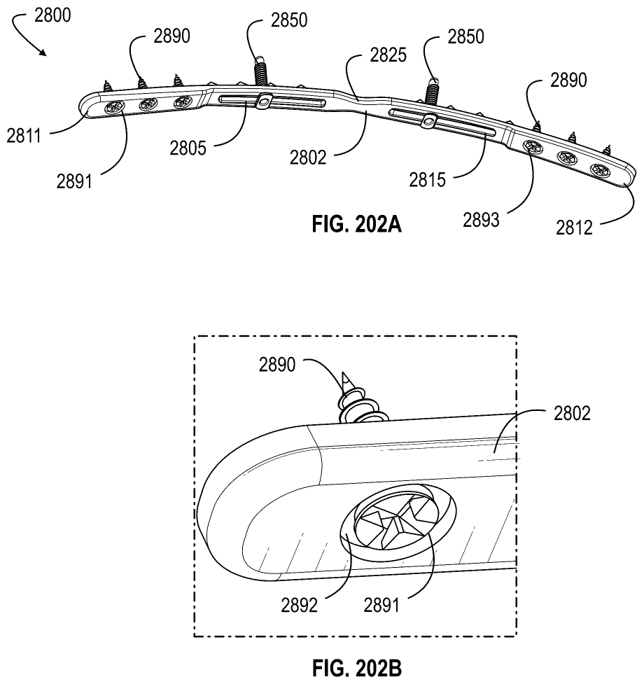

FIG. 202A is a perspective view of a fracture plating system 2800 according to an embodiment of the present disclosure. FIG. 202B is a partial perspective view of the fracture plating system 2800. The fracture plating system 2800 may include similar features and functions as other fracture plating system previously described within the present disclosure. The fracture plating system 2800 may include a plate 2802, one or more fasteners 2850, and one or more locking screws 2890.

The plate 2802 may include a first end 2811, a second end 2812 opposite the first end 2811, and a central portion 2825. The plate 2802 may further include a first slot 2805 positioned between the first end 2811 and the central portion 2825. The plate 2802 may further include a second slot 2815 positioned between the second end 2812 and the central portion 2825. The first slot 2805 and the second slot 2815 may be configured to receive one or more fasteners 2850. Additionally, or alternatively, the first slot 2805 and the second slot 2815 may include similar features as other slots previously described within the present disclosure. Additionally, or alternatively, the first slot 2805 and the second slot 2815 may be configured to receive one or more of the fasteners previously described within the present disclosure.

The plate 2802 may further include a plurality of first screw apertures 2891 positioned between the first end 2811 and the first slot 2805. The plate 2802 may further include a plurality of second screw apertures 2893 positioned between the second end 2812 and the second slot 2815. Each of the plurality of first screw apertures 2891 and the plurality of second screw apertures 2893 may be configured to receive a locking screw 2890. Additionally, each of the plurality of first screw apertures 2891 and the plurality of second screw apertures 2893 may include a screw aperture recess 2892 configured to receive a screw guide and/or a screw forceps.

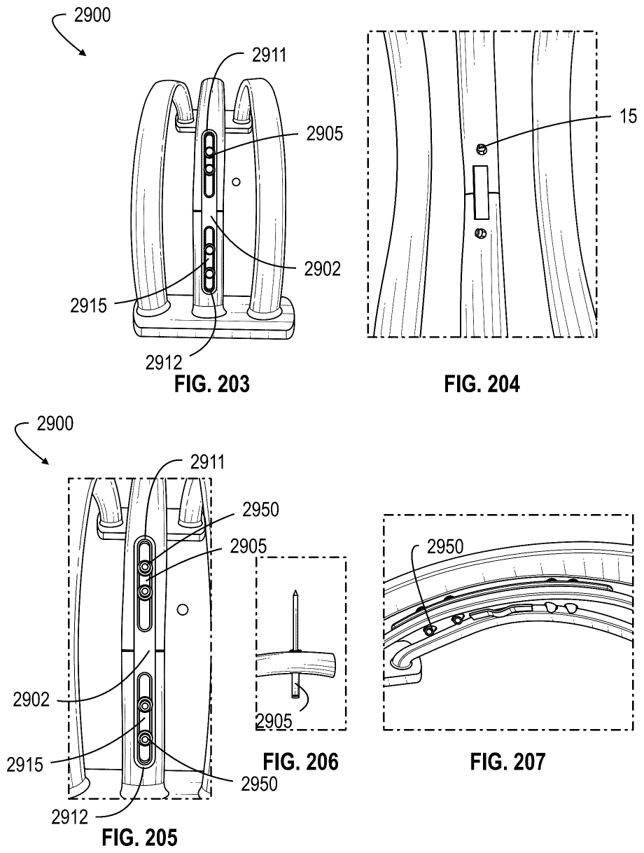

FIG. 203 is a front view of a fracture plating system 2900 in a deployed configuration spanning an exemplary fracture according to an embodiment of the present disclosure. FIG. 204 is a back view of the fracture plating system 2900. FIG. 205 is a front view of a fracture plating system 2900 in a deployed configuration spanning an exemplary fracture according to an embodiment of the present disclosure. FIG. 206 is a side view of a fasten of the fracture plating system 2900 deployed in a hole in an exemplary bone according to an embodiment of the present disclosure. FIG. 207 is a perspective view of the fracture plating system 2900. The fracture plating system 2900 may include similar features as other fracture plating system previously described within the present disclosure

The fracture plating system 2900 may include a fastener 2950. The fastener 2950 may be configured as a pop rivet. A pop rivets, also known as blind rivets, may be a mechanical fastener used to join materials, particularly when access to only one side of the work is available. A pop rivet may consist of a rivet body and a mandrel (or stem) that, when pulled with a rivet gun, may expand the rivet body, clamping the materials together and then breaking off.

The fracture plating system 2900 may include a plate 2902 having a first slot 2905, a first end 2911, a second slot 2915, and a second end 2912. With the plate 2902 positioned on an exterior surface of a bone and spanning one or more fractures, the fastener 2950 may be configured to be received in a slot of the plate 2902 and into a hole 15 in the bone. The fastener 2950 may be actuated from the exterior portion of the body to secure the plate 2902 to the bone.

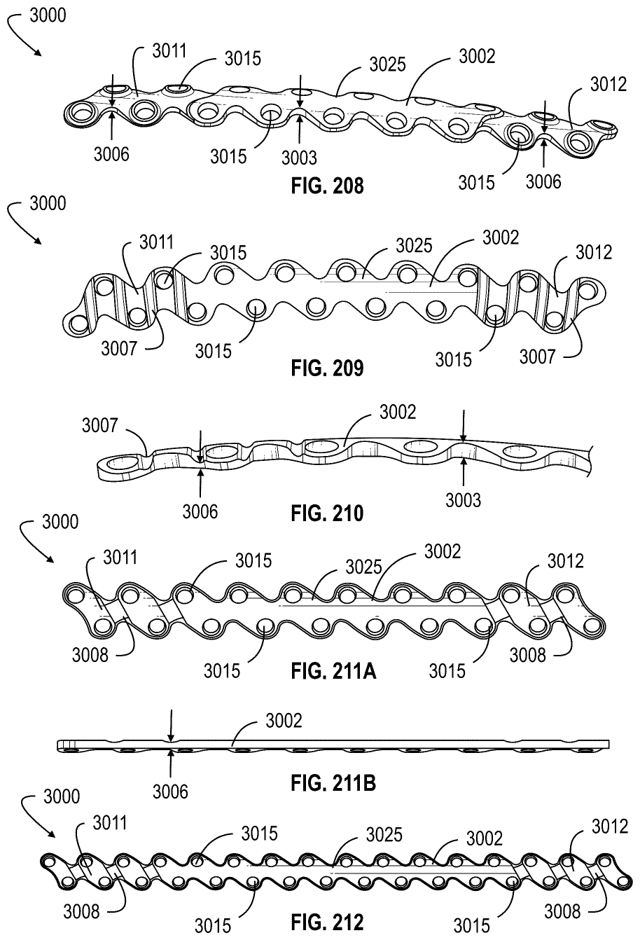

FIG. 208 is a perspective view of a plate 3002 of a fracture plating system 3000 according to an embodiment of the present disclosure. The fracture plating system 3000 may be configured to secure one or more fractures of a bone. The plate 3002 may be configured to span the one or more fractures of the bone. The plate 3002 may be configured to be secured to the bone using a fastener, for example the locking screw 2890 and/or another locking screw previously described with the present disclosure.

The plate 3002 may include a first end 3011, a second end 3012, a central portion 3025 extending between the first end 3011 and the second end 3012, and a plurality of screw apertures 3015 configured to receive the locking screw 2890. The plurality of screw apertures 3015 may be positioned in the first end 3011, the second end 3012, and the central portion 3025.

The central portion 3025 may include a plate thickness 3003. The first end 3011 and/or the second end 3012 may include an end thickness 3006 that may be less than the plate thickness 3003. The plate 3002 may be thinner at the first end 3011 and/or the second end 3012 so that the plate 3002 may be more flexible at the first end 3011 and/or the second end 3012 and thereby easier to yield. The plate 3002 may be configured to be deformed to conform to a surface of a bone that includes a fracture. The plate 3002 may be further configured so that the central portion 3025 may be positioned proximate the fracture, where the stress loads may be greater. The plate 3002 may also be configured so that, with the central portion 3025 proximate the fracture, the first end 3011 and/or the second end 3012 may be deformable to generally conform to a contour of the bone.

FIG. 209 is a top view of a plate 3002 of a fracture plating system 3000 according to an embodiment of the present disclosure. FIG. 210 is a partial front view of the plate 3002. In another embodiment, the first end 3011 and/or the second end 3012 may include one or more vertical grooves 3007. Each of the one or more vertical grooves 3007 may include the end thickness 3006. The one or more vertical grooves 3007 may be configured to facilitate deformation of the first end 3011 and/or the second end 3012 to allow the plate 3002 to generally conform to the contour of the bone. The one or more vertical grooves 3007 may be positioned on an exterior facing side of the plate 3002 and may extend generally perpendicular to a longitudinal axis of the plate 3002.

FIG. 211A is a top view of a plate 3002 of a fracture plating system 3000 according to an embodiment of the present disclosure. FIG. 211B is a front view of the plate 3002. FIG. 212 is a top view of a plate 3002 of a fracture plating system 3000 according to an embodiment of the present disclosure. In another embodiment, the first end 3011 and/or the second end 3012 may include one or more angled grooves 3008. Each of the one or more angled grooves 3008 may include the end thickness 3006. The one or more angled grooves 3008 may be configured to facilitate deformation of the first end 3011 and/or the second end 3012 to allow the plate 3002 to generally conform to the contour of the bone. The one or more angled grooves 3008 may be positioned on an exterior facing side of the plate 3002 and may be angled to align with a narrowest part of the plate 3002 which may increase the flexibility of the first end 3011 and/or the second end 3012 and may make it easier to yield.

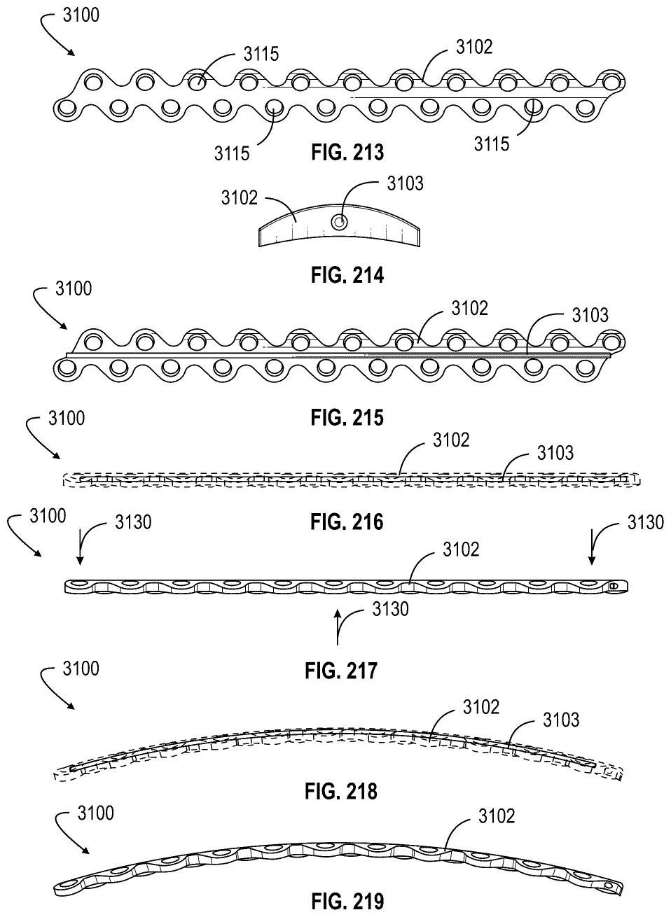

FIG. 213 is a top view of a plate 3102 of a fracture plating system 3100 according to an embodiment of the present disclosure. FIG. 214 is a side view of the plate 3102. FIG. 215 is a transparent top view of the plate 3102. FIG. 216 is a transparent front view of the plate 3102. The fracture plating system 3100 may be configured to secure one or more fractures of a bone. The plate 3102 may be configured to span the one or more fractures of the bone. The plate 3102 may be configured to be secured to the bone using a fastener, for example the locking screw 2890 and/or another locking screw previously described with the present disclosure.

The plate 3102 may include a plurality of screw apertures 3115 configured to receive the locking screw 2890. The plate 3102 may further include a central component 3103. The plate 3102 may be formed of a pliable material such as plastic, rubber, and/or silicone. The central component 3103 may be formed of a metal, such as stainless steel, titanium, titanium alloy, and/or another metal or alloy. The central component 3103 may be configured to be received in the plate 3102 along a longitudinal axis of the plate 3102. The central component 3103 may be configured so that the plate 3002 may be deformed to conform to a surface of a bone that includes a fracture.

FIG. 217 is a front view of the plate 3102 in a pre-bent configuration according to an embodiment of the present disclosure. FIG. 218 is a transparent front view of the plate 3102 in a bent configuration according to an embodiment of the present disclosure. FIG. 219 is a front view of the plate 3102 in a bent configuration. The central component 3103 may be configured so that the central component 3103 may be plastically deformed to maintain a bend profile of the plate 3102. A bending force 3130 may be applied to the plate 3102 to plastically deform the plate 3102 and the central component 3103 to generally match a contour of a bone. The material of the plate 3102 may not be plastically deformed and, without the central component 3103, the plate 3102 may not maintain a bent profile, however the central component 3103 within the plate 3102 may maintain a bent profile of the plate 3102 and central component 3103.

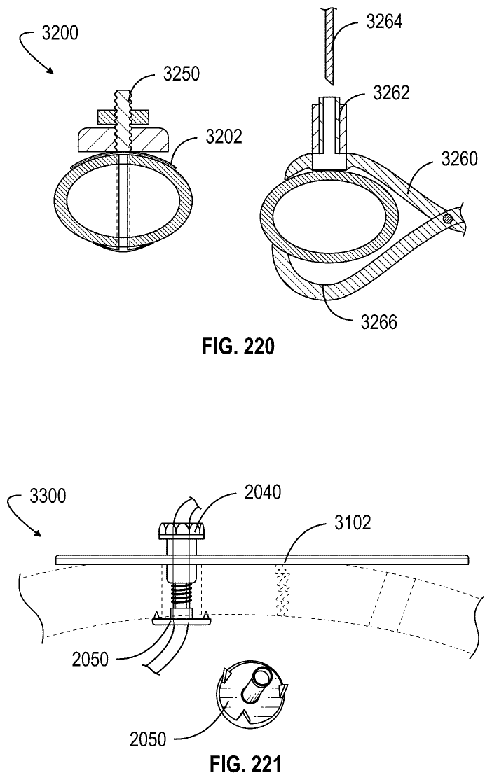

FIG. 220 is a side view of a fracture plating system 3200 in a partially deployed configuration and a deployed configuration according to an embodiment of the present disclosure. The fracture plating system 3200 may be configured to reduce a number of method steps required to secure a fracture of a bone. The fracture plating system 3200 may include a clamp 3260 having a drill guide 3262, and a drill stop 3266. The clamp 3260 may be configured to simultaneously engage an interior portion of the bone and an exterior portion of the bone. The clamp 3260 may be further configured to reduce one or more fractures in the bone. The drill guide 3262 may be positioned on a first side of the clamp 3260 that engages the exterior portion of the bone. The drill guide 3262 may be configured to guide a drill 3264 into the bone to create a pilot hole configured to receive a fastener. A second side of the clamp 3260 may include a drill stop 3266 configured to prevent the drill 3264 may damaging soft tissue adjacent to the pilot hole after the drill 3264 has passed through the bone.

A fracture plating system previously described with the present disclosure may be used in conjunction with the fracture plating system 3200 to secure the fracture. The fracture plating system 3200 may include a plate 3202 and a fastener 3250 that may be a plate and/or or a fastener previously described within the present disclosure.

FIG. 221 is a front view of a fracture plating system 3300 according to an embodiment of the present disclosure. The fracture plating system 3300 may include a pliable and/or deformable and/or bendable plate as previously described with in the present disclosure, such as the plate 3102. The fracture plating system 3300 may further include a fastener and cap previously described within the present disclosure such as fastener 2050 and the cap 2040. A method of deploying the fracture plating system 3300 may include the following steps:
1. In a bone having a fracture, drilling a pilot hole on a first side of the fracture.
2. Passing a tether through the pilot hole to an exterior of a body.
3. Using the tether, a fastener through an interior cavity of the body, to the bone so that the fastener is received in the pilot hole.
4. Using the tether to guide a plate to the bone so that the fattener may be receive in a slot in the plate.
5. Using the tether to guide a cap from the exterior of the body into the pilot hole to engage the fastener.
6. Positioning the plate on the bone. The slot in the plate may allow the plate to translate relative to the fastener.
7. Using the tether, secure an engagement feature of the fastener into the bone.
8. Using a driver, tighten the cap onto the fastener to secure the plate to the bone.
9. Repeating steps 1-7 wherein each subsequent fastener and cap may be used to bend and/or deform the plate to generally conform to a contour of the bone.

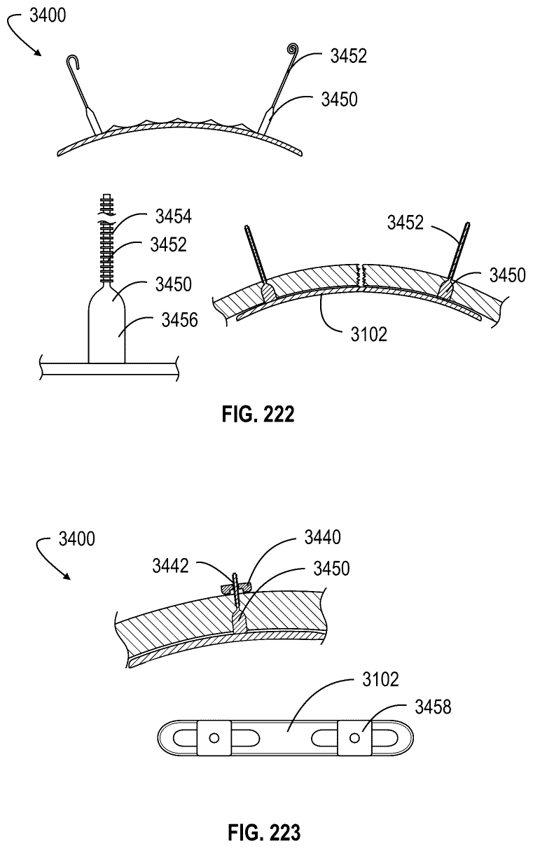

FIG. 222 is front views of a fracture plating system 3400 in a partially deployed configuration according to an embodiment of the present disclosure. FIG. 223 is perspective views of the fracture plating system 3400 according to an embodiment of the present disclosure. The fracture plating system 3400 may include similar function and features as other fracture plating system previously described within the present disclosure.

The fracture plating system 3400 may include a fastener 3450 and a cap 3440. The fastener 3450 and the cap 3440 may be configured as a self-locking ratchet mechanism, for example as a cable-tie mechanism. The fastener 3450 may include a shaft 3452 having a plurality of circumferential ridges 3454. The shaft 3452 may be configured to be received in the cap 3440. The cap 3440 may include a one-way ratchet 3442 so that as the cap 3440 is advanced along the shaft 3452, the cap 3440 may be prevented from receding relative to fastener 3450. The fracture plating system 3400 may be configured to facilitate bending and/or contouring on a plate through, with the fastener 3450 received in the bone and a plate 3102, advancing the cap 3440 along the shaft 3452 to bend and/or deform the plate 3102 toward the bone.

The fastener 3450 may also include a head 3458 and a post 3456.b The head 3458 may be configured to inhibit the fastener 3450 from passing all the through a slot in the plate 3102. The head 3458 may configured and/or sized so that at least on dimension of the head 3458 is greater than a width of a slot in which the fastener 3450 is received. The post 3456 may extend from the head 3458 to the shaft 3452. The post 3456 may be configured to provide a larger cross section between the shaft 3452 and the head 3458 and may provide additional material strength to the fastener 3450.

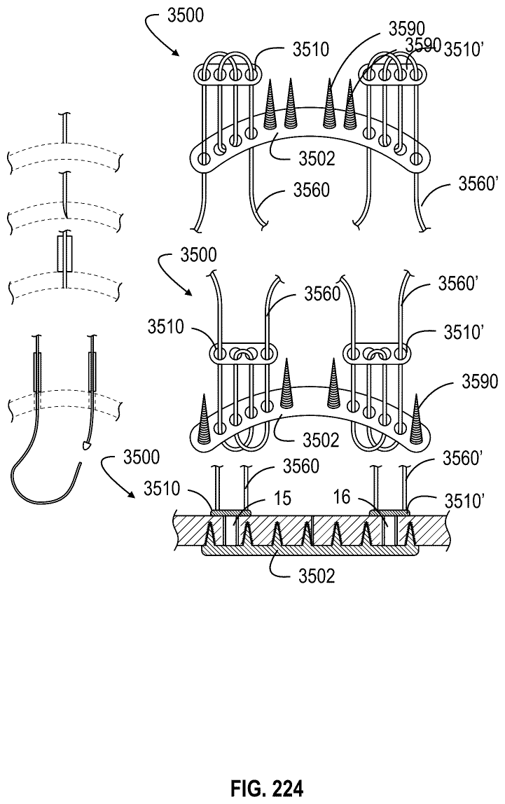

FIG. 224 is a sequence of perspective views of a fracture plating system 3500 and method for deploying the same according to an embodiment of the present disclosure. The fracture plating system 3500 may include similar features and function to other fracture plating systems previously described within the present disclosure. The fracture plating system 3500 may be configured to secure one or more fractures of a bone. The fracture plating system 3500 may include a plate 3502, a toggle 3510, a tether 3560, and a locking screw 3590. The tether 3560 may include similar features and functions as the tether 1960 previously described. The locking screw 3590 may include similar features and functions as the locking screw 1790 previously described.

The fracture plating system 3500 may be configured so that the tether 3560 may be interwoven between the plate 3502 and the toggle 3510. The fracture plating system 3500 may be further configured so that applying a tension force to the tether 3560 may result in the plate 3502 advancing toward a first side of the bone and the toggle advancing to a second side of the bone, opposite the first side.

A method for deploying the fracture plating system 3500 may include:
1. Drill a hole 15 in the bone on a first side of a fracture and drilling a hole 16 in the bone on a second side of the fracture.
2. Passing a first tether 3560 through one or more apertures in the plate 3502 and one or more apertures in a first toggle 3510.
3. Passing a second tether 3560′ through one or more apertures in the plate 3502 and one or more apertures in a second toggle 3510′.
4. Orienting the first toggle 3510 so that a first longitudinal axis of the first toggle 3510 aligns with a first axis of the hole 15. Passing the first toggle 3510 through the hole 15.
5. Orienting the second toggle 3510′ so that a second longitudinal axis of the second toggle 3510′ aligns with a second axis of the hole 16. Passing the second toggle 3510′ through the hole 16.
6. Rotating the first toggle 3510 so that the first longitudinal axis aligns with a first surface of the bone.
7. Rotating the second toggle 3510′ so that the second longitudinal axis aligns with the first surface of the bone.
8. Pulling the first tether 3560 and the second tether 3560′ on the free ends to pull out all the laxity. Thereby pulling the plate 3502 to a second surface of the bone and pulling the first toggle 3510 and the second toggle 3510′ to the first side of the bone.
9. Securing the plate 3502 to the second side of the bone using one or more locking screws 3590.
10. Using the first tether 3560 to secure the first toggle 3510 to the first side of the bone. Using the second tether 3560′ to secure the second toggle 3510′ to the first side of the bone.

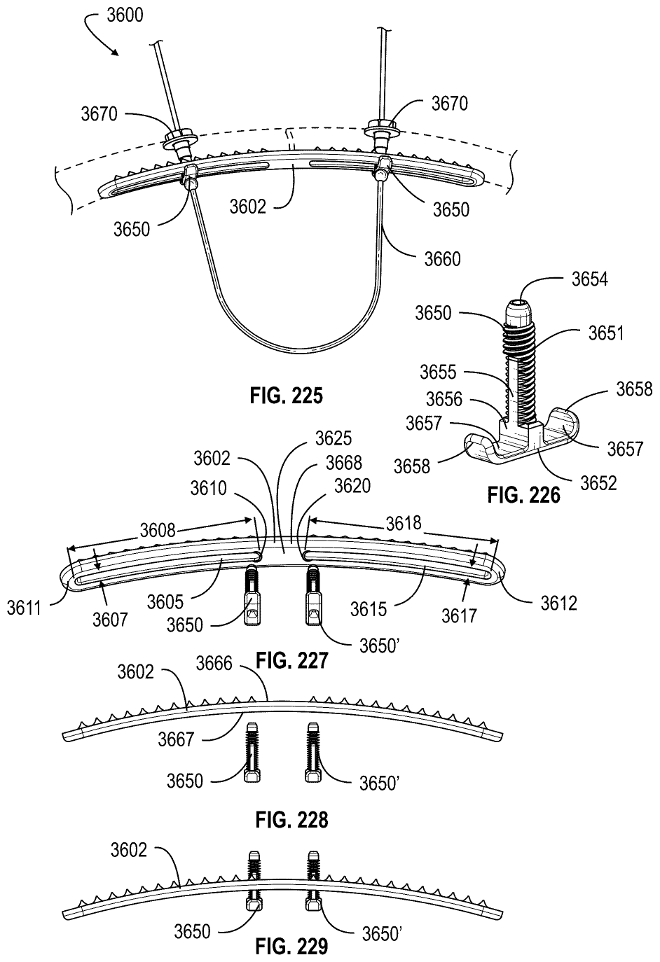

FIG. 225 is a perspective view of a fracture plating system 3600 in a partially deployed configuration on an exemplary bone according to an embodiment of the present disclosure. The fracture plating system 3600 may include similar features and functions as other fracture plating systems previously described within the present disclosure. The fracture plating system 3600 may be configured to secure one or more fractures of a bone. The fracture plating system 3600 may include a plate 3602, a fastener 3650, a cap 3670, and a tether 3660. The tether 3660 may include similar features and functions as tether 1960 previously described.

The plate 3602 may be configured to be secured to an interior portion of a bone and to span one or more fractures of the bone. The plate 3602 may include a first end 3611, a second end 3612, and a central portion 3625. The plate 3602 may further include a first slot 3605 extending between the first end 3611 and the central portion 3625, and a second slot 3615 extending between the second end 3612 and the central portion 3625. The plate 3602 may be configured to receive a fastener 3650 in the first slot 3605 and/or the second slot 3615.

The fastener 3650 may be configured to be captive within the first slot 3605 and/or the second slot 3615. The plate 3602 may be configured so that the first slot 3605 may be expanded in a direction generally perpendicular to a longitudinal axis of the first slot 3605 so that the fastener 3650 may be received in the expanded first slot 3605. The plate 3602 may be further configured so that the first slot 3605 may return to an unexpanded position after the fastener 3650 is received in the first slot 3605 thereby captively retaining the fastener 3650 within the first slot 3605. The fastener 3650 and the plate 3602 may be configured so that, with the fastener captively received in the first slot 3605, the fastener 3650 may be translatable within the first slot 3605 along the longitudinal axis of the first slot 3605.

The plate 3602 may be further configured so that the second slot 3615 may be expanded in a direction generally perpendicular to a longitudinal axis of the second slot 3615 so that the fastener 3650 may be received in the expanded second slot 3615. The plate 3602 may be further configured so that the second slot 3615 may return to an original, unexpanded configuration after the fastener 3650 is received in the second slot 3615 thereby captively retaining the fastener 3650 within the second slot 3615. The fastener 3650 and the plate 3602 may be configured so that, with the fastener 3650 captively received in the second slot 3615, the fastener 3650 may be translatable within the second slot 3615 along the longitudinal axis of the second slot 3615.

FIG. 226 is a perspective view of a fastener 3650 of the fracture plating system 3600 according to an embodiment of the present disclosure. The fastener 3650 may include a bracing member 3652, a wing feature 3657 extending from the bracing member 3652, a base portion 3656 extending from the bracing member 3652, and a threaded portion 3651 extending from the base portion 3656. The threaded portion 3651 may include one or more flats 3655 aligned with the base portion 3656. The base portion 3656 and the one or more flats 3655 may be sized to be generally equal to or slightly smaller than a width of the first slot 3605 and/or the second slot 3615. With the fastener 3650 captively received within the first slot 3605 and/or the second slot 3615, the base portion 3656 and/or the one or more flats 3655 may inhibit the fastener 3650 from rotating about a longitudinal axis of the fastener 3650 relative to the plate 3602. The fastener 3650 may further include a cannulation 3654 extending along the longitudinal axis of the fastener 3650. The cannulation 3654 may be configured to receive the tether 3660.

The plate 3602 may further include a bone facing side 3666 configured to engage an interior side of the bone, an interior facing side 3667 opposite the bone facing side 3666, and a side portion 3668 extending from the bone facing side 3666 to the interior facing side 3667. The wing feature 3657 may be configured to engage the interior facing side 3667 of the plate 3602. The wing feature 3657 may include one or more wing tips 3658. The one or more wing tips 3658 may be configured to engage the side portion 3668. Additionally, the one or more wing tips 3658 may be configured to maintain the first slot 3605 and/or the second slot 3615 in the unexpanded position and inhibit the first slot 3605 and/or the second slot 3615 from splaying open to an expanded position.

The plate 3602 may include features and functions similar to the plate 102 previously described. The first slot 3605 may include a first slot width 3607, a first slot length 3608, and a first threaded feature 3610. The second slot 3615 may include a second slot width 3617, a second slot length 3618, and a second threaded feature 3620.

FIG. 227 is a perspective view of a step in a method of deploying the fracture plating system 3600 which may include selecting the fasteners 3650 and a plate 3602 according to an embodiment of the present disclosure. The method may include selecting a plate from a set of plates each having a different length, a different width, a different number of slots, and/or a different configuration of slots. The selection of the plate may be based on patient anatomy, number of fractures, location of the fractures, and/or surgeon discretion. The method may further include selecting two or more fasteners from a set of fasteners each having a different length, wing feature, and/or head portion. The selection of the two or more fasteners may be based on the selected plate, patient anatomy, and/or surgeon discretion.

FIG. 228 is a perspective view of a step in a method of deploying the fracture plating system 3600 which may include positioning a first fastener 3650 and a second fastener 3650′ proximate the plate 3602 according to an embodiment of the present disclosure. The method may further include positioning the first fastener 3650 so that it may align with the first threaded feature 3610 and positioning the second fastener 3650′ so that it may align with the second threaded feature 3620.

FIG. 229 is a perspective view of a step in a method of deploying the fracture plating system 3600 which may include passing the first fastener 3650 and the second fastener 3650′ through the plate 3602 according to an embodiment of the present disclosure. The method may further include threadably passing the first fastener 3650 through the first threaded feature 3610 and threadably passing the second fastener 3650′ through the second threaded feature 3620, thereby making the first fastener 3650 slidably captive within the first slot 3605 and the second fastener 3650′ slidably captive within the second slot 3615.

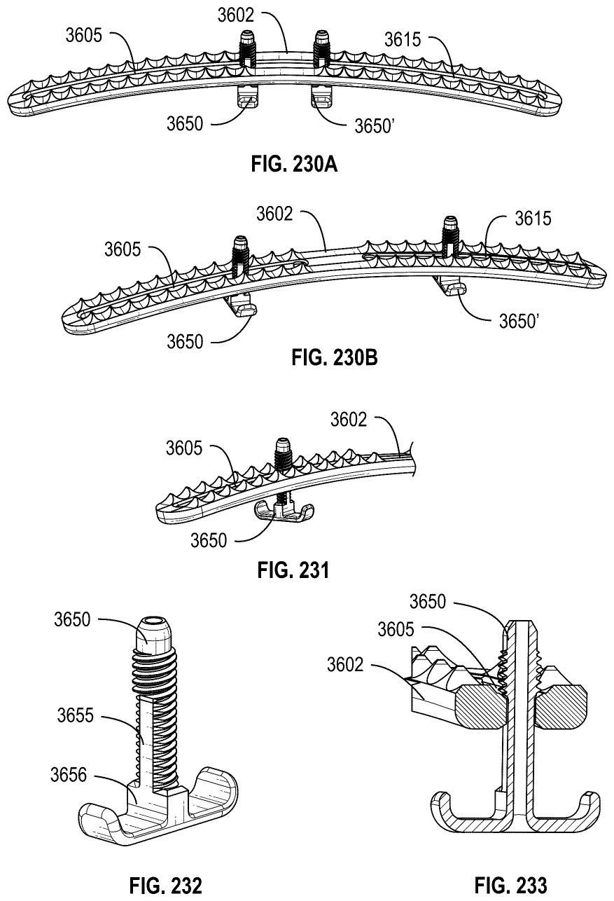

FIG. 230A is a perspective view of a step in a method of deploying the fracture plating system 3600 which may include positioning the first fastener 3650 and the second fastener 3650′ in the plate 3602 according to an embodiment of the present disclosure. FIG. 230B is a perspective view of a step in a method of deploying the fracture plating system 3600 which may include positioning the first fastener 3650 and the second fastener 3650′ in the plate 3602 according to an embodiment of the present disclosure. The method may further include sliding the first fastener 3650 away from the first threaded feature 3610 within the first slot and sliding the second fastener 3650′ away from the second threaded feature 3620 within the second slot 3615.

FIG. 231 is a partial perspective view of the fracture plating system 3600. FIG. 232 is a perspective view of the fastener 3650. FIG. 233 is a section view of the fracture plating system 3600. The fastener 3650 may be configured to be captively received with the first slot 3605 and/or the second slot 3615. The one or more flats 3655 may be configured to inhibit the fastener 3650 from rotating about a longitudinal axis of the fastener 3650 relative to the plate 3602. The fastener 3650 may be configured to translate along a longitudinal axis of the first slot and/or the second slot. Additionally, or alternatively, the fastener 3650 may be configured to translate along the longitudinal axis of the fastener 3650 while being captive within the first slot and/or the second slot. Additionally, or alternatively, the fastener 3650 may be configured to rotate in a plane defined by the one or more flats 3655 while being captive within the first slot and/or the second slot.

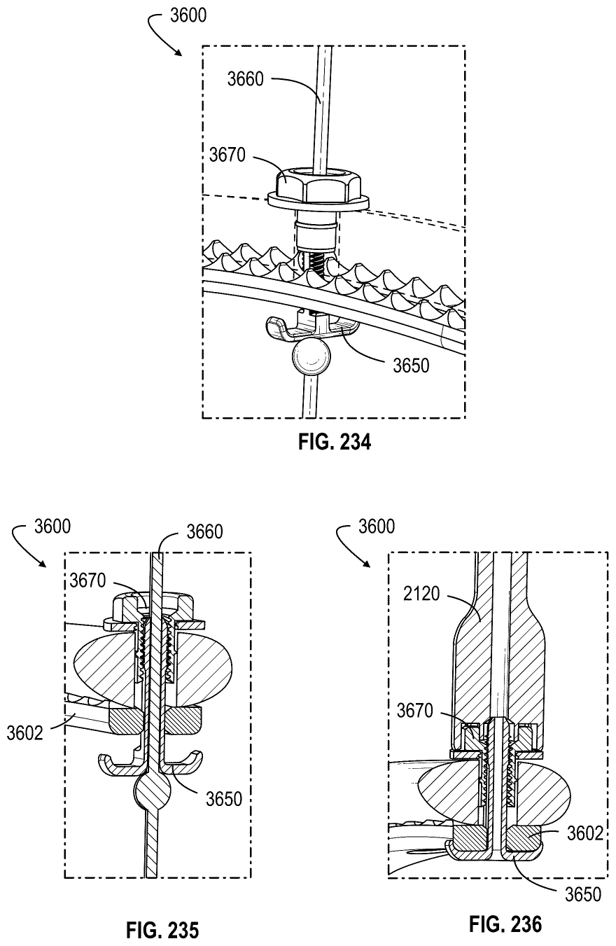

FIG. 234 is a perspective view of a step in a method of deploying the fracture plating system 3600 according to an embodiment of the present disclosure. The method may further include using a tether 3660 to guide the fastener 3650 and the plate 3602 to an interior portion of the bone. The method may further include using the tether 3660 to guide the fastener 3650 into a hole in the bone. The method may further include using the tether 3660 to guide a cap 3670 to engage the fastener 3650.

FIG. 235 is a perspective view of a step in a method of deploying the fracture plating system 3600 according to an embodiment of the present disclosure. The method may further include threadably engaging the cap 3670 with the fastener 3650.

FIG. 236 a perspective view of a step in a method of deploying the fracture plating system 3600 which may include using a driver 2120 to engage the cap 3670 according to an embodiment of the present disclosure. The method may further include using the driver 2120 to secure the cap 3670 to the fastener 3650 thereby securing the plate 3602 to the bone.

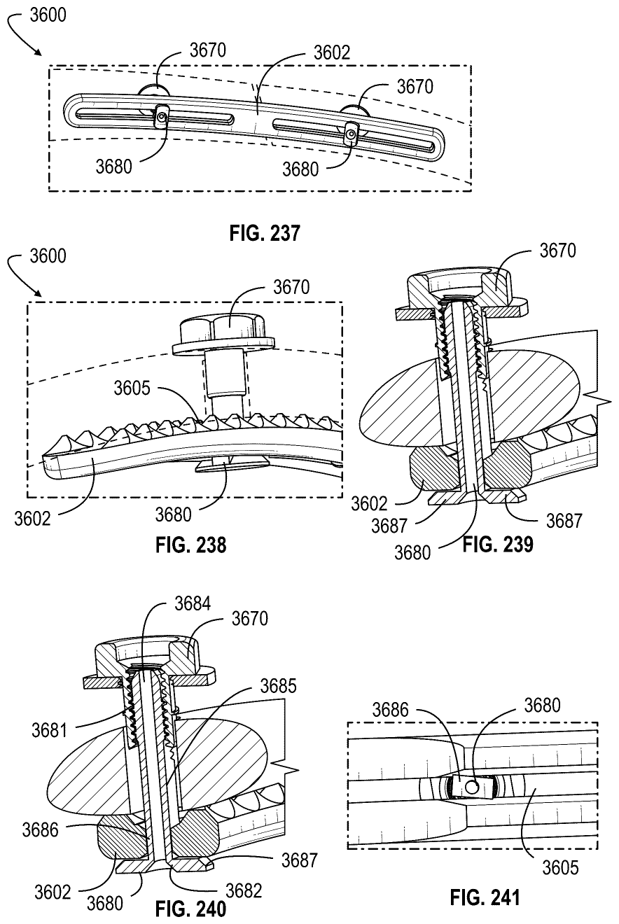

FIG. 237 is a perspective view of a fracture plating system 3600 according to an embodiment of the present disclosure. The fracture plating system 2600 may include a plate 3602, a fastener 3680, a cap 3670, and a tether 3660. The tether 3660 may include similar features and functions as tether 1960 previously described.

The plate 3602 may have reduced flexural rigidity to enable the plate 3602 to more readily conform to the curvature of the bone. This may facilitate use of the plate 3602 in a larger variety of bone fractures. The ribs, for example, do not have uniform curvature, but each rib may rather have a smaller radius of curvature around the sides, and a larger radius of curvature around the front and back of the body. A more flexible plate such as the plate 3602 may more readily adapt to a tighter (i.e., lower radius) curve on the bone. Further, such a plate may also be usable with fractures on bone segments with a larger radius of curvature.

The plate 3602 may, in some embodiments, be made of a metal such as Titanium, with a lower thickness configured to provide higher flexure. In alternative embodiments, the plate 3602 may be made of a material with a lower elastic modulus, such as a biocompatible polymer. PEEK (polyether ether ketone) is one example. Use of a biocompatible polymer may also provide benefits in terms of radiolucency. For example, when formed of a polymer such as PEEK, the plate 3602 may provide enhanced X-ray visualization of other injuries within the chest cavity. This may be particularly beneficial if multiple ribs are fractured, or if there are other injuries, related to trauma to a rib, that require medical intervention.

FIG. 238 is a partial perspective view of the fracture plating system 3600. FIG. 239 is a section view of the fracture plating system 3600. FIG. 240 is a section view of the fracture plating system 3600. The fastener 3680 may be configured to be captive within the first slot 3605 and/or the second slot 3615. The plate 3602 may be configured so that the first slot 3605 may be expanded in a direction generally perpendicular to a longitudinal axis of the first slot 3605 so that the fastener 3680 may be received in the expanded first slot 3605. The plate 3602 may be further configured so that the first slot 3605 may return to an unexpanded position after the fastener 3680 is received in the first slot 3605 thereby captively retaining the fastener 3680 within the first slot 3605. The fastener 3680 and the plate 3602 may be configured so that, with the fastener 3680 captively received in the first slot 3605, the fastener 3680 may be translatable within the first slot 3605 along the longitudinal axis of the first slot 3605.

The fastener 3680 may include a head portion 3682, a wing feature 3687 extending from the head portion 3682, a base portion 3686 extending from the head portion 3682, and a threaded portion 3681 extending from the base portion 3686. The threaded portion 3681 may include one or more flats 3685 aligned with the base portion 3686.

FIG. 241 is a partial bottom view of the fracture plating system 3600. The base portion 3686 and the one or more flats 3685 may be sized to be generally equal to or slightly smaller than a width of the first slot 3605 and/or the second slot 3615. With the fastener 3680 captively received within the first slot 3605 and/or the second slot 3615, the base portion 3686 and/or the one or more flats 3685 may inhibit the fastener 3680 from rotating about a longitudinal axis of the fastener 3680 relative to the plate 3602. The fastener 3680 may further include a cannulation 3684 extending along the longitudinal axis of the fastener 3680. The cannulation 3684 may be configured to receive the tether 3660.

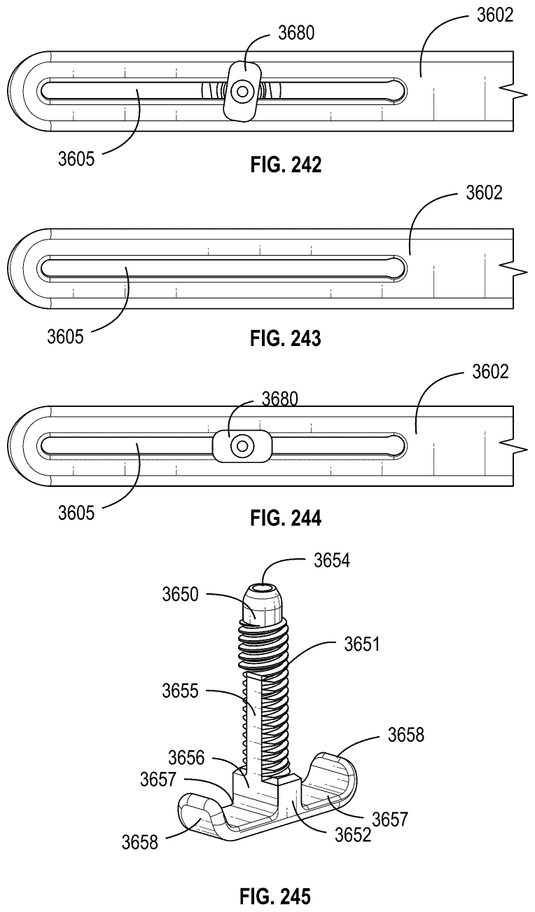

FIG. 242 is a partial bottom view of the fracture plating system 3600. FIG. 243 is a partial bottom view of the fracture plating system 3600. FIG. 244 is a partial bottom view of the fracture plating system 3600. The wing feature 3657 may be configured to engage the interior facing side 3667 of the plate 3602. With a torque applied to the fastener 3680, the one or more flats 3685 and/or the base portion 3686 may exert a force on the plate 3602 that may result in the plate 3602 expanding in a direction generally perpendicular to a longitudinal axis of the first slot 3605. Use of a lower thickness or less rigid material for the plate 3602 may cause such expansion to be more likely. Consequently, with a torque applied to the fastener 3680, the fastener 3680 may rotate about a longitudinal axis of the fastener 3680 relative to the plate 3602.

This rotation may not be desirable, as keeping the fastener 3680 from rotating about its longitudinal axis may provide helpful counter-torque as the cap 3670 is rotated onto the fastener 3680. Hence, a fastener 3650 may be used to provide support against undesired expansion of the slot 3605 and/or other flexure of the plate 3602, thereby avoiding rotation of the fastener 3650 within the slot 3605, as will be described starting with FIG. 245.

FIG. 245 is a perspective view of a fastener 3650 of a fracture plating system 3600 according to an embodiment of the present disclosure. The fastener 3650 may include wing tips 3658 to prevent rotation of the fastener 3650 about the longitudinal axis of the fastener 3650 relative to the plate 3602, with a torque applied to the fastener 3650.

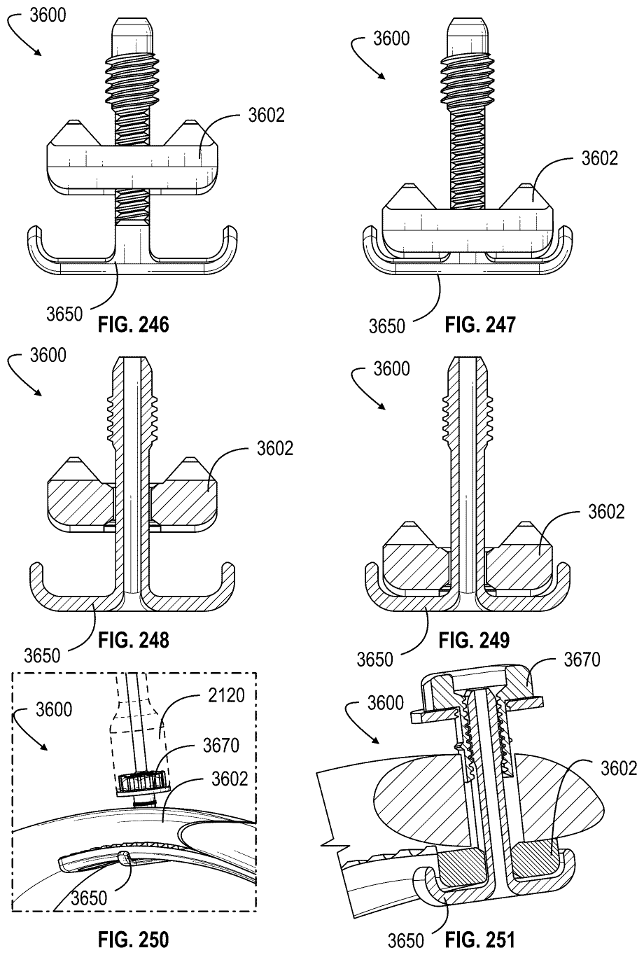

FIG. 246 is a perspective view of the fracture plating system 3600 in a partially deployed configuration according to an embodiment of the present disclosure. FIG. 247 is a perspective view of the fracture plating system 3600 in a deployed configuration according to an embodiment of the present disclosure. FIG. 248 is a section view of the fracture plating system 3600. FIG. 249 is a section view of the fracture plating system 3600. FIG. 250 is a perspective view of the fracture plating system 3600 according to an embodiment of the present disclosure. FIG. 251 is a section view of the fracture plating system 3600.

As shown in FIG. 249, FIG. 250, and FIG. 251, the wing tips 3658 may extend along either side of the plate 3602 to prevent expansion of the plate 3602 in a direction perpendicular to the length of the slot 3605, thus preventing expansion that would tend to expand the slot 3605. Thus, the wing features 3657 and wing tips 3658 may act as stabilizing members to help the plate 3602 retain its desired shape and prevent undesired rotation of the fastener 3650 within the slot 3605.

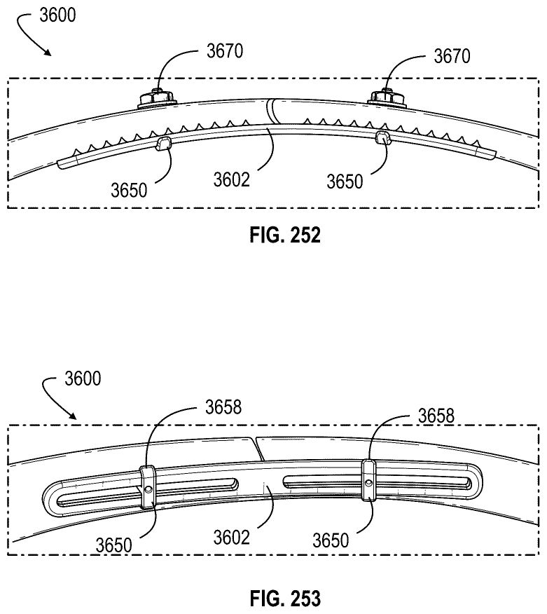

FIG. 252 is a perspective view of the fracture plating system of FIG. 246 in a deployed configuration according to an embodiment of the present disclosure. FIG. 253 is a perspective view of the fracture plating system of FIG. 252. The cap 3670 may be configured to threadably engage the fastener 3650 and may draw the fastener 3650 toward the cap 3670 thereby engaging the wing tips 3658 with the plate 3602 to prevent the first slot and/or the second slot from expanding with a torque applied to the cap 3670 and/or the fastener 3650.

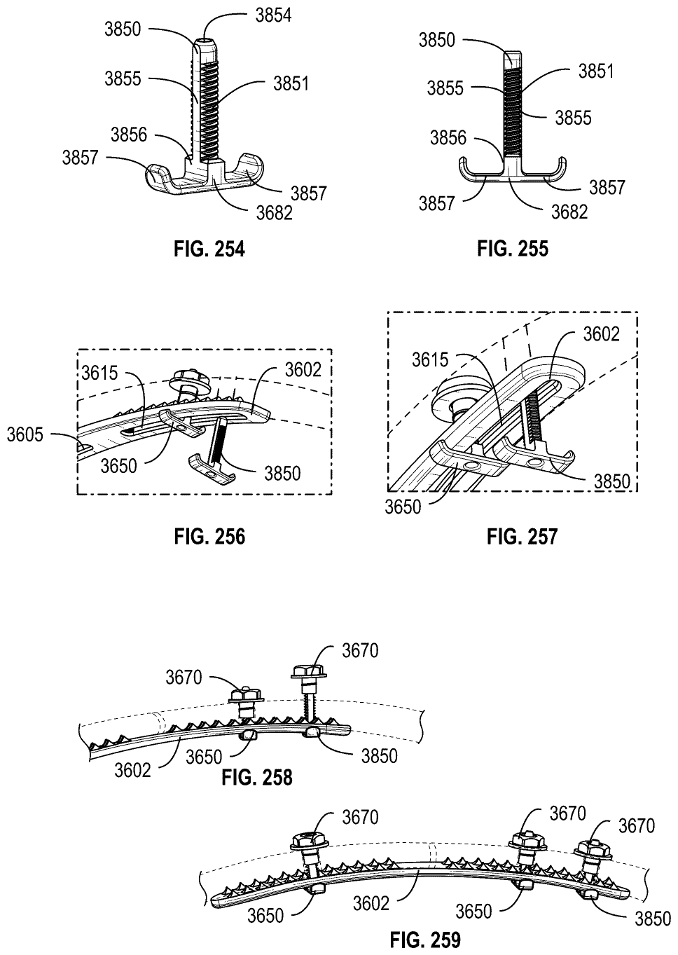

FIG. 254 is a perspective view of a fastener 3850 of a fracture plating system 3600 according to an embodiment of the present disclosure. FIG. 255 is a side view of the fastener 3850. The fastener 3850 may be configured as a rescue fastener. The fastener 3850 may further be configured to receive a tether 3660 and may be guided by the tether 3660 through an interior portion of a body to a bone having a fracture and a pilot hole proximate the fracture.

The fastener 3850 may further be configured to be slidably received in the first slot 3605 and/or the second slot 3615. The fastener 3850 may further be configured to secure the plate 3602 to the bone.

The fastener 3850 may include a wing feature 3857, a base portion 3856 extending from the wing feature 3857, and a threaded portion 3851 extending from the base portion 3856. The threaded portion 3851 may include one or more flats 3855 aligned with the base portion 3856. The one or more flats 3855 may extend the length of the threaded portion 3851.

The base portion 3856 and the one or more flats 3855 may be sized to be generally equal to or slightly smaller than a width of the first slot 3605 and/or the second slot 3615. With the fastener 3850 captively received within the first slot 3605 and/or the second slot 3615, the base portion 3856 and/or the one or more flats 3855 may inhibit the fastener 3850 from rotating about a longitudinal axis of the fastener 3850 relative to the plate 3602. The fastener 3850 may further include a cannulation 3854 extending along the longitudinal axis of the fastener 3850. The cannulation 3854 may be configured to receive the tether 3660.

FIG. 256 is a perspective view of the fracture plating system 3600 in a partially deployed configuration according to an embodiment of the present disclosure. FIG. 257 is a perspective view of the fracture plating system 3600 in a partially deployed configuration according to an embodiment of the present disclosure. FIG. 258 is a perspective view of the fracture plating system 3600 in a partially deployed configuration according to an embodiment of the present disclosure. FIG. 259 is a perspective view of the fracture plating system 3600 in a partially deployed configuration according to an embodiment of the present disclosure. With the plate 3602 secured to the bone with one or more fasteners 3650 received in the first slot 3605 and/or the second slot 3615, the fastener 3850 may be guided to the first slot 3605 and/or the second slot 3615, slidably pass through the first slot 3605 and/or the second slot 3615, and into a pilot hole in the bone. The fastener 3850 may be configured to receive a cap 3670 to secure the fastener 3850 and the plate 3602 to the bone.

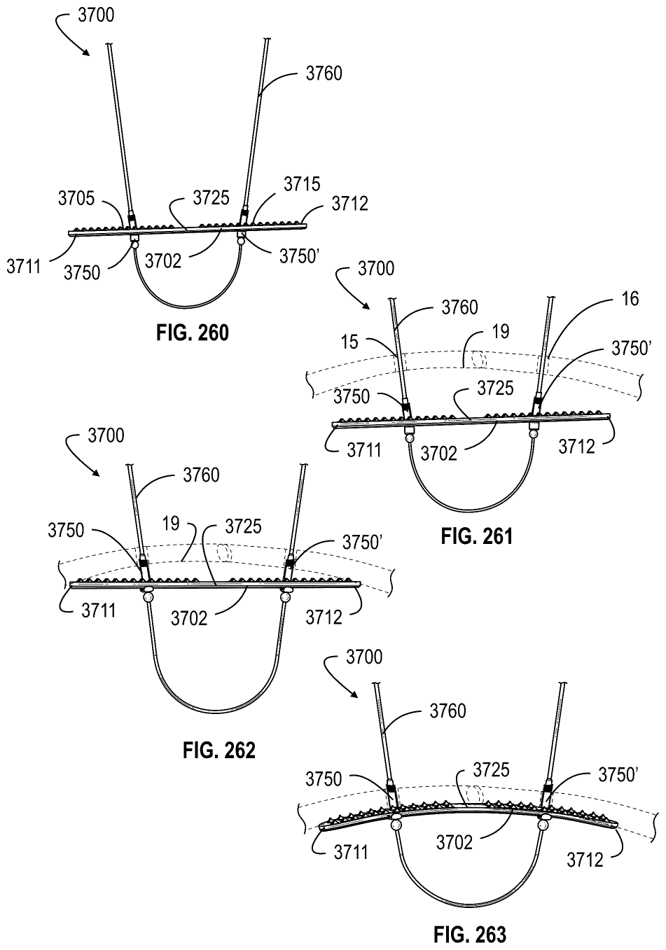

FIG. 260 is a perspective view of a fracture plating system 3700 according to an embodiment of the present disclosure. The fracture plating system 3700 may include similar features and functions as other fracture plating system previously described with the present disclosure. The fracture plating system 3700 may include a plate 3702, a fastener 3750, a cap 3740, and a tether 3760. The tether 3760 may include similar features and functions as tether 1960 previously described. The plate 3702 may be formed of malleable material. The plate 3702 may be configured to bend and/or deform to generally conform to a contour of a bone.

The plate 3702 may include a first end 3711, a second end 3712, and a central portion 3725. The plate 3702 may further include a first slot 3705 extending between the first end 3711 and the central portion 3725 and a second slot 3715 extending between the second end 3712 and the central portion 3725. The fracture plating system 3700 may be configured so that, with a fastener 3750 received on the tether 3760, and the fastener 3750 received within the plate 3702, applying a force to the tether 3760 may guide the plate 3702 to a concave portion 19 of the bone. Additionally, with the first end 3711 and the second end 3712 abutting the concave portion 19 of the bone, continuing to apply a force to the tether 3760 may bend the plate 3702 toward the concave portion 19 of the bone.

FIG. 261 is a perspective view of a step in a method of deploying the fracture plating system 3700 which may include passing the tether 3760 through the plate 3702 and the bone according to an embodiment of the present disclosure. The method may include passing a tether 3760 through a first fastener 3750 and a second fastener 3750′ wherein the first fastener 3750 and the second fastener 3750′ are captively received within the plate 3702. Passing the tether 3760 through a hole 15 and a hole 16 in the bone.

FIG. 262 is a perspective view of a step in a method of deploying the fracture plating system 3700 which may include drawing the plate 3702 toward the bone according to an embodiment of the present disclosure. The method may further include using the tether 3760 to draw the plate 3702 toward a concave portion 19 of the bone until the first end 3711 and the second end 3712 abut the bone.

FIG. 263 is a perspective view of a step in a method of deploying the fracture plating system 3700 which may include using the tether 3760 to bend the plate 3702 to engage the bone according to an embodiment of the present disclosure. The method may further include applying a tension force to the tether 3760 to bend the plate 3702 toward the concave portion 19 of the bone to engage the bone.

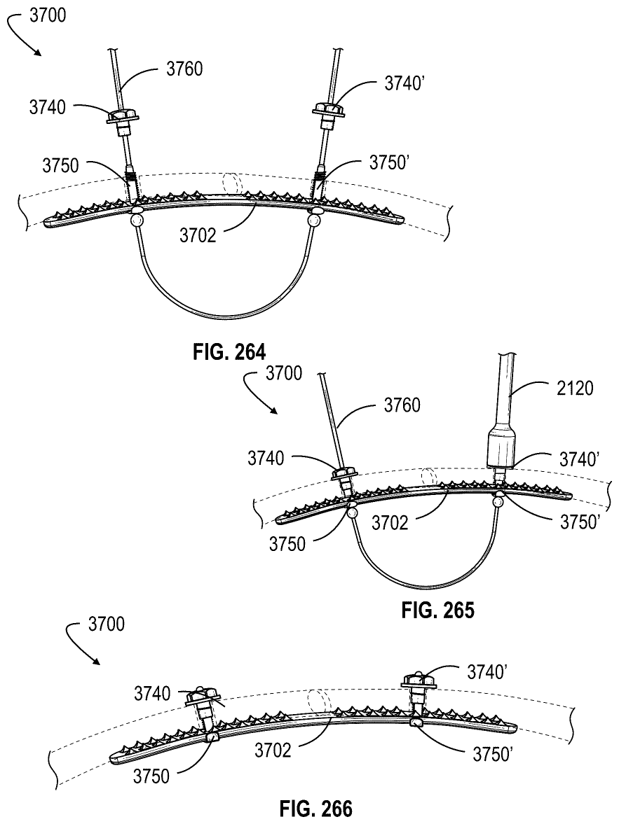

FIG. 264 is a perspective view of a step in a method of deploying the fracture plating system 3700 which may include passing the cap 3740 onto the tether 3760 according to an embodiment of the present disclosure. The method may further include using the tether 3760 to guide a first cap 3740 to the first fastener 3750 and to guide a second cap 3740′ to the second fastener 3750′

FIG. 265 is a perspective view of a step in a method of deploying the fracture plating system 3700 which may include using a driver 2120 to engage the cap 3740 according to an embodiment of the present disclosure. The method may further include using a driver 2120 to engage the first cap 3740 to threadably secure the first cap 3740 to the first fastener 3750 and using the driver 2120 to engage the second cap 3740′ to threadably secure the second cap 3740′ to the second fastener 3750′.

FIG. 266 is a perspective view of the fracture plating system 3700 in a deployed configuration according to an embodiment of the present disclosure. With the plate 3702 conforming to a contour of the bone and with the plate 3702 secured to the bone, the driver 2120 may be withdraw and the tether 3760 may be withdrawn.

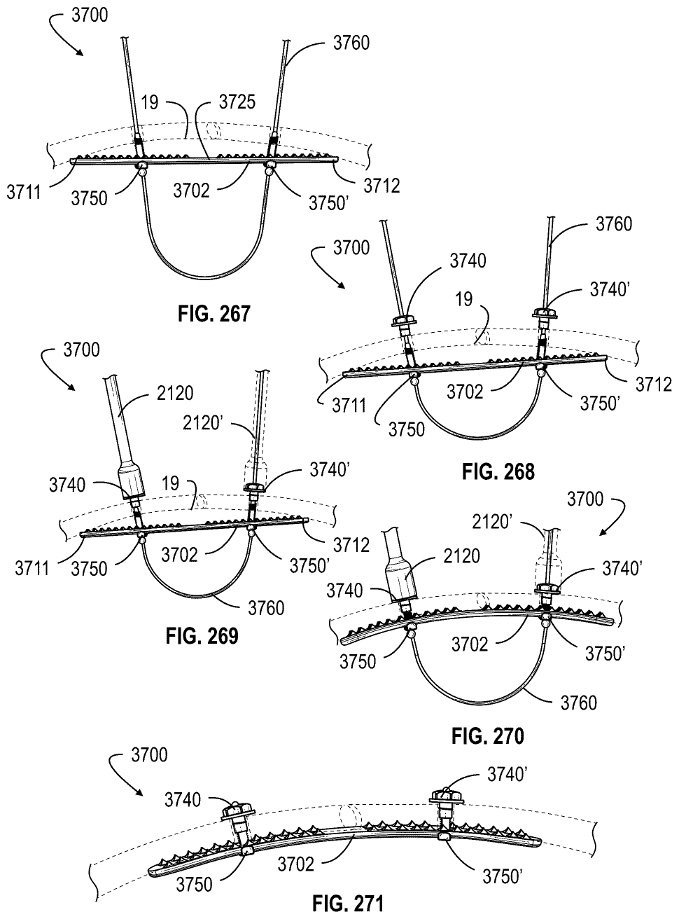

FIG. 267 is a perspective view of the fracture plating system 3700 which may include the plate 3702, the cap 3740, the fastener 3750, and the tether 3760 according to an embodiment of the present disclosure. A method for deploying the fracture plating system 3700 may include passing the tether 3760 through a first fastener 3750 and a second fastener 3750′ wherein the first fastener 3750 and the second fastener 3750′ are captively received within the plate 3702. Passing the tether 3760 through a hole 15 and a hole 16 in the bone. The method may further include using the tether 3760 to draw the plate 3702 toward a concave portion 19 of the bone until the first end 3711 and the second end 3712 abut the bone.

FIG. 268 is a perspective view of a step in a method of deploying the fracture plating system 3700 which may include engaging the cap 3740 with the fastener 3750. The method may further include threadably engaging a first cap 3740 with the first fastener 3750 and threadably engaging a second cap 3740′ with the second fastener 3750′.

FIG. 269 is a perspective view of a step in a method of deploying the fracture plating system 3700 which may include engaging a driver 2120 with the cap 3740 according to an embodiment of the present disclosure. The method may further include using a first driver 2120 to engage the first cap 3740 and a second driver 2120′ to engage the second cap 3740′.

FIG. 270 is a perspective view of a step in a method of deploying the fracture plating system 3700 which may include using the driver 2120, the cap 3740, and the fastener 3750 to bend the plate 3702 to engage the bone according to an embodiment of the present disclosure. The method may further include using the first driver 2120 to engage the first cap 3740 to draw the first fastener 3750 toward the first cap 3740 thereby drawing and/or bending the plate 3702 toward the bone. The method may further include using the second driver 2120′ to engage the second cap 3740′ to draw the second fastener 3750′ toward the second cap 3740′ thereby drawing and/or bending the plate 3702 toward the bone.

FIG. 271 is a perspective view of the fracture plating system 3700 in a deployed configuration according to an embodiment of the present disclosure. With the plate 3702 conforming to a contour of the bone and with the plate 3702 secured to the bone, the first driver 2120 and the second driver 2120′ may be withdraw and the tether 3760 may be withdrawn.

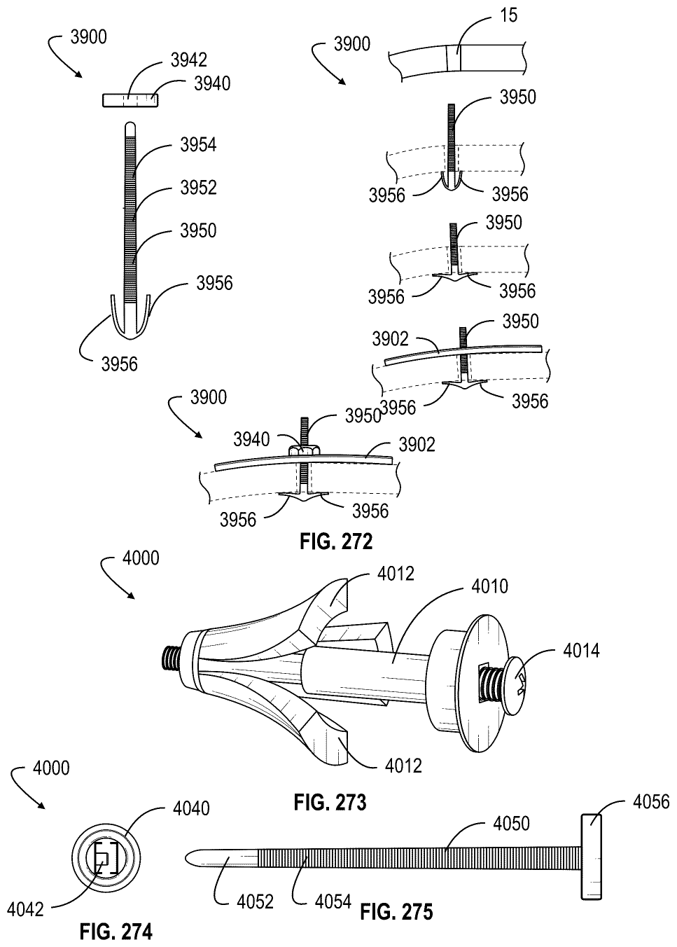

FIG. 272 is a method of deploying a fracture plating system 3900 according to an embodiment of the present disclosure. The fracture plating system 3900 may include similar features and functions as other fracture plating system previously described within the present disclosure. The fracture plating system 3900 may be used with any of the plates previously described within the present disclosure.

The fracture plating system 3900 may include a fastener 3950 and a cap 3940. The fastener 3950 and the cap 3940 may be configured as a self-locking ratchet mechanism, for example similar to a cable-tie mechanism. The fastener 3950 may include a shaft 3952 having a plurality of circumferential ridges 3954. The shaft 3952 may be configured to be received in the cap 3940. The cap 3940 may include a one-way ratchet 3942 so that as the cap is advanced along the shaft 3952, the cap may be prevented from receding relative to fastener 3950. The fastener 3950 may further include wings 3956 configured to be collapsible to pass through a hole 15 in the bone. The wings 3956 may further be configured to expand after exiting the hole to inhibit the fastener 3950 from being removed from the bone. The method of deploying the fracture plating system 3900 may include drilling a hole in a bone proximate a fracture in the bone.

The method may further include passing the wings 3956 through the hole until the wings 3956 may expand on a distal side of the hole. In an expanded or a deployed state, the wings 3956 may inhibit the fastener 3950 from being removed from the hole.

The method may further include passing a plate 3902 over the shaft 3952. The method may further include advancing the cap 3940 along the shaft 3952 to secure the plate to the bone. The plate 3902 may include similar features and functions as other plates previously described within the present disclosure.

FIG. 273 is a perspective view of a threaded anchor 4010 of the fracture plating system 4000 according to an embodiment of the present disclosure. FIG. 274 is a top view of a cap 4040 of the of the fracture plating system 4000 according to an embodiment of the present disclosure. FIG. 275 is a front view of a ratchet anchor 4050 of the of the fracture plating system 4000 according to an embodiment of the present disclosure.

The fracture plating system 4000 may include similar features and functions as other fracture plating system previously described within the present disclosure. The fracture plating system 4000 may be used with any of the plates previously described within the present disclosure. The Fracture plating system 4000 may include the threaded anchor 4010, the cap 4040, and the ratchet anchor 4050. The threaded anchor 4010 may be configured to be passed through a hole in a bone proximate a fracture in the bone. The threaded anchor 4010 may include wings 4012 that may be compressed while passing through the hole and may expand after exiting the hole. The wings 4012 may be flexible and/or hinged and may be actuated via rotation of a screw 4014.

The ratchet anchor 4050 and the cap 4040 may be configured as a self-locking ratchet mechanism, for example similar to a cable-tie mechanism. The ratchet anchor 4050 may include a shaft 4052 having a plurality of circumferential ridges 4054. The shaft 4052 may extend from a head 4056. The shaft 4052 may be configured to be received in the cap 4040. The cap 4040 may include a one-way ratchet 4042 so that as the cap 4040 is advanced along the shaft 4052, the cap 4040 may be prevented from receding relative to ratchet anchor 4050.

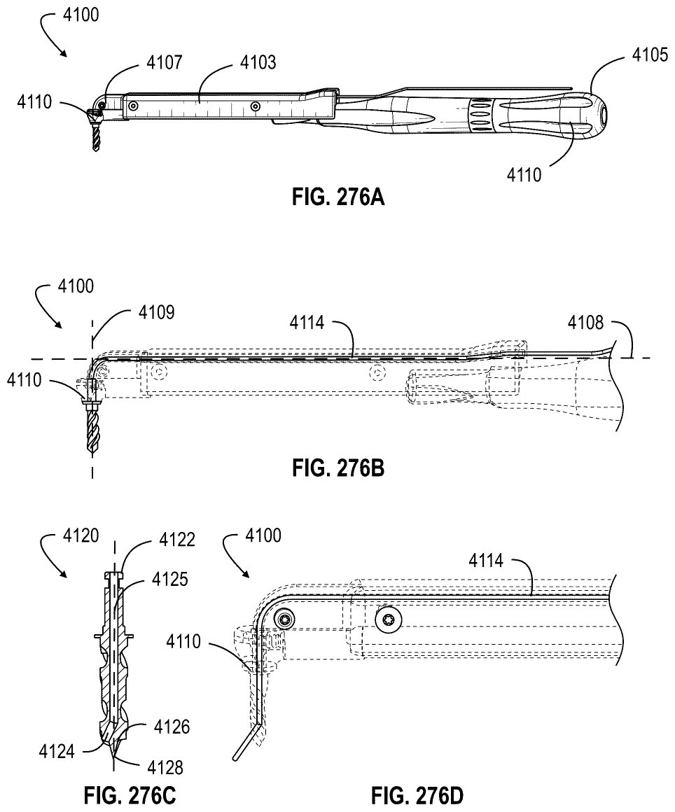

FIG. 276A is a perspective view of an angled driver 4100 of a fracture plating system 4200 (as shown in FIG. 284) according to an embodiment of the present disclosure. FIG. 276B is a perspective view of the angled driver 4100. FIG. 276C is a section view of a drill bit 4120 of the fracture plating system 4200. FIG. 276D is a perspective view of the angled driver 4100. The fracture plating system 4200 may include similar features and functions as other fracture plating system preciously described within the present disclosure. The fracture plating system 4200 may include the angled driver 4100, the drill bit 4120, a plate 4202, a cap 4240, a fastener 4250, a first tether 4210, and a second tether 4220.

The angled driver 4100 is one type of low-profile driver that may be used to provide fixation of a fracture lying under a scapula, without requiring sufficient distraction of the space under the scapula to cause damage to tissues within the space. The angled driver 4100 may thus have a relatively small height along the axis of output rotation (i.e., the vertical height of the leftmost portion of the angled driver 4100 as shown in FIG. 276A). This is portion of the angled driver 4100 that is inserted into the space; thus, the small height provided by the angulation between the output axis and the shaft of the angled driver 4100 (for example, 90° as shown) may enable a less invasive technique for surgical approaches with limited headroom, such as subscapular rib fracture repair.

The angled driver 4100 may be configured to removably receive the drill bit 4120, a hex driver bit 4130, another cutting tool bit, another driver bit, and/or another tool bit. The angled driver 4100 may be further configured to convert a first torque along a first axis 4108 to a second torque along a second axis 4109 generally perpendicular to the first axis 4108. Additionally, or alternatively, the angled driver 4100 may be configured so that the second axis is adjustable relative to the first axis in a range from 0° to 120°.

The angled driver 4100 may be further configured to facilitate drilling, driving, passing a tether, and/or other functions in a restricted area. Additionally, or alternatively, the angled driver 4100 may be configured to facilitate drilling, driving, and passing a tether to a rib positioned under a scapula. The angled driver 4100 may be configured for use in a method for a subscapular approach for deploying a plate to secure a rib having a fracture positioned under the scapula.

The angled driver 4100 may include a proximal end 4105, a distal end 4107, the first axis 4108, the second axis 4109, and a body portion extending between the distal end 4107 and the proximal end 4105. The angled driver 4100 may further include an actuation knob 4110 on the proximal end 4105, a receiving portion 4112 on the distal end 4107, and a channel 4114 extending from the distal end 4107 through the body portion 4103.

The actuation knob 4110 may be configured to rotate relative to the body portion 4103 about the first axis 4108. Rotation of the actuation knob 4110 may result in rotation of the receiving portion 4112 about the second axis 4109. The angled driver 4100 may be configured so that one rotation of the actuation knob 4110 may result in one rotation of the receiving portion 4112. Additionally, or alternatively, the angled driver 4100 may be configured so that one rotation of the actuation knob 4110 may result in more than one rotation of the receiving portion 4112. Additionally, or alternatively, the angled driver 4100 may be configured so that one rotation of the actuation knob 4110 may result in less than one rotation of the receiving portion 4112.

The channel 4114 may extend through the receiving portion 4112 and the body portion 4103. The channel 4114 may be configured to receive a first tether 4210, a second tether 4220, a third tether 4230, and/or another tether previously described within the present disclosure. The channel 4114 may be configured to receive the tether proximate the body portion 4103 and guide the tether through the receiving portion 4112. Additionally, or alternatively, the channel 4114 may be configured to receive the tether proximate the receiving portion 4112 and guide the tether through the body portion 4103. The angled driver 4100 may be configured so that the actuation knob 4110 may actuate the receiving portion 4112 with the tether received in the channel 4114.

The drill bit 4120 may be configured to be removably received in the receiving portion 4112. The drill bit 4120 may be further configured to be rotated be the angled driver 4100 to drill a hole in a bone. The drill bit 4120 may include a connection portion 4122, a cannulation 4124, and a tip portion 4126 including a brad point 4128. The connection portion 4122 may be configured to be received in the receiving portion 4112. The connection portion 4122 and the receiving portion 4112 may be configured so that with the connection portion 4122 received in the receiving portion 4112, the drill bit 4120 is captive in the angled driver 4100. Additionally, the connection portion 4122 and the receiving portion 4112 may be configured so that the drill bit 4120 is removable from the angled driver 4100.

The cannulation 4124 may be configured to receive the first tether 4210, the second tether 4220, the third tether 4230, and/or another tether previously described within the present disclosure. The cannulation 4124 may extend from the connection portion 4122 toward the tip portion 4126 along a drill bit axis 4125. The cannulation 4124 may diverge from the drill bit axis 4125 proximate the tip portion 4126 so that the cannulation 4124 does not extend through the brad point 4128.

The tip portion 4126 may include one or more cutting flutes. The brad point 4128 may be configured to engage the bone at a single point to help prevent the drill from walking when engaging an uneven surface and/or engaging a surface at an oblique angle.

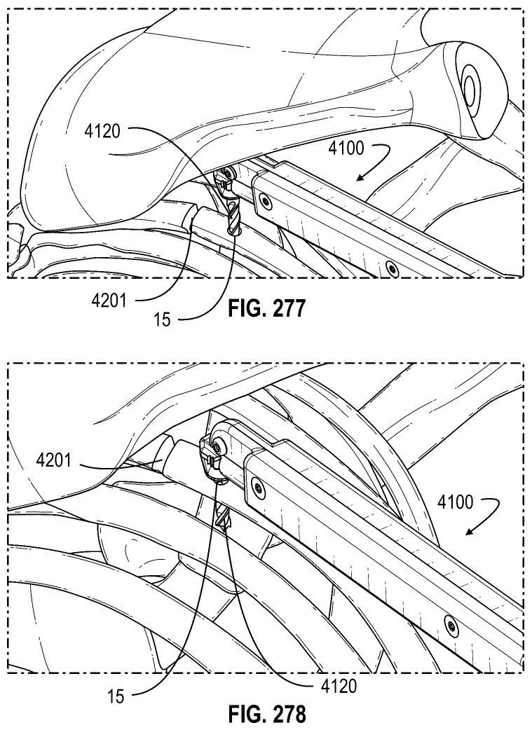

FIG. 277 is a perspective view of a step in a method of deploying the fracture plating system 4200 which may include positioning a first angled driver 4100 proximate a subscapular rib fracture 4201 according to an embodiment of the present disclosure. The method of deploying the fracture plating system 4200 may include coupling a first drill bit 4120 with the first angled driver 4100. The method may further include positioning the first angled driver 4100 under the scapula and proximate the subscapular rib fracture 4201 and positioning the first drill bit 4120 a small distance away from the subscapular rib fracture 4201.

FIG. 278 is a perspective view of a step in a method of deploying the fracture plating system 4200 which may include drilling a first hole 15 in a rib proximate the subscapular rib fracture 4201 according to an embodiment of the present disclosure. The method may further include using the first angled driver 4100 with the first drill bit 4120 to drill the first hole 15 through the rib.

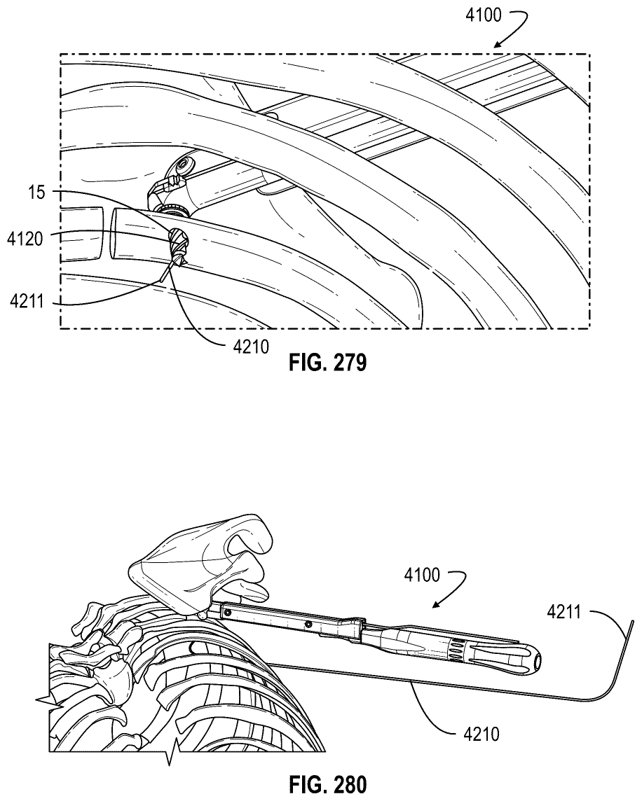

FIG. 279 is a perspective view of a step in a method of deploying the fracture plating system 4200 which may include passing a first tether end 4211 of a first tether 4210 through the first hole 15 according to an embodiment of the present disclosure. The method may further include passing the first tether end 4211 through the channel 4114 and through the first drill bit 4120 thereby passing the first tether end 4211 through the first hole 15 and into a chest cavity of the patient.

FIG. 280 is a perspective view of a step in a method of deploying the fracture plating system 4200 which may include passing the first tether end 4211 through the chest cavity to an exterior portion of the patient according to an embodiment of the present disclosure. The method may further include grasping the first tether end 4211 with a surgical instrument, such as an endoscopic grasper and/or a similar instrument, and using the surgical instrument to pull the first tether end 4211 through the chest cavity to the exterior portion of the patient. The first tether end 4211 may be pulled out to the exterior portion through a VATS portal and/or a skin incision.

FIG. 281 is a perspective view of a step in a method of deploying the fracture plating system 4200 which may include drilling a second hole 16 in the rib and passing a first tether end 4221 of a second tether 4220 through the second hole 16 according to an embodiment of the present disclosure. The method may further include positioning a second angled driver 4100′ proximate the subscapular rib fracture 4201, drilling a second hole 16, and passing the first tether end 4221 of the second tether 4220 through the second angled driver 4100′ and into the chest cavity, similar to the steps described previously.

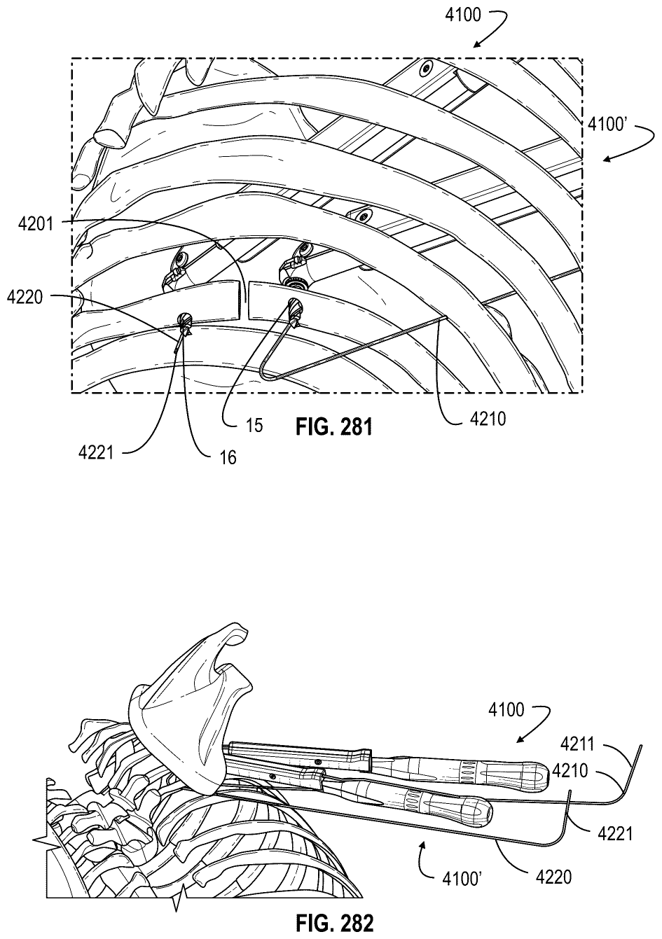

FIG. 282 is a perspective view of a step in a method of deploying the fracture plating system 4200 which may include passing the first tether end 4221 through the chest cavity to the exterior portion of the patient according to an embodiment of the present disclosure. The method may further include grasping the first tether end 4221 with a surgical instrument, such as an endoscopic grasper and/or a similar instrument, and using the surgical instrument to pull the first tether end 4221 through the chest cavity to the exterior portion of the patient. The first tether end 4221 may be pulled out to the exterior portion through a VATS portal and/or a skin incision.

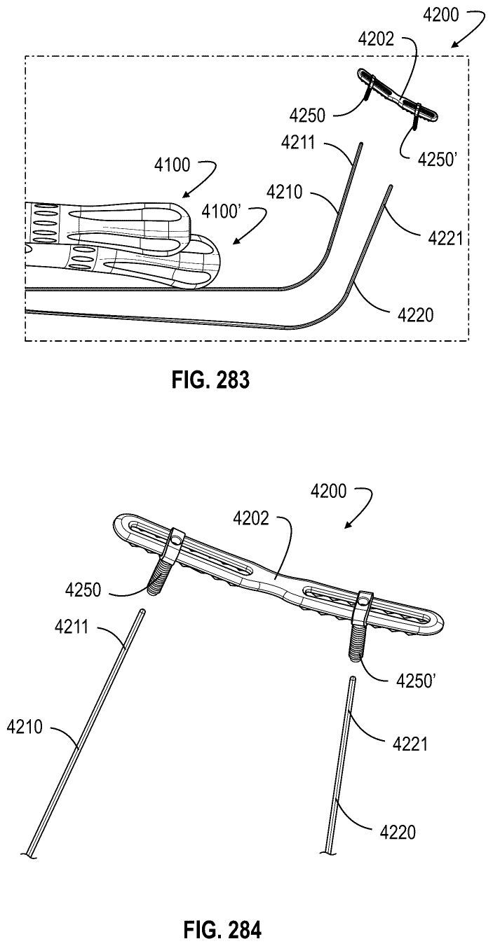

FIG. 283 is a perspective view of a step in a method of deploying the fracture plating system 4200 which may include positioning a first tether end 4211 of the first tether 4210 and a first tether end 4221 of the second tether 4220 proximate a first fastener 4250 and a second fastener 4250′ of the fracture plating system 4200 according to an embodiment of the present disclosure. FIG. 284 is a perspective view the step of FIG. 283. The plate 4202 may include similar features and functions as other plates previously described within the present disclosure, for example the plate 3602. The fastener 4250 may include similar features and functions as other fasteners previously described within the present disclosure, for example the fastener 3650.

The method may further include selecting a plate 4202 from a set of plates each having a different length, a different width, a different number of slots, and/or a different configuration of slots. The selection of the plate 4202 may be based on patient anatomy, number of fractures, location of the fractures, and/or surgeon discretion. The method may further include selecting the first fastener 4250 and the second fastener 4250′ from a set of fasteners each having a different length, wing feature, and/or head portion. The selection of the first fastener 4250 and/or the second fastener 4250′ may be based on the selected plate 4202, patient anatomy, and/or surgeon discretion. The method may further include positioning the first fastener 4250 proximate the first tether end 4211 and the second fastener 4250′ proximate the first tether end 4221.

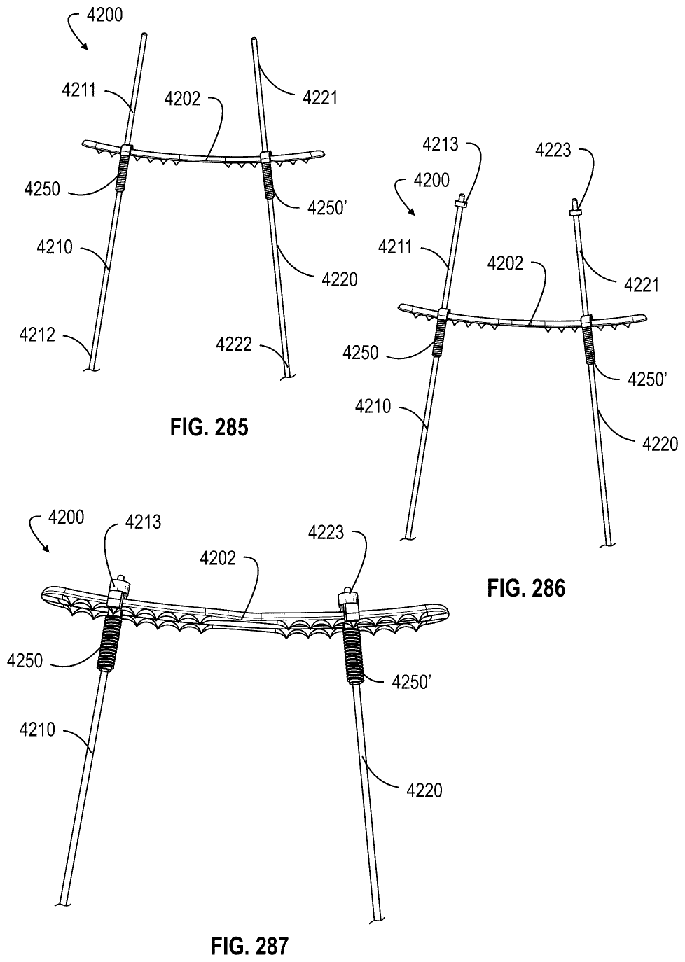

FIG. 285 is a perspective view of a step in a method of deploying the fracture plating system 4200 which may include passing the second tether end 4212 of the first tether 4210 and the second tether end 4222 of the second tether 4220 through the first fastener 4250 and the second fastener 4250′ of the fracture plating system 4200 according to an embodiment of the present disclosure. The method may further include passing the second tether end 4212 of the first tether 4210 and the second tether end 4222 of the second tether 4220 through a first cannulation 4254 of the first fastener 4250 and a second cannulation 4254′ of the second fastener 4250′.

FIG. 286 is a perspective view of a step in a method of deploying the fracture plating system 4200 which may include securing a first bead 4213 and a second bead 4223 on the first tether 4210 and the second tether 4220 according to an embodiment of the present disclosure. The first bead 4213 and the second bead 4223 may include similar features and functions as the first bead 1963 and the second bead 1965. The first bead 4213 and/or the second bead 4223 may be configured to be mechanically fixed to the first tether 4210 and/or the second tether 4220.

The method may further include fixing the first bead 4213 and/or the second bead 4223 to the first tether 4210 and/or the second tether 4220 after the first tether 4210 is received in the first fastener 4250 and/or the second tether 4220 is received in the second fastener 4250′. Additionally, or alternatively, the method may further include fixing the first bead 4213 and/or the second bead 4223 to the first tether 4210 and/or the second tether 4220 before the first tether 4210 is received in the first fastener 4250 and/or the second tether 4220 is received in the second fastener 4250′.

In an embodiment, the first bead 4213 and/or the second bead 4223 may be configured as a sleeve having a cannulation. The sleeve may be passed over the first tether 4210 and/or the second tether 4220 and crimped in place. Additionally, or alternatively, the first tether 4210 and/or the second tether 4220 may be tied in a knot so that the knot may function as the first bead 4213 and/or the second bead 4223.

FIG. 287 is a perspective view of a step in a method of deploying the fracture plating system 4200 which may include advancing the first tether 4210 and the second tether 4220 so that the first bead 4213 and the second bead 4223 contact the first fastener 4250 and the second fastener 4250′ according to an embodiment of the present disclosure. The method may further include pulling the first tether 4210 and/or the second tether 4220 so that the first bead 4213 and/or the second bead 4223 advance toward and engage the first fastener 4250 and/or the second fastener 4250′. The plate 4202, the first fastener 4250, and the second fastener 4250′ may be captive on the first tether 4210 and/or the second tether 4220.

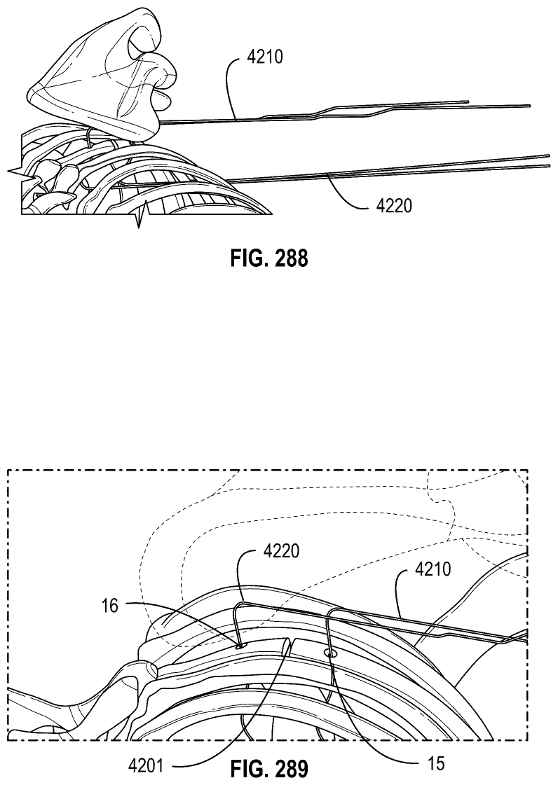

FIG. 288 is a perspective view of a step in a method of deploying the fracture plating system 4200 which may include withdrawing the first angled driver 4100 and the second angled driver 4100′ from the first tether 4210 and the second tether 4220 according to an embodiment of the present disclosure. The method may further include withdrawing the first angled driver 4100 along the first tether 4210. The method may further include withdrawing the second angled driver 4100′ along the second tether 4220.

FIG. 289 is a perspective view of a step in a method of deploying the fracture plating system 4200 which may include the first tether 4210 being received in the first hole 15 and the second tether 4220 being received in the second hole 16 according to an embodiment of the present disclosure.

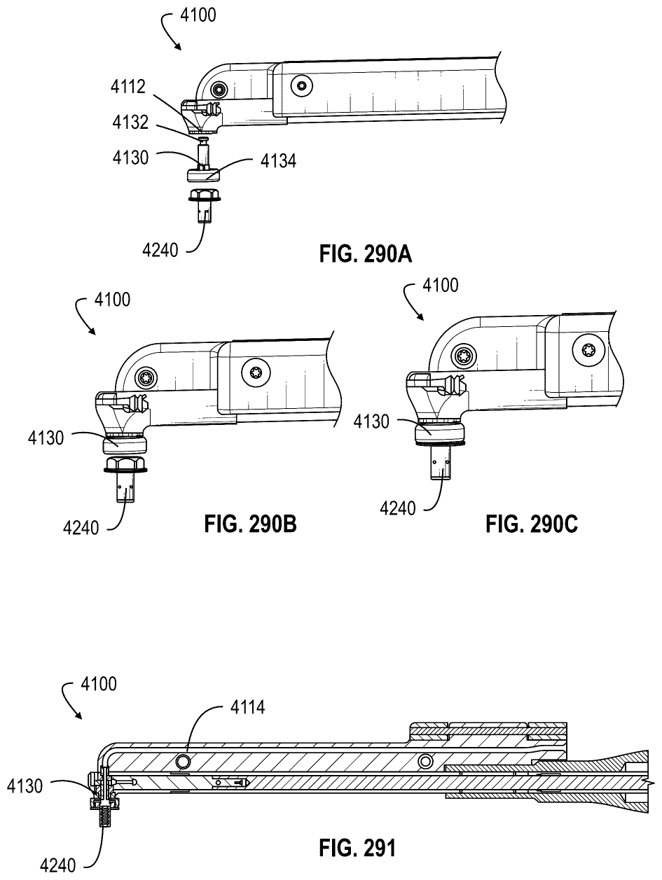

FIG. 290A is a perspective view of a step in a method of deploying the fracture plating system 4200 which may include coupling a first hex driver bit 4130 to the first angled driver 4100 according to an embodiment of the present disclosure. The hex driver bit 4130 may be configured to engage the cap 4240 to apply torque to the cap 4240. The hex driver bit 4130 may include a connection portion 4132 and a driver portion 4134.

The connection portion 4132 may be configured to be received in the receiving portion 4112 of the angled driver 4100. The connection portion 4132 and the receiving portion 4112 may be configured so that with the connection portion 4132 received in the receiving portion 4112, the hex driver bit 4130 is captive in the angled driver 4100. Additionally, the connection portion 4132 and the receiving portion 4112 may be configured so that the hex driver bit 4130 is removable from the angled driver 4100.

The driver portion 4134 may be configured to receive the cap 4240 to facilitate engagement of the cap 4240 with the fastener 4250. The driver portion 4134 may be configured a non-circular recess. The driver portion 4134 may be shaped as a hex, a square, a hexalobe, or another non-circular shape. The driver portion 4134 may include a shape and depth that is complementary to the cap 4240.

FIG. 290B is a perspective view of a step in a method of deploying the fracture plating system 4200 which may include coupling a first cap 4240 with the first hex driver bit 4130 according to an embodiment of the present disclosure. FIG. 290C is a perspective view of the first cap 4240 coupled with the first hex driver bit 4130 according to an embodiment of the present disclosure. The method may further include coupling the first hex driver bit 4130 to the first angled driver 4100 and receiving the first cap 4240 in the hex driver bit 4130. The method may further include coupling a second hex driver bit 4130′ with a second angled driver 4100′ and receiving a second cap 4240′ in the second hex driver bit 4130′.

FIG. 291 is a cross section view of the first cap 4240 coupled with the first hex driver bit 4130 according to an embodiment of the present disclosure. A first channel 4114 may extend continuously through the first cap 4240, the first hex driver bit 4130, and the first angled driver 4100.

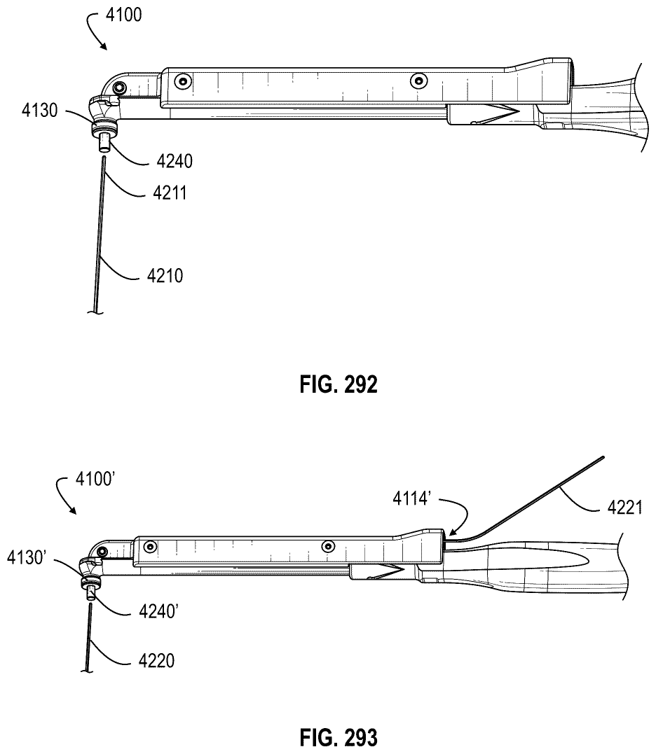

FIG. 292 is a perspective view of a step in a method of deploying the fracture plating system 4200 which may include passing the first tether 4210 into the first cap 4240 and first hex driver bit 4130 according to an embodiment of the present disclosure. The method may further include passing the first tether end 4211 into the first cap 4240 and the first hex driver bit 4130 and passing the first tether end 4221 into the second cap 4240′ and the second hex driver bit 4130′.

FIG. 293 is a perspective view of a step in a method of deploying the fracture plating system 4200 which may include passing the first tether end 4211 through the second angled driver 4100′ according to an embodiment of the present disclosure. The method may further include passing the first tether end 4211 through the first channel 4114 and out of the first angled driver 4100. The method may further include passing the first tether end 4221 through a second channel 4114′ of the second angled driver 4100′ and out of the second angled driver 4100′.

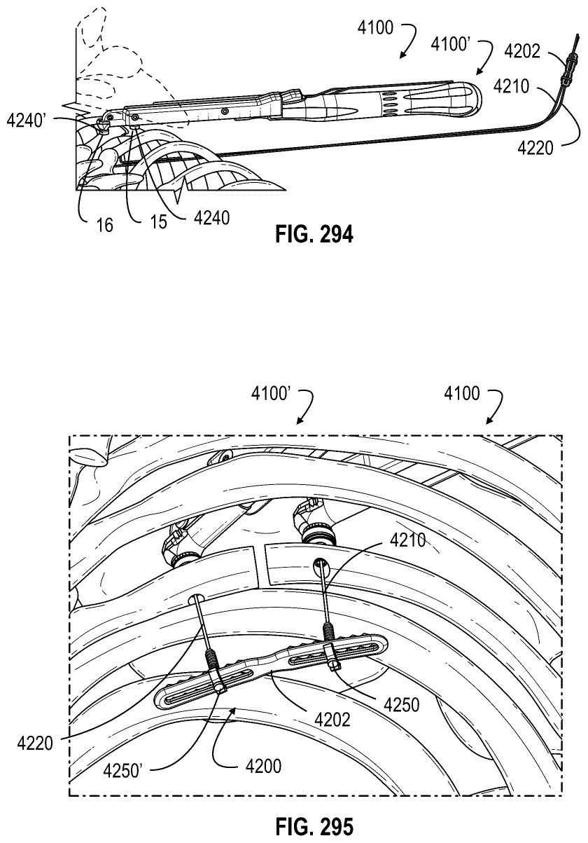

FIG. 294 is a perspective view of a step in a method of deploying the fracture plating system 4200 which may include using the first tether 4210 to guide the first angled driver 4100 and the first cap 4240 to the first hole 15 according to an embodiment of the present disclosure. The method may further include passing the first angled driver 4100 along the first tether 4210 to the first hole 15. The method may further include passing the second angled driver 4100′ along the second tether 4220 to the second hole 16. The first tether 4210 and/or the second tether 4220 may be configured to guide the first cap 4240 and/or the second cap 4240′ into the first hole 15 and/or the second hole 16.

FIG. 295 is a perspective view of a step in a method of deploying the fracture plating system 4200 which may include using the first tether 4210 and second tether 4220 to draw the plate 4202 to an interior cavity according to an embodiment of the present disclosure. The method may further include pulling the first tether 4210 and/or the second tether 4220 through the first angled driver 4100 and/or the second angled driver 4100′ to draw the plate 4202, the first fastener 4250 and/or the second fastener 4250′ through a skin incision and/or a VATS port and through the chest cavity. The first cap 4240 and/or the second cap 4240′ may be positioned in the first hole 15 and/or the second hole 16 so that the first tether 4210 and/or the second tether 4220 may pass through the first hole 15 and/or the second hole 16 without causing damage to the bone.

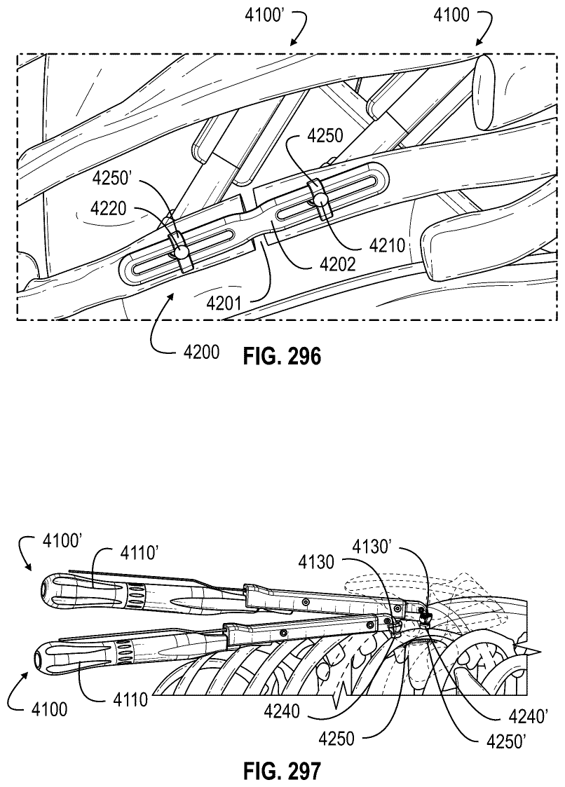

FIG. 296 is a perspective view of a step in a method of deploying the fracture plating system 4200 which may include using the first tether 4210 and second tether 4220 to draw the plate 4202 to the subscapular rib fracture 4201 according to an embodiment of the present disclosure. The method may further include pulling the first tether 4210 and/or the second tether 4220 through the first angled driver 4100 and/or the second angled driver 4100′ to draw the plate 4202, the first fastener 4250 and/or the second fastener 4250′ to the subscapular rib fracture 4201.

FIG. 297 is a perspective view of a step in a method of deploying the fracture plating system 4200 which may include using the first angled driver 4100 and the second angled driver 4100′ to threadably engage the first cap 4240 and the second cap 4240′ with the first fastener 4250 and the second fastener 4250′ according to an embodiment of the present disclosure. The method may further include actuating a first actuation knob 4110 of the first angled driver 4100 to rotate the first hex driver bit 4130 to engage the first cap 4240 with the first fastener 4250. The method may further include actuating a second actuation knob 4110′ of the second angled driver 4100′ to rotate the second hex driver bit 4130′ to engage the second cap 4240′ with the second fastener 4250′.

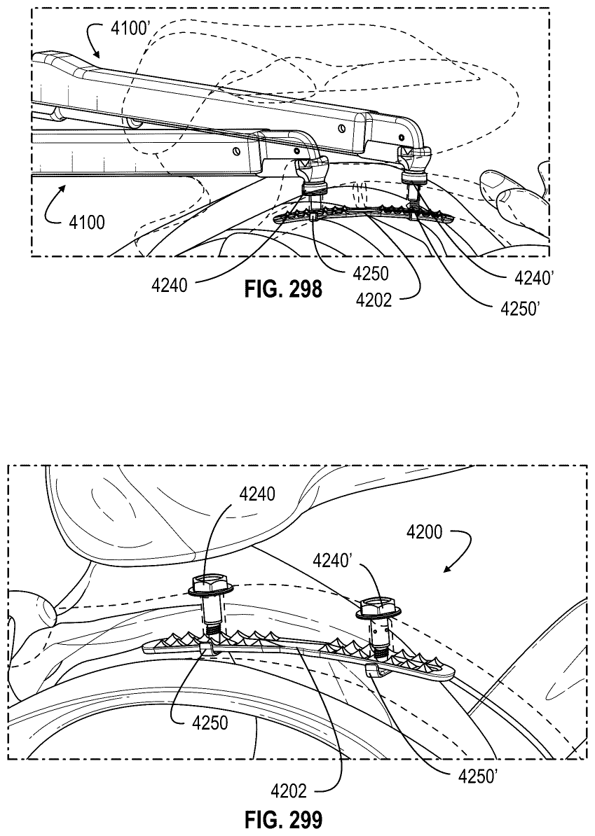

FIG. 298 is a perspective view of a step in a method of deploying the fracture plating system 4200 which may include using the first angled driver 4100 and the second angled driver 4100′ to secure the first cap 4240 and the second cap 4240′ to the first fastener 4250 and the second fastener 4250′ according to an embodiment of the present disclosure. The method may further include actuating the first actuation knob 4110 to rotate the first hex driver bit 4130 to secure the first cap 4240 to the first fastener 4250. The method may further include actuating the second actuation knob 4110′ to rotate the second hex driver bit 4130′ to secure the second cap 4240′ to the second fastener 4250′, thereby securing the plate 4202 to the bone.

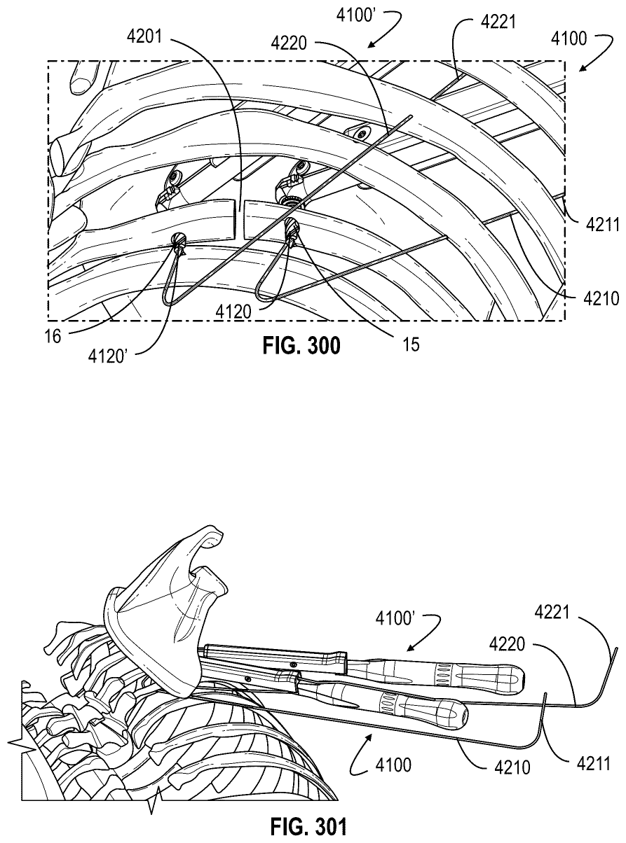

FIG. 299 is a perspective view of a step in a method of deploying the fracture plating system 4200 which may include withdrawing the first tether 4210, the second tether 4220, the first angled driver 4100, and the second angled driver 4100′ according to an embodiment of the present disclosure. The method may further include withdrawing the first tether 4210 through the first angled driver 4100 and withdrawing the second tether 4220 through the second angled driver 4100′. The method may further include withdrawing the first angled driver 4100 and the second angled driver 4100′.

FIG. 300 is a perspective view of a step in a method of deploying the fracture plating system 4200 which may include drilling a first hole 15 and a second hole 16 in the rib and passing a first tether 4210 and a second tether 4220 through the first hole 15 and the second hole 16 according to an embodiment of the present disclosure. The method of deploying the fracture plating system 4200 may include using a first angled driver 4100 and a first drill bit 4120 to drill the first hole 15 proximate a subscapular rib fracture 4201 and passing a first tether end 4211 of the first tether 4210 through the first angled driver 4100 and into the chest cavity. The method of deploying the fracture plating system 4200 may include using a second angled driver 4100′ and a second drill bit 4120′ to drill the second hole 16 proximate the subscapular rib fracture 4201 and passing a first tether end 4221 of the second tether 4220 through the second angled driver 4100′ and into the chest cavity.

FIG. 301 is a perspective view of a step in a method of deploying the fracture plating system 4200 which may include passing the first tether end 4211 and the first tether end 4221 through the chest cavity to an exterior portion of the patient according to an embodiment of the present disclosure. The method may further include grasping the first tether end 4221 and/or the first tether end 4221 with a surgical instrument, such as an endoscopic grasper and/or a similar instrument, and using the surgical instrument to pull the first tether end 4221 and/or the first tether end 4221 through the chest cavity to the exterior portion of the patient. The first tether end 4221 and/or the first tether end 4221 may be pulled out to the exterior portion through a VATS portal and/or a skin incision.

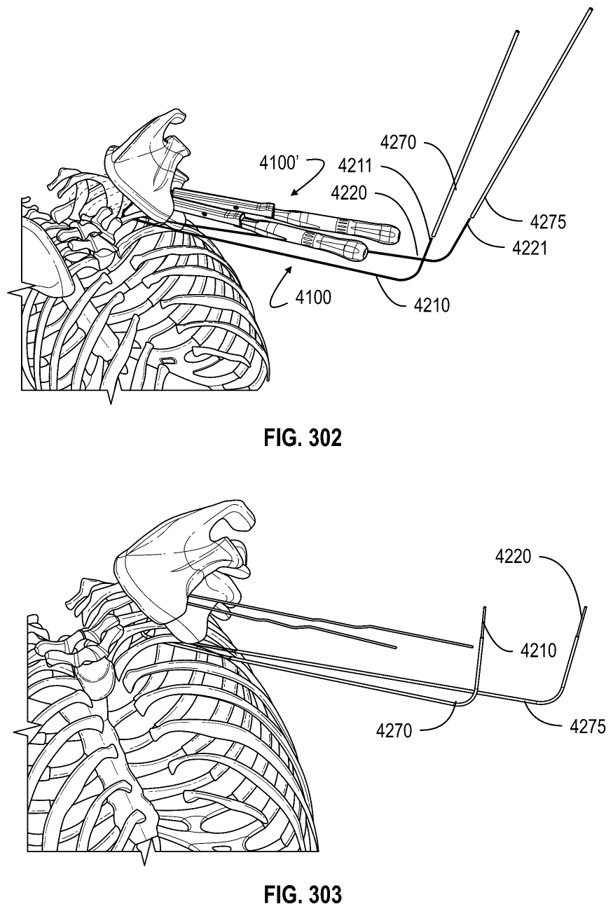

FIG. 302 is a perspective view of a step in a method of deploying the fracture plating system 4200 which may include passing a first tube 4270 over the first tether end 4211 and a second tube 4275 over the first tether end 4221 according to an embodiment of the present disclosure. The first tube 4270 and/or the second tube 4275 may include a soft flexible material, for example, rubber and/or silicone. The method may further include receiving the first tether 4210 and/or the second tether 4220 in the first tube 4270 and/or the second tube 4275.

FIG. 303 is a perspective view of a step in a method of deploying the fracture plating system 4200 which may include advancing the first tube 4270 and the second tube 4275 to the first hole 15 and the second hole 16 according to an embodiment of the present disclosure. The method may further include advancing the first tube 4270 and/or the second tube 4275 along the first tether 4210 and/or the second tether 4220 to the first hole 15 and/or the second hole 16.

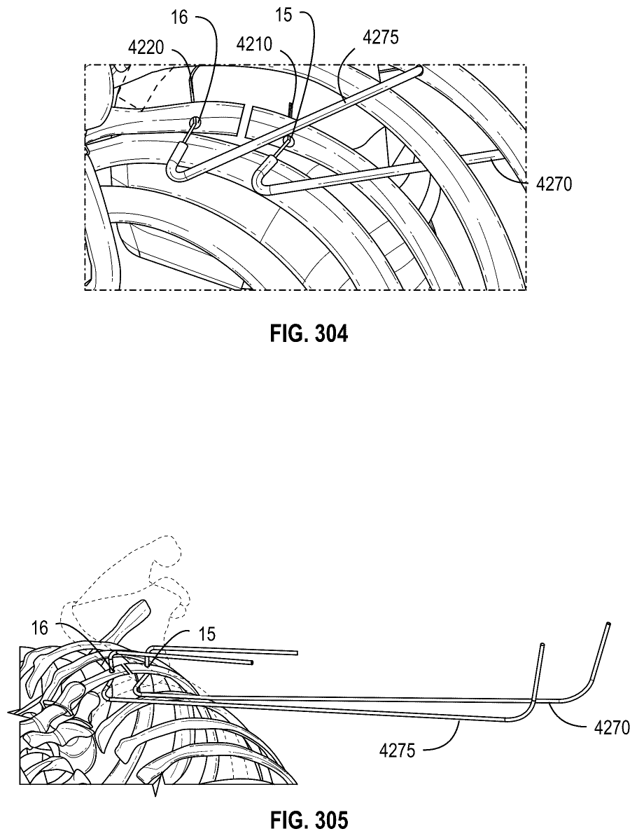

FIG. 304 is a perspective view of a step in a method of deploying the fracture plating system 4200 which may include passing the first tube 4270 and the second tube 4275 through the first hole 15 and the second hole 16 according to an embodiment of the present disclosure. The method may further include advancing the first tube 4270 and/or the second tube 4275 along the first tether 4210 and/or the second tether 4220 through the first hole 15 and/or the second hole 16.

FIG. 305 is a perspective view of a step in a method of deploying the fracture plating system 4200 which may include passing the first tube 4270 and the second tube 4275 to an exterior portion of the patient and withdrawing the first tether 4210 and the second tether 4220 according to an embodiment of the present disclosure. The method may further include advancing the first tube 4270 and/or the second tube 4275 along the first tether 4210 and/or the second tether 4220 to the exterior portion of the patient. The method may further include withdrawing the first tether 4210 and/or the second tether 4220 from the chest cavity.

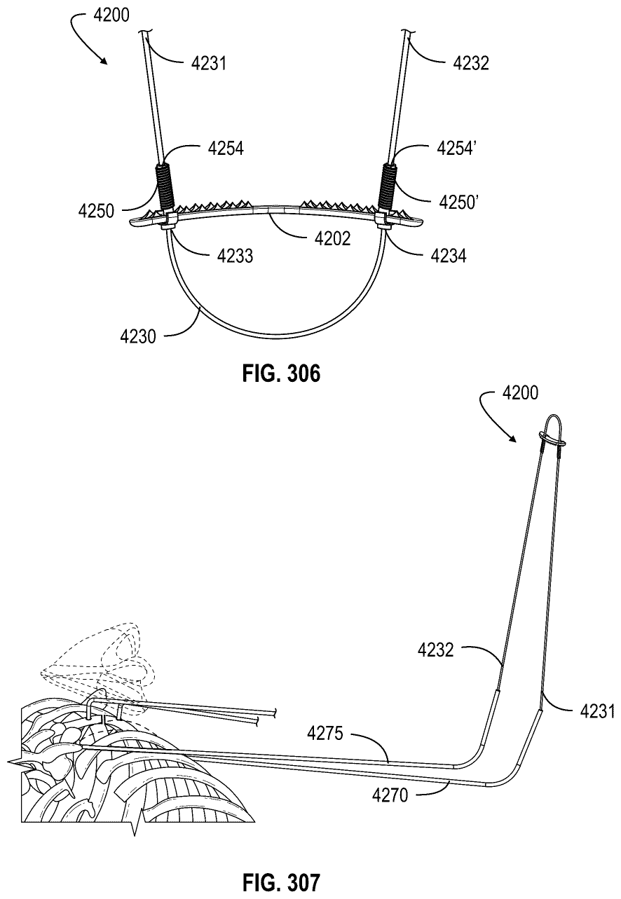

FIG. 306 is a perspective view of a step in a method of deploying the fracture plating system 4200 which may include passing a third tether 4230 through a first fastener 4250 and a second fastener 4250′ of the fracture plating system 4200 according to an embodiment of the present disclosure. The third tether 4230 may include similar features and functions as other tether previously described within the present disclosure, for example the tether 1960. The method may further include passing a first tether end 4231 of the third tether 4230 through a first cannulation 4254 of the first fastener 4250 and a second tether end 4232 of the third tether 4230 through a second cannulation 4254′ of the second fastener 4250′. The method may further include advancing the first tether end 4231 and the second tether end 4232 so that a third bead 4233 of the third tether 4230 abuts the first fastener 4250 and a fourth bead 4234 of the third tether 4230 abuts the second fastener 4250′. The third bead 4233 and/or the fourth bead 4234 may include similar features and functions as the first bead 4213 and/or the second bead 4223 previously described.

FIG. 307 is a perspective view of a step in a method of deploying the fracture plating system 4200 which may include positioning the third tether 4230 proximate the first tube 4270 and the second tube 4275 according to an embodiment of the present disclosure. The method may further include positioning the first tether end 4231 proximate the first tube 4270 and the second tether end 4232 proximate the second tube 4275.

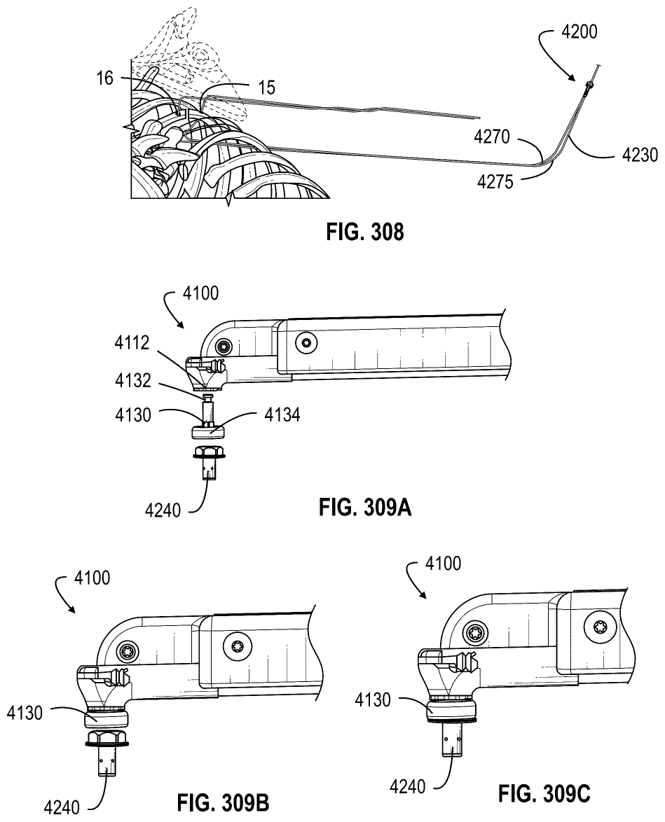

FIG. 308 is a perspective view of a step in a method of deploying the fracture plating system 4200 which may include passing the third tether 4230 through the first tube 4270 and the second tube 4275 according to an embodiment of the present disclosure. The method may further include passing the first tether end 4231 within the first tube 4270 toward the first hole 15 and passing the second tether end 4232 within the second tube 4275 toward the second hole 16.

FIG. 309A is a perspective view of a step in a method of deploying the fracture plating system 4200 which may include coupling a first hex driver bit 4130 to the first angled driver 4100 according to an embodiment of the present disclosure. FIG. 309B is a perspective view of a step in a method of deploying the fracture plating system 4200 which may include coupling a first cap 4240 with the first hex driver bit 4130 according to an embodiment of the present disclosure. FIG. 309C is a perspective view of the first cap 4240 coupled with the first hex driver bit 4130 according to an embodiment of the present disclosure. The method may further include coupling the first hex driver bit 4130 to the first angled driver 4100 and receiving the first cap 4240 in the hex driver bit 4130. The method may further include coupling a second hex driver bit 4130′ with a second angled driver 4100′ and receiving a second cap 4240′ in the second hex driver bit 4130′

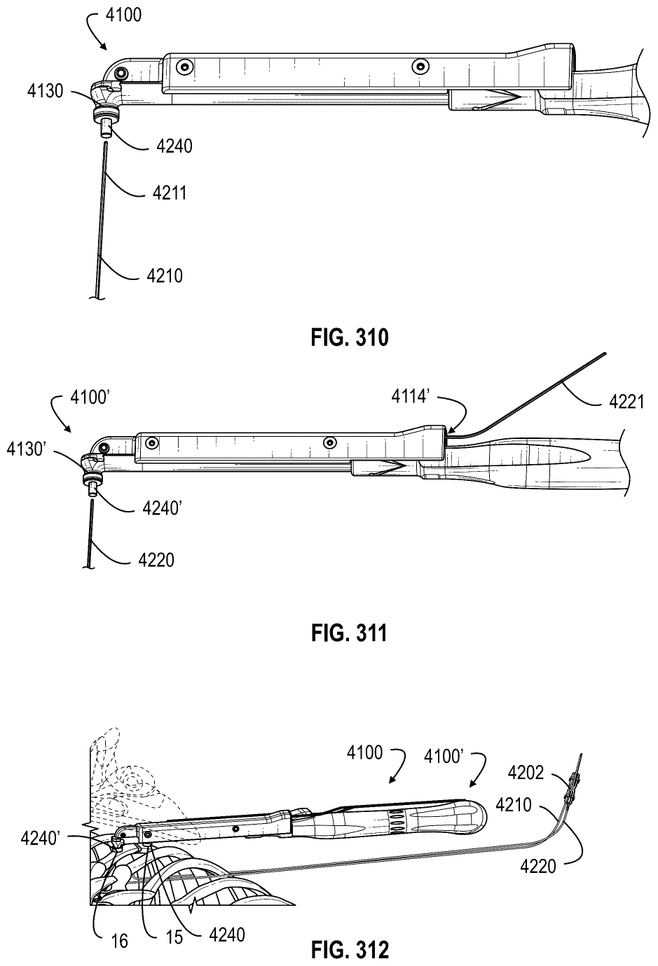

FIG. 310 is a perspective view of a step in a method of deploying the fracture plating system 4200 which may include passing the first tether end 4231 into the first cap 4240 and first hex driver bit 4130 according to an embodiment of the present disclosure. The method may further include passing the first tether end 4231 into the first cap 4240 and the first hex driver bit 4130 and passing the second tether end 4232 into the second cap 4240′ and the second hex driver bit 4130′.

FIG. 311 is a perspective view of a step in a method of deploying the fracture plating system 4200 which may include passing the third tether 4230 through the first angled driver 4100 according to an embodiment of the present disclosure. The method may further include passing the first tether end 4231 through the first channel 4114 and out of the first angled driver 4100. The method may further include passing the second tether end 4232 through a second channel 4114′ of the second angled driver 4100′ and out of the second angled driver 4100′.

FIG. 312 is a perspective view of a step in a method of deploying the fracture plating system 4200 which may include using the third tether 4230 to guide the first angled driver 4100 and the first cap 4240 to the first hole 15 according to an embodiment of the present disclosure. The method may further include passing the first angled driver 4100 along the first tether end 4231 to the first hole 15. The method may further include passing the second angled driver 4100′ along the second tether end 4232 to the second hole 16. The third tether 4230 may be configured to guide the first cap 4240 and/or the second cap 4240′ into the first hole 15 and/or the second hole 16.

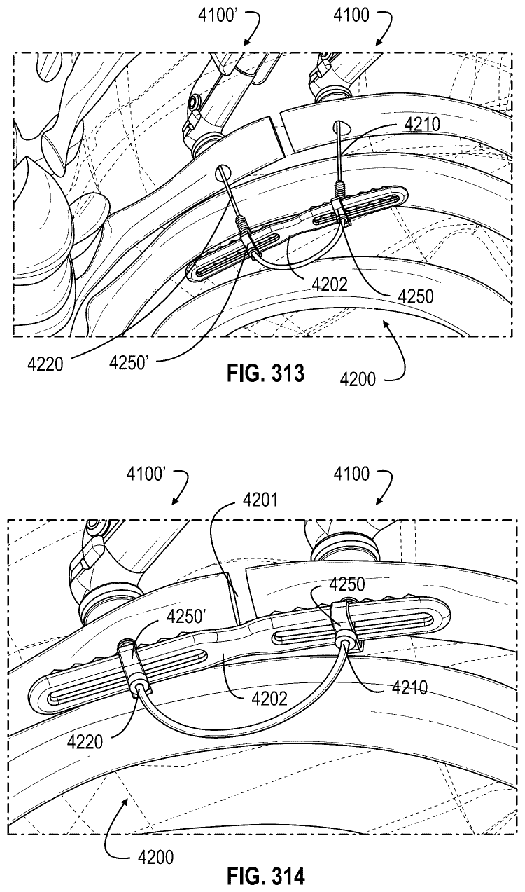

FIG. 313 is a perspective view of a step in a method of deploying the fracture plating system 4200 which may include using the third tether 4230 to draw the plate 4202 to an interior cavity according to an embodiment of the present disclosure. The method may further include pulling the first tether end 4231 and the second tether end 4232 through the first angled driver 4100 and the second angled driver 4100′ to draw the plate 4202, the first fastener 4250 and/or the second fastener 4250′ through a skin incision and/or a VATS port and through the chest cavity. The first cap 4240 and/or the second cap 4240′ may be positioned in the first hole 15 and/or the second hole 16 so that the third tether 4230 may pass through the first hole 15 and/or the second hole 16 without causing damage to the bone.

FIG. 314 is a perspective view of a step in a method of deploying the fracture plating system 4200 which may include using the third tether 4230 to draw the plate 4202 to the subscapular rib fracture 4201 according to an embodiment of the present disclosure. The method may further include pulling the first tether end 4231 and the second tether end 4232 through the first angled driver 4100 and the second angled driver 4100′ to draw the plate 4202, the first fastener 4250 and/or the second fastener 4250′ to the subscapular rib fracture 4201.

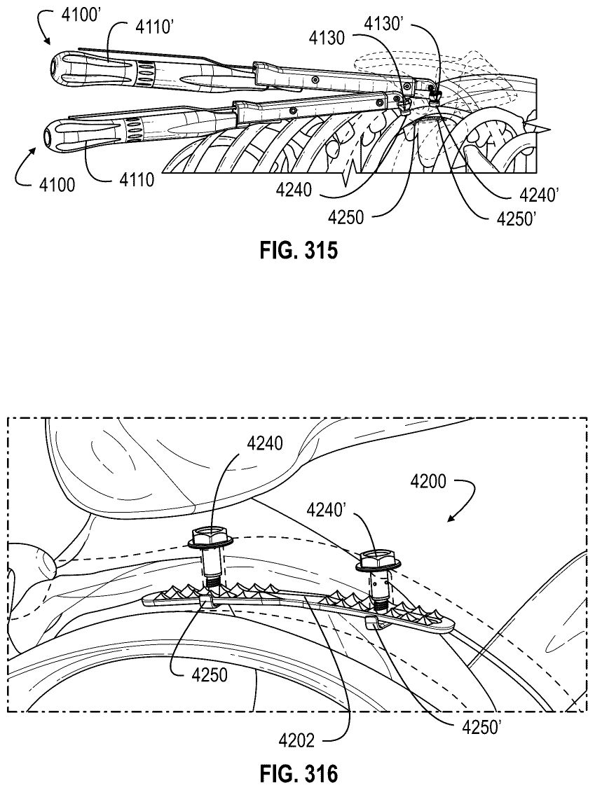

FIG. 315 is a perspective view of a step in a method of deploying the fracture plating system 4200 which may include using the first angled driver 4100 and the second angled driver 4100′ to threadably engage and secure the first cap 4240 and the second cap 4240′ with the first fastener 4250 and the second fastener 4250′ according to an embodiment of the present disclosure. The method may further include actuating a first actuation knob 4110 of the first angled driver 4100 to rotate the first hex driver bit 4130 to engage and secure the first cap 4240 with the first fastener 4250. The method may further include actuating a second actuation knob 4110′ of the second angled driver 4100′ to rotate the second hex driver bit 4130′ to engage and secure the second cap 4240′ with the second fastener 4250′.

FIG. 316 is a perspective view of a step in a method of deploying the fracture plating system 4200 which may include withdrawing the third tether 4230, the first angled driver 4100, and the second angled driver 4100′ according to an embodiment of the present disclosure. The method may further include withdrawing the third tether 4230 through the first angled driver 4100 and withdrawing the second tether 4220 through the second angled driver 4100′. The method may further include withdrawing the first angled driver 4100 and the second angled driver 4100′. The method may further include withdrawing the third tether 4230 through the skin incision and/or the VATS port.

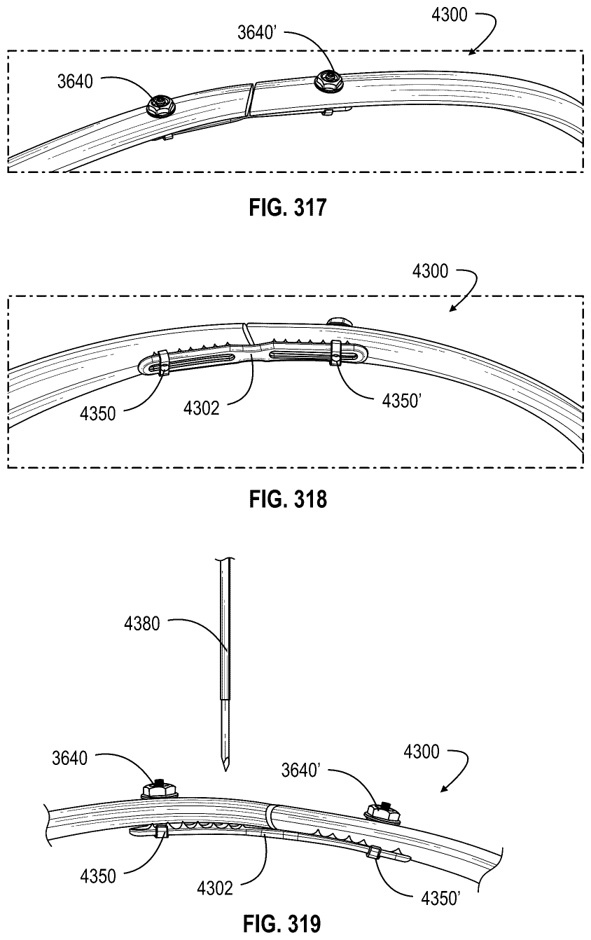

FIG. 317 is a perspective view of a fracture plating system 4300 deployed on an exemplary fracture of a rib according to an embodiment of the present disclosure. FIG. 318 is a perspective view of the fracture plating system 4300. The fracture plating system 4300 may include similar features and functions as other fracture plating system previously described within the present disclosure. The fracture plating system 4300 may include a plate 4302, a cap 4340, a fastener 4350, a tether 4360, and a rescue fastener 4370. The plate 4302 may include similar features and functions as the plate 3602. The cap 4340 may include similar features and functions as the cap 3640. The fastener 4350 may include similar features and function as the fastener 3650. The tether 4360 may include similar features and functions as the first tether 4210. The fracture plating system 4300 may include the plate 4302, a first fastener 4350, a first cap 3640, a second fastener 4350′, and a second cap 3640′.

The rescue fastener 4370 may include similar features and functions as the fastener 3850 previously described, except that the rescue fastener 4370 may be designed to be inserted through a slot of the plate 4302 after the plate 4302 has already been placed and/or secured against the bone (for example, via the fastener 4350, the fastener 4350′, the cap 3640 and the cap 3640′). This may be useful in a variety of surgical scenarios, such as when the surgeon believes that additional fixation to the bone is needed after the plate 4302 has been secured to the bone.

For example, additional fixation may be desirable if the plate 4302 is not sufficiently conformed to the shape of the bone, if the quality of the bone is believed to be too poor to retain the plate 4302 without supplemental fixation, or if the bone has multiple fractures (for example, defining one or more flail segments) that require additional fixation. Use of the rescue fastener 4370 may be planned preoperatively, or interoperatively, for example, if any of the conditions listed above is first observed by the surgeon during surgery. The term “rescue” may relate to any situation in which additional fixation is deemed desirable, even if it is not deemed necessary for the success of the procedure.

FIG. 319 is a perspective view of a step in a method of deploying the fracture plating system 4300 which may include inserting a guide wire 4380 into the rib according to an embodiment of the present disclosure. The method of deploying a rescue fastener 4370 of the fracture plating system 4300 may include inserting the guide wire 4380 into the rib proximate a fracture. The guide wire 4380 may be positioned so that, with the plate 4302 secured to the rib with a first fastener 4350 and a second fastener 4350′, the guide wire 4380 contact the rib between the first fastener 4350 and the second fastener 4350′. Additionally, the guide wire 4380 may be positioned so that the guide wire 4380 may align with a slot and/or an opening in the plate 4302.

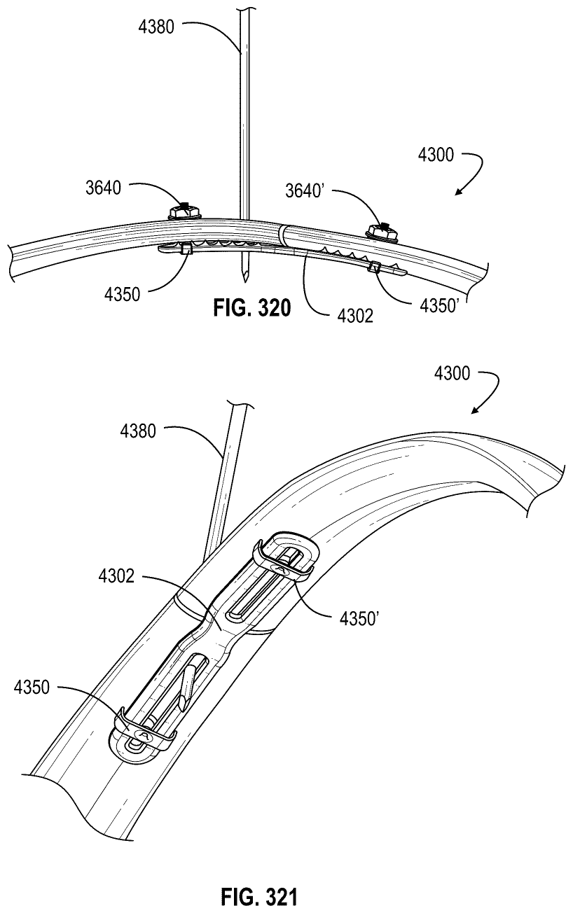

FIG. 320 is a perspective view of a step in a method of deploying the fracture plating system 4300 which may include advancing the guide wire 4380 through the rib according to an embodiment of the present disclosure. FIG. 321 is a perspective view of the method step of FIG. 320. The method may further include advancing the guide wire 4380 through the rib so that the guide wire 4380 also advances through a slot and/or opening in the plate 4302.

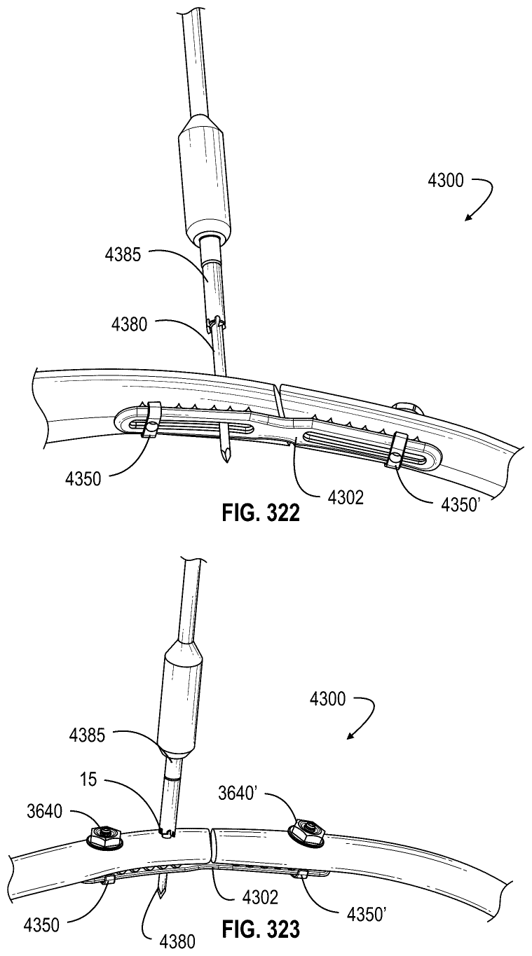

FIG. 322 is a perspective view of a step in a method of deploying the fracture plating system 4300 which may include advancing a cannulated drill bit 4385 along the guide wire 4380 according to an embodiment of the present disclosure. The method may further include advancing the cannulated drill bit 4385 along the guide wire 4380 toward the rib.

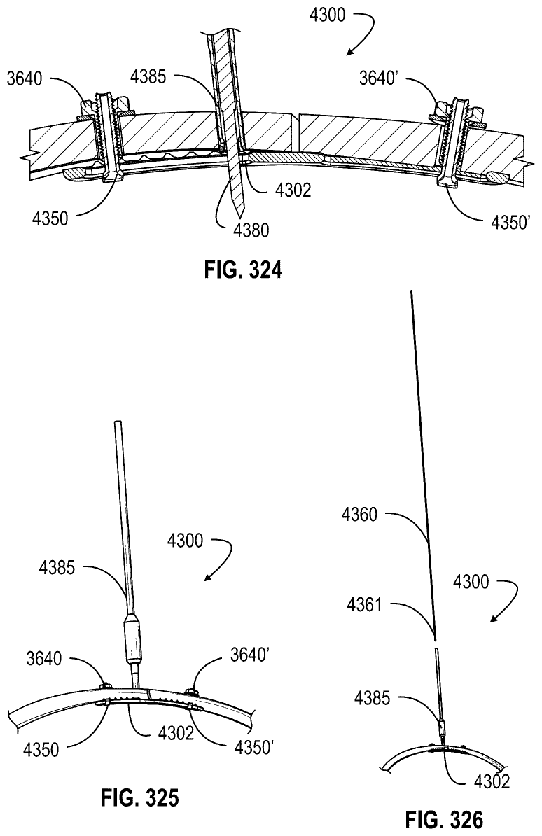

FIG. 323 is a perspective view of a step in a method of deploying the fracture plating system 4300 which may include using the cannulated drill bit 4385 to drill a hole 15 through the rib according to an embodiment of the present disclosure. FIG. 324 is a cross section view of the method step of FIG. 323. The method may further include continuing to advance the cannulated drill bit 4385 along the guide wire 4380 so that the cannulated drill bit 4385 drills a hole 15 in the rib.

FIG. 325 is a perspective view of a step in a method of deploying the fracture plating system 4300 which may include withdrawing the guide wire 4380 from the cannulated drill bit 4385 according to an embodiment of the present disclosure. The method may further include withdrawing the guide wire 4380 from the cannulated drill bit 4385 and from the rib while leaving the cannulated drill bit 4385 in place within the hole 15.

FIG. 326 is a perspective view of a step in a method of deploying the fracture plating system 4300 which may include positioning the tether 4360 proximate the cannulated drill bit 4385 according to an embodiment of the present disclosure. The method may further include positioning a first tether end 4361 of the tether 4360 proximate the cannulated drill bit 4385.

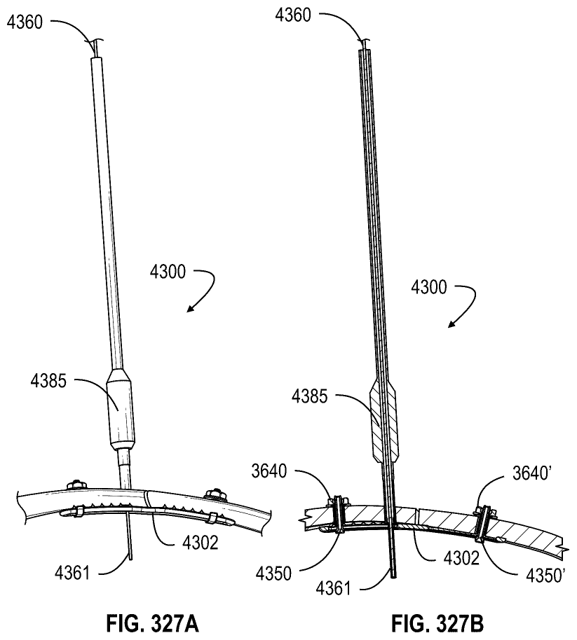

FIG. 327A is a perspective view of a step in a method of deploying the fracture plating system 4300 which may include passing the first tether end 4361 through the cannulated drill bit 4385 and through the rib according to an embodiment of the present disclosure. FIG. 327B is a cross section view of the method step of FIG. 327A. the method may further include advancing the first tether end 4361 through a cannulation of the cannulated drill bit 4385, through the hole 15, and through the plate 4302.

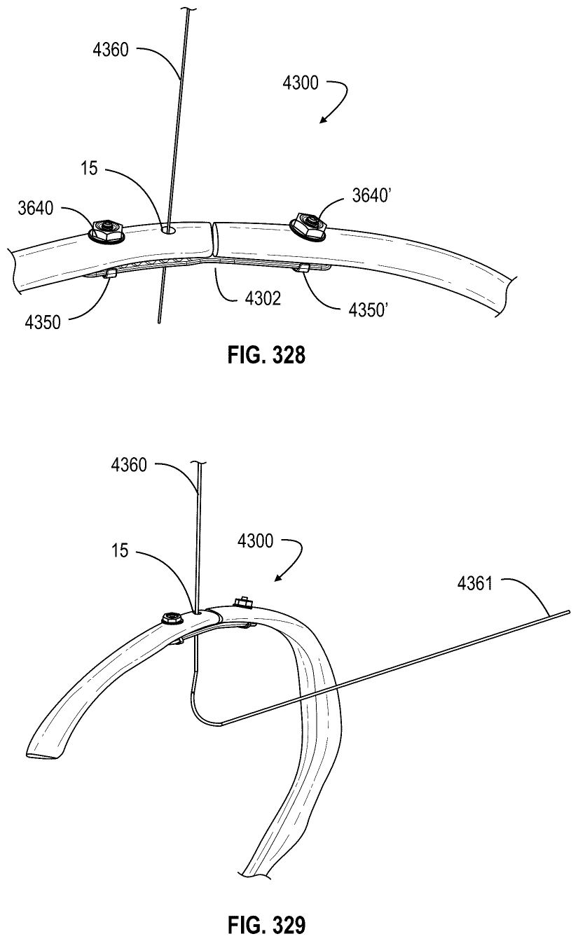

FIG. 328 is a perspective view of a step in a method of deploying the fracture plating system 4300 which may include withdrawing the cannulated drill bit 4385 according to an embodiment of the present disclosure. The method may further include withdrawing the cannulated drill bit 4385 along the tether 4360 while leaving the tether 4360 in place.

FIG. 329 is a perspective view of a step in a method of deploying the fracture plating system 4300 which may include passing the first tether end 4361 through a chest cavity to an exterior portion of the patient according to an embodiment of the present disclosure. The method may further include grasping the first tether end 4361 with a surgical instrument, such as an endoscopic grasper and/or a similar instrument, and using the surgical instrument to pull the first tether end 4361 through the chest cavity to the exterior portion of the patient. The first tether end 4361 may be pulled out to the exterior portion through a VATS portal and/or a skin incision.

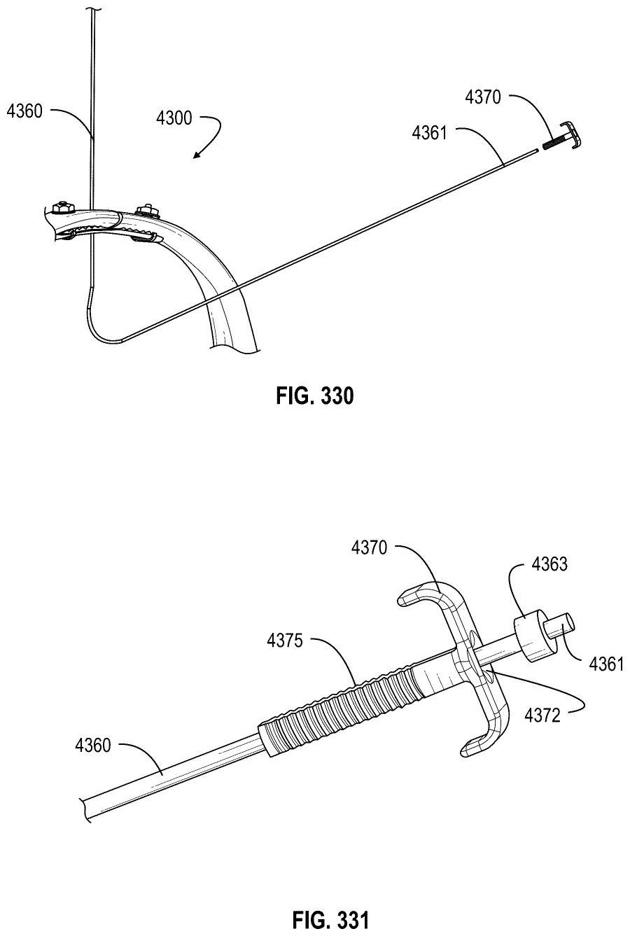

FIG. 330 is a perspective view of a step in a method of deploying the fracture plating system 4300 which may include positioning a rescue fastener 4370 proximate the first tether end 4361 according to an embodiment of the present disclosure. The method may further include selecting the rescue fastener 4370 from a set of rescue fasteners each having a different length, wing feature, and/or head portion. The selection of the rescue fastener 4370 may be based on the selected plate 4302, patient anatomy, and/or surgeon discretion. The method may further include positioning the rescue fastener 4370 proximate the first tether end 4361.

FIG. 331 is a perspective view of a step in a method of deploying the fracture plating system 4300 which may include passing the first tether end 4361 through the rescue fastener according to an embodiment of the present disclosure. The method may further include passing the first tether end 4361 through a cannulation 4372 of the rescue fastener 4370. The method may further include securing a bead 4363 on the first tether end 4361. The bead 4363 may include similar features and functions as the first bead 1963. The bead 4363 may be configured to be mechanically fixed to the tether 4360.

Additionally, or alternatively, the method may further include tying a knot, similar to knot 1961, in the tether 4360. The knot 1961 may be configured to function in a manner similar to the bead 4363. The knot may be tied in the tether 4360 after the tether 4360 is passed through the cannulation 4372 of the rescue fastener 4370. The knot 1961 may be further configured to inhibit the rescue fastener 4370 from disengaging from the tether 4360 when the knot 1961 is present. Additionally, the knot 1961 may be configured so that it may be untied, thereby allowing the first tether end 4361 to be removed from the rescue fastener 4370.

In an embodiment, the cannulation 4372 may include a non-circular profile and the tether 4360 may include a corresponding non-circular profile. The non-circular profile may inhibit rotation of the rescue fastener 4370 relative to the tether 4360. Additionally, the non-circular profile may facilitate alignment of a flat 4375 of the rescue fastener 4370 with a slot in the plate 4302 so that the fastener may be received in the slot but may be inhibited from rotating about a longitudinal axis of the rescue fastener 4370 within the slot relative to the plate 4302.

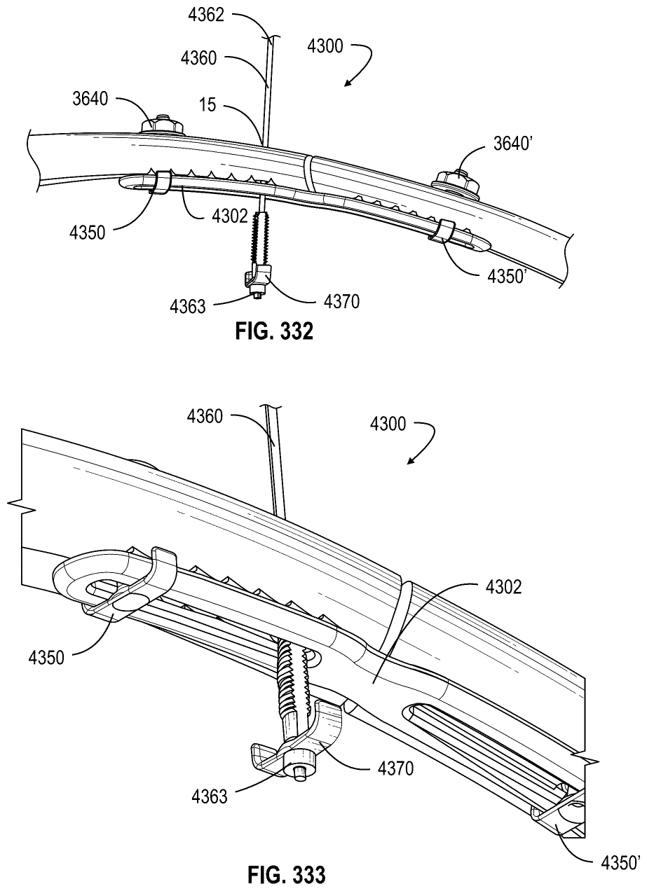

FIG. 332 is a perspective view of a step in a method of deploying the fracture plating system 4300 which may include using the tether 4360 to guide the rescue fastener 4370 to the rib according to an embodiment of the present disclosure. The method may further include pulling a second tether end 4362 to draw the rescue fastener 4370 through a skin incision and/or a VATS port and through the chest cavity.

FIG. 333 is a perspective view of a step in a method of deploying the fracture plating system 4300 which may include using the tether 4360 to guide the rescue fastener 4370 through the hole 15 according to an embodiment of the present disclosure. The method may further include pulling the second tether end 4362 to guide the rescue fastener 4370 through the plate 4302 and through the hole 15.

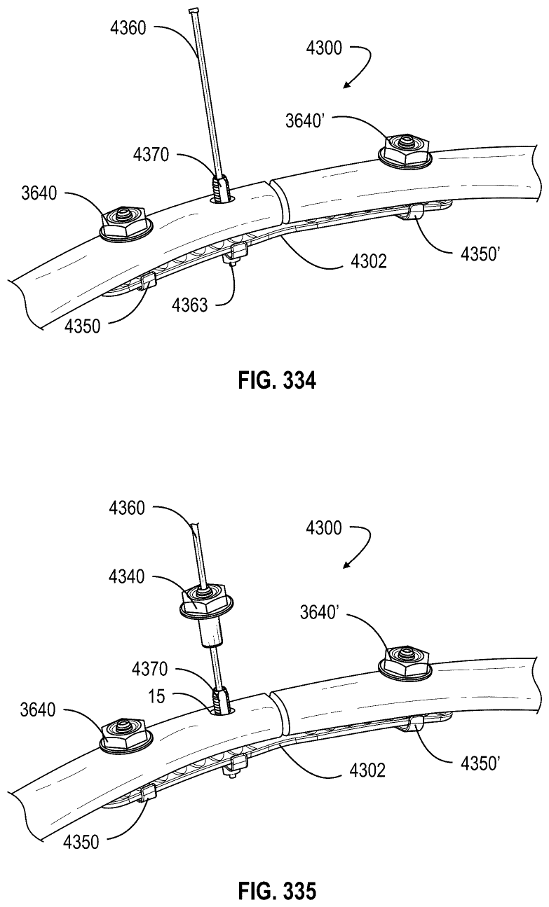

FIG. 334 is a perspective view of a step in a method of deploying the fracture plating system 4300 which may include using the tether 4360 to guide the rescue fastener 4370 to engage the plate according to an embodiment of the present disclosure. The method may further include continuing to pull the second tether end 4362 until the rescue fastener 4370 engages the plate 4302.

FIG. 335 is a perspective view of a step in a method of deploying the fracture plating system 4300 which may include using the tether 4360 to guide a cap 4340 to the rescue fastener 4370 according to an embodiment of the present disclosure. The method may further include receiving a cap 4340 on the second tether end 4362 and guiding the cap 4340 along the tether 4360 to the rescue fastener 4370.

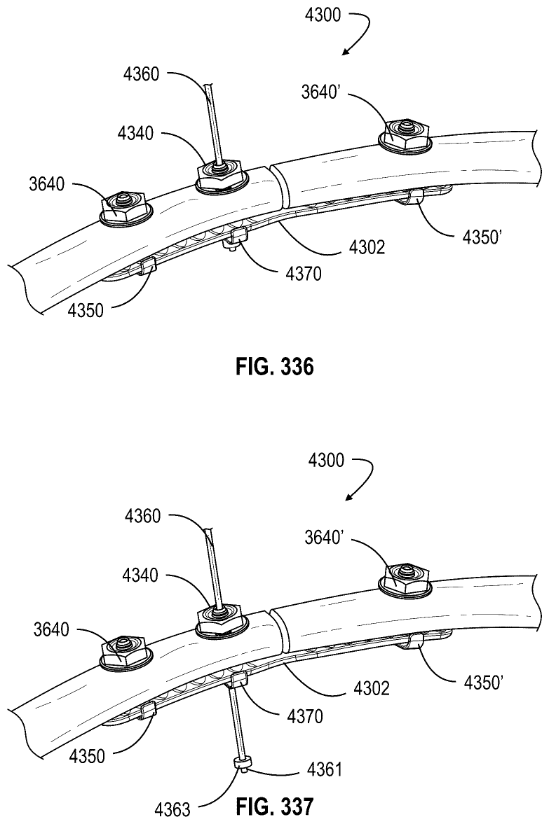

FIG. 336 is a perspective view of a step in a method of deploying the fracture plating system 4300 which may include engaging the cap 4340 with the rescue fastener 4370 according to an embodiment of the present disclosure. The method may further include threadably engaging the cap 4340 with the rescue fastener 4370 to secure the rescue fastener 4370 to the plate 4302 and the rib.

FIG. 337 is a perspective view of a step in a method of deploying the fracture plating system 4300 which may include withdrawing the tether 4360 according to an embodiment of the present disclosure. The method may further include withdrawing the tether 4360 through the rescue fastener 4370 and the cap 4340.

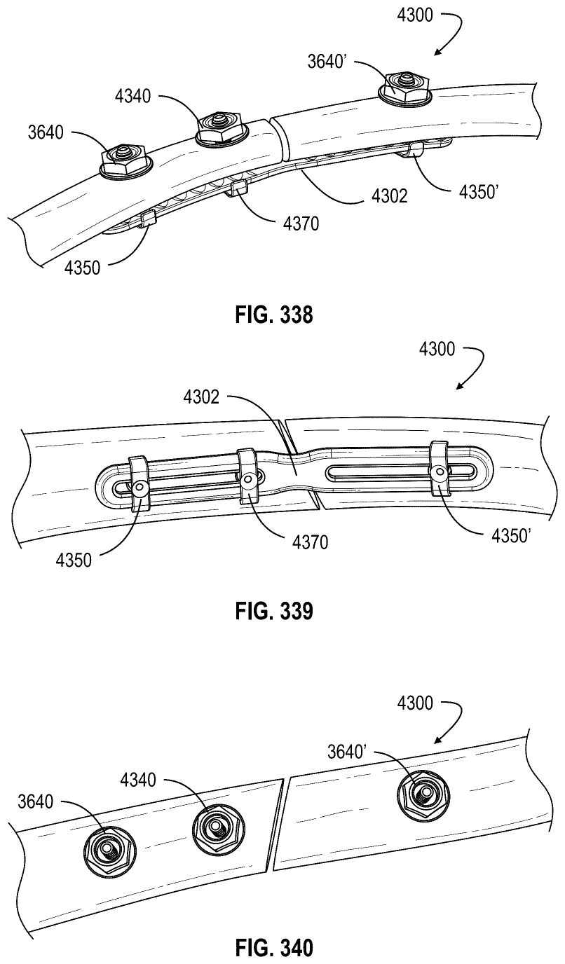

FIG. 338 is a perspective view of the fracture plating system 4300 according to an embodiment of the present disclosure. FIG. 339 is a perspective view of the fracture plating system 4300. FIG. 340 is a perspective view of the fracture plating system 4300. The method may further include using a driver (not shown) to secure the cap 4340 to the rescue fastener 4370. The rescue fastener 4370 may allow for additional fixation without disturbing the initial construct. The rescue fastener 4370 may be used to fix flail segments of bone as well as segments that require additional fixation beyond two posts.

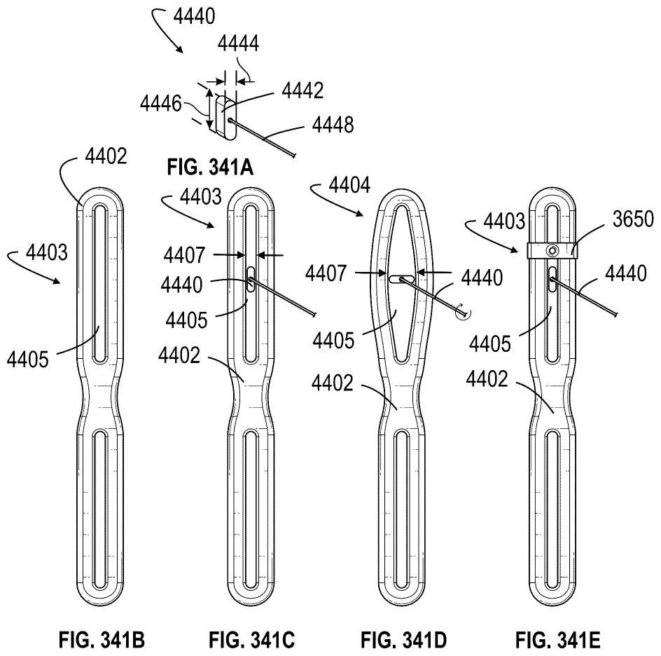

FIG. 341A is a perspective view of an expansion tool 4440 according to an embodiment of the present disclosure. FIG. 341B is a top view of a plate 4402 in an unexpanded configuration 4403 according to an embodiment of the present disclosure. FIG. 341C is a top view of the plate 4402 in the unexpanded configuration 4403 and the expansion tool 4440. FIG. 341D is a top view of the plate 4402 in an expanded configuration 4404 and the expansion tool 4440 according to an embodiment of the present disclosure. FIG. 341E is a top view of the plate 4402 in the unexpanded configuration 4403, the expansion tool 4440, and a fastener 3650 according to an embodiment of the present disclosure.

The plate 4402 may include similar function and features as other plates previously described within the present disclosure. The plate 4402 may be configured to receive a fastener, such as fastener 4350, and/or other fasteners previously described within the present disclosure. The plate 4402 may include an unexpanded configuration 4403 and an expanded configuration 4404. The plate may further include a slot 4405 configured to receive a fastener 3650.

With the plate 4402 in the expanded configuration 4404, the plate 4402 may be configured to receive the fastener 3650 in the slot 4405. With the plate 4402 in the unexpanded configuration 4403, the plate 4402 may be configured so that the fastener 3650 may be captive within the slot 4405.

The plate 4402 may be configured so that the slot 4405 may be expanded in a direction generally perpendicular to a longitudinal axis of the slot 4405 so that the plate 4402 may be in the expanded configuration 4404 and the fastener 3650 may be received in the slot 4405. The plate 4402 may be further configured so that the slot 4405 may return to the unexpanded configuration after the fastener 3650 is received in the slot 4405 thereby captively retaining the fastener 3650 within the slot 4405. The fastener 3650 and the plate 4402 may be configured so that, with the fastener 3650 captively received in the slot 4405, the fastener 3650 may be translatable within the slot 4405 along the longitudinal axis of the slot 4405.

Additionally, with the fastener 3650 received in the slot 4405, the one or more wing tips 3658 of the fastener 3650 may be configured to maintain the slot 4405 in the unexpanded configuration 4403 and may inhibit the slot 4405 from splaying open to the expanded configuration 4404.

The expansion tool 4440 may be configured to transform the plate 4402 from the unexpanded configuration 4403 to the expanded configuration 4404. The expansion tool 4440 may include a shank 4448 and a cam surface 4442, having a cam width 4444 and a cam length 4446. The shank 4448 may be configured to facilitate rotation and/or actuation of the cam surface 4442. The cam width 4444 may be sized generally equally to, or slightly smaller than, a slot width 4407 of the slot 4405. The cam width 4444 may be configured so that the cam surface 4442 may be received within the slot 4405. The cam length 4446 may be sized to be greater than the cam width 4444 so that, with the cam surface 4442 rotated relative to the slot, the cam length 4446 may cause the plate to transform from the unexpanded configuration 4403 to the expanded configuration 4404.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

1. A fracture plating system configured to stabilize a first fracture of a bone of a patient, the bone comprising an interior surface facing toward an interior body cavity of the patient, and an exterior surface facing away from the interior body cavity, the fracture plating system comprising:
a plate formed of a biocompatible polymer and configured to span the first fracture, the plate comprising:
an exterior plate surface configured to abut the interior surface;
an interior plate surface opposite the exterior plate surface; and
a slot passing between the exterior plate surface and the interior plate surface;
a tether configured to apply a force to the plate to draw the exterior plate surface against the interior surface of the bone; and
a bracing member securable to the plate to restrict flexure of the plate parallel to a width of the slot.

2. The fracture plating system of claim 1, wherein the bracing member is incorporated into a first fastener comprising:
a proximal end comprising:
a head portion; and
the bracing member comprising a first wing extending from the head portion; and
a distal end;
wherein the distal end is insertable through the slot to secure the plate to the interior surface such that the first wing abuts the interior plate surface.

3. The fracture plating system of claim 2, wherein:
the bracing member further comprises a second wing extending from the head portion; and
the first wing and the second wing cooperate to extend along substantially an entire width of the of the interior plate surface, transverse to the slot.

4. The fracture plating system of claim 3, wherein each of the first wing and the second wing comprises a tip that extends along a longitudinal axis of the first fastener such that, with the first wing and the second wing abutting the interior plate surface, the tips extend alongside the plate such that the plate is positioned between the tips to prevent flexure of the plate in a manner that would cause the slot to expand.

5. A fracture plating system configured to stabilize a first fracture of a bone of a patient, the bone comprising an interior surface facing toward an interior body cavity of the patient, and an exterior surface facing away from the interior body cavity, the fracture plating system comprising:
a plate configured to span the first fracture, the plate comprising:
an exterior plate surface configured to abut the interior surface;
an interior plate surface opposite the exterior plate surface; and
a slot passing between the exterior plate surface and the interior plate surface;
a first fastener comprising:
a proximal end comprising a head portion;
a distal end; and
a shank that extends between the head portion and the distal end; and
a tether configured to apply a force to guide the first fastener and the plate through the interior body cavity to the interior surface of the bone;
wherein the slot comprises:
an expanded configuration in which the distal end is insertable through the slot; and
an unexpanded configuration in which the shank is accommodated within the slot but the distal end is not withdrawable through the slot.

6. The fracture plating system of claim 5, wherein the plate is configured to flex such that the slot is deformable along a direction transverse to the slot, from the unexpanded configuration to the expanded configuration.

7. The fracture plating system of claim 6, further comprising an expansion tool configured to engage the plate to deform the slot from the unexpanded configuration to the expanded configuration.

8. The fracture plating system of claim 7, wherein the expansion tool comprises a cam surface comprising:
a cam width smaller than a slot width of the slot; and
a cam length longer than the slot width such that rotation of the cam surface within the slot urges deformation of the slot from the unexpanded configuration to the expanded configuration.

9. The fracture plating system of claim 5, wherein:
the distal end is threaded; and
the shank has a smallest shank width smaller than a distal end width of the distal end.

10. The fracture plating system of claim 9, wherein the shank comprises:
threads; and
unthreaded flats that interrupt the threads and define the smallest shank width such that, in the unexpanded configuration, the unthreaded flats abut interior walls of the slot to prevent rotation of the shank within the slot.

11. A method for stabilizing a first fracture of a bone of a patient, the bone comprising an interior surface facing toward an interior body cavity of the patient, and an exterior surface facing away from the interior body cavity, the method comprising:
positioning a plate near the bone, wherein the plate is formed of a biocompatible polymer and is configured to span the first fracture, the plate comprising:
an exterior plate surface;
an interior plate surface opposite the exterior plate surface; and
a slot passing between the exterior plate surface and the interior plate surface;
urging the exterior plate surface against the interior surface of the bone such that the plate flexes to cause the exterior plate surface to conform to the interior surface; and
with at least a first fastener, securing the plate to the bone.

12. The method of claim 11, further comprising passing a first end of a tether through the bone;
wherein urging the exterior plate surface against the interior surface of the bone comprises using the tether to pull exterior plate surface against the interior surface of the bone.

13. The method of claim 11, wherein the first fastener comprises:
a proximal end comprising:
a head portion; and
wings extending from the head portion; and
a distal end;
wherein securing the plate to the bone comprises:
inserting the distal end through the slot; and
further advancing the first fastener toward the bone such that the wings abut the plate interior surface.

14. The method of claim 13, wherein each of the wings comprises a tip that extends along a longitudinal axis of the first fastener such that, with the wings abutting the interior plate surface, the tips extend alongside the plate such that the plate is positioned between the tips to prevent flexure of the plate in a manner that would cause the slot to expand.

15. The method of claim 11, wherein:
the interior surface of the bone comprises a bone curvature; and
the exterior plate surface comprises one of:
a planar shape; and
a plate curvature with a larger radius than the bone curvature.

16. The method of claim 15, wherein:
the plate further comprises:
a first end;
a second end opposite the first end; and
a central portion that connects the first end and the second end; and
urging the exterior plate surface against the interior surface of the bone comprises:
contacting the interior surface of the bone with the first end and the second end; and
bending the plate to cause the central portion to come into contact with the interior surface.

17. A method for assembling a system for stabilizing a first fracture of a bone of a patient, the bone comprising an interior surface facing toward an interior body cavity of the patient, and an exterior surface facing away from the interior body cavity, the method comprising:
positioning a first fastener proximate a plate;
wherein the plate is configured to span the first fracture and comprises:
an exterior plate surface configured to abut the interior surface of the bone;
an interior plate surface opposite the exterior plate surface; and
a slot passing between the exterior plate surface and the interior plate surface;
wherein the first fastener comprises:
a proximal end comprising a head portion;
a distal end; and
a shank that extends between the head portion and the distal end; and
moving the slot from an unexpanded configuration to an expanded configuration;
with the slot in the expanded configuration, inserting the distal end through the slot; and
allowing the slot to return to the unexpanded configuration such that the shank is accommodated within the slot but the distal end is not withdrawable through the slot.

18. The method of claim 17, wherein moving the slot from the unexpanded configuration to the expanded configuration comprises:
inserting a cam surface of a tool into the slot, the cam surface comprising:
a cam width smaller than a slot width of the slot; and
a cam length longer than the slot width; and
rotating the cam surface within the slot to urge deformation of the slot from the
unexpanded configuration to the expanded configuration.

19. The method of claim 17, wherein:
the distal end is threaded; and
the shank has a smallest shank width smaller than a distal end width of the distal end.

20. The method of claim 19, wherein:
the shank comprises:
threads; and
unthreaded flats that interrupt the threads and define the smallest shank width;
and
allowing the slot to return to the unexpanded configuration comprises positioning the unthreaded flats to abut interior walls of the slot to prevent rotation of the shank within the slot.
